(19) United States
(12) Patent Application Publication
(10) Pub. No.: US 2010/0297020 A1
(43) Pub. Date: Nov. 25, 2010

(54) PHOSPHORYLATED AND PHOSPHONATED PYRONE ANALOGS FOR THERAPEUTIC TREATMENT

(75) Inventors: May Dean-Ming Lee, Los Altos, CA (US); Ving Lee, Los Altos, CA (US); Wendye Robbins, South San Francisco, CA (US)

Correspondence Address: SEED INTELLECTUAL PROPERTY LAW GROUP PLLC, 701 FIFTH AVE, SUITE 5400, SEATTLE, WA, 98104, US

(73) Assignee: Limerick Biopharma, Inc., South San Francisco, CA (US)

(21) Appl. No.: 12/757,817

(22) Filed: Apr. 9, 2010

Related U.S. Application Data
(63) Continuation-in-part of application No. 12/587,704, filed on Oct. 9, 2009.
(60) Provisional application No. 61/208,812, filed on Feb. 26, 2009.
(60) Provisional application No. 61/104,647, filed on Oct. 10, 2008.
(60) Provisional application No. 61/243,111, filed on Sep. 16, 2009.
(60) Provisional application No. 61/225,183, filed on Jul. 13, 2009.
(60) Provisional application No. 61/173,169, filed on Apr. 27, 2009.

(51) Int. Cl. A61K 49/00; A61K 31/665; C07F 9/40; A61K 31/7088; A61K 38/00; A61P 3/06; A61P 3/08; A61P 3/00; A61P 1/18

(52) U.S. Cl. 424/9.2; 514/100; 549/216; 514/44 R; 514/1.1

(57) ABSTRACT

Methods are described for the treatment and prevention of metabolic disorders or other diseases by administering a pyrone analog or a derivative thereof. Methods are also described for the treatment and prevention of metabolic disorders and other diseases by administering a pyrone analog, or a derivative thereof, in combination with one or more additional agents such as, for example, lipid lowering agents or glucose lowering agents. Methods are described for the modulation of lipid transporter activity to increase the efflux of lipid from a physiological compartment into an external environment. Methods disclosed herein may be used to assess treatment or prevention of a metabolic disorder following administration of a pyrone analog or a derivative thereof.

### CROSS-REFERENCE TO RELATED APPLICATIONS

This application is a continuation-in-part of U.S. application Ser. No. 12/587,704, filed Oct. 9, 2009, which claims the benefit of U.S. Provisional Application Nos. 61/104,647, filed Oct. 10, 2008 and 61/208,812 filed Feb. 26, 2009, each of which is herein incorporated by reference in its entirety. This application also claims the benefit of U.S. Provisional Application Nos. 61/243,111, filed Sep. 16, 2009, 61/173,169, filed Apr. 27, 2009, and 61/225,183, filed Jul. 13, 2009, each of which is herein incorporated by reference in its entirety.

### BACKGROUND OF THE INVENTION

Polyphenols such as flavonoids have been shown to have beneficial health effects. In particular, polyphenols can provide beneficial effects by lowering the side effects of co-administered therapeutic agents, in some cases acting as tissue transport protein modulators. While blood tissue barrier (BTB) structures, such as the blood-brain barrier (BBB), blood pancreas barrier, blood kidney barrier, and blood-placenta barrier, function as an obstacle to isolate tissues from the systemic blood circulation, some pharmaceutical agents, such as anesthetic and pharmaceutical agents, cross tissues selectively causing tissue specific toxicity or side-effects rather than a desired localized action. In addition, blood tissue barriers may be compromised by disease states and therapeutic treatments, causing barrier laxity and then permitting unwanted agents to cross the barrier and adversely affect tissue structures. Thus, there is a continued need in the field for compounds that will lower side effects of co-administered therapeutic agents, such as new tissue transport protein modulators, and for compositions and methods for improved delivery of polyphenols, flavonoids, and related compounds.

Diabetes mellitus has become one of the most prevalent diseases in industrialized countries. In the United States alone, about 23.6 million people (about 8% of the population) have diabetes with an additional 57 million people at risk. Because of such a large prevalence and impact upon the health and economy of a society, diabetes is a subject of intense interest by academics and pharmaceutical industry.

Insulin is a hormone that is produced by beta cells of the islets of Langerhans in the pancreas, and functions to facilitate glucose uptake in the cells. In Type 1 diabetes, a majority of beta cells are destroyed and rendered nonfunctional by autoimmune inflammation resulting in no insulin production. Triggers for the autoimmune response are not yet known, but it has been contemplated that viruses and environmental factors in genetically susceptible individuals play a factor.

Type 2 diabetes is characterized by the onset of insulin resistance or reduced sensitivity in peripheral tissues in combination with impaired insulin secretion. The impaired insulin secretion results from progressive degeneration and dysfunction of pancreatic alpha and beta cells as well as a significant reduction in cell mass, and is typically associated with obese conditions. Obesity is now a world wide epidemic, and is one of the most serious contributors to increased morbidity and mortality. Obesity, which is an excess of body fat relative to lean body mass, is a chronic disease. Obesity is also a multiple etiology problem. The prevalence of obesity has risen significantly in the past decade in the United States and many other developed countries (Fiegal et al, Int. J. Obesity 22:39-47 (1998), Mokdad et al, JAMA 282:1519-1522 (1999)).

Obesity is associated not only with a social stigma, but also with decreased life span and numerous medical problems, including adverse psychological development, stroke, hyperlipidemia, some cancers, type 2 diabetes, coronary heart disease, hypertension, numerous other major illnesses, and overall mortality from all causes (see, e.g., Nishina, et al., Metab. 43:554-558, 1994; Grundy and Barnett, Dis. Mon. 36:641-731 (1990); Rissanen, et al., British Medical Journal, 301:835-837 (1990); Must et al, JAMA 282:1523-1529 (1999); Calle et al, N. Engl. J. Med. 341:1097-1105 (1999)). Weight reduction and improved control of lipid, blood pressure, and sugar levels is critical for the obese patient (Blackburn, Am. J. Clin. Nutr. 69:347-349 (1999); and Galuska et al, JAMA 282:1576 (1999)).

### SUMMARY OF THE INVENTION

In one embodiment, the invention encompasses compositions comprising a phosphonated pyrone analog or a pharmaceutically or veterinary acceptable salt, glycoside, ester, or prodrug thereof.

In another embodiment, the invention encompasses methods of treating an animal comprising administering to an animal in need of treatment, a composition comprising a phosphonated pyrone analog or a pharmaceutically or veterinary acceptable salt, glycoside, ester, or prodrug thereof, and a pharmaceutically acceptable excipient.

In another embodiment, the invention encompasses kits comprising: (a) a therapeutic agent or a pharmaceutically or veterinary acceptable salt, glycoside, ester, or prodrug thereof; and (b) a phosphonated pyrone analog or a pharmaceutically or veterinary acceptable salt, glycoside, ester, or prodrug thereof.

In another embodiment, the invention encompasses methods of treating an animal comprising administering to an animal in need of treatment, (a) a therapeutic agent or a pharmaceutically or veterinary acceptable salt, glycoside, ester, or prodrug thereof; and (b) a phosphonated pyrone analog or a pharmaceutically or veterinary acceptable salt, glycoside, ester, or prodrug thereof.

In another embodiment, the invention encompasses methods of maintaining cellular physiological conditions for cell survival, comprising administering to a subject an effective amount of a phosphonated pyrone analog that modulates activity of a cellular transporter.

In another embodiment, the invention encompasses methods of treating a disease, comprising administering to a subject an effective amount of a phosphonated pyrone analog, wherein the phosphonated pyrone analog modulates activity of a cell surface transporter.

In another embodiment, the invention encompasses methods of modulating transport of a lipophilic molecule, the method comprising administering an effective amount of a phosphonated pyrone analog to a subject, wherein the phosphonated pyrone analog modulates activity of a cellular transporter.

In another embodiment, the invention encompasses methods of modulating a lipid, cholesterol, triglyceride, insulin or glucose level in a subject, the method comprising administering an effective amount of a phosphonated pyrone analog to the subject, wherein the phosphonated pyrone analog modulates activity of a cellular transporter.

In another embodiment, the invention encompasses methods of assessing a cellular protective effect in a pancreatic islet cell, comprising: i) selecting a patient for treatment based on one or more biomolecule levels in a sample compared to a control sample; ii) administering an effective amount of a phosphonated pyrone analog to the patient; and iii) monitoring said one or more biomolecule levels in the patient.

In another embodiment, the invention encompasses methods of treating pancreatic cell stress or injury comprising administering to a subject an effective amount of at least one phosphonated pyrone analog, wherein at least one effect of stress or injury is improved in one or more cell types of the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the embodiments are set forth in the appended claims. A better understanding of the features and advantages of the present embodiments will be obtained by reference to the following detailed description that sets forth illustrative embodiments, in which the principles of the embodiments are utilized, and the accompanying drawings of which:

FIG. 1 shows that pyrone analogs LIM-0705 and LIM-0741 have little impact on weight gain of ZDF rats over 6 weeks of daily treatment.

FIG. 2 shows that pyrone analogs LIM-0705 (high dose) and LIM-0741 impact glucose levels in ZDF rats over 6 weeks of daily treatment.

FIG. 3 shows that pyrone analogs LIM 0705 and LIM-0741 impact glucose levels in produces elevated insulin levels in ZDF rodents. Bars from left to right at each day of measurement are as follows: V/V, V/C, Rosy, LIM-0705 high dose (HD), LIM-0705 low dose (LD), and LIM-0741.

FIG. 4 shows that pyrone analogs LIM-0705 and LIM-0741 impact glycated hemoglobin levels (% HbA1c) levels in ZDF rats following 6 weeks of daily treatment.

FIG. 5 shows that pyrone analogs LIM-0705 and LIM-0741 impact insulin levels in ZDF rats following 5 and 6 weeks of daily treatment.

FIG. 6 shows the effect of pyrone analogs LIM-0705 and LIM-0741 on cholesterol levels in ZDF rats over 6 weeks of daily treatment.

FIG. 7 illustrates cholesterol levels at days 0, 7 and 14.

FIG. 8 shows the effect of pyrone analogs LIM-0705 and LIM-0741 on triglyceride levels in ZDF rats over 6 weeks of daily treatment.

FIG. 9 shows the effect of pyrone analogs on triglyceride levels.

FIG. 10 shows that pyrone analogs LIM-0705 and LIM-0741 impact adiponectin levels in ZDF rats following 6 weeks of daily treatment.

FIG. 11 shows that pyrone analogs LIM-0705 and LIM-0741 impact glucagon levels in ZDF rats following 6 weeks of daily treatment.

FIG. 12 shows AST levels in ZDF rodents at 14 weeks of age.

FIG. 13 shows ALT levels in ZDF rodents at 14 weeks of age.

FIG. 14 shows that liver weight is not effected in response to LIM-0705 and LIM-0741 in ZDF rodents.

FIG. 15 shows that kidney weight is not effected in response to LIM-0705 and LIM-0741 in ZDF rodents.

FIG. 16 shows that pyrone analogs LIM-0705 and LIM-0741 impact fat weight in ZDF rats following 6 weeks of daily treatment.

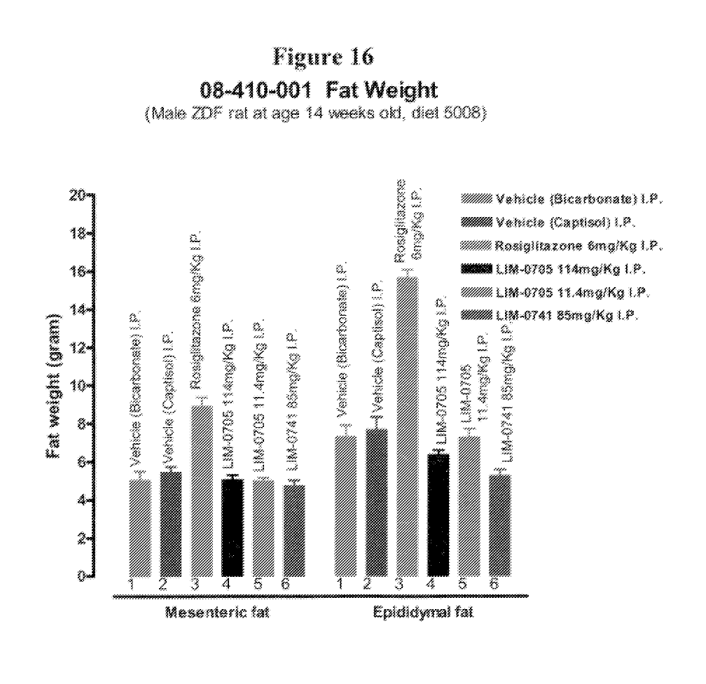

FIG. 17 shows the effect of pyrone analog LIM-0742 on glucose levels in aging ZDF rats during 6 weeks of daily treatment.

FIG. 18 shows the effect of pyrone analog LIM 0742 on fad insulin levels in aging ZDF rats during 6 weeks of daily treatment

FIG. 19 shows the effect of pyrone analog LIM-0742 on circulating triglyceride levels in aging ZDF rats during 6 weeks of daily treatment.

FIG. 20 shows the effect of pyrone analog LIM-0742 on weight gain in ZDF rats during 6 weeks of daily treatment

FIG. 21 shows the effect of pyrone analog LIM 0742 on plasma glucose following oral glucose load.

FIG. 22 shows the effect of pyrone analog LIM 0742 on insulin production following oral glucose load.

FIG. 23 shows the effect of pyrone analog LIM 0742 on total plasma cholesterol during 6 weeks of daily treatment.

FIG. 24 shows that pyrone analogs LIM-0705 and LIM-0741 have little impact on weight gain of ZDF rats over 2 weeks of daily treatment.

FIG. 25 shows the effect of pyrone analogs LIM-0705 and LIM-0741 on cholesterol levels in ZDF rats over 2 weeks of daily treatment.

FIG. 26 shows that pyrone analogs LIM-0705 (high dose) and LIM-0741 impact glucose levels in ZDF rats over 2 weeks of daily treatment.

FIG. 27 shows that phosphonated pyrone analogs compound 2 and compound 3 impact plasma glucose levels in ZDF rats over four weeks of daily treatment.

FIG. 28 shows that phosphonated pyrone analogs compound 2 and compound 3 impact plasma insulin levels in ZDF rats over four weeks of daily treatment.

FIG. 29 shows that phosphonated pyrone analogs compound 2 and compound 3 impact glycated hemoglobin levels (% HbA1c levels) in ZDF rats following four weeks of daily treatment.

FIG. 30 shows that phosphonated pyrone analogs compound 2 and compound 3 impact triglyceride levels in ZDF rats.

FIG. 31 shows that phosphonated pyrone analogs compound 2 and compound 3 protect against hyperglycemia during oral glucose tolerance test (OGTT) in ZDF rats following four weeks of daily treatment.

FIG. 32 shows that phosphonated pyrone analog compound 3 raises insulin output in response to oral glucose tolerance test (OGTT) in ZDF rats following four weeks of daily treatment.

FIG. 33 shows that phosphonated pyrone analogs compound 2 and compound 3 decrease pancreatic inflammation (by 60% and 35% inhibition respectively) in ZDF rats following four weeks of daily treatment.

FIG. 34 shows that phosphonated pyrone analogs compound 2 and compound 3 decrease pancreatic islet vacuolar degeneration (by 43% for both compounds) in ZDF rats following four weeks of daily treatment.

FIG. 35 shows that phosphonated pyrone analogs compound 2 and compound 3 decrease islet cell apoptosis in ZDF rats following four weeks of daily treatment.

FIG. 36 demonstrates that following four weeks of daily treatment, phosphonated pyrone analog compound 3 improves response to IP glucose load in diet-induced obese mice.

FIG. 37 demonstrates that less insulin output is required to lower plasma glucose in response to IP glucose load in compound 3-treated diet-induced obese mice.

FIG. 38 demonstrates that following four weeks of daily treatment, phosphonated pyrone analog compound 3 enhances glucose uptake in diet-induced obese mice.

FIG. 39 demonstrates that following four weeks of daily treatment, phosphonated pyrone analog compound 3 reduces hepatic glucose output in diet-induced obese mice.

FIG. 40 demonstrates that following four weeks of daily treatment, phosphonated pyrone analog compound 3 reduces basal glucose in diet-induced obese mice.

FIG. 41 shows that phosphonated pyrone analog compound 1 reduces fed plasma glucose levels in ZDF rats over four weeks of daily treatment.

FIG. 42 shows that phosphonated pyrone analog compound 1 improves glucose tolerance in ZDF rats following four weeks of daily treatment.

FIG. 43 shows that phosphonated pyrone analog compound 1 maintains higher fed and fasting insulin level following four weeks of daily treatment.

FIG. 44 shows that phosphonated pyrone analog compound 1 reduces glycated hemoglobin levels (% HbA1c levels) in ZDF rats following four weeks of daily treatment.

FIG. 45 shows that effective doses of phosphonated pyrone analog compound 1 increases pancreatic insulin levels following four weeks of daily treatment.

FIG. 46 shows that phosphonated pyrone analog compound 1 does not increase terminal liver triglyceride following four weeks of daily treatment, contrary to Rosiglitazone.

FIG. 47 shows that phosphonated pyrone analog compound 1 reduces plasma glucose levels in cyclosporin treated Wistar rats over two weeks of daily treatment.

FIG. 48 shows that phosphonated pyrone analog compound 1 improves glucose tolerance in cyclosporin treated Wistar rats following two weeks of daily treatment.

FIG. 49 shows that phosphonated pyrone analog compound 1 reduces cyclosporne induced islet cell injury in cyclosporin treated Wistar rats following two weeks of daily treatment.

FIG. 50 shows that phosphonated pyrone analog compound 1 reduces cyclosporine induced islet cell apoptosis in cyclosporin treated Wistar rats following two weeks of daily treatment.

FIG. 51 shows that phosphonated pyrone analog compound 1 reduces plasma glucose levels in tacrolimus treated Wistar rats over two weeks of daily treatment.

FIG. 52 shows that phosphonated pyrone analog compound 1 improves glucose tolerance in tacrolimus treated Wistar rats following two weeks of daily treatment.

FIG. 53 shows that phosphonated pyrone analog compound 1 increases insulin level in tacrolimus treated Wistar rats (during oral glucose tolerance test) following two weeks of daily treatment.

FIG. 54 shows that phosphonated pyrone analog compound 3 reduces plasma triglyceride levels in C57BL/6 mice over 13 days of daily treatment.

FIG. 55 shows that phosphonated pyrone analog compound 3 reduces liver triglyceride levels in C57BL/6 mice over 13 days of daily treatment.

FIG. 56 shows that phosphonated pyrone analog compound 3 reduces plasma glucose levels in db/db mice over 4 weeks of daily treatment.

FIG. 57 shows that phosphonated pyrone analog compound 3 reduces plasma triglyceride levels in db/db mice over 4 weeks of daily treatment.

FIG. 58 shows that phosphonated pyrone analog compound 3 reduces plasma cholesterol levels in db/db mice over 4 weeks of daily treatment.

FIG. 59 shows that phosphonated pyrone analog compound 3 reduces liver triglyceride levels in db/db mice over 4 weeks of daily treatment.

FIG. 60 shows that phosphonated pyrone analog compound 3 does not produce excessive body weight gain in db/db mice over 4 weeks of daily treatment.

### DETAILED DESCRIPTION OF THE INVENTION

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents. It should also be noted that use of “or” means “and/or” unless stated otherwise. Furthermore, use of the term “including” as well as other forms, such as “include,” “includes,” and “included” is not limiting. Thus, for example, reference to “a compound” includes a plurality of such compounds, and reference to “the cell” includes reference to one or more cells (or to a plurality of cells) and equivalents thereof known to those skilled in the art, and so forth.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range.

An “average” as used herein is preferably calculated in a set of normal subjects, this set being at least about 3 subjects, at least about 5 subjects, at least about 10 subjects, at least about 25 subjects, or at least about 50 subjects.

The terms “effective amount” or “pharmaceutically effective amount” refer to a nontoxic but sufficient amount of the agent to provide the desired biological, therapeutic, and/or prophylactic result. That result can be reduction and/or alleviation of the signs, symptoms, or causes of a disease, or any other desired alteration of a biological system. For example, an “effective amount” for therapeutic uses is the amount of a pyrone analog as disclosed herein per se or a composition comprising the pyrone analog required to provide a therapeutically significant decrease in a disease. An appropriate effective amount in any individual case may be determined by one of ordinary skill in the art using routine experimentation.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

The term “treating” and its grammatical equivalents as used herein include achieving a therapeutic benefit and/or a prophylactic benefit. By therapeutic benefit is meant eradication or amelioration of the underlying disorder being treated. Treating also refers to obtaining a desired pharmacologic and/or physiologic effect. The effect may be prophylactic in terms of completely or partially preventing a condition or disease or symptom thereof and/or may be therapeutic in terms of a partial or complete cure for a condition or disease and/or adverse affect attributable to the condition or disease. “Treatment,” thus, for example, covers any treatment of a condition or disease in a mammal, particularly in a human, and includes: (a) preventing the condition or disease from occurring in a subject which may be predisposed to the condition or disease but has not yet been diagnosed as having it; (b) inhibiting the condition or disease, such as, arresting its development; and (c) relieving, alleviating or ameliorating the condition or disease, such as, for example, causing regression of the condition or disease. Also, a therapeutic benefit may be achieved with the eradication or amelioration of one or more of the physiological symptoms associated with the underlying disorder such that an improvement is observed in the patient, notwithstanding the fact that the patient may still be afflicted with the underlying disorder. For prophylactic benefit, a method may be performed on, or a composition administered to a patient at risk of developing a disease (condition), or to a patient reporting one or more of the physiological symptoms of such conditions, even though a diagnosis of the condition may not have been made. In some instances, treating means stasis (i.e., that the disease does not get worse) and survival of the patient is prolonged. A dose to be administered depends on the subject to be treated, such as the general health of the subject, the age of the subject, the state of the disease or condition, the weight of the subject, the size of a tumor, for example.

The term “subject,” “patient” or “individual” as used herein in reference to individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the mammal is a human.

The terms “co-administration,” “administered in combination with,” and their grammatical equivalents, as used herein, encompass administration of two or more agents to a subject so that both agents and/or their metabolites are present in the animal at the same time. Co-administration includes simultaneous administration in separate compositions, administration at different times in separate compositions, or administration in a composition in which both agents are present.

The term “pharmaceutical composition,” as used herein, refers to a biologically active compound, optionally mixed with at least one pharmaceutically acceptable chemical component, such as, though not limited to carriers, stabilizers, diluents, dispersing agents, suspending agents, thickening agents, and/or excipients.

The term “carrier” as used herein, refers to relatively nontoxic chemical compounds or agents that facilitate the incorporation of the compound into cells or tissues.

The term “pharmaceutically acceptable excipient,” includes vehicles, adjuvants, or diluents or other auxiliary substances, such as those conventional in the art, which are readily available to the public. For example, pharmaceutically acceptable auxiliary substances include pH adjusting and buffering agents, tonicity adjusting agents, stabilizers, wetting agents and the like.

The term “metabolite,” as used herein, refers to a derivative of the compound which is formed when the compound is metabolized.

The term “active metabolite,” as used herein, refers to a biologically active derivative of the compound that is formed when the compound is metabolized.

The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to the compound. Further information on metabolism may be obtained from The Pharmacological Basis of Therapeutics, 9th Edition McGraw-Hill (1996).

The term “unit dosage form,” as used herein, refers to physically discrete units suitable as unitary dosages for human and animal subjects, each unit containing a predetermined quantity of API calculated in an amount sufficient to produce the desired effect in association with a pharmaceutically acceptable diluent, carrier or vehicle. The specifications for the novel unit dosage forms of the present compounds depend on the particular compound employed and the effect to be achieved, and the pharmacodynamics associated with each compound in the host.

As used herein, “percent,” “percentage” or the symbol “%” means the percent of the component indicated in the composition based on the amount of the carrier present in the composition, on a weight/weight (w/w), weight/volume (w/v) or volume/volume (v/v), as indicated with respect to any particular component, all based on the amount of the carrier present in the composition. Thus, different types of carriers may be present in an amount of up to 100% as indicated, which does not preclude the presence of the API, the amount of which may be indicated as a % or as a certain number of mg present in the composition or a certain number of mg/mL present, where the % or mg/mL is based on the amount of the total carrier present in the composition. Certain types of carriers may be present in combination to make up 100% of the carrier.

A “substantially purified” compound in reference to the pyrone analogs or derivatives thereof is one that is substantially free of materials that are not the pyrone analogs or derivatives thereof. By way of example, substantially free is meant at least about 50% free of non-pyrone analog materials, at least about 70%, at least about 80%, at least about 90% free or at least about 95% free of non-pyrone analog materials.

### I. Pyrone Analogs

One class of compounds useful in the compositions and methods described herein are pyrone analogs. In some embodiments, the pyrone analog is phosphorylated or phosphonated.

A phosphorylated or phosphonated pyrone analog may be converted in vivo to metabolites that have differing activities in the modulation of one or more cholesterol, glucose, lipid and/or triglyceride transporters, and these metabolites are also encompassed by the compositions and methods described herein.

In some cases the phosphorylated pyrone analogs described herein comprise polyphosphate derivatives. Polyphosphate derivatives are those in which more than one phosphate is connected in a linear chain. Suitable polyphosphate derivatives include, for example, diphosphates (pyrophosphates), and triphosphates.

In some cases the phosphonated polyphenols of the invention comprise polyphosphonate derivatives. Polyphosphonate derivatives are those in which more than one phosphonate is connected in a linear chain. Suitable polyphosphonate derivatives include, for example, diphosphonates (pyrophosphonates), and triphosphonates.

As used herein, “Acyl” refers to a —(C═O)— radical which is attached to two other moieties through the carbon atom. Those groups may be chosen from alkyl, alkenyl, alkynyl, aryl, heterocyclic, heteroaliphatic, heteroaryl, and the like. Unless stated otherwise specifically in the specification, an acyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Acyloxy” refers to a R(C═O)O— radical wherein R is alkyl, aryl, heteroaryl or heterocyclyl. Unless stated otherwise specifically in the specification, an acyloxy group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2) —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkylaryl” refers to an (alkyl)aryl-radical, where alkyl and aryl are as defined herein.

“Aralkyl” refers to an (aryl)alkyl-radical where aryl and alkyl are as defined herein.

“Alkoxy” refers to a (alkyl)O-radical, where alkyl is as described herein and contains 1 to 10 carbons (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms. In some embodiments, it is a C₁-C₄ alkoxy group. An alkoxy moiety is optionally substituted by one or more of the substituents described as suitable substituents for an alkyl radical.

“Alkyl” refers to a straight or branched hydrocarbon chain radical, having from one to ten carbon atoms (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like. Unless stated otherwise specifically in the specification, an alkyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkenyl” refers to a straight or branched hydrocarbon chain radical group, containing at least one double bond, and having from two to ten carbon atoms (ie. C₂-C₁₀ alkenyl). Whenever it appears herein, a numerical range such as “2 to 10” refers to each integer in the given range; e.g., “2 to 10 carbon atoms” means that the alkenyl group may consist of 2 carbon atoms, 3 carbon atoms, etc., up to and including 10 carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Alkynyl” refers to a straight or branched hydrocarbon chain radical group, containing at least one triple bond, having from two to ten carbon atoms (i.e., C₂-C₁₀ alkynyl). Whenever it appears herein, a numerical range such as “2 to 10” refers to each integer in the given range; e.g., “2 to 10 carbon atoms” means that the alkynyl group may consist of 2 carbon atoms, 3 carbon atoms, etc., up to and including 10 carbon atoms. In certain embodiments, an alkynyl comprises two to eight carbon atoms. In other embodiments, an alkynyl has two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Amine” refers to a —N(R^{a})₂ radical group, where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl. Unless stated otherwise specifically in the specification, an amino group is optionally substituted by one or more substituents which independently are: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

An “amide” refers to a chemical moiety with formula —C(O)NR^{a}R^{b} or —NR^{a}C(O)R^{b}, where R^{a} or R^{b} is independently selected from the group consisting of hydrogen, alkyl, cycloalkyl, aryl, heteroaryl (bonded through a ring carbon) and heterocyclic (bonded through a ring carbon). An amide may be an amino acid or a peptide molecule attached to a compound of Formula I, thereby forming a prodrug. Any amine or carboxyl side chain on the compounds described herein can be amidified. The procedures and specific groups to make such amides are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety.

“Aromatic” or “aryl” refers to an aromatic radical with six to fourteen ring carbon atoms (e.g., C₆-C₁₄ aromatic or C₆-C₁₄ aryl). The term includes monocyclic or fused-ring polycyclic (i.e., rings which share adjacent pairs of ring atoms) groups. It has at least one ring having a conjugated pi electron system. Whenever it appears herein, a numerical range such as “6 to 14” refers to each integer in the given range; e.g., “6 to 14 ring atoms” means that the aryl group may consist of 6 ring atoms, 7 ring atoms, etc., up to and including 14 ring atoms. Unless stated otherwise specifically in the specification, an aryl moiety is optionally substituted by one or more substituents which are independently: hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, heterocyclic, C₃-C₁₀cycloalkyl, —CN —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Carboxaldehyde” refers to a —(C═O)H radical.

“Carboxyl” refers to a —(C═O)OH radical.

“Carbohydrate” as used herein, includes, but not limited to, monosaccharides, disaccharides, oligosaccharides, or polysaccharides. Monosaccharide for example includes, but not limited to, aldotrioses such as glyceraldehyde, ketotrioses such as dihydroxyacetone, aldotetroses such as erythrose and threose, ketotetroses such as erythrulose, aldopentoses such as arabinose, lyxose, ribose and xylose, ketopentoses such as ribulose and xylulose, aldohexoses such as allose, altrose, galactose, glucose, gulose, idose, mannose and talose, ketohexoses such as fructose, psicose, sorbose and tagatose, heptoses such as mannoheptulose, sedoheptulose, octoses such as octolose, 2-keto-3-deoxy-manno-octonate, nonoses such as sialoseallose. Disaccharides for example includes, but not limited to, glucorhamnose, trehalose, sucrose, lactose, maltose, galactosucrose, N-acetyllactosamine, cellobiose, gentiobiose, isomaltose, melibiose, primeverose, hesperodinose, and rutinose. Oligosaccharides for example includes, but not limited to, raffinose, nystose, panose, cellotriose, maltotriose, maltotetraose, xylobiose, galactotetraose, isopanose, cyclodextrin (α-CD) or cyclomaltohexaose, β-cyclodextrin (β-CD) or cyclomaltoheptaose and γ-cyclodextrin (γ-CD) or cyclomaltooctaose. Polysaccharide for example includes, but not limited to, xylan, mannan, galactan, glucan, arabinan, pustulan, gellan, guaran, xanthan, and hyaluronan. Some examples include, but not limited to, starch, glycogen, cellulose, inulin, chitin, amylose and amylopectin.

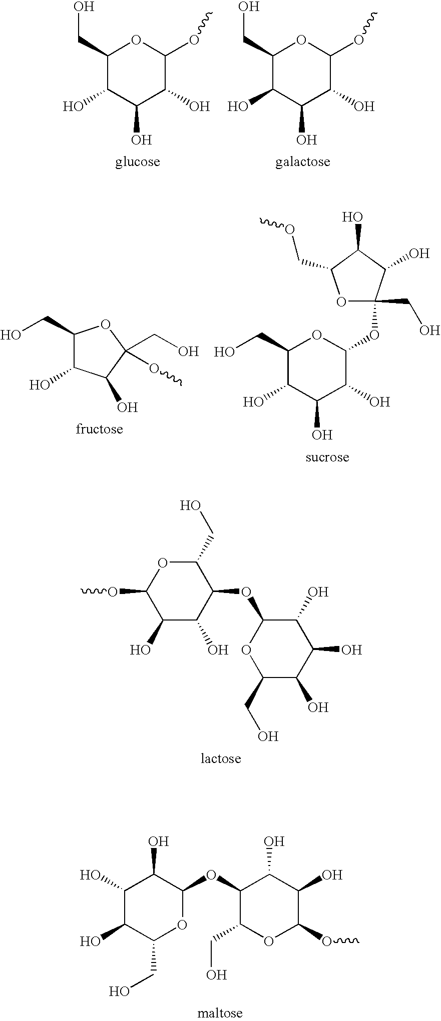

A compound of Formula I having a carbohydrate moiety can be referred to as the pyrone analog glycoside or the pyrone analog saccharide. As used herein, “carbohydrate” further encompasses the glucuronic as well as the glycosidic derivative of compounds of Formula I. Where the phosphorylated pyrone analog has no carbohydrate moiety, it can be referred to as the aglycone. Further, where a phenolic hydroxy is derivatized with any of the carbohydrates described above, the carbohydrate moiety is referred to as a glycosyl residue. Unless stated otherwise specifically in the specification, a carbohydrate group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Cyano” refers to a —CN moiety.

“Cycloalkyl” or “carbocyclyl” refers to a monocyclic or polycyclic non-aromatic radical that contains 3 to 10 ring carbon atoms (ie. C₃-C₁₀ cycloalkyl). It may be saturated or unsaturated. Whenever it appears herein, a numerical range such as “3 to 10” refers to each integer in the given range; e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclohexyl, cycloseptyl, cyclooctyl, cyclononyl, cyclodecyl, norbornyl, and the like. Unless stated otherwise specifically in the specification, a cycloalkyl group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Ester” refers to a chemical radical of formula —COOR, where R is selected from the group consisting of alkyl, cycloalkyl, aryl, heteroaryl (bonded through a ring carbon) and heterocyclic (bonded through a ring carbon). Any hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3.sup.rd Ed., John Wiley & Sons, New York, N.Y., 1999, which is incorporated herein by reference in its entirety. Unless stated otherwise specifically in the specification, an ester group is optionally substituted by one or more substituents which are independently: halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Fluoroalkyl” refers to an alkyl radical, as defined above, that is substituted by one or more fluoro radicals, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” are included in haloalkyl and haloalkoxy groups, respectively, in which the halo is fluorine.

The terms “heteroalkyl” “heteroalkenyl” and “heteroalkynyl” include optionally substituted alkyl, alkenyl and alkynyl radicals and which have one or more skeletal chain atoms selected from an atom other than carbon, e.g., oxygen, nitrogen, sulfur, phosphorus or a combination thereof.

“Heteroaryl” or, alternatively, “heteroaromatic” refers to a 5- to 18-membered aryl group that includes one or more ring heteroatoms selected from nitrogen, oxygen and sulfur, and which may be a monocyclic, bicyclic, tricyclic or tetracyclic fused ring system. Whenever it appears herein, a numerical range such as “5 to 18” refers to each integer in the given range; e.g., “5 to 18 ring atoms” means that the heteroaryl group may consist of 5 ring atoms, 6 ring atoms, etc., up to and including 18 ring atoms. An “N-containing heteroaromatic” or “N-containing heteroaryl” moiety refers to an aromatic group in which at least one of the skeletal atoms of the ring is a nitrogen atom. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, a heteroaryl moiety is optionally substituted by one or more substituents which are independently: hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, heterocyclic, C₃-C₁₀ cycloalkyl, —CN, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R³ is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Heterocyclyl” or “heterocyclic” refers to a stable 3- to 18-membered non-aromatic ring radical that comprises one to six heteroatoms selected from nitrogen, oxygen and sulfur. Whenever it appears herein, a numerical range such as “3 to 18” refers to each integer in the given range; e.g., “3 to 18 ring atoms” means that the heteroaryl group may consist of 3 ring atoms, 4 ring atoms, etc., up to and including 18 ring atoms. In some embodiments, it is a C₅-C₁₀ heterocyclyl. In some embodiments, it is a C₄-C₁₀ heterocyclyl. In some embodiments, it is a C₃-C₁₀ heterocyclyl. Unless stated otherwise specifically in the specification, the heterocyclyl radical is a monocyclic, bicyclic, tricyclic or tetracyclic ring system, which may include fused or bridged ring systems. The heteroatoms in the heterocyclyl radical may be optionally oxidized. One or more nitrogen atoms, if present, are optionally quaternized. The heterocyclyl radical is partially or fully saturated. The heterocyclyl may be attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocylyl moiety is optionally substituted by one or more substituents which are independently: hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, heterocyclic, C₃-C₁₀cycloalkyl, —CN, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), —PO₃WY (where W and Y are hydrogen, methyl, ethyl, alkyl, carbohydrate, lithium, sodium or potassium) or —PO₃Z (where Z is calcium, magnesium or iron) where R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl.

“Imino” refers to the ═N—H radical.

“Isocyanato” refers to a —N═C═O radical.

“Isothiocyanato” refers to a —N═C═S radical.

“Mercapto” refers to a (alkyl)S— or (H)S— radical.

“Moiety” refers to a specific segment or functional group of a molecule. Chemical moieties are often recognized chemical entities embedded in or appended to a molecule.

“Nitro” refers to the —NO₂ radical.

“Oxa” refers to the —O— radical.

“Oxo” refers to the ═O radical.

“Phosphorylated compound” or “phosphate” refers to compounds comprising at least one phosphate group. As used herein, a phosphate group includes but not limited to the groups —OCH₂OPO₃WY (also known as —OCH₂PO₄WY), or —OCH₂OPO₃Z (also known as —OCH₂PO₄Z), —OPO₃WY, or —OPO₃Z, wherein W and Y are independently selected from hydrogen, methyl, ethyl, alkyl, carbohydrate, and a cation, and wherein Z is a multivalent cation. Phosphorylated compounds, as used herein, include compounds having a phosphate group on polyphenol, hydroxylated or polyhdroxylated aromatic compound, or phosphorylated pyrone analog. For example, a phosphorylated compound would include a compound with an inositol phosphate group. Examples of phosphorylated compounds are, but in no way limited to, phosphorylated quercetin, phosphorylated isoquercetin, phosphorylated quercitin, phosphorylated flavone, phosphorylated chrysin, phosphorylated apigenin, phosphorylated rhoifolin, phosphorylated diosmin, phosphorylated galangin, phosphorylated fisetin, phosphorylated morin, phosphorylated rutin, phosphorylated kaempferol, phosphorylated myricetin, phosphorylated taxifolin, phosphorylated naringenin, phosphorylated naringin, phosphorylated hesperetin, phosphorylated hesperidin, phosphorylated chalcone, phosphorylated phloretin, phosphorylated phlorizdin, phosphorylated genistein, phosphorylated 5,7-dideoxyquercetin, phosphorylated biochanin A, phosphorylated catechin, and phosphorylated epicatechin.

“Phosphonated compound” or “phosphonate” “refers to compounds comprising at least one phosphonate group. A phosphonate group includes the groups —PO₃WY, —OCH₂PO₃WY, —OCH₂PO₃Z, —OLPO₃WY, —OLPO₃Z or —PO₃Z, wherein the group is attached to a carbon atom, wherein W and Y are independently selected from hydrogen, methyl, ethyl, alkyl, carbohydrate, and a cation, L is methyl, ethyl, alkyl or a carbohydate and wherein Z is a multivalent cation. Phosphonated compounds, as used herein, include compounds having a phosphonate group on polyphenol, polyhdroxylated aromatic compound, or flavonoid. For example, a phosphonated compound would include a compound with an inositol phosphonate group. Examples of phosphonated compounds are, but in no way limited to, phosphonated quercetin, phosphonated isoquercetin, phosphonated quercitrin, phosphonated flavone, phosphonated chrysin, phosphonated apigenin, phosphonated rhoifolin, phosphonated diosmin, phosphonated galangin, phosphonated fisetin, phosphonated morin, phosphonated rutin, phosphonated kaempferol, phosphonated myricetin, phosphonated taxifolin, phosphonated naringenin, phosphonated naringin, phosphonated hesperetin, phosphonated hesperidin, phosphonated chalcone, phosphonated phloretin, phosphonated phlorizdin, phosphonated genistein, phosphonated 5,7-dideoxyquercetin, phosphonated biochanin A, phosphonated catechin, and phosphonated epicatechin.

“Prodrug”, “prodrugs”, and “pharmaceutically or veterinarily acceptable prodrugs” refer to a derivative of an active compound (drug) that undergoes a transformation under the conditions of use, such as within the body, to release an active drug or an active metabolite thereof. Prodrugs are frequently, but not necessarily, pharmacologically inactive until converted into the active drug or an active metabolite thereof. Prodrugs are typically obtained by masking one or more functional groups in the drug believed to be in part required for activity with a prodrug group to form a prodrug moiety which undergoes a transformation, such as cleavage, under the specified conditions of use to release the functional group, and hence the active drug. The cleavage of the prodrug moiety may proceed spontaneously, such as by way of a hydrolysis reaction, or it may be catalyzed or induced by another agent, such as by an enzyme, by light, by acid, or by a change of or exposure to a physical or environmental parameter, such as a change of temperature or pH. The agent may be endogenous to the conditions of use, such as an enzyme present in the cells to which the prodrug is administered or the acidic conditions of the stomach, or it may be supplied exogenously.

A wide variety of prodrug groups, as well as the resultant prodrug moieties, suitable for masking functional groups in active compounds to yield prodrugs are well-known in the art. For example, a hydroxyl functional group may be masked as a sulfonate, ester or carbonate prodrug moiety, which may be hydrolyzed in vitro to provide the hydroxyl group. An amino functional group may be masked as an amide, imine, or sulfenyl promoiety, which may be hydrolyzed in vivo to provide the amino group. A carboxyl group may be masked as an ester (including silyl esters and thioesters), amide or hydrazide prodrug moiety, which may be hydrolyzed in vivo to provide the carboxyl group. Other specific examples of suitable prodrug groups and their respective prodrug moieties will be apparent to those of skill in the art.

“Sulfonyl” refers to a —S(═O)—R radical, where R is selected from the group consisting of alkyl, cycloalkyl, aryl, heteroaryl (bonded through a ring carbon) and heterocyclic (bonded through a ring carbon)

“Sulfonyl” refers to a —S(═O)₂—R radical, where R is selected from the group consisting of alkyl, cycloalkyl, aryl, heteroaryl (bonded through a ring carbon) and heterocyclic (bonded through a ring carbon).

“Sulfonamidyl” refers to a —S(═O)₂—NRR radical, where R is selected independently from the group consisting of hydrogen, alkyl, cycloalkyl, aryl, heteroaryl (bonded through a ring carbon) and heterocyclic (bonded through a ring carbon).

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heterocyclic (bonded through a ring carbon).

“Thiocyanato” refers to a —C═N═S radical.

“Thioxo” refers to the ═S radical.

“Substituted” means that the referenced group may be substituted with one or more additional group(s) individually and independently selected from acyl, alkyl, alkylaryl, cycloalkyl, aralkyl, aryl, carbohydrate, heteroaryl, heterocyclic, hydroxy, alkoxy, aryloxy, mercapto, alkylthio, arylthio, cyano, halo, carbonyl, ester, thiocarbonyl, isocyanato, thiocyanato, isothiocyanato, nitro, perhaloalkyl, perfluoroalkyl, phosphate, silyl, sulfinyl, sulfonyl, sulfonamidyl, sulfoxyl, sulfonate, and amino, including mono- and di-substituted amino groups, and the protected derivatives thereof. The subsituents themselves may be substituted, for example, a cycloakyl substituent may have a halide substituted at one or more ring carbons, and the like. The protecting groups that may form the protective derivatives of the above substituents are known to those of skill in the art and may be found in references such as Greene and Wuts, above.

The terms “methylphosphonate” and “methyl phosphonic acid” are used interchangeably herein to refer to the group “—CH₂—P(O)(OH)₂.”

The compounds presented herein may possess one or more chiral centers and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds having the structure of Formula I, as well as active metabolites of these compounds having the same type of activity. In addition, the compounds described herein can exist in unsolvated as well as solvated forms with pharmaceutically acceptable solvents such as water, ethanol, and the like. The solvated forms of the compounds presented herein are also considered to be disclosed herein.

A pyrone analog of Formula I and its pharmaceutically/veterinarily acceptable salt or esters is provided herein.

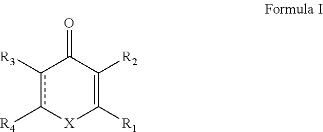

wherein X is O, S, or NR′ wherein R′ is hydrogen, C₁-C₁₀ alkyl, C₂-C₁₀ alkynyl, C₂-C₁₀ alkenyl, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, or C₃-C₁₀ cycloalkyl;

R₁, and R₂ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₄-C₁₀ heterocyclyl, heteroaryl, C₃-C₁₀cycloalkyl, —OCH₂OPO₃WY, —OCH₂OPO₃Z, —OPO₃WY, —OPO₃Z, —OLPO₃WY, or —OLPO₃Z;

R₃ and R₄ are independently hydrogen, hydroxyl, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl C₆-C₁₀ aralkyl acyl, C₆-C₁₀ alkylaryl acyl, alkoxy, amine, aryl, C₄-C₁₀ heterocyclyl, heteroaryl, C₃-C₁₀cycloalkyl, —OCH₂OPO₃WY, —OCH₂OPO₃Z, —OPO₃WY, —OPO₃Z, —OLPO₃WY, or —OLPO₃Z; or R₃ and R₄ are taken together to form a C₅-C₁₀ heterocyclyl, C₅-C₁₀ cycloalkyl, aryl, or heteroaryl;

W and Y are independently hydrogen, methyl, ethyl, alkyl, carbohydrate, or a cation, and Z is a multivalent cation; and

L is methyl, ethyl, alkyl or a carbohydate.

In various embodiments, W is potassium. In various embodiments, W is sodium. In various embodiments, W is lithium. In various embodiments, Y is potassium. In various embodiments, Y is sodium. In various embodiments, Y is lithium.

In various embodiments, Z is calcium. In various embodiments, Z is magnesium. In various embodiments, Z is iron.

In various embodiments, L is methyl or ethyl.

The 2,3 bond may be saturated or unsaturated in the compounds of Formula I.

In some embodiments, the pyrone analog of Formula I is of Formula II:

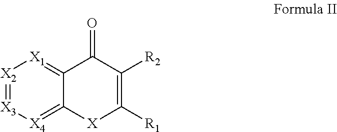

wherein X, R₁, R₂, W, Y, Z, and L are defined as in Formula I;

X₁, X₂, X₃, and X₄ are independently CR₅, O, S, or N;

R₅ is independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀ cycloalkyl, —OCH₂OPO₃WY, —OCH₂OPO₃Z, —OPO₃WY, or —OPO₃Z, —OLPO₃WY, or —OLPO₃Z.

In some embodiments, X₁ is CR₅.

In other embodiments, X₁ is O.

In yet other embodiments, X₁ is S.

In further embodiments, X₁ is N.

In some embodiments, X₂ is CR₅.

In other embodiments, X₂ is O.

In yet other embodiments, X₂ is S.

In further embodiments, X₂ is N.

In some embodiments, X₃ is CR₅.

In other embodiments, X₃ is O.

In yet other embodiments, X₃ is S.

In further embodiments, X₃ is N.

In other embodiments, X₄ is CR₅.

In some embodiments, X₄ is O.

In yet other embodiments, X₄ is S.

In some embodiments, X₄ is N.

In some embodiments, X₁, X₂, X₃, and X₄ are CR₅.

In some embodiments, X₁ and X₃ are CR₅ and X₂ and X₄ are N.

In some embodiments, X₂ and X₄ are CR₅ and X₁ and X₃ are N.

In some embodiments, X₂ and X₃ are CR₅ and X₁ and X₄ are N.

In various embodiments, R₁ is one of the following formulae:

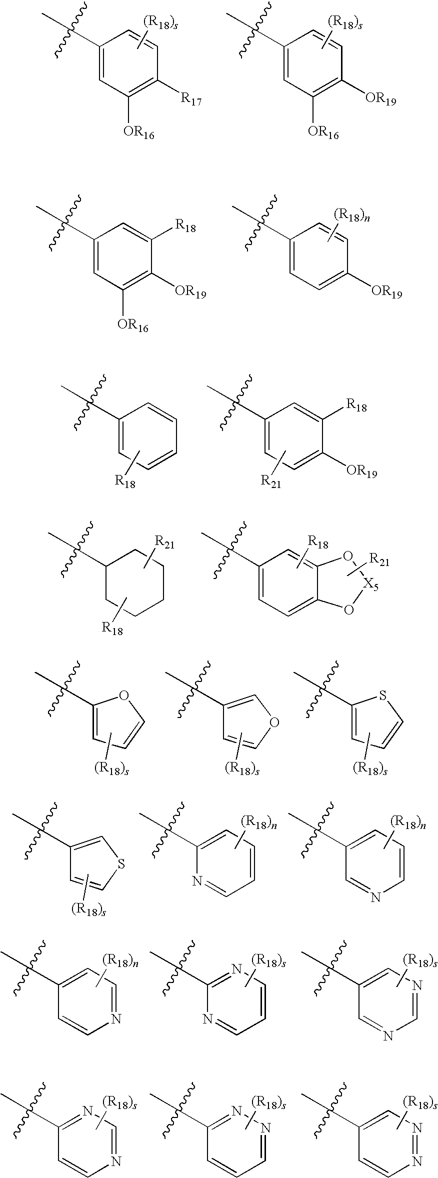

wherein R₁₆ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, C₃-C₁₀cycloalkyl, —CH₂OPO₃WY, —CH₂OPO₃Z, —PO₃WY, —PO₃Z, —OLPO₃WY, or —OLPO₃Z;

R₁₇ is hydrogen, hydroxy, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, aryl, C₃-C₁₀ heterocyclyl, heteroaryl, or C₃-C₁₀ cycloalkyl, —OCH₂OPO₃WY, —OCH₂OPO₃Z, —OPO₃WY, —OPO₃Z, —OLPO₃WY, or —OLPO₃Z;

R₁₈ and R₂₁ are independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, heterocyclic, C₃-C₁₀cycloalkyl, —OCH₂OPO₃WY, —OCH₂OPO₃Z, —OPO₃WY, —OPO₃Z, —OLPO₃WY, or —OLPO₃Z;

R₁₉ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —CH₂OPO₃WY, —CH₂OPO₃Z, —PO₃WY, —PO₃Z—OLPO₃WY, or —OLPO₃Z;

s is an integer of 0, 1, 2, or 3; and

n is an integer of 0, 1, 2, 3, or 4.

In various embodiments, W and Y are independently potassium, sodium, or lithium.

In various embodiments, Z is calcium, magnesium or iron.

In various embodiments, the pyrone analog is of Formulae III, IV, V, or VI as illustrated in Scheme I.

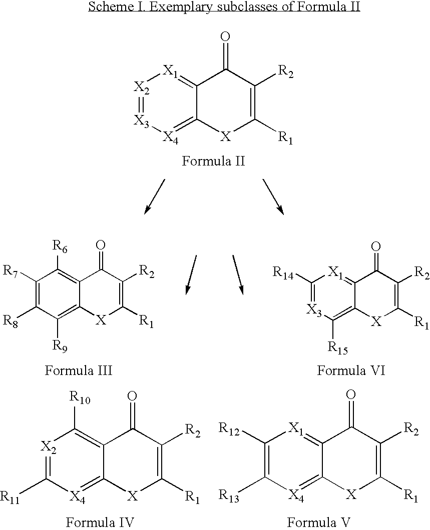

In some embodiments where the X₁, X₂, X₃, and X₄ of the compounds of Formula II are CR₅, the compound is of Formula III:

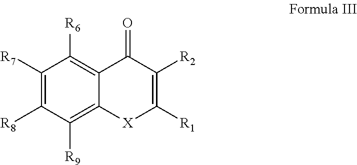

wherein X, R₁, R₂, W, Y, Z, and L are defined as in Formula I and Formula II;

R₆, R₇, R₈, and R₉ are independently hydrogen, hydroxyl, carboxaldehyde, amino, C₁-C₁₀ alkyl, C₂-C₁₀ alkynyl, C₂-C₁₀alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OCH₂OPO₃WY, —OCH₂OPO₃Z, —OPO₃WY, —OPO₃Z, —OLPO₃WY, or —OLPO₃Z.

In various embodiments, the pyrone analog of Formula III is of Formula VII:

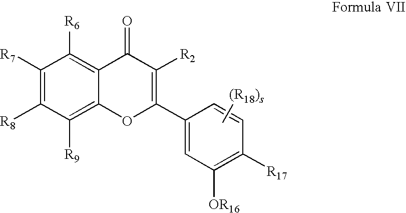

wherein R₂, R₁₆, R₁₇, R₁₈, and s are as defined in Formula II and R₆, R₇, R₈, and R₉ are as defined in Formula III.

In other embodiments, the pyrone analog of Formula III is a compound of Formula VIII:

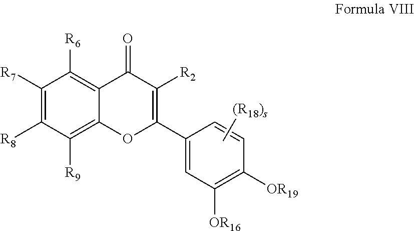

wherein R₂, R₁₆, R₁₈, R₁₉, and s are as defined in Formula II and R₆, R₇, R₈, and R₉ are as defined in Formula III.

In some embodiments, the pyrone analog of Formula III is of Formula IX:

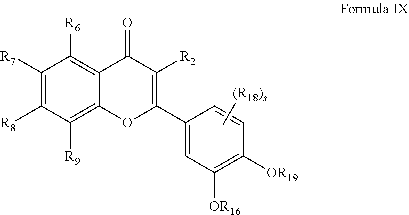

wherein R₂, R₁₆, R₁₈, R₁₉, and s are as defined in Formula II; and

R₆, R₇, R₈, and R₉ are independently hydrogen, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OCH₂OPO₃WY, OCH₂OPO₃Z, —OPO₃WY, —OPO₃Z, —OLPO₃WY, or —OLPO₃Z. In this embodiment, none of R₆-R₉ is OH.

In some embodiments, the pyrone analog of Formula III is of Formula X:

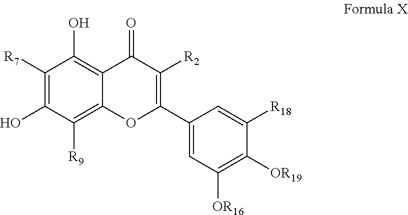

wherein R₂, R₁₆, R₁₈, and R₁₉ are as defined in Formula II and R₇ and R₉ are as defined in Formula III.

In other embodiments, the pyrone analog of Formula III is of Formula XI:

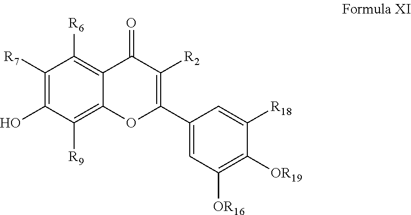

wherein R₂, R₁₆, R₁₈, and R₁₉ are as defined in Formula II and R₆, R₇, and R₉ are as defined in Formula III.

In some embodiments, compounds of the following Formulae VIII-A, VIII-B, and VIII-C, are useful in the embodiments described herein, where R_{c} and R_{d} are independently hydrogen, —CH₂OPO₃WY, —CH₂OPO₃Z, —PO₃WY, —PO₃Z, —OLPO₃WY, or —OLPO₃Z, where W and Y are hydrogen, methyl, ethyl, alkyl, carbohydrate, lithium, sodium or potassium, Z is calcium, magnesium or iron, and L is methyl, ethyl, alkyl or a carbohydate, and wherein at least one of the R_{c} or R_{d} is a phosphate or phosphonate.

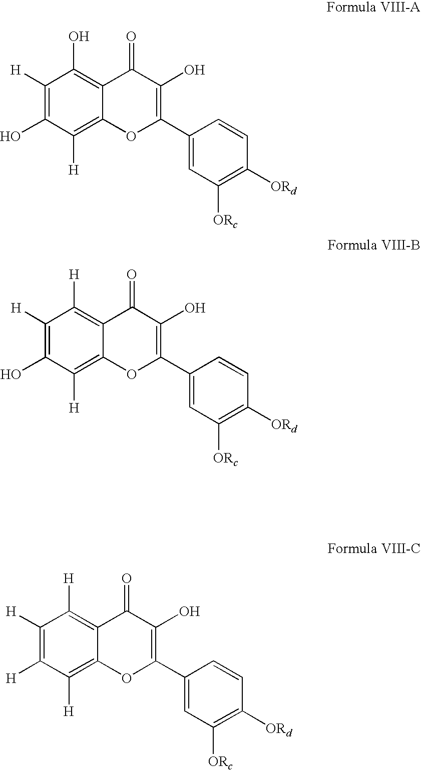

In some embodiments, for a compound of Formulae VIII-A, VIII-B, or VIII-C, one of R_{c} and R_{d} is hydrogen. In some embodiments, R_{c} is —PO₃WY and R_{d} is hydrogen. In some embodiments, R_{c} is —PO₃WY and R_{d} is —PO₃WY. In some embodiments, R_{c} is a mixture of hydrogen and —PO₃WY and R_{d} is —PO₃WY. In some embodiments, R_{c} is hydrogen and R_{d} is a mixture of hydrogen and —PO₃WY. In some embodiments, R_{c} is —PO₃Z and R_{d} is hydrogen. In some embodiments, R_{c} is —PO₃Z and R_{d} is —PO₃Z. In some embodiments, R_{c} is a mixture of hydrogen and —PO₃Z and R_{d} is —PO₃Z. In some embodiments, R_{c} is hydrogen and R_{d} is a mixture of hydrogen and —PO₃Z. In some embodiments, R_{c} is —CH₂OPO₃Z and R_{d} is hydrogen. In some embodiments, R_{c} is —CH₂OPO₃Z and R_{d} is —CH₂OPO₃Z. In some embodiments, R_{c} is a mixture of hydrogen and —CH₂OPO₃Z and R_{d} is —CH₂OPO₃Z. In some embodiments, R_{c} is hydrogen and R_{d} is a mixture of hydrogen and —CH₂OPO₃Z.

In some embodiments of the invention, for a compound of Formulae VIII-A, VIII-B, or VIII-C, one of Rc and Rd is hydrogen. In some embodiments of the invention, for a compound of Formulae VIII-A, VIII-B, or VIII-C, R_{c} is -LPO₃WY and R_{d} is hydrogen. In some embodiments of the invention, for a compound of Formulae VIII-A, VIII-B, or VIII-C, R_{c} is -LPO₃WY and R_{d} is -LPO₃WY. In some embodiments of the invention, for a compound of Formulae VIII A, VIII-B, or VIII-C, R_{c} is a mixture of hydrogen and -LPO₃WY and R_{d} is -LPO₃WY. In some embodiments of the invention, for a compound of Formulae VIII-A, VIII-B, or VIII-C, R_{c} is hydrogen and R_{d} is a mixture of hydrogen and -LPO₃Z. In some embodiments of the invention, for a compound of Formulae VIII-A, VIII-B, or VIII-C, R_{c} is -LPO₃Z and R_{d} is hydrogen. In some embodiments of the invention, for a compound of Formulae VIII-A, VIII-B, or VIII-C, R_{c} is -LPO₃Z and R_{d} is -LPO₃Z. In some embodiments of the invention, for a compound of Formulae VIII-A, VIII-B, or VIII-C, R_{c} is a mixture of hydrogen and -LPO₃Z and R_{d} is -LPO₃Z. In some embodiments of the invention, for a compound of Formulae VIII-A, VIII-B, or VIII-C, R_{c} is hydrogen and R_{d} is a mixture of hydrogen and -LPO₃Z.

In other embodiments, the pyrone analog of Formula III is of Formula XII:

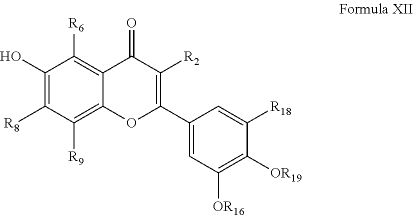

wherein R₂, R₁₆, R₁₈, and R₁₉ are as defined in Formula II and R₆, R₈, and R₉ are as defined in Formula III.

In other embodiments, the pyrone analog of Formula III is of Formula XIII:

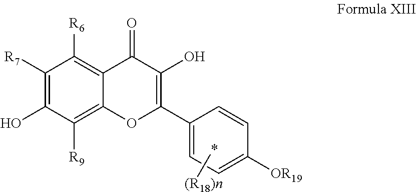

wherein n, R₁₈, and R₁₉ are as defined in Formula II and R₆, R₇, and R₉ are as defined in Formula III.

In some embodiments, the pyrone analog of Formula III is of Formula XIV:

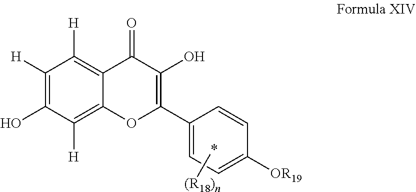

wherein R₁₈, R₁₉, and n are as defined in Formula II.

In some embodiments, the pyrone analog of Formula III is of Formula XV:

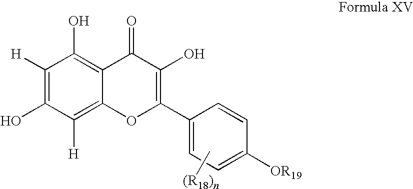

wherein R₁₈, R₁₉, and n are as defined in Formula II.

In some embodiments, the pyrone analog of Formula III is of Formula XVI:

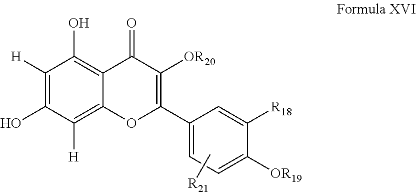

wherein R₁₈, R₁₉, and R₂₁ are as defined in Formula II;

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —CH₂OPO₃WY, —CH₂OPO₃Z, —PO₃WY, —PO₃Z, —OLPO₃WY, or —OLPO₃Z; and

W and Y are independently hydrogen, methyl, ethyl, alkyl, carbohydrate, or a cation, Z is a multivalent cation, and L is methyl, ethyl, alkyl or a carbohydate.

In some embodiments, the pyrone analog of Formula III is of Formula XVII:

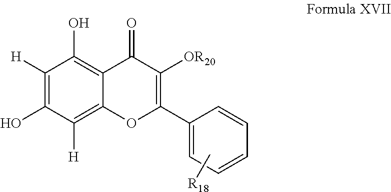

wherein R₁₈ is as defined in Formula II; and

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —CH₂OPO₃WY, —CH₂OPO₃Z, —PO₃WY, —PO₃Z, —OLPO₃WY, or —OLPO₃Z.

In some embodiments, the pyrone analog of Formula III is of Formula XVIII:

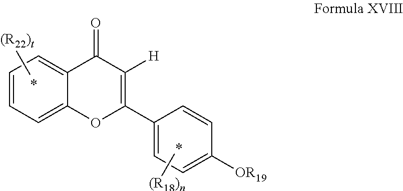

wherein n, R₁₈ and R₁₉ are as defined in Formula II;

wherein R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, heterocyclic, C₃-C₁₀cycloalkyl, —OCH₂OPO₃WY, —OCH₂OPO₃Z, —OPO₃WY, —OPO₃Z, —OLPO₃WY, or —OLPO₃Z; and

t is an integer of 0, 1, 2, 3, or 4

In some embodiments, the pyrone analog of Formula III is of Formula XIX:

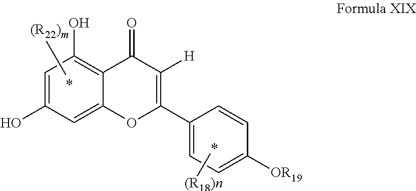

wherein n, R₁₈ and R₁₉ are as defined in Formula II;

wherein R₂₂ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, heterocyclic, C₃-C₁₀cycloalkyl, —OCH₂OPO₃WY, —OCH₂OPO₃Z, —OPO₃WY, —OPO₃Z, —OLPO₃WY, or —OLPO₃Z; and

m is an integer of 0, 1, or 2.

In some embodiments, the pyrone analog of Formula M is of Formula XX:

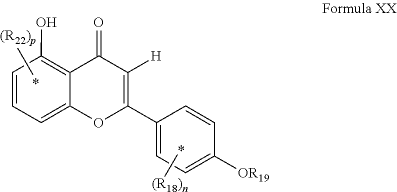

wherein n, R₁₈ and R₁₉ are as defined in Formula II;

wherein R₂₂ is independently hydrogen, hydroxyl, carboxaldehyde, amine, C₁-C₁₀ alkyl, C₂-C₁₀alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, heterocyclic, C₃-C₁₀cycloalkyl, —OCH₂OPO₃WY, —OCH₂OPO₃Z, —OPO₃WY, —OPO₃Z, —OLPO₃WY, or —OLPO₃Z; and

p is an integer of 0, 1, 2 or 3.

In some embodiments, the pyrone analog of Formula III is of Formula XXI:

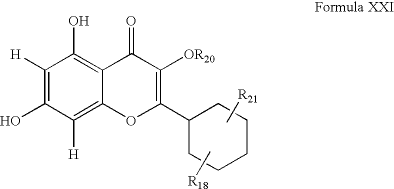

wherein R₁₈ and R₂₁ are as defined in Formula II; and

R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —CH₂OPO₃WY, —CH₂OPO₃Z, —PO₃WY, —PO₃Z, -LPO₃WY, or -LPO₃Z.

In some embodiments, the pyrone analog of Formula III is of Formula XXII:

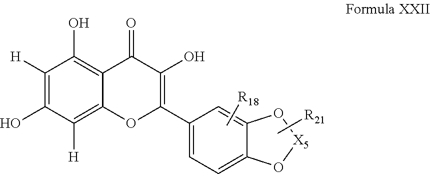

wherein R₁₈ and R₂₁ are as defined in Formula II;

wherein X₅ is a C₁ to C₄ group, optionally interrupted by O, S, NR₂₃, or NR₂₃R₂₃ as valency permits, forming a ring which is aromatic or nonaromatic;

R₂₃ is independently hydrogen, C₁-C₁₀ alkyl, C₂-C₁₀ alkynyl, C₂-C₁₀ alkenyl, carbohydrate, acyloxy, C₁-C₁₀ aliphatic acyl, C₆-C₁₀ aromatic acyl, C₆-C₁₀ aralkyl acyl, C₆-C₁₀ alkylaryl acyl, alkoxy, aryl, heteroaryl, C₅-C₁₀heterocyclyl, C₃-C₁₀cycloalkyl, —CH₂OPO₃WY, —CH₂OPO₃Z, —PO₃WY, —PO₃Z, -LPO₃WY, or -LPO₃Z.

In some embodiments, the pyrone analog of Formula III is of Formula XXIII:

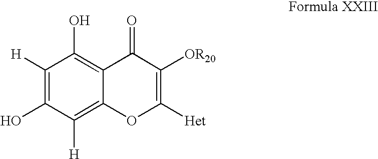

wherein R₂₀ is hydrogen, C₁-C₁₀ alkyl, C₂-C₁₀ alkynyl, C₂-C₁₀ alkenyl, carbohydrate, C₁-C₁₀ aliphatic acyl, C₆-C₁₀ aromatic acyl, C₆-C₁₀ aralkyl acyl, C₆-C₁₀ alkylaryl acyl, aryl, C₃-C₁₀ heterocyclyl, heteroaryl, optionally substituted C₃-C₁₀cycloalkyl, —PO₃WY, —CH₂OPO₃WY, —CH₂OPO₃Z, —PO₃Z, -LPO₃WY, or -LPO₃Z;

Het is a 3 to 10 membered optionally substituted monocyclic or bicyclic heteroaromatic or heterocyclic ring system containing 1, 2, 3, 4, or 5 heteroatoms selected from the group of O, S, and N, with the proviso that no two adjacent ring atoms are O or S, wherein the ring system is unsaturated, partially unsaturated or saturated, wherein any number of the ring atoms have substituents as valency permits which are hydrogen, hydroxyl, carboxyaldehyde, alkylcarboxaldehyde, imino, C₁-C₁₀ alkyl, C₂-C₁₀ alkynyl, C₂-C₁₀ alkenyl, carboxyl, carbohydrate, acyloxy, nitro, halogen, C₁-C₁₀ aliphatic acyl, C₅-C₁₀ aromatic acyl, C₆-C₁₀ aralkyl acyl, C₆-C₁₀alkylaryl acyl, alkoxy, amine, aryl, heteroaryl, C₅-C₁₀ heterocyclyl, C₅-C₁₀cycloalkyl, —OCH₂OPO₃WY, —OCH₂OPO₃Z, —OPO₃WY, —OPO₃Z, —OLPO₃WY, or —OLPO₃Z and

W and Y are independently hydrogen, methyl, ethyl, alkyl, carbohydrate, or a cation, Z is a multivalent cation, and L is methyl, ethyl, alkyl or a carbohydate.

In some embodiments, Het is one of the following formulae:

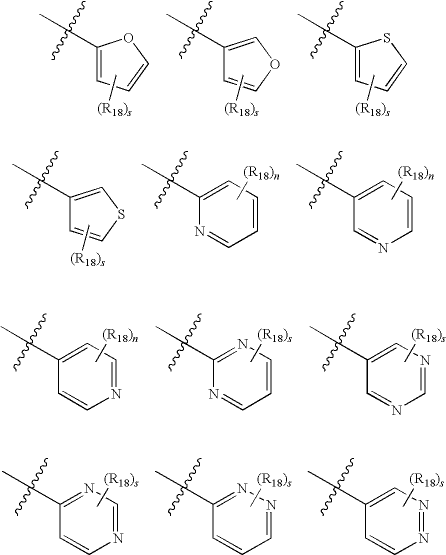

wherein R₁₈ is independently hydrogen, hydroxyl, carboxaldehyde, amine, C₁-C₁₀ alkyl, C₂-C₁₀alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, heterocyclic, C₃-C₁₀cycloalkyl, —OCH₂OPO₃WY, —OCH₂OPO₃Z, —OPO₃WY, —OPO₃Z, —OLPO₃WY, or —OLPO₃Z;

s is an integer of 0, 1, 2, or 3; and

n is an integer of 0, 1, 2, 3, or 4.

In some embodiments, the pyrone analog of Formula II is of Formula IV:

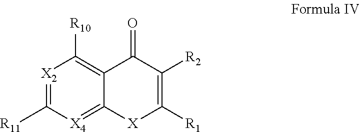

wherein X, X₂, X₄, R₁, and R₂ are as defined for Formula II; and

R₁₀ and R₁₁ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OCH₂OPO₃WY, —OCH₂OPO₃Z, —OPO₃WY, —OPO₃Z, —OLPO₃WY, or —OLPO₃Z.

In some embodiments, the pyrone analog of Formula IV is of Formula XXIV or Formula XXV:

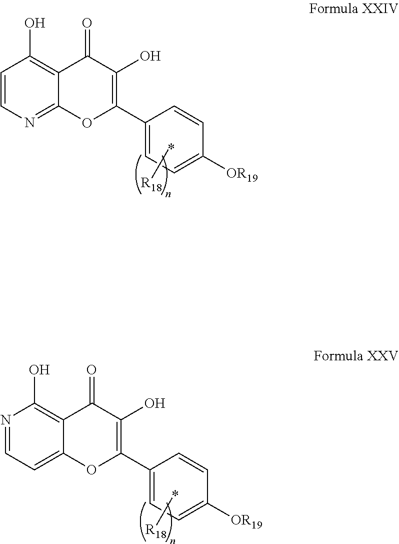

wherein R₁₈, R₁₉, and n are as defined in Formula II.

In some embodiments, the pyrone analog of Formula IV is of Formula XXVI or Formula XXVII:

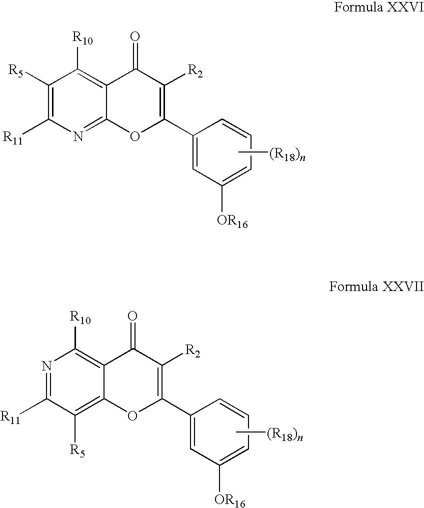

wherein R₂, and R₅ are as defined for Formula II and R₁₀ and R₁₁ are as defined for Formula IV;

R₁₆ is hydrogen, —CH₂OPO₃WY, —CH₂OPO₃Z, —PO₃WY, —PO₃Z, —OLPO₃WY, or —OLPO₃Z;

wherein R₁₈ is independently hydrogen, hydroxyl, carboxaldehyde, amine, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, alkyl, phosphate, aryl, heteroaryl, heterocyclic, C₃-C₁₀cycloalkyl, —OCH₂OPO₃WY, —OCH₂OPO₃Z, —OPO₃WY, —OPO₃Z, —OLPO₃WY, or —OLPO₃Z; and

n is an integer of 0, 1, 2, 3, or 4.

In some embodiments, the pyrone analog of Formula IV is of Formula XXVIII:

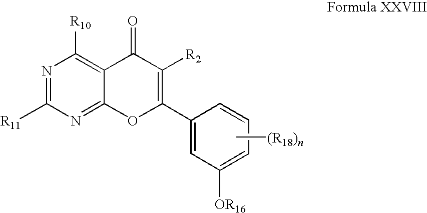

wherein R₂ is as defined for Formula II and R₁₀ and R₁₁ are as defined for Formula IV;

R₁₆ is hydrogen, —CH₂OPO₃WY, —CH₂OPO₃Z, —PO₃WY or —PO₃Z;

wherein R₁₈ is independently hydrogen, hydroxyl, carboxaldehyde, amine, C₁-C₁₀ alkyl, C₂-C₁₀alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀alkylaryl acyl, alkoxy, alkyl, phosphate, aryl, heteroaryl, heterocyclic, C₃-C₁₀cycloalkyl, —OCH₂OPO₃WY, —OCH₂OPO₃Z, —OPO₃WY or —OPO₃Z; and

n is an integer of 0, 1, 2, 3, or 4.

In some embodiments, the pyrone analog of Formula II is of Formula V:

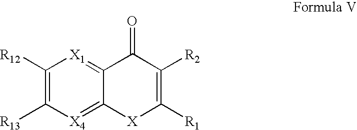

wherein X, X₁, X₄, R₁, and R₂ are as defined for Formula II; and R₁₂ and R₁₃ are independently hydrogen, hydroxyl, carboxaldehyde, amino, C₁-C₁₀ alkyl, C₂-C₁₀ alkynyl, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OCH₂OPO₃WY, —OCH₂OPO₃Z, —OPO₃WY, —OPO₃Z, —OLPO₃WY, or —OLPO₃Z.

In some embodiments, the pyrone analog of Formula V is of Formula XXIX or Formula XXX wherein the compound comprises at least one phosphate group:

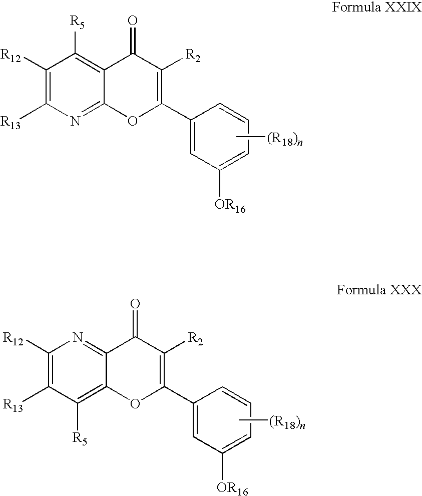

wherein R₂, R₅, R₁₈ and n are as defined for Formula II and R₁₂ and R₁₃ are as defined for Formula V; and

R₁₆ is hydrogen, —CH₂OPO₃WY, —CH₂OPO₃Z, —PO₃WY, —PO₃Z, -LPO₃WY, or -LPO₃Z.

In some embodiments, the pyrone analog of Formula V is of Formula XXXI:

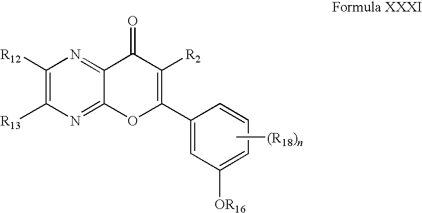

wherein R₂, R₁₈ and n are as defined for Formula II and R₁₂ and R₁₃ are as defined for Formula V; and

R₁₆ is hydrogen, —CH₂OPO₃WY, —CH₂OPO₃Z, —PO₃WY, —PO₃Z, -LPO₃WY, or -LPO₃Z.

In some embodiments, the pyrone analog of Formula II is of Formula VI:

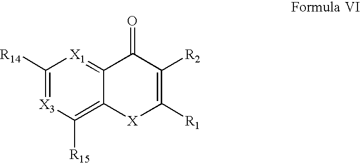

wherein X, X₁, X₃, R₁, and R₂ are as defined for Formula II; and

R₁₄ and R₁₅ are independently hydrogen, hydroxyl, carboxaldehyde, amino, C₁-C₁₀ alkyl, C₂-C₁₀ alkynly, C₂-C₁₀ alkenyl, carboxyl, carbohydrate, ester, acyloxy, nitro, halogen, C₁-C₁₀ aliphatic acyl, C₆-C₁₀ aromatic acyl, C₆-C₁₀ aralkyl acyl, C₆-C₁₀ alkylaryl acyl, alkoxy, amine, aryl, C₃-C₁₀ heterocyclyl, heteroaryl, C₃-C₁₀cycloalkyl, —OCH₂OPO₃WY, —OCH₂OPO₃Z, —OPO₃WY, —OPO₃Z, —OLPO₃WY, or —OLPO₃Z.

In some embodiments, the pyrone analog of Formula VI is of Formula XXXII or Formula XXXIII:

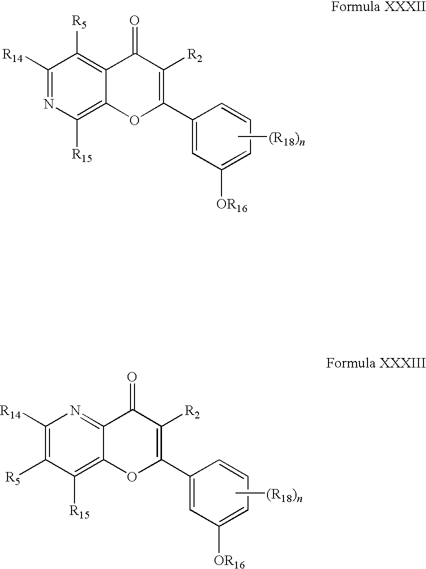

wherein R₂, R₅, R₁₈, and n are as defined for Formula II and R₁₄ and R₁₅ are as defined for Formula VI; and

R₁₆ is hydrogen, —CH₂OPO₃WY, —CH₂OPO₃Z, —PO₃WY, —PO₃Z, -LPO₃WY, or -LPO₃Z.

In some embodiments, the pyrone analog of Formula VI is of Formula XXXIV:

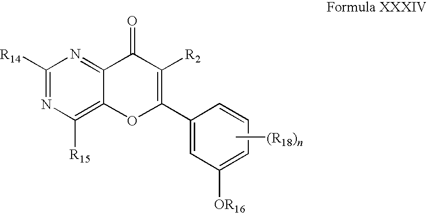

wherein R₂, R₁₈, and n are as defined for Formula II and R₁₄ and R₁₅ are as defined for Formula VI; and

R₁₆ is hydrogen, —CH₂OPO₃WY, —CH₂OPO₃Z, —PO₃WY, —PO₃Z, -LPO₃WY, or -LPO₃Z.

A useful class of pyrone analogs is the flavonoids. Flavonoids, the most abundant polyphenols in the diet, can be classified into subgroups based on differences in their chemical structures. The basic flavonoid structure is shown below as Formula XXXV. Compounds useful in the invention include phosphorylated compounds of the basic flavonoid structure, also shown below as Formula XXXV, and its pharmaceutically acceptable salts, esters, prodrugs, analogs, isomers, stereoisomers or tautomers thereof.

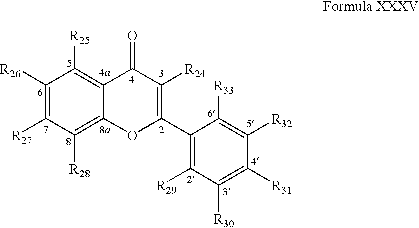

wherein the 2,3 bond may be saturated or unsaturated, and wherein R₂₄, R₂₅, R₂₆, R₂₇, R₂₈, R₂₉, R₃₀, R₃₁, R₃₂, and R₃₃ can be independently selected from the group consisting of hydrogen, halogen, hydroxyl, amine, thiol, C₁-C₁₀ alkyl, C₂-C₁₀ alkynyl, C₂-C₁₀ alkenyl, aryl, heteroaryl, C₃-C₁₀ cycloalkyl, heterocycloalkyl, C₁-C₁₀ aliphatic acyl, C₆-C₁₀ aromatic acyl, trialkylsilyl, ether, carbohydrate, —OPO₃WY, —OPO₃Z, —OLPO₃WY, and —OLPO₃Z, wherein W and Y are independently selected from hydrogen, methyl, ethyl, alkyl, carbohydrate, and a cation, and wherein Z is a multivalent cation.

In some embodiments, a flavonoid is utilized where the molecule is planar. In some embodiments, a flavonoid is utilized where the 2,3 bond is unsaturated. In some embodiments, a flavonoid is utilized where the 3-position is hydroxylated, phosphorylated, or phosphonated. In some embodiments, a flavonoid is utilized where the 2-3 bond is unsaturated and the 3-position is hydroxylated, phosphorylated, or phosphonated (e.g., flavonols).

In some embodiments, a phosphorylated flavonoid is utilized where the molecule is planar. In some embodiments, a phosphorylated flavonoid is utilized where the 2,3 bond is unsaturated. In some embodiments, a phosphorylated flavonoid is utilized where the 3-position is hydroxylated or phosphorylated. In some embodiments, a phosphorylated flavonoid is utilized where the 2-3 bond is unsaturated and the 3-position is hydroxylated or phosphorylated (e.g., flavonols).

In some embodiments, a phosphonated flavonoid is utilized where the molecule is planar. In some embodiments, a phosphonated flavonoid is utilized where the 2,3 bond is unsaturated. In some embodiments, a phosphonated flavonoid is utilized where the 3-position is hydroxylated or phosphonated. In some embodiments, a phosphonated flavonoid is utilized where the 2-3 bond is unsaturated and the 3-position is hydroxylated or phosphonated (e.g., flavonols).

Flavonoids include, but are not limited to, quercetin, isoquercetin, flavone, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, epicatechin, and a mixture (combination) thereof. In one embodiment, one or more flavonoids utilized in the methods described herein include, but are not limited to, apigenin, rhoifolin, galangin, fisetin, morin, rutin, kaempferol, myricetin, naringenin, hesperetin, phloretin, genistein, and a mixture (combination) thereof. Structures of these compounds are well-known in the art. See, e.g., Critchfield et al. (1994) Biochem. Pharmacol 7:1437-1445.

In some embodiments, one or more phosphorylated flavonoids may be utilized in the methods described herein. Phosphorylated flavonoids include, but are not limited to, phosphorylated quercetin, phosphorylated isoquercetin, phosphorylated fisetin, phosphorylated flavone, phosphorylated chrysin, phosphorylated apigenin, phosphorylated rhoifolin, phosphorylated diosmin, phosphorylated galangin, phosphorylated morin, phosphorylated rutin, phosphorylated kaempferol, phosphorylated myricetin, phosphorylated taxifolin, phosphorylated naringenin, phosphorylated naringin, phosphorylated hesperetin, phosphorylated hesperidin, phosphorylated chalcone, phosphorylated phloretin, phosphorylated phlorizdin, phosphorylated genistein, phosphorylated biochanin A, phosphorylated catechin, phosphorylated and phosphorylated epicatechin, and a mixture (combination) thereof. In one embodiment, the one or more phosphorylated flavonoids utilized in the methods described herein include, but are not limited to, phosphorylated quercetin, phosphorylated fisetin, phosphorylated apigenin, phosphorylated rhoifolin, phosphorylated galangin, phosphorylated fisetin, phosphorylated morin, phosphorylated rutin, phosphorylated kaempferol, phosphorylated myricetin, phosphorylated naringenin, phosphorylated hesperetin, phosphorylated phloretin, and phosphorylated genistein, and a mixture (combination) thereof. Structures of the un-phosphorylated versions of these compounds are well-known in the art. See, e.g., Critchfield et al. (1994) Biochem. Pharmacol 7:1437-1445.

In some embodiments, one or more phosphonated flavonoids may be utilized in the methods described herein. Phosphonated flavonoids include, but are not limited to, phosphonated quercetin, phosphonated isoquercetin, phosphonated fisetin, phosphonated flavone, phosphonated chrysin, phosphonated apigenin, phosphonated rhoifolin, phosphonated diosmin, phosphonated galangin, phosphonated morin, phosphonated rutin, phosphonated kaempferol, phosphonated myricetin, phosphonated taxifolin, phosphonated naringenin, phosphonated naringin, phosphonated hesperetin, phosphonated hesperidin, phosphonated chalcone, phosphonated phloretin, phosphonated phlorizdin, phosphonated genistein, phosphonated biochanin A, phosphonated catechin, and phosphonated epicatechin, and a mixture (combination) thereof. In one embodiment, the one or more phosphonated flavonoids utilized in the methods described herein include, but are not limited to, phosphonated quercetin, phosphonated fisetin, phosphonated apigenin, phosphonated rhoifolin, phosphonated galangin, phosphonated fisetin, phosphonated morin, phosphonated phosphonated kaempferol, phosphonated myricetin, phosphonated naringenin, phosphonated hesperetin, phosphonated phloretin, and phosphonated genistein, and a mixture (combination) thereof. Structures of the un-phosphonated versions of these compounds are well-known in the art. See, e.g., Critchfield et al. (1994) Biochem. Pharmacol 7:1437-1445.

In some embodiments, a flavonol is utilized in the methods described herein. In some embodiments, the flavonol is selected from the group consisting of quercetin, fisetin, morin, rutin, myricetin, galangin, and kaempferol, and combinations thereof. In some embodiments, the flavonol is selected from the group consisting of quercetin, fisetin, galangin, and kaempferol, and combinations thereof. In other embodiments, the flavonol is quercetin or a substituted analog thereof. In other embodiments, the flavonol is fisetin or a substituted analog thereof. In some embodiments, the flavonol is galangin or a substituted analog thereof. In some embodiments, the flavonol is kaempferol or a substituted analog thereof.

In some embodiments, a compound of Formula I to XXXV above contains at least one phosphate group. In other embodiments, a compound of Formula I to XXXV above contains at least one phosphonate group.

A. Phosphorylated Pyrone Analogs

In some embodiments a phosphorylated flavonol is utilized in the methods described herein. In some embodiments, the phosphorylated flavonol is selected from the group consisting of phosphorylated quercetin, phosphorylated fisetin, phosphorylated morin, phosphorylated rutin, phosphorylated myricetin, phosphorylated galangin, phosphorylated kaempferol, and combinations thereof. In some embodiments, the phosphorylated flavonol is selected from the group consisting of phosphorylated quercetin, phosphorylated fisetin, phosphorylated galangin, and phosphorylated kaempferol, and combinations thereof. In some embodiments, the phosphorylated flavonol is phosphorylated galangin or a phosphorylated galangin derivative. In some embodiments, the phosphorylated flavonol is phosphorylated kaempferol or a phosphorylated kaempferol derivative. In some embodiments, the phosphorylated flavonol is phosphorylated fisetin or a phosphorylated fisetin derivative. In some embodiments, the phosphorylated flavonol is phosphorylated quercetin or a phosphorylated quercetin derivative.

In some embodiments, the phosphorylated pyrone analog comprises a compound with the structure of Formula XXXV, its pharmaceutically or veterinarily acceptable salts, esters, or prodrugs: wherein R₂₄, R₂₅, R₂₆, R₂₇, R₂₈, R₂₉, R₃₀, R₃₁, R₃₂, and R₃₃ are independently selected from the group of hydrogen, hydroxyl, —OPO₃WY, or —OPO₃Z, wherein W and Y are independently selected from hydrogen, methyl, ethyl, alkyl, carbohydrate, and a cation, Z is a multivalent cation, and wherein at least one of the R₂₄, R₂₅, R₂₆, R₂₇, R₂₈, R₂₉, R₃₀, R₃₁, R₃₂, or R₃₃ is —OPO₃WY, or —OPO₃Z.

In some embodiments, the phosphorylated pyrone analog can have the structure shown below as Formula

XXXVI and its pharmaceutically acceptable salts, esters, prodrugs, analogs, isomers, stereoisomers or tautomers thereof:

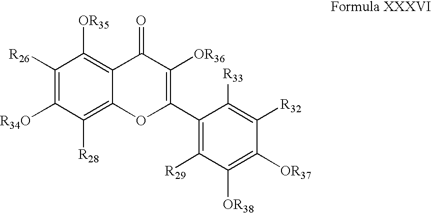

wherein R₂₆, R₂₈, R₂₉, R₃₂, and R₃₃ can be independently selected from the group consisting of hydrogen, C₁-C₁₀ alkyl, aryl, C₁-C₁₀ aliphatic acyl, C₆-C₁₀ aromatic acyl, trialkylsilyl, ether, and carbohydrate;

wherein R₃₄, R₃₅, R₃₆, R₃₇, and R₃₈ can be independently selected from the group consisting of hydrogen, C₁-C₁₀ alkyl, aryl, C₁-C₁₀ aliphatic acyl, C₆-C₁₀ aromatic acyl, trialkylsilyl, ether, carbohydrate; wherein at least one of the R₃₄, R₃₅, R₃₆, R₃₇, or R₃₈ is —PO₃WY, or —PO₃Z, wherein W and Y are independently selected from hydrogen, methyl, ethyl, alkyl, carbohydrate, and a cation, and Z is a multivalent cation.

A useful phosphorylated flavonol is phosphorylated quercetin. Quercetin may be used to illustrate formulations and methods useful in the invention, however, it is understood that the discussion of quercetin applies equally to other phosphorylated pyrone analogs, flavonols, and pyrone analogs useful in the invention, e.g., kaempferol and galangin. The basic structure of quercetin is the structure of Formula XXXVII where R₃₄-R₃₈ are hydrogen. This form of quercetin can also be referred to as quercetin aglycone. Unless otherwise specified the term “quercetin”, as used herein, can also refer to glycosides of quercetin, wherein one or more of the R₃₄-R₃₈ comprise a carbohydrate.

Useful phosphorylated pyrone analogs of the present invention are phosphorylated pyrone analogs of the structure of Formula XXXVII or its pharmaceutically or veterinarily acceptable salts, glycosides, esters, or prodrugs:

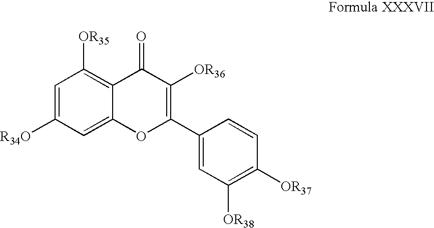

wherein R₃₄, R₃₅, R₃₆, R₃₇, and R₃₈ are independently selected from the group of hydrogen, —PO₃WY, and —PO₃Z, wherein W and Y are independently selected from hydrogen, methyl, ethyl, alkyl, carbohydrate, and a cation, and Z is a multivalent cation; and wherein at least one of the R₃₄-R₃₈ is —PO₃WY, or —PO₃Z.

In some embodiments, the phosphorylated pyrone analog can comprise a cyclic phosphate. In some embodiments, the invention is a composition comprising a compound of Formula XXXVIII or its pharmaceutically or veterinarily acceptable salts, glycosides, esters, or prodrugs:

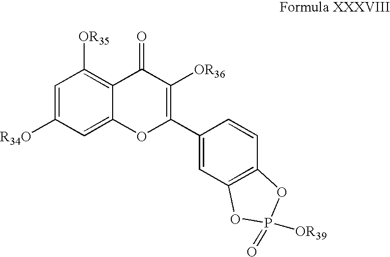

wherein R₃₄, R₃₅, and R₃₆ are independently selected from the group of hydrogen, —PO₃WY, and —PO₃Z, wherein W and Y are independently selected from hydrogen, methyl, ethyl, alkyl, carbohydrate, and a cation, and Z is a multivalent cation; and wherein R₃₉ is selected from the group of hydrogen, methyl, ethyl, alkyl, carbohydrate, and a cation.

A useful phosphorylated pyrone analog comprises a compound of Formula XXXIX, XXXIXa, or its pharmaceutically or veterinarily acceptable salts, glycosides, esters, or prodrugs:

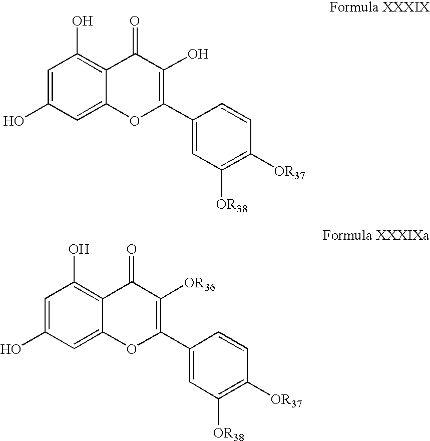

wherein R₃₆, R₃₇ and R₃₈ are independently selected from the group consisting of hydrogen, —PO₃WY, and —PO₃Z, wherein W and Y are independently selected from hydrogen, methyl, ethyl, alkyl, carbohydrate, and a cation, and Z is a multivalent cation; and wherein at least one of the R₃₆, R₃₇ or R₃₈ is —PO₃WY or —PO₃Z.

Some Examples of phosphorylated pyrone analogs are quercetin-3′-O-phosphate and quercetin-4′-O-phosphate. Another useful phosphorylated flavonol is phosphorylated fisetin. Fisetin may be used to illustrate compositions, formulations and methods described herein. However, it is understood that the discussion of fisetin applies equally to other phosphorylated pyrone analogs, flavonols, and pyrone analogs described herein, e.g., kaempferol and galangin. The basic structure of fisetin is the structure of Formula XXXX where R₃₄, R₃₆, R₃₇ and R₃₈ are hydrogen. This form of fisetin can also be referred to as fisetin aglycone. Unless otherwise specified the term “fisetin”, as used hₑreᵢn, can also refer to glycosides of fisetin, wherein one or more of the R₃₄, R₃₆, R₃₇ or R₃₈comprise a carbohydrate.

Useful phosphorylated pyrone analogs of the present invention are phosphorylated pyrone analogs of the structurₑ o_{f} Formula XXXX or its pharmaceutically or veterinarily acceptable salts, glycosides, esters, or prodrugs:

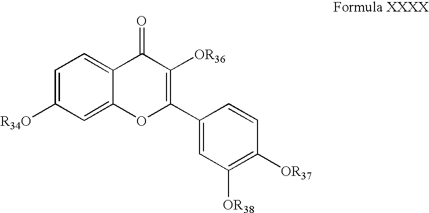

wherein R₃₄, R₃₆, R₃₇, and R₃₈ are independently selected from the group of hydrogen, —PO₃WY, and —PO₃Z, wherein W and Y are independently selected from hydrogen, methyl, ethyl, alkyl, carbohydrate, and a cation, and Z is a multivalent cation, and wherein at least one of the R₃₄, R₃₆, R₃₇, or R₃₈ is —PO₃WY, or —PO₃Z.

In some embodiments, the phosphorylated pyrone analog can comprise a cyclic phosphate. In some embodiments, the invention is a composition comprising a compound of Formula XXXXI or its pharmaceutically or veterinarily acceptable salts, glycosides, esters, or prodrugs:

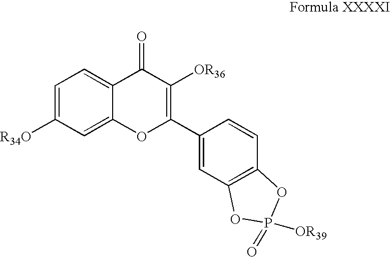

wherein R₃₄ and R₃₆ are independently selected from the group of hydrogen, —PO₃WY, and —PO₃Z, wherein W and Y are independently selected from hydrogen, methyl, ethyl, alkyl, carbohydrate, and a cation, and Z is a multivalent cation; and wherein R₃₉ is selected from the group of hydrogen, methyl, ethyl, alkyl, carbohydrate, and a cation.

A useful phosphorylated pyrone analog comprises a compound of Formula XXXXII, or its pharmaceutically or veterinarily acceptable salts, glycosides, esters, or prodrugs:

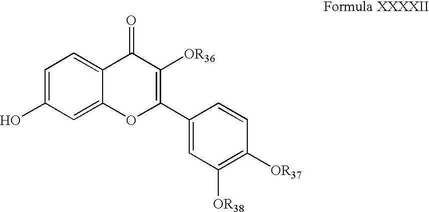

wherein R₃₆, R₃₇ and R₃₈ are independently selected from the group consisting of hydrogen, —PO₃WY, and —PO₃Z, wherein W and Y are independently selected from hydrogen, methyl, ethyl, alkyl, carbohydrate, and a cation, and Z is a multivalent cation; and wherein at least one of the R₃₆, R₃₇, or R₃₈ is —PO₃WY, or —PO₃Z.

Some Examples of phosphorylated pyrone analogs are fisetin-3′-O-phosphate, fisetin-4′-O-phosphate, or fisetin-3-O-phosphate.

In some cases, the level of purity of the compound can affect its performance. In some embodiments the invention comprises quercetin-3′-O-phosphate at a purity of between about 90% and about 99.999%; in some embodiments at a purity of between about 95% and about 99.99%; in some embodiments at a purity of between about 98% and about 99.99%; in some embodiments at a purity of between about 99% and about 99.9%; in some embodiments at a purity of between about 99.5% and about 99.9%; and in some embodiments at a purity of between about 99.8% and about 99.9%. In some embodiments the invention comprises quercetin-3′-O-phosphate at a purity greater than about 90%, 95%, 96%, 97%, 98%. 98.5%, 99%, 99.5%, 99.8%, 99.9%, 99.99%, 99.999% or greater.

In some cases, the level of purity of the compound can affect its performance. In some embodiments the invention comprises quercetin-4′-O-phosphate at a purity of between about 90% and about 99.999%; in some embodiments at a purity of between about 95% and about 99.99%; in some embodiments at a purity of between about 98% and about 99.99%; in some embodiments at a purity of between about 99% and about 99.9%; in some embodiments at a purity of between about 99.5% and about 99.9%; and in some embodiments at a purity of between about 99.8% and about 99.9%. In some embodiments the invention comprises quercetin-4′-O-phosphate at a purity greater than about 90%, 95%, 96%, 97%, 98%. 98.5%, 99%, 99.5%, 99.8%, 99.9%, 99.99%, 99.999% or greater.

In some cases mixtures of quercetin-3′-O-phosphate and quercetin-4′-O-phosphate can be useful in the invention. The invention can comprise mixtures wherein quercetin-3′-O-phosphate is present at about 50% to about 100% and quercetin-4′-O-phosphate is present between about 50% and about 0%. The invention can comprise mixtures wherein quercetin-4′-O-phosphate is present at about 50% to about 100% and quercetin-3′-O-phosphate is present between about 50% and about 0%. In some cases the quercetin-3′-O-phosphate is present at about 80% to about 100% and the quercetin-4′-O-phosphate is present at between about 20% and about 0%. In some cases the quercetin-3′-O-phosphate is present at about 85% to about 100% and the quercetin-4′-O-phosphate is present at between about 15% and about 0%. In some cases the quercetin-3′-O-phosphate is present at about 90% to about 100% and the quercetin-4′-O-phosphate is present at between about 10% and about 0%. In some cases the quercetin-3′-O-phosphate is present at about 95% to about 100% and the quercetin-4′-O-phosphate is present at between about 5% and about 0%. In some cases the quercetin-3′-O-phosphate is present at about 97% to about 100% and the quercetin-4′-O-phosphate is present at between about 3% and about 0%. In some cases the quercetin-3′-O-phosphate is present at about 98% to about 100% and the quercetin-4′-O-phosphate is present at between about 2% and about 0%. In some cases the quercetin-3′-O-phosphate is present at about 99% to about 100% and the quercetin-4′-O-phosphate is present at between about 1% and about 0%.

In some cases, the level of purity of the compound can affect its performance. In some embodiments the invention comprises fisetin-3′-O-phosphate at a purity of between about 90% and about 99.999%; in some embodiments at a purity of between about 95% and about 99.99%; in some embodiments at a purity of between about 98% and about 99.99%; in some embodiments at a purity of between about 99% and about 99.9%; in some embodiments at a purity of between about 99.5% and about 99.9%; and in some embodiments at a purity of between about 99.8% and about 99.9%. In some embodiments the invention comprises fisetin-3′-O-phosphate at a purity greater than about 90%, 95%, 96%, 97%, 98%. 98.5%, 99%, 99.5%, 99.8%, 99.9%, 99.99%, 99.999% or greater.

In some cases, the level of purity of the compound can affect its performance. In some embodiments the invention comprises fisetin-4′-O-phosphate at a purity of between about 90% and about 99.999%; in some embodiments at a purity of between about 95% and about 99.99%; in some embodiments at a purity of between about 98% and about 99.99%; in some embodiments at a purity of between about 99% and about 99.9%; in some embodiments at a purity of between about 99.5% and about 99.9%; and in some embodiments at a purity of between about 99.8% and about 99.9%. In some embodiments the invention comprises fisetin-4′-O-phosphate at a purity greater than about 90%, 95%, 96%, 97%, 98%. 98.5%, 99%, 99.5%, 99.8%, 99.9%, 99.99%, 99.999% or greater.

In some cases, the level of purity of the compound can affect its performance. In some embodiments the invention comprises fisetin-3-O-phosphate at a purity of between about 90% and about 99.999%; in some embodiments at a purity of between about 95% and about 99.99%; in some embodiments at a purity of between about 98% and about 99.99%; in some embodiments at a purity of between about 99% and about 99.9%; in some embodiments at a purity of between about 99.5% and about 99.9%; and in some embodiments at a purity of between about 99.8% and about 99.9%. In some embodiments the invention comprises fisetin-3-O-phosphate at a purity greater than about 90%, 95%, 96%, 97%, 98%. 98.5%, 99%, 99.5%, 99.8%, 99.9%, 99.99%, 99.999% or greater.

In some cases mixtures of fisetin-3′-O-phosphate and fisetin-4′-O-phosphate can be useful in the invention. The invention can comprise mixtures wherein fisetin-3′-O-phosphate is present at about 50% to about 100% and fisetin-4′-O-phosphate is present between about 50% and about 0%. The invention can comprise mixtures wherein fisetin-4′-O-phosphate is present at about 50% to about 100% and fisetin-3′-O-phosphate is present between about 50% and about 0%. In some cases the fisetin-3′-O-phosphate is present at about 80% to about 100% and the fisetin-4′-O-phosphate is present at between about 20% and about 0%. In some cases the fisetin-3′-O-phosphate is present at about 85% to about 100% and the fisetin-4′-O-phosphate is present at between about 15% and about 0%. In some cases the fisetin-3′-O-phosphate is present at about 90% to about 100% and the fisetin-4′-O-phosphate is present at between about 10% and about 0%. In some cases the fisetin-3′-O-phosphate is present at about 95% to about 100% and the fisetin-4′-O-phosphate is present at between about 5% and about 0%. In some cases the fisetin-3′-O-phosphate is present at about 97% to about 100% and the fisetin-4′-O-phosphate is present at between about 3% and about 0%. In some cases the fisetin-3′-O-phosphate is present at about 98% to about 100% and the fisetin-4′-O-phosphate is present at between about 2% and about 0%. In some cases the fisetin-3′-O-phosphate is present at about 99% to about 100% and the fisetin-4′-O-phosphate is present at between about 1% and about 0%.

In some embodiments, the phosphorylated quercetin is in a carbohydrate-derivatized form, e.g., a phosphorylated quercetin-O-saccharide. Phosphorylated quercetin-O-saccharides useful in the invention include, but are not limited to, phosphorylated quercetin 3-O-glycoside, phosphorylated quercetin 3-O-glucorhamnoside, phosphorylated quercetin 3-O-galactoside, phosphorylated quercetin 3-O-xyloside, and phosphorylated quercetin 3-O-rhamnoside. In some embodiments, the invention utilizes a phosphorylated quercetin 7-O-saccharide. The phosphorylated quercetin-O-saccharide may be phosphorylated on the hydroxyl positions directly attached to quercetin, or it may be phosphorylated on hydroxyl positions of the carbohydrate.

In some embodiments, the phosphorylated fisetin is in a carbohydrate-derivatized form, e.g., a phosphorylated fisetin-O-saccharide. Phosphorylated fisetin-O-saccharides useful in the invention include, but are not limited to, phosphorylated fisetin 3-O-glycoside, phosphorylated fisetin 3-O-glucorhamnoside, phosphorylated fisetin 3-O-galactoside, phosphorylated fisetin 3-O-xyloside, and phosphorylated fisetin 3-O-rhamnoside. In some embodiments, the invention utilizes a phosphorylated fisetin 7-O-saccharide. The phosphorylated fisetin-O-saccharide may be phosphorylated on the hydroxyl positions directly attached to fisetin, or it may be phosphorylated on hydroxyl positions of the carbohydrate.

B. Phosphonated Pyrone Analogs

In some embodiments, the invention utilizes a phosphonated flavonol. In some embodiments, the phosphonated flavonol is selected from the group consisting of phosphonated quercetin, phosphonated fisetin, phosphonated morin, phosphonated rutin, phosphonated myricetin, phosphonated galangin, phosphonated and phosphonated kaempherol, and a combination thereof. In some embodiments, the phosphonated flavonol is selected from the group consisting of phosphonated quercetin, phosphonated fisetin, phosphonated 5,7-dideoxyquercetin, phosphonated galangin, and phosphonated kaempherol, and a combination thereof. In some embodiments, the phosphonated flavonol is phosphonated quercetin. In some embodiments, the phosphonated flavonol is phosphonated galangin. In some embodiments, the phosphonated flavonol is phosphonated kaempherol. In some embodiments, the phosphonated flavonol is phosphonated fisetin. In some embodiments, the phosphonated phosphonated pyrone arialog is O-alkylphosphonated phosphonated pyrone analog. In some embodiments, the phosphonated flavonol is phosphonated 5,7-dideoxyquercetin. In some embodiments, the phosphonated flavonol is quercetin-3′-O-methylphosphonate. In some embodiments, the phosphonated flavonol is quercetin-4′-O-methylphosphonate. In some embodiments, the phosphonated flavonol is quercetin-3-O-methylphosphonate. In some embodiments, the phosphonated flavonol is fisetin-3′-O-methylphosphonate. In some embodiments, the phosphonated flavonol is fisetin-4′-O-methylphosphonate. In some embodiments, the phosphonated flavonol is fisetin-3-O-methylphosphonate.

In some embodiments, the phosphonated pyrone analog comprises a compound with the structure of Formula XXXV, its pharmaceutically or veterinarily acceptable salts, esters, or prodrugs: wherein R₂₄, R₂₅, R₂₆, R₂₇, R₂₈, R₂₉, R₃₀, R₃₁, R₃₂, and R₃₃ are independently selected from the group of hydrogen, halogen, hydroxyl, —OLPO₃WY, or —OLPO₃Z, wherein W and Y are independently selected from hydrogen, methyl, ethyl, alkyl, carbohydrate, and a cation, L is methyl, ethyl, alkyl or a carbohydate, Z is a multivalent cation, and wherein at least one of R₂₄, R₂₅, R₂₆, R₂₇, R₂₈, R₂₉, R₃₀, R₃₁, R₃₂, and R₃₃ is —OLPO₃WY, or —OLPO₃Z.

In some embodiments, the phosphonated pyrone analog can have the structure shown below as Formula XXXVI-i and its pharmaceutically acceptable salts, esters, prodrugs, analogs, isomers, stereoisomers or tautomers thereof:

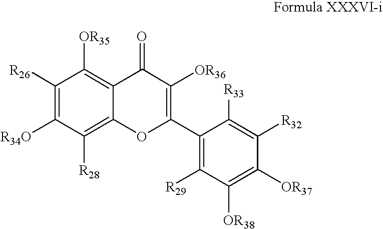

wherein R₂₆, R₂₈, R₂₉, R₃₂, and R₃₃ can be independently selected from the group consisting of hydrogen, C₁-C₁₀ alkyl, aryl, C₁-C₁₀ aliphatic acyl, C₆-C₁₀ aromatic acyl, trialkylsilyl, ether, and carbohydrate;

wherein R₃₄, R₃₅, R₃₆, R₃₇, and R₃₈ can be independently selected from the group consisting of hydrogen, C₁-C₁₀ alkyl, aryl, C₁-C₁₀ aliphatic acyl, C₆-C₁₀ aromatic acyl, trialkylsilyl, ether, carbohydrate; wherein at least one of the R₃₄, R₃₅, R₃₆, R₃₇, or R₃₈ is -LPO₃WY, or -LPO₃Z, wherein W and Y are independently selected from hydrogen, methyl, ethyl, alkyl, carbohydrate, and a cation, L is methyl, ethyl, alkyl or a carbohydate, and Z is a multivalent cation.

A useful phosphonated flavonol is phosphonated quercetin. Quercetin may be used to illustrate formulations and methods useful in the invention, however, it is understood that the discussion of quercetin applies equally to other phosphorylated pyrone analogs, flavonols, and pyrone analogs useful in the invention, e.g., kaempferol and galangin. The basic structure of quercetin is the structure of Formula XXXVII where R₃₄-R₃₈ are hydrogen. This form of quercetin can also be referred to as quercetin aglycone. Unless otherwise specified the term “quercetin”, as used herein, can also refer to glycosides of quercetin, wherein one or more of the R₃₄-R₃₈ comprise a carbohydrate.

Useful phosphonated pyrone analogs of the present invention are phosphonated pyrone analogs of the structure of Formula XXXVII-i or its pharmaceutically or veterinarily acceptable salts, glycosides, esters, or prodrugs:

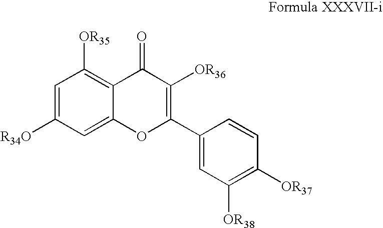

wherein R₃₄, R₃₅, R₃₆, R₃₇, and R₃₈ are independently selected from the group of hydrogen, -LPO₃WY and -LPO₃Z, wherein W and Y are independently selected from hydrogen, methyl, ethyl, alkyl, carbohydrate, and a cation, L is methyl, ethyl, alkyl or a carbohydate, and Z is a multivalent cation; and wherein at least one of the R₃₄-R₃₈ is -LPO₃WY or -LPO₃Z.

In some embodiments, the phosphonated pyrone analog can comprise a cyclic phosphonate. In some embodiments, the invention is a composition comprising a compound of Formula XXXVIIIa-i or Formula XXXVIIIb-i, its pharmaceutically or veterinarily acceptable salts, glycosides, esters, or prodrugs:

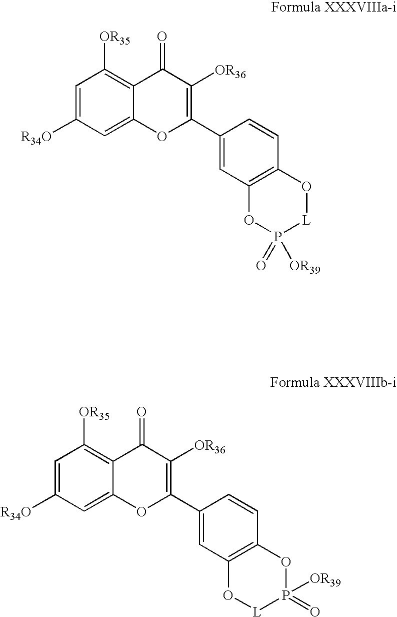

wherein R₃₄, R₃₅, and R₃₆ are each independently selected from the group of hydrogen, -LPO₃WY, and -LPO₃Z, wherein W and Y are independently selected from hydrogen, methyl, ethyl, alkyl, carbohydrate, and a cation, L is alkyl, L is methyl, ethyl, alkyl or a carbohydate, and Z is a multivalent cation; and wherein R₃₉ is selected from the group of hydrogen, methyl, ethyl, alkyl, carbohydrate, and a cation.

A useful phosphonated pyrone analog comprises a compound of Formula XXXIX-i, XXXIXa-i, or its pharmaceutically or veterinarily acceptable salts, glycosides, esters, or prodrugs:

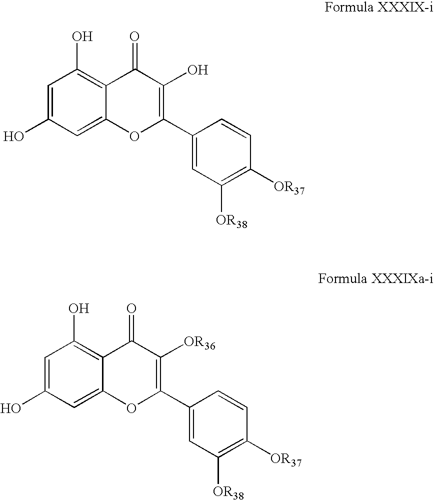

wherein R₃₆, R₃₇ and R₃₈ are independently selected from the group consisting of hydrogen, -LPO₃WY, and -LPO₃Z, wherein W and Y are independently selected from hydrogen, methyl, ethyl, alkyl, carbohydrate, and a cation, L is methyl, ethyl, alkyl or a carbohydate, and Z is a multivalent cation; and wherein at least one of the R₃₆, R₃₇ or R₃₈ is -LPO₃WY or -LPO₃Z. Thus, the compounds quercetin-3′-O-methylphosphonate, quercetin-4′-O-methylphosphonate, or quercetin-3-O-methylphosphonate can be useful in the invention.

Another useful phosphonated flavonol is phosphonated fisetin. Fisetin may be used to illustrate compositions, formulations and methods described herein. However, it is understood that the discussion of fisetin applies equally to other phosphorylated pyrone analogs, flavonols, and pyrone analogs described herein, e.g., kaempferol and galangin. The basic structure of fisetin is the structure of Formula XXXX where R₃₄, R₃₆, R₃₇ and R₃₈ are hydrogen. This form of fisetin can also be referred to as fisetin aglycone. Unless otherwise specified the term “fisetin”, as used herein can also refer to glycosides of fisetin, wherein one or more of the R₃₄, R₃₆, R₃₇ or R₃₈ comprise a carbohydrate.

Useful phosphonated pyrone analogs of the present invention are phosphonated pyrone analogs of the structure of Formula XXXX-i or its pharmaceutically or veterinarily acceptable salts, glycosides, esters, or prodrugs:

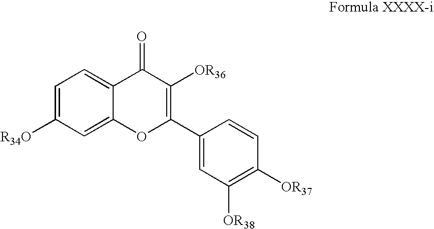

wherein R₃₄, R₃₆, R₃₇, and R₃₈ are independently selected from the group of hydrogen, -LPO₃WY and -LPO₃Z, wherein W and Y are independently selected from hydrogen, methyl, ethyl, alkyl, carbohydrate, and a cation, L is methyl, ethyl, alkyl or a carbohydate, and Z is a multivalent cation, and wherein at least one of the R₃₄, R₃₆, R₃₇, or R₃₈ is -LPO₃WY or -LPO₃Z.

In some embodiments of the invention, the phosphonated pyrone analog can comprise a cyclic phosphonate. In some embodiments, the invention is a composition comprising a compound of Formula XXXXIa-i or Formula XXXXIb-i, its pharmaceutically or veterinarily acceptable salts, glycosides, esters, or prodrugs:

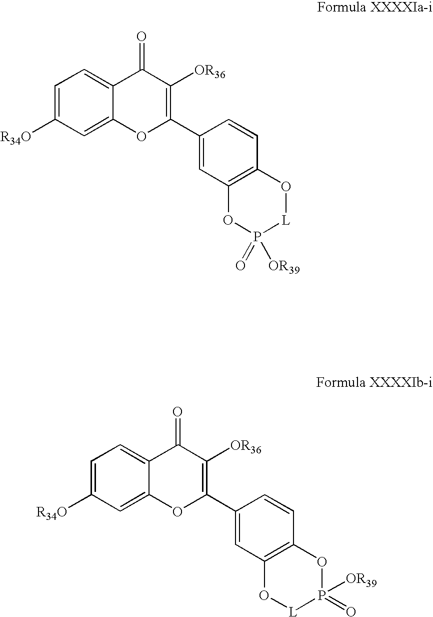

wherein R₃₄ and R₃₆ are independently selected from the group of hydrogen, -LPO₃WY, and -LPO₃Z, wherein W and Y are independently selected from hydrogen, methyl, ethyl, alkyl, carbohydrate, and a cation, L is methyl, ethyl, alkyl or a carbohydate, and Z is a multivalent cation; and wherein R₃₉ is selected from the group of hydrogen, methyl, ethyl, alkyl, carbohydrate, and a cation.

A useful phosphonated pyrone analog comprises a compound of Formula XXXXII-i, or its pharmaceutically or veterinarily acceptable salts, glycosides, esters, or prodrugs:

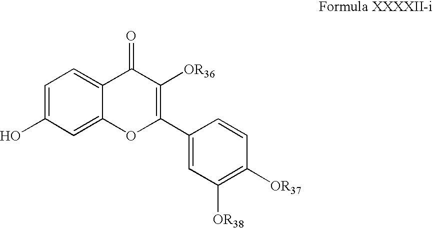

wherein R₃₆, R₃₇ and R₃₈ are independently selected from the group consisting of hydrogen, -LPO₃WY, and -LPO₃Z, wherein W and Y are independently selected from hydrogen, methyl, ethyl, alkyl, carbohydrate, and a cation, L is methyl, ethyl, alkyl or a carbohydate, and Z is a multivalent cation; and wherein at least one of the R₃₆, R₃₇, or R₃₈ is -LPO₃WY or -LPO₃Z.

In one embodiment, the phosphonated pyrone analog is a compound of the following Formula XXXXIII-i

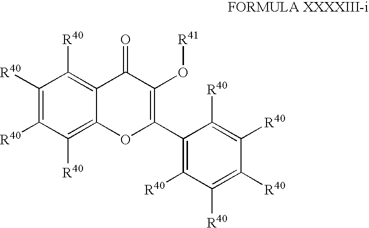

or a pharmaceutically acceptable salt thereof, wherein:

each R⁴⁰ is independently —H, —OH, or —O—(C₁-C₁₀ alkyl)-P(O)(OR⁴²)₂;

R⁴¹ is —H or —(C₁-C₁₀ alkyl)-P(O)(OR⁴²)₂;

each R⁴² is independently —H or —(C₁-C₁₀ alkyl); and

at least one R⁴⁰ is —O—(C₁-C₁₀ alkyl)-P(O)(OR⁴²)₂ or R⁴¹ is —(C₁-C₁₀ alkyl)-P(O)(OR⁴²)₂.

In some embodiments of the compound of Formula XXXXIII-i, each R⁴⁰ is independently —H or —OH. In some embodiments of the compound of Formula XXXXIII-i, at least one R⁴⁰ is —OH. In some embodiments of the compound of Formula XXXXIII-i, R⁴¹ is —(C₁-C₁₀ alkyl)-P(O)(OR⁴²)₂, and R⁴² is H or —CH₂CH₃.

Illustrative Examples of phosphonated pyrone analogs are depicted in the Table below:

| | |
|---|---|
| 1 | 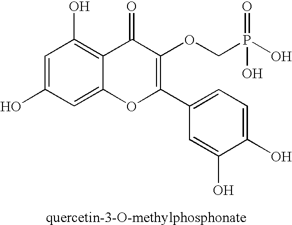 |
| 2 | 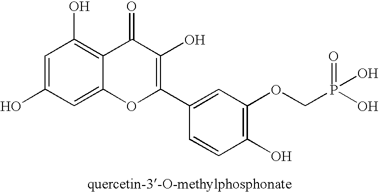 |
| 3 | 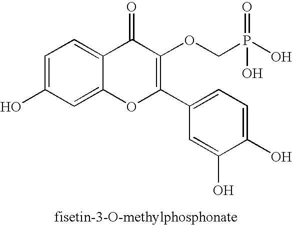 |
| 4 | 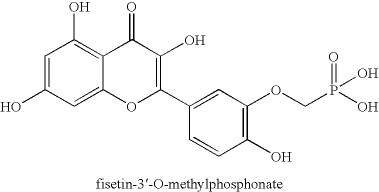 |
| 5 | 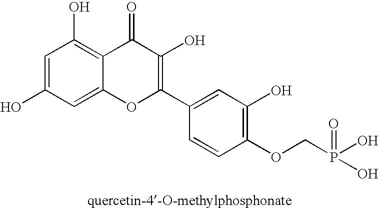 |
| 6 | 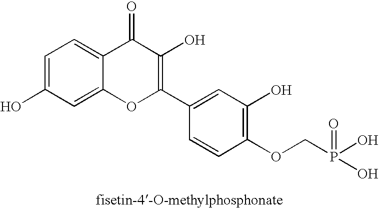 |
| 7 | 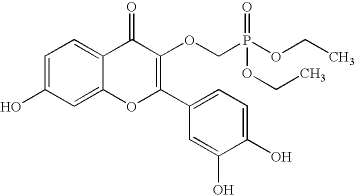 |
| 8 | 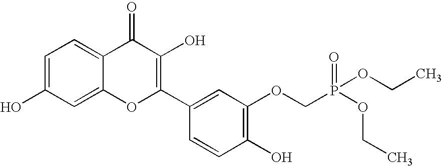 |
| 9 | 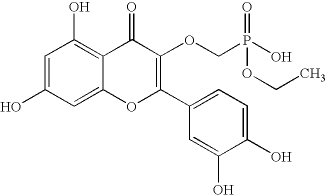 |
| 10 | 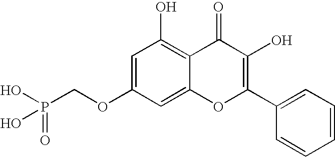 |
| 11 | 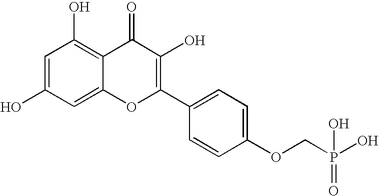 |
| 12 | 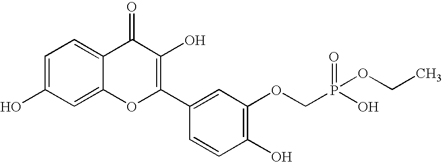 |
| 13 | 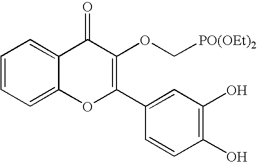 |
| 14 | 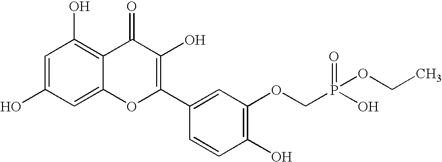 |
| 15 | 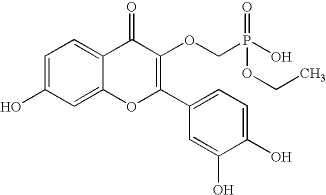 |
| 16 | 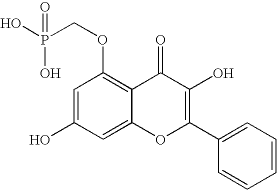 |
| 17 | 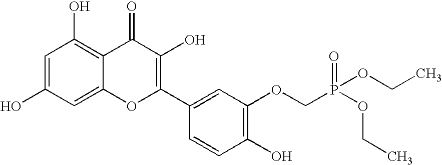 |
| 18 | 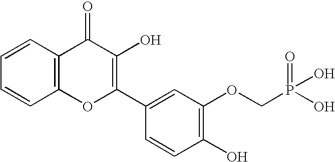 |
| 19 | 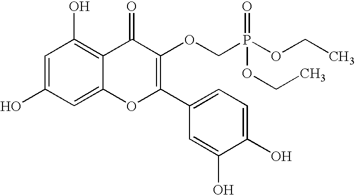 |
| 20 | 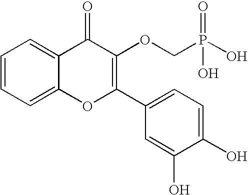 |
| 21 | 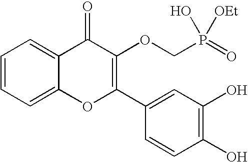 |

Some Examples of phosphonated pyrone analogs are fisetin-3′-O-phosphonate, fisetin-4′-O-phosphonate, or fisetin-3-O-phosphonate.

In some cases, the level of purity of the compound can affect its performance. In some embodiments the invention comprises quercetin-3′-O-methylphosphonate at a purity of between about 90% and about 99.999%; in some embodiments at a purity of between about 95% and about 99.99%; in some embodiments at a purity of between about 98% and about 99.99%; in some embodiments at a purity of between about 99% and about 99.9%; in some embodiments at a purity of between about 99.5% and about 99.9%; and in some embodiments at a purity of between about 99.8% and about 99.9%. In some embodiments the invention comprises quercetin-3′-O-methylphosphonate at a purity greater than about 90%, 95%, 96%, 97%, 98%. 98.5%, 99%, 99.5%, 99.8%, 99.9%, 99.99%, 99.999% or greater.

In some cases, the level of purity of the compound can affect its performance. In some embodiments the invention comprises quercetin-4′-O-methylphosphonate at a purity of between about 90% and about 99.999%; in some embodiments at a purity of between about 95% and about 99.99%; in some embodiments at a purity of between about 98% and about 99.99%; in some embodiments at a purity of between about 99% and about 99.9%; in some embodiments at a purity of between about 99.5% and about 99.9%; and in some embodiments at a purity of between about 99.8% and about 99.9%. In some embodiments the invention comprises quercetin-4′-O-methylphosphonate at a purity greater than about 90%, 95%, 96%, 97%, 98%. 98.5%, 99%, 99.5%, 99.8%, 99.9%, 99.99%, 99.999% or greater.

In some cases, the level of purity of the compound can affect its performance. In some embodiments the invention comprises quercetin-3-O-methylphosphonate at a purity of between about 90% and about 99.999%; in some embodiments at a purity of between about 95% and about 99.99%; in some embodiments at a purity of between about 98% and about 99.99%; in some embodiments at a purity of between about 99% and about 99.9%; in some embodiments at a purity of between about 99.5% and about 99.9%; and in some embodiments at a purity of between about 99.8% and about 99.9%. In some embodiments the invention comprises quercetin-3-O-methylphosphonate at a purity greater than about 90%, 95%, 96%, 97%, 98%. 98.5%, 99%, 99.5%, 99.8%, 99.9%, 99.99%, 99.999% or greater.

In some cases mixtures of quercetin-3′-O-methylphosphonate and quercetin-4′-O-methylphosphonate can be useful in the invention. The invention can comprise mixtures wherein quercetin-3′-O-methylphosphonate is present at about 50% to about 100% and quercetin-4′-O-methylphosphonate is present between about 50% and about 0%. The invention can comprise mixtures wherein quercetin-4′-O-methylphosphonate is present at about 50% to about 100% and quercetin-3′-O-methylphosphonate is present between about 50% and about 0%. In some cases the quercetin-3′-O-methylphosphonate is present at about 80% to about 100% and the quercetin-4′-O-methylphosphonate is present at between about 20% and about 0%. In some cases the quercetin-3′-O-methylphosphonate is present at about 85% to about 100% and the quercetin-4′-O-methylphosphonate is present at between about 15% and about 0%. In some cases the quercetin-3′-O-methylphosphonate is present at about 90% to about 100% and the quercetin-4′-O-methylphosphonate is present at between about 10% and about 0%. In some cases the quercetin-3′-O-methylphosphonate is present at about 95% to about 100% and the quercetin-4′-O-methylphosphonate is present at between about 5% and about 0%. In some cases the quercetin-3′-O-methylphosphonate is present at about 97% to about 100% and the quercetin-4′-O-methylphosphonate is present at between about 3% and about 0%. In some cases the quercetin-3′-O-methylphosphonate is present at about 98% to about 100% and the quercetin-4′-O-methylphosphonate is present at between about 2% and about 0%. In some cases the quercetin-3′-O-methylphosphonate is present at about 99% to about 100% and the quercetin-4′-O-methylphosphonate is present at between about 1% and about 0%.

In some cases, the level of purity of the compound can affect its performance. In some embodiments the invention comprises fisetin-3′-O-methylphosphonate at a purity of between about 90% and about 99.999%; in some embodiments at a purity of between about 95% and about 99.99%; in some embodiments at a purity of between about 98% and about 99.99%; in some embodiments at a purity of between about 99% and about 99.9%; in some embodiments at a purity of between about 99.5% and about 99.9%; and in some embodiments at a purity of between about 99.8% and about 99.9%. In some embodiments the invention comprises fisetin-3′-O-methylphosphonate at a purity greater than about 90%, 95%, 96%, 97%, 98%. 98.5%, 99%, 99.5%, 99.8%, 99.9%, 99.99%, 99.999% or greater.

In some cases, the level of purity of the compound can affect its performance. In some embodiments the invention comprises fisetin-4′-O-methylphosphonate at a purity of between about 90% and about 99.999%; in some embodiments at a purity of between about 95% and about 99.99%; in some embodiments at a purity of between about 98% and about 99.99%; in some embodiments at a purity of between about 99% and about 99.9%; in some embodiments at a purity of between about 99.5% and about 99.9%; and in some embodiments at a purity of between about 99.8% and about 99.9%. In some embodiments the invention comprises fisetin-4′-O-methylphosphonate at a purity greater than about 90%, 95%, 96%, 97%, 98%. 98.5%, 99%, 99.5%, 99.8%, 99.9%, 99.99%, 99.999% or greater.

In some cases, the level of purity of the compound can affect its performance. In some embodiments the invention comprises fisetin-3-O-methylphosphonate at a purity of between about 90% and about 99.999%; in some embodiments at a purity of between about 95% and about 99.99%; in some embodiments at a purity of between about 98% and about 99.99%; in some embodiments at a purity of between about 99% and about 99.9%; in some embodiments at a purity of between about 99.5% and about 99.9%; and in some embodiments at a purity of between about 99.8% and about 99.9%. In some embodiments the invention comprises fisetin-3-O-methylphosphonate at a purity greater than about 90%, 95%, 96%, 97%, 98%. 98.5%, 99%, 99.5%, 99.8%, 99.9%, 99.99%, 99.999% or greater.

In some cases mixtures of fisetin-3′-O-methylphosphonate and fisetin-4′-O-methylphosphonate can be useful in the invention. The invention can comprise mixtures wherein fisetin-3′-O-methylphosphonate is present at about 50% to about 100% and fisetin-4′-O-methylphosphonate is present between about 50% and about 0%. The invention can comprise mixtures wherein fisetin-4′-O-methylphosphonate is present at about 50% to about 100% and fisetin-3′-O-methylphosphonate is present between about 50% and about 0%. In some cases the fisetin-3′-O-methylphosphonate is present at about 80% to about 100% and the fisetin-4′-O-methylphosphonate is present at between about 20% and about 0%. In some cases the fisetin-3′-O-methylphosphonate is present at about 85% to about 100% and the fisetin-4′-O-methylphosphonate is present at between about 15% and about 0%. In some cases the fisetin-3′-O-methylphosphonate is present at about 90% to about 100% and the fisetin-4′-O-methylphosphonate is present at between about 10% and about 0%. In some cases the fisetin-3′-O-methylphosphonate is present at about 95% to about 100% and the fisetin-4′-O-methylphosphonate is present at between about 5% and about 0%. In some cases the fisetin-3′-O-methylphosphonate is present at about 97% to about 100% and the fisetin-4′-O-methylphosphonate is present at between about 3% and about 0%. In some cases the fisetin-3′-O-methylphosphonate is present at about 98% to about 100% and the fisetin-4′-O-methylphosphonate is present at between about 2% and about 0%. In some cases the fisetin-3′-O-methylphosphonate is present at about 99% to about 100% and the fisetin-4′-O-methylphosphonate is present at between about 1% and about 0%.

In some embodiments, the phosphonated quercetin is in a carbohydrate-derivatized form, e.g., a phosphonated quercetin-O-saccharide. Phosphonated quercetin-O-saccharides useful in the invention include, but are not limited to, phosphonated quercetin 3-O-glycoside, phosphonated quercetin 3-O-glucorhamnoside, phosphonated quercetin 3-O-galactoside, phosphonated quercetin 3-O-xyloside, and phosphonated quercetin 3-O-rhamnoside. In some embodiments, the invention utilizes a phosphonated quercetin 7-O-saccharide. The phosphonated quercetin-O-saccharide may be phosphonated on the hydroxyl positions directly attached to quercetin, or it may be phosphonated on hydroxyl positions of the carbohydrate.

In some embodiments, the phosphonated fisetin is in a carbohydrate-derivatized form, e.g., a phosphonated fisetin-O-saccharide. Phosphonated fisetin-O-saccharides useful in the invention include, but are not limited to, phosphonated fisetin 3-O-glycoside, phosphonated fisetin 3-O-glucorhamnoside, phosphonated fisetin 3-O-galactoside, phosphonated fisetin 3-O-xyloside, and phosphonated fisetin 3-O-rhamnoside. In some embodiments, the invention utilizes a phosphonated fisetin 7-O-saccharide. The phosphonated fisetin-O-saccharide may be phosphonated on the hydroxyl positions directly attached to fisetin, or it may be phosphonated on hydroxyl positions of the carbohydrate.

The term “pharmaceutically acceptable cation” as used herein refers to a positively charged inorganic or organic ion that is generally considered suitable for human consumption. Examples of pharmaceutically acceptable cations are hydrogen, alkali metal (lithium, sodium and potassium), magnesium, calcium, ferrous, ferric, ammonium, alkylammonium, dialkylammonium, trialkylammonium, tetraalkylammonium, and guanidinium ions and protonated forms of lysine, choline and procaine.

The compounds presented herein may possess one or more chiral centers and each center may exist in the R or S configuration. The compounds presented herein include all diastereomeric, enantiomeric, and epimeric forms as well as the appropriate mixtures thereof. Stereoisomers may be obtained, if desired, by methods known in the art as, for example, the separation of stereoisomers by chiral chromatographic columns.

The methods and formulations described herein include the use of N-oxides, crystalline forms (also known as polymorphs), or pharmaceutically acceptable salts of compounds having the structure of Formula I, as well as active metabolites of these compounds having the same type of activity. In addition, the compounds described herein can exist in unsolvated as well as solvated forms with pharmaceutically acceptable solvents such as water, ethanol, and the like. The solvated forms of the compounds presented herein are also considered to be disclosed herein.

### II. Pharmaceutical Compositions, Formulations, and Dosages

Pharmaceutical compositions may also be prepared from compounds described herein and one or more pharmaceutically acceptable excipients suitable for rectal, buccal, sublingual, intranasal, transdermal, intravenous, intraperitoneal, parenteral, intramuscular, subcutaneous, oral, or topical administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety.

In some embodiments the composition is a solid formulation. In some embodiments the composition is a dry powder formulation. In some embodiments the composition is a liquid formulation.

In some embodiments, a compound described herein is administered with an excipient to increase the solubility of the compound. In some embodiments, the excipient is an oligosaccharide. In other embodiments, the excipient is a cyclic oligosaccharide, such as cyclodextrin. In some embodiments, the excipient is a sulfo-alkyl ether substituted cyclodextrin, or a sulfobutyl-ether substituted cyclodextrin. In some embodiments, the excipient is hydroxypropyl-β-cyclodextrin, hydroxypropyl-γ-cyclodextrin, sulfobutylether-β-cyclodextrin, sulfobutylether-7-β-cyclodextrin, or a combination thereof. In some embodiments, the excipient is Captisol®.

In some embodiments, the pharmaceutical composition comprises a flavonoid, a cyclodextrin, a basic amino acid or a sugar-amine and a pharmaceutically or veterinarily acceptable carrier. In some embodiments the basic amino acid is arginine. In some embodiments the basic amino acid is lysine. In some embodiments the sugar-amine is meglumine.

In some embodiments the flavonoid is fisetin, fisetin derivative, quercetin or quercetin derivative. In some embodiments the flavonoid is phosphorylated or phosphonated fisetin, phosphorylated or phosphonated fisetin derivative, phosphorylated or phosphonated quercetin or phosphorylated or phosphonated quercetin derivative.

In some embodiments, fisetin or phosphorylated or phosphonated fisetin is in a carbohydrate-derivatized form, e.g., a phosphorylated or phosphonated fisetin-O-saccharide. Phosphorylated or phosphonated fisetin-O-saccharides include, but are not limited to, phosphorylated or phosphonated fisetin 3-O-glycoside, phosphorylated or phosphonated fisetin 3-O-glucorhamnoside, phosphorylated or phosphonated fisetin 3-O-galactoside, phosphorylated or phosphonated fisetin 3-O-xyloside, phosphorylated or phosphonated fisetin 3-O-rhamnoside, and phosphorylated or phosphonated fisetin 7-O-saccharide.

In some embodiments, quercetin or phosphorylated or phosphonated quercetin is in a carbohydrate-derivatized form, e.g., a phosphorylated or phosphonated quercetin-O-saccharide. Phosphorylated or phosphonated quercetin-O-saccharides include, but are not limited to, phosphorylated or phosphonated quercetin 3-O-glycoside, phosphorylated or phosphonated quercetin 3-O-glucorhamnoside, phosphorylated or phosphonated quercetin 3-O-galactoside, phosphorylated or phosphonated quercetin 3-O-xyloside, phosphorylated or phosphonated quercetin 3-O-rhamnoside, and phosphorylated or phosphonated quercetin 7-O-saccharide.

In some embodiments, the compound is a phosphorylated or phosphonated fisetin aglycone or a phosphorylated or phosphonated quercetin aglycone. In some embodiments, a combination of aglycone and carbohydrate-derivatized phosphorylated or phosphonated fisetin can be used. In some embodiments, a combination of aglycone and carbohydrate-derivatized phosphorylated or phosphonated quercetin can be used. It will be appreciated that the various forms of phosphorylated or phosphonated fisetin or various forms of phosphorylated or phosphonated quercetin may have different properties useful in the compositions and methods described herein, and that the route of administration can determine the choice of forms, or combinations of forms, used in the composition or method. Choice of a single form, or of combinations, may be determined empirically.

In some embodiments, fisetin or a phosphorylated or phosphonated fisetin derivative, or quercetin or a phosphorylated or phosphonated quercetin derivative, is provided in a form for oral consumption. In some embodiments, phosphorylated or phosphonated fisetin-3-O-glycoside is used in an oral preparation. In some embodiments, phosphorylated or phosphonated fisetin 3-O-glucorhamnoside is used in an oral preparation of phosphorylated or phosphonated fisetin. In some embodiments, a combination of phosphorylated or phosphonated fisetin-3-O-glycoside and phosphorylated or phosphonated fisetin 3-O-glucorhamnoside is used in an oral preparation. Other carbohydrate-derivatized forms of phosphorylated or phosphonated fisetin, or other forms of phosphorylated or phosphonated fisetin which are derivatives as described above, can also be used based on their oral bioavailability, their metabolism, their incidence of gastrointestinal or other side effects, and other factors known in the art. In some embodiments, phosphorylated or phosphonated quercetin-3-O-glycoside is used in an oral preparation. In some embodiments, phosphorylated or phosphonated quercetin 3-O-glucorhamnoside is used in an oral preparation of phosphorylated or phosphonated quercetin. In some embodiments, a combination of phosphorylated or phosphonated quercetin-3-O-glycoside and phosphorylated or phosphonated quercetin 3-O-glucorhamnoside is used in an oral preparation. Other carbohydrate-derivatized forms of phosphorylated or phosphonated quercetin, or other forms of phosphorylated or phosphonated quercetin which are derivatives as described above, can also be used based on their oral bioavailability, their metabolism, their incidence of gastrointestinal or other side effects, and other factors known in the art. Determining the bioavailability of phosphorylated or phosphonated fisetin or phosphorylated or phosphonated quercetin in the form of their corresponding derivatives including aglycones and glycosides may be determined empirically. See, e.g., Graefe et al., J. Clin. Pharmacol. (2001) 451:492-499; Arts et al. (2004) Brit. J. Nutr. 91:841-847; Moon et al. (2001) Free Rad. Biol. Med. 30:1274-1285; Hollman et al. (1995) Am. J. Clin. Nutr. 62:1276-1282; Jenaelle et al. (2005) Nutr. J. 4:1, and Cermak et al. (2003) J. Nutr. 133: 2802-2807, all of which are incorporated by reference herein in their entirety.

In some embodiments, administration is rectal, buccal, sublingual, intranasal, transdermal, intravenous, intraperitoneal, parenteral, intramuscular, subcutaneous, oral, topical, as an inhalant, or via an impregnated or coated device such as a stent. In some embodiments the administration is intravenous. In some embodiments administration is transdermal. In other embodiments the administration is oral.

A pharmaceutically acceptable excipient may also be included.

In some embodiments, the lipid transport protein modulator comprises a phosphorylated or phosphonated pyrone analog. A phosphorylated or phosphonated pyrone analog can be phosphorylated or phosphonated fisetin, phosphorylated or phosphonated isofisetin, phosphorylated or phosphonated flavon, phosphorylated or phosphonated chrysin, phosphorylated or phosphonated apigenin, phosphorylated or phosphonated rhoifolin, phosphorylated or phosphonated diosmin, phosphorylated or phosphonated galangin, phosphorylated or phosphonated morin, phosphorylated or phosphonated rutin, phosphorylated or phosphonated kaempferol, phosphorylated or phosphonated myricetin, phosphorylated or phosphonated taxifolin, phosphorylated or phosphonated naringenin, phosphorylated or phosphonated naringin, phosphorylated or phosphonated hesperetin, phosphorylated or phosphonated hesperidin, phosphorylated or phosphonated chalcone, phosphorylated or phosphonated phloretin, phosphorylated or phosphonated phlorizdin, phosphorylated or phosphonated genistein, phosphorylated or phosphonated biochanin A, phosphorylated or phosphonated catechin, and phosphorylated or phosphonated epicatechin, or a combination thereof. In some embodiments a phosphorylated or phosphonated pyrone analog can be phosphorylated or phosphonated fisetin, phosphorylated or phosphonated quercetin, or a combination thereof.

In some embodiments, the symptom of hyperglycemia, hyperlipidemia, hypercholesterolemia, or hypertriglyceridemia that is reduced upon administration of the phosphorylated or phosphonated pyrone analog includes, but are not limited to, xanthoma, skin lesion, pancreatitis, enlargement of liver and spleen, chest pain, heart attack or a combination thereof.

In some embodiments, the symptom of hyperglycemia that is reduced includes, but is not limited to, glucosuria, polyphagia, polyuria, polydipsia, loss of consciousness, blurred vision, headaches, coma, ketoacidosis, decrease in blood volume, decrease in renal blood flow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In some embodiments, the symptom of hyperglycemia is glucosuria.

In some embodiments, the phosphorylated or phosphonated pyrone analog is present in an amount sufficient to exert a therapeutic effect and decrease hyperlipidemia, hypercholesterolemia, hypertriglyceridemia, or hyperglycemia, and/or one or more symptoms thereof, by a measurable amount, compared to no treatment. In some embodiments, the measurable amount is by an average of at least about 5, 10, 15, 20, 25, 30, 35, 40, 45, 50, 55, 60, 65, 70, 75, 80, 85, 90, 95, or more than 95%, compared to no treatment. In some embodiments, the measurable amount is by an average of at least about 5%, about 10%, about 15%, or about 20%, compared to no treatment.

In some embodiments, the phosphorylated or phosphonated pyrone analog is present in an amount sufficient to exert a therapeutic effect and decrease hyperlipidemia, hypercholesterolemia, hypertriglyceridemia, or hyperglycemia, and/or one or more symptoms thereof, by a measurable amount, compared to treatment without the lipid transport protein modulator, i.e. a phosphorylated or phosphonated pyrone analog, when the composition is administered to an animal. In some embodiments, the measurable amount is by an average of at least about 5, 10, 15, 20, 25, 30, 35, 40, 45, 50, 55, 60, 65, 70, 75, 80, 85, 90, 95, or more than 95%, compared to treatment without the phosphorylated or phosphonated pyrone analog. In some embodiments, the measurable amount is by an average of at least about 5%, about 10%, about 15%, or about 20%, compared to that without the phosphorylated or phosphonated pyrone analog.

“Substantially eliminated” as used herein encompasses no measurable or no statistically significant symptom (one or more symptoms) of hyperlipidemia, hypercholesterolemia, hypertriglyceridemia, or hyperglycemia as disclosed herein. In some embodiments the phosphorylated or phosphonated pyrone analog is phosphorylated or phosphonated fisetin. In some embodiments the phosphorylated or phosphonated pyrone analog is phosphorylated or phosphonated fisetin derivative. In some embodiments the phosphorylated or phosphonated pyrone analog is phosphorylated or phosphonated quercetin. In some embodiments the phosphorylated or phosphonated pyrone analog is phosphorylated or phosphonated quercetin derivative.

The amount of one or more phosphorylated or phosphonated pyrone analogs for use in such compositions may be equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

Alternatively, the amount of one or more phosphorylated or phosphonated pyrone analogs for use in such compositions may be more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The amount of one or more of the phosphorylated or phosphonated pyrone analogs for use in such compositions may be in the range of 0.0001-10 g, 0.0005-9 g, 0.001-8 g, 0.005-7 g, 0.01-6 g, 0.05-5 g, 0.1-4 g, 0.5-4 g, or 1-3 g.

The amount of one or more of the phosphorylated or phosphonated pyrone analogs for use in such compositions may be in the range of about 1-1000 mg, about 10-1000 mg, about 50-1000 mg, about 100-1000 mg, about 1-500 mg, about 5-500 mg, about 50-500 mg, about 100-500 mg, about 200-1000 mg, about 200-800 mg, or about 200-700 mg. one or more phosphorylated or phosphonated pyrone analogs may present in an amount of about 10 mg, about 25 mg, about 50 mg, about 100 mg, about 200 mg, about 250 mg, about 300 mg, about 400 mg, about 500 mg, about 600 mg, about 700 mg, about 800 mg, about 900 mg, or about 1000 mg. In some embodiments, the compositions disclosed herein further include a pharmaceutical excipient. The composition may include phosphorylated or phosphonated fisetin, a phosphorylated or phosphonated fisetin derivative, phosphorylated or phosphonated quercetin, or a phosphorylated or phosphonated quercetin derivative.

More than one phosphorylated or phosphonated pyrone analog may be formulated in a composition for administration to a subject. The phosphorylated or phosphonated pyrone analog may be any compound within the phosphorylated or phosphonated pyrone family having the formula as described herein. The phosphorylated or phosphonated pyrone analogs in a combination (mixture) may be administered to a subject simultaneously (e.g., same or different compositions) or sequentially in separate composition. When administered sequentially, the phosphorylated or phosphonated pyrone analog may be administered prior to, or after, a second agent in the combination. The phosphorylated or phosphonated pyrone analogs may interact with each other in a synergistic or additive manner to exert a biological effect or effects, for example, reducing lipid and glucose levels in the subject. The synergy between phosphorylated or phosphonated pyrone analogs can potentially allow a reduction in the dose required for each phosphorylated or phosphonated pyrone analog, leading to a reduction in the side effects and enhancement of the clinical utility of these phosphorylated or phosphonated pyrone analogs. The combination of phosphorylated or phosphonated pyrone analogs may also comprise one or more phosphorylated or phosphonated pyrone analogs in particular proportions, depending on the relative potencies of each phosphorylated or phosphonated pyrone analog and the intended indication.

In some embodiments, provided herein is a solid pharmaceutical composition for oral administration. In some embodiments, the solid pharmaceutical composition for oral administration contains a phosphorylated or phosphonated pyrone analog at about 5-1000 mg and a pharmaceutically acceptable excipient. In some embodiments, provided herein is a liquid pharmaceutical composition for oral administration. In some embodiments, the liquid pharmaceutical composition for oral administration contains a phosphorylated or phosphonated pyrone analog at about 5-1000 mg and a pharmaceutically acceptable excipient.

Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

Further encompassed herein are anhydrous pharmaceutical compositions and dosage forms containing an active ingredient. Water may be added (e.g., 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. Pharmaceutical compositions and dosage forms which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The tablet can be prepared for immediate-release. For example, the tablet can be an erodible tablet. A solubilizer, such as Captisol® when compressed, that erodes rather than disintegrates can be mixed with the active ingredient to form the erodible tablet. Formulation for oral use can also be present as a hard gelatin capsule using suboptimal lyophilization process.

Surfactant which can be used to form pharmaceutical compositions and dosage forms include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, preferred ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides; citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the phosphorylated or phosphonated pyrone analog and to minimize precipitation of the phosphorylated or phosphonated pyrone analog. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Cyclodextrins and their derivatives can be used to enhance the aqueous solubility of hydrophobic compounds. Cyclodextrins are cyclic carbohydrates derived from starch. The unmodified cyclodextrins differ by the number of glucopyranose units joined together in the cylindrical structure. The parent cyclodextrins typically contain 6, 7, or 8 glucopyranose units and are referred to as alpha-, beta-, and gamma-cyclodextrin respectively. Each cyclodextrin subunit has secondary hydroxyl groups at the 2 and 3-positions and a primary hydroxyl group at the 6-position. The cyclodextrins may be pictured as hollow truncated cones with hydrophilic exterior surfaces and hydrophobic interior cavities. In aqueous solutions, these hydrophobic cavities can incorporate hydrophobic organic compounds, which can fit all, or part of their structure into these cavities. This process, sometimes referred to as inclusion complexation, may result in increased apparent aqueous solubility and stability for the complexed drug. The complex is stabilized by hydrophobic interactions and does not generally involve the formation of any covalent bonds.

Cyclodextrins can be derivatized to improve their properties. Cyclodextrin derivatives that are useful for pharmaceutical applications include the hydroxypropyl derivatives of alpha-, beta- and gamma-cyclodextrin, sulfoalkylether cyclodextrins such as sulfobutylether beta-cyclodextrin, alkylated cyclodextrins such as the randomly methylated beta.-cyclodextrin, and various branched cyclodextrins such as glucosyl- and maltosyl-beta.-cyclodextrin. Chemical modification of the parent cyclodextrins (usually at the hydroxyl moieties) has resulted in derivatives with sometimes improved safety while retaining or improving the complexation ability of the cyclodextrin. The chemical modifications, such as sulfoalkyl ether and hydroxypropyl, can result in rendering the cyclodextrins amorphous rather than crystalline, leading to improved solubility.

Examples of additional suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methylpyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

In some embodiments, the phosphorylated or phosphonated pyrone analog may be administered to an animal alone or in combination with one or more other agents of one or more other forms to have a biological effect on lipid, triglyceride or glucose levels in the animal. Such combination may comprise agents including but not limited to chemical compounds, nucleic acids (i.e., DNA, RNA), proteins, peptides, peptidomimetics, peptoids, or any other forms of a molecule. The agents in a combination may be administered to an animal simultaneously or sequentially. These agents in a combination may be of any category of agents mentioned herein, and may interact with each other in a synergistic or additive manner to exert a biological effect or effects. The synergy between the phosphorylated or phosphonated pyrone analog and the agents can potentially allow a reduction in the dose required for each agent, leading to a reduction in the side effects and enhancement of the clinical utility of these agents. The combination of the phosphorylated or phosphonated pyrone analog and the agents may also comprise one or more phosphorylated or phosphonated pyrone analogs and agents in particular proportions, depending on the relative potencies of each phosphorylated or phosphonated pyrone analog or agent and the intended indication.

In other embodiments, compositions comprise a phosphorylated or phosphonated pyrone analog with a compound that lowers lipid levels (i.e. lipid-lowering compound). The lipid-lowering compound may be present in an amount sufficient to exert an therapeutic effect and the phosphorylated or phosphonated pyrone analog may be present in an amount sufficient to decrease hyperlipidemia, hypercholesterolemia, hypertriglyceridemia and/or one or more symptoms thereof by a measurable amount, compared to treatment without the phosphorylated or phosphonated pyrone analog when administered to an animal.

The symptom measured may be any symptom as described herein. In some embodiments, the symptom that is reduced includes, but is not limited to, xanthoma, skin lesion, pancreatitis, enlargement of liver and spleen, chest pain, heart attack or a combination thereof. The measurable amount may be an average of at least about 5, 10, 15, 20, 25, 30, 35, 40, 45, 50, 55, 60, 65, 70, 75, 80, 85, 90, 95, or more than 95% as described herein.

A lipid-lowering compound may be a compound that lowers the level of cholesterol in a subject (i.e. cholesterol-lowering compound). Cholesterol-lowering compounds include, but are not limited to, clofibrate, gemfibrozil, and fenofibrate, nicotinic acid, mevinolin, mevastatin, pravastatin, simvastatin, fluvastatin, lovastatin, cholestyrine, colestipol or probucol.

A lipid-lowering compound may be a compound that lowers the level of triglyceride in a subject (i.e. triclyceride-lowering compounds). Triglyceride-lowering compounds include, but are not limited to, ascorbic acid, asparaginase, clofibrate, colestipol, fenofibrate mevastatin, pravastatin, simvastatin, fluvastatin, or omega-3 fatty acid. A lipid-lowering compound may also be a compound that lowers the level of LDL-cholesterol in a subject. In some embodiments triglyceride is plasma triglyceride and/or liver triglyceride. In some embodiments triglyceride is plasma triglyceride. In some embodiments triglyceride is liver triglyceride.

Compositions may comprise a phosphorylated or phosphonated pyrone analog and a lipid-lowering compound wherein the phosphorylated or phosphonated pyrone analog is, for example, phosphorylated or phosphonated fisetin, phosphorylated or phosphonated isofisetin, phosphorylated or phosphonated flavon, phosphorylated or phosphonated chrysin, phosphorylated or phosphonated apigenin, phosphorylated or phosphonated rhoifolin, phosphorylated or phosphonated diosmin, phosphorylated or phosphonated galangin, phosphorylated or phosphonated morin, phosphorylated or phosphonated rutin, phosphorylated or phosphonated kaempferol, phosphorylated or phosphonated myricetin, phosphorylated or phosphonated taxifolin, phosphorylated or phosphonated naringenin, phosphorylated or phosphonated naringin, phosphorylated or phosphonated hesperetin, phosphorylated or phosphonated hesperidin, phosphorylated or phosphonated chalcone, phosphorylated or phosphonated phloretin, phosphorylated or phosphonated phlorizdin, phosphorylated or phosphonated genistein, phosphorylated or phosphonated biochanin A, phosphorylated or phosphonated catechin, or phosphorylated or phosphonated epicatechin, or a combination thereof. In some embodiments, compositions comprise phosphorylated or phosphonated fisetin or a phosphorylated or phosphonated fisetin derivative, phosphorylated or phosphonated quercetin or a phosphorylated or phosphonated quercetin derivative, or a combination thereof, and a lipid-lowering compound.

The lipid-lowering compound may be present in an amount sufficient to exert a therapeutic effect and the phosphorylated or phosphonated pyrone analogs may be present in an amount sufficient to decrease hyperlipidemia, hypercholesterolemia, hypertriglyceridemia and/or one or more symptoms of thereof by a measurable amount, compared to treatment without the phosphorylated or phosphonated pyrone analogs when administered to an animal. The measurable amount may be an average of at least about 5, 10, 15, 20, 25, 30, 35, 40, 45, 50, 55, 60, 65, 70, 75, 80, 85, 90, 95, or more than 95% as described herein.

In some embodiments, compositions comprise a phosphorylated or phosphonated pyrone analog wherein the phosphorylated or phosphonated pyrone analog is present in an amount sufficient to decrease the concentration of lipid including but not limited to cholesterol and triglyceride in a physiological compartment by a measurable amount, compared to the concentration without the phosphorylated or phosphonated pyrone analog when the phosphorylated or phosphonated pyrone analog is administered to an animal. In other embodiments, compositions comprise a phosphorylated or phosphonated pyrone analog which is phosphorylated or phosphonated fisetin, a phosphorylated or phosphonated fisetin derivative, phosphorylated or phosphonated quercetin or a phosphorylated or phosphonated quercetin derivative, in an amount sufficient to decrease the concentration of lipid including but not limited to cholesterol and triglyceride in a physiological compartment by a measurable amount, compared to the concentration without the phosphorylated or phosphonated pyrone analog, when administered to an animal. The measurable amount may be an average of at least about 5%, 10%, 15%, 20%, or more than 20%. In some embodiments, the physiological compartment is a lipid accumulating cell or cell membrane including but not limited to macrophage, muscle cell, or adipocyte. In other embodiments, the physiological compartment is a pancreatic islet cell including β cell. In still other embodiments, the physiological compartment is a hepatocyte. Other examples of physiological compartments include, but are not limited to, blood, brain, liver, lymph nodes, spleen, Peyer's patches, intestines, lungs, heart, pancreas and kidney.

In some embodiments, a composition comprises a lipid-lowering compound as described herein, and a phosphorylated or phosphonated pyrone analog. In some embodiments, a composition comprises a cholesterol-lowering compound and a phosphorylated or phosphonated pyrone analog. In other embodiments, a composition comprises a triglyceride-lowering compound and a phosphorylated or phosphonated pyrone analog. The concentration of one or more of the lipid-lowering compounds and/or phosphorylated or phosphonated pyrone analog may be less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

Alternatively, the concentration of one or more of the lipid-lowering compounds and/or phosphorylated or phosphonated pyrone analog may be greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In other embodiments, compositions comprise a phosphorylated or phosphonated pyrone analog with a compound that lowers glucose levels (i.e. a glucose-lowering compound). In such compositions, the phosphorylated or phosphonated pyrone analog can be any of those described herein. In one embodiment, compositions comprise a phosphorylated or phosphonated pyrone analog and a glucose-lowering compound wherein the phosphorylated or phosphonated pyrone analog is, for example, phosphorylated or phosphonated fisetin, phosphorylated or phosphonated isofisetin, phosphorylated or phosphonated flavon, phosphorylated or phosphonated chrysin, phosphorylated or phosphonated apigenin, phosphorylated or phosphonated rhoifolin, phosphorylated or phosphonated diosmin, phosphorylated or phosphonated galangin, phosphorylated or phosphonated morin, phosphorylated or phosphonated rutin, phosphorylated or phosphonated kaempferol, phosphorylated or phosphonated myricetin, phosphorylated or phosphonated taxifolin, phosphorylated or phosphonated naringenin, phosphorylated or phosphonated naringin, phosphorylated or phosphonated hesperetin, phosphorylated or phosphonated hesperidin, phosphorylated or phosphonated chalcone, phosphorylated or phosphonated phloretin, phosphorylated or phosphonated phlorizdin, phosphorylated or phosphonated genistein, phosphorylated or phosphonated biochanin A, phosphorylated or phosphonated catechin, or phosphorylated or phosphonated epicatechin, or a combination thereof. In some embodiments, compositions comprise phosphorylated or phosphonated fisetin or a phosphorylated or phosphonated fisetin derivative, phosphorylated or phosphonated quercetin or a phosphorylated or phosphonated quercetin derivative, or a combination thereof, and a glucose-lowering compound.

The glucose-lowering compound may be present in an amount sufficient to exert a therapeutic effect and the phosphorylated or phosphonated pyrone analog may be present in an amount sufficient to decrease hyperglycemia and/or one or more symptoms thereof by a measurable amount, compared to treatment without the phosphorylated or phosphonated pyrone analog when the composition is administered to an animal. The measurable amount may be an average of at least about 5, 10, 15, 20, 25, 30, 35, 40, 45, 50, 55, 60, 65, 70, 75, 80, 85, 90, 95, or more than 95%.

The symptom of hyperglycemia may be any symptom as described herein including, but not limited to, glucosuria, polyphagia, polyuria, polydipsia, loss of consciousness, blurred vision, headaches, coma, ketoacidosis, decrease in blood volume, decrease in renal blood flow, accelerated lipolysis, weight loss, stomach problems, intestinal problems, poor wound healing, dry mouth, nausea, vomiting, dry skin, itchy skin, impotence, hypeventilation, ketoanemia, fatigue, weakness on one side of the body, hallucinations, impairment in cognitive function, increase sadness, anxiety, recurrent genital infections, increase sugar in urine, retinopathy, nepropathy, arteriosclerotic disorders, cardiac arrhythmia, stupor, susceptibility to infection, neuropathy, nerve damages causing cold feet, nerve damage causing insensitive feet and loss of hair. In one embodiment, the symptom of hyperglycemia is glucosuria.

Glucose-lowering compounds include, but are not limited to, glipizide, exenatide, incretins, sitagliptin, pioglitizone, glimepiride, rosiglitazone, metformin, exantide, vildagliptin, sulfonylurea, glucosidase inhibitor, biguanide, repaglinide, acarbose, troglitazone, nateglinide, or a variant thereof.

The glucose-lowering compound may be present in a composition in an amount sufficient to exert a therapeutic effect and the phosphorylated or phosphonated pyrone analog may be present in an amount sufficient to decrease hyperglycemia and/or one or more symptoms thereof by a measurable amount, compared to treatment without the phosphorylated or phosphonated pyrone analog when administered to an animal. The measurable amount may be an average of at least about 5, 10, 15, 20, 25, 30, 35, 40, 45, 50, 55, 60, 65, 70, 75, 80, 85, 90, 95, or more than 95%. The symptom of hyperglycemia may be any symptom as described herein.

In some embodiments, a composition comprises a glucose-lowering compound and a phosphorylated or phosphonated pyrone analog. In some embodiments, the concentration of one or more of the glucose-lowering compounds and/or phosphorylated or phosphonated pyrone analog may be less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

Alternatively, the concentration of one or more of the glucose-lowering compounds and/or phosphorylated or phosphonated pyrone analog may be greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

Lipid transport modulators, i.e., phosphorylated or phosphonated pyrone analogs may be administered in the form of pharmaceutical compositions. Lipid or glucose lowering compounds described above may also be administered in the form of pharmaceutical compositions.

When the phosphorylated or phosphonated pyrone analogs and the lipid or glucose lowering compounds are used in combination, both components may be mixed into a preparation or both components may be formulated into separate preparations to use them in combination separately or at the same time.

In one embodiment, pharmaceutical compositions contain, as the active ingredient, a phosphorylated or phosphonated pyrone analog or a pharmaceutically acceptable salt and/or coordination complex thereof, and one or more pharmaceutically acceptable excipients, carriers, including inert solid diluents and fillers, diluents including sterile aqueous solution and various organic solvents, permeation enhancers, solubilizers and adjuvants.

The phosphorylated or phosphonated pyrone analog and/or the lipid or glucose lowering compound may be prepared into pharmaceutical compositions in dosages as described herein. Such compositions are prepared in a manner well known in the pharmaceutical art.

In some embodiments, a pharmaceutical composition for injection comprises a phosphorylated or phosphonated pyrone analog that reduces or eliminates hyperlipidemia, hypercholesterolemia, hypertriglyceridemia, or hyperglycemia and/or one or more symptoms thereof, and a pharmaceutical excipient suitable for injection. In some embodiments, a pharmaceutical composition comprises a combination of a phosphorylated or phosphonated pyrone analog, a lipid lowering compound and a pharmaceutical excipient suitable for injection. In other embodiments, a pharmaceutical composition comprises a combination of a phosphorylated or phosphonated pyrone analog, a glucose lowering compound and a pharmaceutical excipient suitable for injection. In some embodiments, the pharmaceutical composition comprises cyclodextrin-phosphorylated or phosphonated pyrone analog, and a suitable pharmaceutical excipient. Components and amounts of phosphorylated or phosphonated pyrone analogs in the compositions are as described herein.

In some embodiments, the pharmaceutical composition for injection is made using an aqueous composition comprising a phosphorylated or phosphonated pyrone analog, and a pharmaceutically or veterinarily acceptable aqueous carrier wherein the phosphorylated or phosphonated pyrone analog is present in a concentration of greater than 0.5 mM, 1 mM, 5 mM, 10 mM, 15 mM, 20 mM, 30 mM, 33 mM, 40 mM, 50 mM, 60 mM, or 80 mM.

The forms in which the compositions may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the transport protein modulator in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Pharmaceutical composition for injection can be made into a solid formulation that is produced by drying the aqueous composition, for example by freeze drying or lyophilization. Having a dried, solid formulation can be advantageous for increasing the shelf-life. The solid formulation can then be re-dissolved into solution for injection. The dried powder can be further formulated into pharmaceutical composition for injection as described herein.

In some embodiments, a pharmaceutical composition for topical (e.g., transdermal) delivery comprising a phosphorylated or phosphonated pyrone analog reduces or eliminates one or more symptoms of hyperlipidemia, hypercholesterolemia, hypertriglyceridemia, or hyperglycemia, and a pharmaceutical excipient suitable for transdermal delivery. In some embodiments, a pharmaceutical composition for transdermal delivery comprises a combination of a phosphorylated or phosphonated pyrone analog, a lipid lowering compound and a pharmaceutical excipient suitable for transdermal delivery. In other embodiments, a pharmaceutical composition for transdermal delivery comprises a combination of a phosphorylated or phosphonated pyrone analog, a glucose lowering compound that reduces or eliminates hyperglycemia and/or one or more symptoms of hyperglycemia, and a pharmaceutical excipient suitable for transdermal delivery. In some embodiments, the pharmaceutical composition for transdermal delivery comprises a cyclodextrin-phosphorylated or phosphonated pyrone analog, and a pharmaceutical excipient suitable for transdermal delivery. Components and amounts of agents in the compositions are as described herein.

Compositions for inhalation or insufflation include solutions and suspensions in pharmaceutically acceptable, aqueous or organic solvents, or mixtures thereof, and powders. The liquid or solid compositions may comprise suitable pharmaceutically acceptable excipients as described supra. The compositions may be administered by an oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

In some embodiments, provided herein is a pharmaceutical composition for oral administration comprising a phosphorylated or phosphonated pyrone analog that reduces or eliminates hyperlipidemia, hypercholesterolemia, hypertriglyceridemia, or hyperglycemia and/or one or more symptoms thereof, and a pharmaceutical excipient suitable for oral administration. In some embodiments, provided herein is a pharmaceutical composition for oral administration comprising a combination of a phosphorylated or phosphonated pyrone analog and a lipid lowering compound that reduces or eliminates hyperlipidemia, hypercholesterolemia, hypertriglyceridemia and/or one or more symptoms thereof and a pharmaceutical excipient suitable for oral administration. In other embodiments, provided herein is a pharmaceutical composition for oral administration comprising a combination of a phosphorylated or phosphonated pyrone analog and a glucose lowering compound that reduces or eliminates hyperglycemia and/or one or more symptoms of hyperglycemia and a pharmaceutical excipient suitable for oral administration.

Provided herein is a pharmaceutical composition for oral administration comprising:
(i) an effective amount of a phosphorylated pyrone analog capable of reducing or eliminating hyperlipidemia, hypercholesterolemia, hypertriglyceridemia, or hyperglycemia; and
(ii) a pharmaceutical excipient suitable for oral administration.

Also provided herein is a pharmaceutical composition for oral administration comprising:
(i) an effective amount of a phosphonated pyrone analog capable of reducing or eliminating hyperlipidemia, hypercholesterolemia, hypertriglyceridemia, or hyperglycemia; and
(ii) a pharmaceutical excipient suitable for oral administration.

The above compositions may further comprise: (iii) an effective amount of a lipid lowering compound. Alternatively, the above compositions may further comprise: (iii) an effective amount of a glucose lowering compound.

In some embodiments, the above pharmaceutical compositions may be liquid pharmaceutical compositions suitable for oral consumption. In some embodiments, the above pharmaceutical compositions may be solid pharmaceutical compositions suitable for oral consumption.

Provided herein is a pharmaceutical composition for oral administration comprising:
(i) an effective amount of a phosphorylated pyrone analog that is phosphorylated fisetin, phosphorylated isofisetin, phosphorylated flavon, phosphorylated chrysin, phosphorylated apigenin, phosphorylated rhoifolin, phosphorylated diosmin, phosphorylated galangin, phosphorylated morin, phosphorylated rutin, phosphorylated kaempferol, phosphorylated myricetin, phosphorylated taxifolin, phosphorylated naringenin, phosphorylated naringin, phosphorylated hesperetin, phosphorylated hesperidin, phosphorylated chalcone, phosphorylated phloretin, phosphorylated phlorizdin, phosphorylated genistein, phosphorylated biochanin A, phosphorylated catechin, or phosphorylated epicatechin; and
(ii) a pharmaceutical excipient suitable for oral administration.

Also provided herein is a pharmaceutical composition for oral administration comprising:
(i) an effective amount of a phosphonated pyrone analog that is phosphonated fisetin, phosphonated isofisetin, phosphonated flavon, phosphonated chrysin, phosphonated apigenin, phosphonated rhoifolin, phosphonated diosmin, phosphonated galangin, phosphonated morin, phosphonated rutin, phosphonated kaempferol, phosphonated myricetin, phosphonated taxifolin, phosphonated naringenin, phosphonated naringin, phosphonated hesperetin, phosphonated hesperidin, phosphonated chalcone, phosphonated phloretin, phosphonated phlorizdin, phosphonated genistein, phosphonated biochanin A, phosphonated catechin, or phosphonated epicatechin; and
(ii) a pharmaceutical excipient suitable for oral administration.

The above compositions may further comprise: (iii) an effective amount of a lipid lowering compound. Alternatively, the above compositions may further comprise: (iii) an effective amount of a glucose lowering compound.

Provided herein is a pharmaceutical composition for oral administration comprising:
(i) an effective amount of a phosphorylated pyrone analog that is phosphorylated fisetin, or phosphorylated quercetin; and
(ii) a pharmaceutical excipient suitable for oral administration.

Also provided herein is a pharmaceutical composition for oral administration comprising:
(i) an effective amount of a phosphonated pyrone analog that is phosphonated fisetin, or phosphonated quercetin; and
(ii) a pharmaceutical excipient suitable for oral administration.

The above compositions may further contain: (iii) an effective amount of a lipid lowering compound. Alternatively, the above compositions may further contain: (iii) an effective amount of a glucose lowering compound.

In one aspect the invention provides compositions that include a phosphonated phosphorylated polyphenol e.g. phosphonated pyrone analog such as a phosphonated flavonoid, such as a phosphonated quercetin, phosphonated fisetin, or phosphonated 5,7-dideoxyquercetin, that reduces or eliminates side effect of one or more substances. In some embodiments, the substance is a therapeutic agent with which the phosphonated polyphenol e.g. phosphonated pyrone analog such as a phosphonated flavonoid, such as a phosphonated quercetin, phosphonated fisetin, or phosphonated 5,7-dideoxyquercetin is co-administered. “Co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present, and combinations thereof.

In some embodiments, the invention provides compositions containing a combination of a therapeutic agent and an agent that reduces or eliminates a side effect of the therapeutic agent. In some embodiments the invention provides pharmaceutical compositions that further include a pharmaceutically acceptable excipient. In some embodiments, the pharmaceutical compositions are suitable for oral administration. In some embodiments, the pharmaceutical compositions are suitable for transdermal administration. In some embodiments, the pharmaceutical compositions are suitable for injection. Other forms of administration are also compatible with embodiments of the pharmaceutical compositions of the invention, as described herein.

In some embodiments, the reduction or elimination of side effects is due to the modulation of a BTB transport protein by a phosphonated polyphenol e.g. phosphonated pyrone analog such as a phosphonated flavonoid, such as a phosphonated quercetin, phosphonated fisetin, or phosphonated 5,7-dideoxyquercetin and/or its metabolite. In some embodiments, the BTB transport protein is an ABC transport protein. In some embodiments, the BTB transport protein modulator is a BTB transport protein activator. In some embodiments, the BTB transport protein modulator is a modulator of P-gP.

In some embodiments, the side effect modulator comprises a phosphonated polyphenol and/or its metabolite that acts as a BTB transport protein modulator. In other embodiments, the side effect modulator comprises a phosphonated polyphenol and/or its metabolite which acts to lower a side effect of a therapeutic agent through a non-BTB transport protein-mediated mechanism, or that acts to lower a side effect of a therapeutic agent through a BTB transport protein-mediated mechanism and a non-BTB transport protein-mediated mechanism, is used. In some embodiments utilizing a phosphonated polyphenol, the phosphonated polyphenol is a phosphonated pyrone analog such as a phosphonated flavonoid. In some embodiments utilizing a phosphonated polyphenol, the phosphonated polyphenol is selected from the group consisting of phosphonated quercetin, phosphonated isoquercetin, phosphonated flavon, phosphonated chrysin, phosphonated apigenin, phosphonated rhoifolin, phosphonated diosmin, phosphonated galangin, phosphonated fisetin, phosphonated morin, phosphonated rutin, phosphonated kaempferol, phosphonated myricetin, phosphonated taxifolin, phosphonated naringenin, phosphonated naringin, phosphonated hesperetin, phosphonated hesperidin, phosphonated chalcone, phosphonated phloretin, phosphonated phlorizdin, phosphonated genistein, phosphonated 5,7-dideoxyquercetin, phosphonated biochanin A, phosphonated catechin, and phosphonated epicatechin. In some embodiments utilizing a polyphenol, the polyphenol is a phosphonated flavonol. In certain embodiments, the phosphonated flavonol is selected from the group consisting of phosphonated quercetin, phosphonated fisetin, phosphonated 5,7-dideoxyquercetin, phosphonated galangin, and phosphonated kaempferol, or combinations thereof. In some embodiments, the phosphonated flavonol is phosphonated quercetin. In some embodiments, the phosphonated flavonol is phosphonated galangin. In some embodiments, the phosphonated flavonol is phosphonated kaempferol. In some embodiments, the phosphonated flavonol is phosphonated fisetin. In some embodiments, the phosphonated flavonol is phosphonated 5,7-dideoxyquercetin. In some embodiments, the phosphonated flavonol is quercetin-3′-O-methylphosphonate. In some embodiments, the phosphonated flavonol is quercetin-4′-O-methylphosphonate. In some embodiments, the phosphonated flavonol is quercetin-3-O-methylphosphonate. In some embodiments, the phosphonated flavonol is fisetin-3′-O-methylphosphonate. In some embodiments, the phosphonated flavonol is fisetin-4′-O-methylphosphonate. In some embodiments, the phosphonated flavonol is fisetin-3-O-methylphosphonate.

In embodiments in which the side effect is a side effect of the therapeutic agent that is reduced is selected from the group consisting of drowsiness, impaired concentration, sexual dysfunction, sleep disturbances, habituation, dependence, alteration of mood, respiratory depression, nausea, vomiting, lowered appetite, lassitude, lowered energy, dizziness, memory impairment, neuronal dysfunction, neuronal death, visual disturbance, impaired mentation, tolerance, addiction, hallucinations, lethargy, myoclonic jerking, endocrinopathies, and combinations thereof. In some embodiments, the side effect of the therapeutic agent that is reduced is selected from the group consisting of impaired concentration and sleep disturbances. In some embodiments, the side effect of the therapeutic agent that is reduced is impaired concentration. In some embodiments, the side effect of the therapeutic agent that is reduced is sleep disturbances. In some embodiments, the side effect is a renal and/or urogenital side effect selected from the group consisting of nephrotoxicity, renal function impairment, creatinine increase, urinary tract infection, oliguria, cystitis haemorrhagic, hemolytic-uremic syndrome or micturition disorder, as well as other effects mention herein, and combinations thereof. In some embodiments, the side effect is a hepatic, pancreatic and/or gastrointestinal side effect selected from the group consisting of hepatic necrosis, hepatotoxicity, fatty liver, venooclusive liver disease, diarrhea, nausea, constipation, vomiting, dyspepsia, anorexia, and abnormal LFT, as well as other effects mention herein, and combinations thereof.

In some embodiments, the therapeutic agent is an immunosuppressant. The immunosuppressant can be, for example, a calcineurin inhibitor, e.g., tacrolimus or a tacrolimus analog. The immunosuppressant can be, for example, sirolimus, tacrolimus, mycophenolate, methadone, cyclosporin, prednisone, or voclosporin. In some embodiments, the therapeutic agent is an agent selected from the group of: antivirals, antibiotics, antineoplastics, amphetamines, antihypertensives, vasodilators, barbiturates, membrane stabilizers, cardiac stabilizers, glucocorticoids, antilipedemics, antiglycemics, cannabinoids, antidepressants, antineuroleptics, and antiinfectives. In some embodiments, the therapeutic agent is an antihypertensive. In some embodiments, the therapeutic agent is an antiinfective.

In some embodiments, the invention provides a composition containing a therapeutic agent and an phosphonated polyphenol e.g. phosphonated pyrone analog such as a phosphonated flavonoid, such as a phosphonated quercetin, phosphonated fisetin, phosphonated 5,7-dideoxyquercetin, where the therapeutic agent is present in an amount sufficient to exert a therapeutic effect and the phosphonated polyphenol e.g. phosphonated pyrone analog such as a phosphonated flavonoid, such as a phosphonated quercetin, phosphonated fisetin, phosphonated 5,7-dideoxyquercetin and/or its metabolite is present in an amount sufficient to decrease a side effect of the therapeutic agent by a measurable amount, compared to the side effect without the phosphonated polyphenol, when the composition is administered to an animal. In some embodiments, a side effect of the therapeutic agent is decreased by an average of at least about 1, 5, 10, 15, 20, 25, 30, 35, 40, 45, 50, 55, 60, 65, 70, 75, 80, 85, 90, 95, or more than 95%, compared to the side effect without the phosphonated polyphenol. In some embodiments, a side effect of the therapeutic agent is decreased by an average of at least about 5%, compared to the side effect without the phosphonated polyphenol. In some embodiments, a side effect of the therapeutic agent is decreased by an average of at least about 10%, compared to the side effect without the phosphonated polyphenol. In some embodiments, a side effect of the therapeutic agent is decreased by an average of at least about 15%, compared to the side effect without the phosphonated polyphenol. In some embodiments, a side effect of the therapeutic agent is decreased by an average of at least about 20%, compared to the side effect without the phosphonated polyphenol. In some embodiments, a side effect is substantially eliminated compared to the side effect without the phosphonated polyphenol. “Substantially eliminated” as used herein encompasses no measurable or no statistically significant side effect (one or more side effects) of the therapeutic agent, when administered in combination with the phosphonated polyphenol, e.g. phosphonated pyrone analog such as a phosphonated flavonoid, such as a phosphonated quercetin, phosphonated fisetin, or phosphonated 5,7-dideoxyquercetin.

Thus, in some embodiments, the invention provides compositions that contain a phosphonated polyphenol, e.g., a phosphonated pyrone analog such as a phosphonated flavonoid, such as a phosphonated quercetin, phosphonated fisetin, or phosphonated 5,7-dideoxyquercetin, and an immunosuppressive agent, e.g., tacrolimus or sirolimus, where the immunosuppressive agent is present in an amount sufficient to exert an immunosuppressive effect and the phosphonated polyphenol, e.g. phosphonated pyrone analog such as a phosphonated flavonoid, such as a phosphonated quercetin, phosphonated fisetin, or phosphonated 5,7-dideoxyquercetin is present in an amount sufficient to decrease side effect of the immunosuppressive agent by a measurable amount, compared to the side effect without the phosphonated polyphenol, when the composition is administered to an animal. For further description of immunosuppressive agents that may be used in the compositions of the invention, see U.S. Patent Publication No. US2006/0111308, particularly at paragraphs [0130]-[0154], and PCT published Patent Application WO/06055672, particularly at paragraphs [00116]-[00136]. The measurable amount may be an average of at least about 1, 5, 10, 15, 20, 25, 30, 35, 40, 45, 50, 55, 60, 65, 70, 75, 80, 85, 90, 95, or more than 95%, compared to the side effect without the phosphonated polyphenol. In some embodiments, the side effect is disturbance of concentration. In some embodiments, the side effect is sleep disturbances.

In some embodiments, the invention provides compositions that contain a phosphonated flavonol that is phosphonated quercetin, phosphonated isoquercetin, phosphonated flavon, phosphonated chrysin, phosphonated apigenin, phosphonated rhoifolin, phosphonated diosmin, phosphonated galangin, phosphonated fisetin, phosphonated morin, phosphonated rutin, phosphonated kaempferol, phosphonated myricetin, phosphonated taxifolin, phosphonated naringenin, phosphonated naringin, phosphonated hesperetin, phosphonated hesperidin, phosphonated chalcone, phosphonated phloretin, phosphonated phlorizdin, phosphonated genistein, phosphonated 5,7-dideoxyquercetin, phosphonated biochanin A, phosphonated catechin, or phosphonated epicatechin, or a combination thereof.

In some embodiments, the invention provides compositions that contains phosphonated quercetin, phosphonated fisetin, or phosphonated 5,7-dideoxyquercetin and an immunosuppressant, e.g., tacrolimus (FK-506) where the immunosuppressant, e.g., tacrolimus, is present in an amount sufficient to exert an immunosuppressive effect and the phosphonated quercetin, phosphonated fisetin, or phosphonated 5,7-dideoxyquercetin is present in an amount sufficient to decrease a side effect, or hyperglycemia, of the immunosuppressant by a measurable amount, compared to the side effect without the phosphonated quercetin, phosphonated fisetin, or phosphonated 5,7-dideoxyquercetin when the composition is administered to an animal. The measurable amount may be an average of at least about 1, 5, 10, 15, 20, 25, 30, 35, 40, 45, 50, 55, 60, 65, 70, 75, 80, 85, 90, 95, or more than 95%, compared to the side effect without the phosphonated polyphenol. The side effect may be any side effect as described herein. In some embodiments, the side effect is hyperglycemia. In some embodiments, the side effect is a tissue specific effect.

In some embodiments, the phosphonated polyphenol, e.g. phosphonated pyrone analog such as a phosphonated flavonoid, such as a phosphonated quercetin, phosphonated fisetin, or phosphonated 5,7-dideoxyquercetin and or its metabolite is a side effect modulator, e.g. BTB transport protein modulator, which is present in an amount sufficient to decrease a side effect of the therapeutic agent by a measurable amount and to increase a therapeutic effect of the therapeutic agent by a measurable amount, compared to the side effect and therapeutic effect without the side effect modulator, e.g. BTB transport protein modulator, when the composition is administered to an animal. In some embodiments, a therapeutic effect of the therapeutic agent is increased by an average of at least about 1, 5, 10, 15, 20, 25, 30, 35, 40, 45, 50, 55, 60, 65, 70, 75, 80, 85, 90, 95, or more than 95%, compared to the therapeutic effect without the side effect modulator, e.g. BTB transport protein modulator. In some embodiments, a therapeutic effect of the therapeutic agent is increased by an average of at least about 5%, compared to the therapeutic effect without the side effect modulator, e.g. BTB transport protein modulator. In some embodiments, a therapeutic effect of the therapeutic agent is increased by an average of at least about 10%, compared to the therapeutic effect without the side effect modulator, e.g. BTB transport protein modulator. In some embodiments, a therapeutic effect of the therapeutic agent is increased by an average of at least about 15%, compared to the therapeutic effect without the side effect modulator, e.g. BTB transport protein modulator. In some embodiments, a therapeutic effect of the therapeutic agent is increased by an average of at least about 20%, compared to the therapeutic effect without the side effect modulator, e.g. BTB transport protein modulator. In some embodiments, a therapeutic effect of the therapeutic agent is increased by an average of at least about 30%, compared to the therapeutic effect without the side effect modulator, e.g. BTB transport protein modulator. In some embodiments, a therapeutic effect of the therapeutic agent is increased by an average of at least about 40%, compared to the therapeutic effect without the side effect modulator, e.g. BTB transport protein modulator. In some embodiments, a therapeutic effect of the therapeutic agent is increased by an average of at least about 50%, compared to the therapeutic effect without the side effect modulator, e.g. BTB transport protein modulator.

In some embodiments, the invention provides compositions containing a phosphonated polyphenol, e.g. phosphonated pyrone analog such as a phosphonated flavonoid, such as phosphonated quercetin, phosphonated fisetin, or phosphonated 5,7-dideoxyquercetin, present in an amount sufficient to decrease side effect of a therapeutic agent by an average of at least about 5% and to increase a therapeutic effect of the therapeutic agent by an average of at least about 5%, when the composition is administered to an animal in combination with the therapeutic agent, compared to the side effect and therapeutic effect without the phosphonated polyphenol. In some embodiments, the invention provides compositions containing a phosphonated polyphenol, e.g. phosphonated pyrone analog such as a phosphonated flavonoid such as phosphonated quercetin, phosphonated fisetin, or phosphonated 5,7-dideoxyquercetin present in an amount sufficient to decrease a side effect of a therapeutic agent by an average of at least about 10% and to increase a therapeutic effect of the therapeutic agent by an average of at least about 10%, when the composition is administered to an animal in combination with the therapeutic agent, compared to the side effect and therapeutic effect when the therapeutic agent is administered without the a phosphonated polyphenol, e.g. phosphonated pyrone analog such as a phosphonated flavonoid such as phosphonated quercetin, phosphonated fisetin, or phosphonated 5,7-dideoxyquercetin. In some embodiments, the invention provides compositions containing a phosphonated polyphenol, e.g. phosphonated pyrone analog such as a phosphonated flavonoid such as phosphonated quercetin, phosphonated fisetin, or phosphonated 5,7-dideoxyquercetin present in an amount sufficient to decrease a side effect of a therapeutic agent by an average of at least about 20% and to increase a therapeutic effect of the therapeutic agent by an average of at least about 20%, when the composition is administered to an animal in combination with the therapeutic agent, compared to the side effect and therapeutic effect when the therapeutic agent is administered without the a phosphonated polyphenol, e.g. phosphonated pyrone analog such as a phosphonated flavonoid such as phosphonated quercetin, phosphonated fisetin, or phosphonated 5,7-dideoxyquercetin. In some embodiments, the invention provides compositions containing a phosphonated polyphenol, e.g. phosphonated pyrone analog such as a phosphonated flavonoid such as phosphonated quercetin, phosphonated fisetin, or phosphonated 5,7-dideoxyquercetin present in an amount sufficient to decrease a side effect of a therapeutic agent by an average of at least about 10% and to increase a therapeutic effect of the therapeutic agent by an average of at least about 20%, when the composition is administered to an animal in combination with the therapeutic agent, compared to the side effect and therapeutic effect when the therapeutic agent is administered without the a phosphonated polyphenol, e.g. phosphonated pyrone analog such as a phosphonated flavonoid such as phosphonated quercetin, phosphonated fisetin, or phosphonated 5,7-dideoxyquercetin. In some embodiments, the invention provides compositions containing a phosphonated polyphenol, e.g. phosphonated pyrone analog such as a phosphonated flavonoid such as phosphonated quercetin, phosphonated fisetin, or phosphonated 5,7-dideoxyquercetin present in an amount sufficient to decrease a side effect of a therapeutic agent by an average of at least about 10% and to increase a therapeutic effect of the therapeutic agent by an average of at least about 30%, when the composition is administered to an animal in combination with the therapeutic agent, compared to the side effect and therapeutic effect when the therapeutic agent is administered without the phosphonated polyphenol. In some embodiments, the invention provides compositions containing a phosphonated polyphenol, e.g. phosphonated pyrone analog such as a phosphonated flavonoid such as phosphonated quercetin, phosphonated fisetin, or phosphonated 5,7-dideoxyquercetin present in an amount sufficient to decrease aside effect of a therapeutic agent by an average of at least about 10% and to increase a therapeutic effect of the therapeutic agent by an average of at least about 40%, when the composition is administered to an animal in combination with the therapeutic agent, compared to the side effect and therapeutic effect when the therapeutic agent is administered without the phosphonated polyphenol. In some embodiments, the invention provides compositions containing a phosphonated polyphenol, e.g. phosphonated pyrone analog such as a phosphonated flavonoid such as phosphonated quercetin, phosphonated fisetin, or phosphonated 5,7-dideoxyquercetin present in an amount sufficient to decrease a side effect of a therapeutic agent by an average of at least about 10% and to increase a therapeutic effect of the therapeutic agent by an average of at least about 50%, when the composition is administered to an animal in combination with the therapeutic agent, compared to the side effect and therapeutic effect when the therapeutic agent is administered without the a phosphonated polyphenol, e.g. phosphonated pyrone analog such as a phosphonated flavonoid such as phosphonated quercetin, phosphonated fisetin, or phosphonated 5,7-dideoxyquercetin.

In exemplary embodiments, the invention provides a composition that contains a phosphonated polyphenol that is phosphonated quercetin, phosphonated isoquercetin, phosphonated flavon, phosphonated chrysin, phosphonated apigenin, phosphonated rhoifolin, phosphonated diosmin, phosphonated galangin, phosphonated fisetin, phosphonated morin, phosphonated rutin, phosphonated kaempferol, phosphonated myricetin, phosphonated taxifolin, phosphonated naringenin, phosphonated naringin, phosphonated hesperetin, phosphonated hesperidin, phosphonated chalcone, phosphonated phloretin, phosphonated phlorizdin, phosphonated genistein, phosphonated 5,7-dideoxyquercetin, phosphonated biochanin A, phosphonated catechin, or phosphonated epicatechin, or combinations thereof, and an immunosuppressive, such as an calcineurin inhibitor, e.g., tacrolimus or sirolimus, where the immunosuppressive agent is present in an amount sufficient to exert an immunosuppressive effect, and the phosphonated polyphenol is present in an amount effective to decrease a side effect of the immunosuppressive agent by a measurable amount (e.g., an average of at least about 1, 5, 10, 15, 20, 25, 30, 35, 40, 45, 50, 55, 60, 65, 70, 75, 80, 85, 90, 95, or more than 95%) and to increase the immunosuppressive effect of the immunosuppressive agent by a measurable amount (e.g., an average of at least about 1, 5, 10, 15, 20, 25, 30, 35, 40, 45, 50, 55, 60, 65, 70, 75, 80, 85, 90, 95, or more than 95%). The side effect may be any side effect as described herein. In some embodiments, the side effect is hyperglycemia. In some embodiments, the side effect is a renal side effect. In some embodiments, the side effect is nephrotoxicity. In some embodiments, the side effect is decrease in metabolic function. In yet further exemplary embodiments, the invention provides a composition that contains a phosphonated flavonol that is phosphonated quercetin, phosphonated fisetin, phosphonated 5,7-dideoxyquercetin, phosphonated galangin, or phosphonated kaempferol and an immunosuppressive that is tacrolimus, sirolimus, mycophenolate, methadone, cyclosporin, prednisone, or voclosporin, where the immunosuppressive is present in an amount sufficient to exert an immunosuppressive effect, and the phosphonated flavonol is present in an amount effective to decrease a side effect of the immunosuppressive agent by a measurable amount (e.g., an average of at least about 1, 5, 10, 15, 20, 25, 30, 35, 40, 45, 50, 55, 60, 65, 70, 75, 80, 85, 90, 95, or more than 95%) and to increase the immunosuppressive effect of the immunosuppressive agent by a measurable amount (e.g., an average of at least about 1, 5, 10, 15, 20, 25, 30, 35, 40, 45, 50, 55, 60, 65, 70, 75, 80, 85, 90, 95, or more than 95%). The side effect may be any side effect as described herein. In some embodiments, the side effect is hyperglycemia. In some embodiments, the side effect is a renal side effect. In some embodiments, the side effect is nephrotoxicity. In some embodiments, the side effect is decrease in metabolic function.

An “average” as used herein is preferably calculated in a set of normal human subjects, this set being at least about 3 human subjects, preferably at least about 5 human subjects, preferably at least about 10 human subjects, even more preferably at least about 25 human subjects, and most preferably at least about 50 human subjects.

In some embodiments, the invention provides a composition that contains a therapeutic agent and a phosphonated polyphenol e.g. phosphonated pyrone analog such as a phosphonated flavonoid, such as a phosphonated quercetin, phosphonated fisetin, or phosphonated 5,7-dideoxyquercetin. In some embodiments, the concentration of the therapeutic agents is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v in the composition. In some embodiments, the concentration of the phosphonated polyphenol e.g. phosphonated pyrone analog such as a phosphonated flavonoid, such as a phosphonated quercetin, phosphonated fisetin, or phosphonated 5,7-dideoxyquercetin is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v in the composition.

In some embodiments, a concentration of the therapeutic agent is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v in the composition. In some embodiments, a concentration of the phosphonated polyphenol e.g. phosphonated pyrone analog such as a phosphonated flavonoid, such as a phosphonated quercetin, phosphonated fisetin, or phosphonated 5,7-dideoxyquercetin is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17 The invention provides methods of treating tissue rejection, using therapeutic agents and the phosphonated compositions of the invention. Any suitable type of tissue rejection, whether acute or chronic, may be treated by the methods of the invention. Thus, in some embodiments, the invention provides a method of treating an animal for graft protection by administering to an animal at risk of tissue rejection an effective amount of an immunosuppressive agent, e.g. an calcineurin inhibitor such as tacrolimus or sirolimus and an amount of a phosphonated polyphenol e.g. phosphonated pyrone analog such as a phosphonated flavonoid, such as a phosphonated quercetin, phosphonated fisetin, or phosphonated 5,7-dideoxyquercetin sufficient to reduce a side effect of the immunosuppressive agent.

In some embodiments, a concentration of the therapeutic agent is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v in the composition. In some embodiments, a concentration of the phosphonated polyphenol e.g. phosphonated pyrone analog such as a phosphonated flavonoid, such as a phosphonated quercetin, phosphonated fisetin, or phosphonated 5,7-dideoxyquercetin is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v. v/v in the composition.

In some embodiments, a concentration of the therapeutic agent is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v in the composition. In some embodiments, a concentration of the phosphonated polyphenol e.g. phosphonated pyrone analog such as a phosphonated flavonoid, such as a phosphonated quercetin, phosphonated fisetin, or phosphonated 5,7-dideoxyquercetin is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v in the composition.

In some embodiments, an amount of the therapeutic agent is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g in the composition. In some embodiments, an amount of the phosphonated polyphenol e.g. phosphonated pyrone analog such as a phosphonated flavonoid, such as a phosphonated quercetin, phosphonated fisetin, or phosphonated 5,7-dideoxyquercetin is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g in the composition.

In some embodiments, an amount of the therapeutic agent is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g in the composition. In some embodiments, an amount of the phosphonated polyphenol e.g. phosphonated pyrone analog such as a phosphonated flavonoid, such as a phosphonated quercetin, phosphonated fisetin, or phosphonated 5,7-dideoxyquercetin is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g in the composition.

In some embodiments, an amount the therapeutic agent is in the range of 0.0001-10 g, 0.0005-9 g, 0.001-8 g, 0.005-7 g, 0.01-6 g, 0.05-5 g, 0.1-4 g, 0.5-4 g, or 1-3 g in the composition. In some embodiments, an amount of the phosphonated polyphenol e.g. phosphonated pyrone analog such as a phosphonated flavonoid, such as a phosphonated quercetin, phosphonated fisetin, or phosphonated 5,7-dideoxyquercetin is in the range of 0.0001-10 g, 0.0005-9 g, 0.001-8 g, 0.005-7 g, 0.01-6 g, 0.05-5 g, 0.1-4 g, 0.5-4 g, or 1-3 g in the composition.

In some embodiments, a molar ratio of the therapeutic agent to the phosphonated polyphenol e.g. phosphonated pyrone analog such as a phosphonated flavonoid, such as a phosphonated quercetin, phosphonated fisetin, or phosphonated 5,7-dideoxyquercetin can be 0.0001:1 to 1:1. Without limiting the scope of the invention, the molar ratio of one or more of the therapeutic agents to the phosphonated polyphenol e.g. phosphonated pyrone analog such as a phosphonated flavonoid, such as a phosphonated quercetin, phosphonated fisetin, or phosphonated 5,7-dideoxyquercetin can be about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1. Without limiting the scope of the present invention, the molar ratio of one or more of the therapeutic agents to the phosphonated pyrone analog such as a phosphonated flavonoid, such as a phosphonated quercetin, phosphonated fisetin, or phosphonated 5,7-dideoxyquercetin can be about 0.03×10⁻⁵:1, 0.04×10⁻⁵:1, 0.1×10⁻⁵:1, 0.2×10⁻⁵:1, 0.3×10⁻⁵:1, 0.4×10⁻⁵:1, 0.5×10⁻⁵:1, 0.8×10⁻⁵:1, 0.1×10⁻⁴:1, 0.2×10⁻⁴:1, 0.3×10⁻⁴:1, 0.4×10⁻⁴:1, 0.5×10⁻⁴:1, 0.8×10⁻⁴:1, 0.1×10⁻³:1, 0.2×10⁻³:1, 0.3×10⁻³:1, 0.4×10⁻³:1, 0.5×10⁻³:1, 0.8×10⁻³:1, 0.1×10⁻²:1, 0.2×10⁻²:1, 0.3×10⁻²:1, 0.4×10⁻²:1, 0.5×10⁻²:1, 0.6×10⁻²:1, 0.8×10⁻²:1, 0.01:1, 0.1:1; 0.2:1, 0.3:1, 0.4:1, 0.5:1, 0.6:1, 0.7:1, 0.8:1, 0.9:1, 1:1, 2:1, 3:1, 4:1, or 5:1.

In some embodiments, the therapeutic agent is tacrolimus, sirolimus, mycophenolate, methadone, cyclosporin, prednisone, or voclosporin

The phosphonated polyphenols of the invention are usually administered in the form of pharmaceutical compositions. The drugs described above are also administered in the form of pharmaceutical compositions. When the transport protein modulators and the drugs are used in combination, both components may be mixed into a preparation or the two components may be formulated into separate preparations to use them in combination separately or at the same time.

This invention therefore provides pharmaceutical compositions that contain, as the active ingredient, a phosphonated polyphenol e.g. phosphonated pyrone analog such as a phosphonated flavonoid, such as a phosphonated quercetin, phosphonated fisetin, or phosphonated 5,7-dideoxyquercetin or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

This invention further provides pharmaceutical compositions that contain, as the active ingredient, a phosphonated polyphenol e.g. phosphonated pyrone analog such as a phosphonated flavonoid, such as a phosphonated quercetin, phosphonated fisetin, or phosphonated 5,7-dideoxyquercetin which acts as a side effect modulator, e.g. BTB transport protein modulator or a pharmaceutically acceptable salt and/or coordination complex thereof, a therapeutic agent or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

Such compositions are prepared in a manner well known in the pharmaceutical art.

In some embodiments, the invention provides a pharmaceutical composition for oral administration containing a combination of a therapeutic agent and a phosphonated polyphenol e.g. phosphonated pyrone analog such as a phosphonated flavonoid, such as a phosphonated quercetin, phosphonated fisetin, or phosphonated 5,7-dideoxyquercetin and a pharmaceutical excipient suitable for oral administration. In some embodiment, the phosphonated polyphenol reduces or eliminates a side effect of the therapeutic agent. In some embodiments, the phosphonated polyphenol reduces or eliminates the side effect of the therapeutic agent is a BTB transport protein modulator, as described elsewhere herein.

In some embodiments, the invention provides a pharmaceutical composition comprising:
(i) an effective amount of a therapeutic agent;
(ii) an effective amount of a phosphonated polyphenol capable of reducing or eliminating one or more side effects of the therapeutic agent; and
(iii) a pharmaceutical excipient.

In some embodiments, the composition further comprising: (iv) an effective amount of a second therapeutic agent.

In some embodiments, the pharmaceutical composition may be a solid pharmaceutical composition suitable for oral consumption.

In some embodiments, the therapeutic agent is an immunosuppressive agent. In some embodiments, the therapeutic agent is a calcineurin inhibitor. In some embodiments, the therapeutic agent is tacrolimus or sirolimus. In some embodiments, the phosphonated polyphenol, capable of reducing or eliminating one or more side effects of the therapeutic agent, is a BTB transport protein modulator. In some embodiments, the BTB transport protein modulator is a BTB transport protein activator.

In some embodiments, the invention provides a pharmaceutical composition comprising:
(i) an effective amount of a therapeutic agent that is tacrolimus, sirolimus, mycophenolate, methadone, cyclosporin, prednisone, voclosporin, oxycodone, gabapentin, pregabalin, hydrocodone, fentanyl, hydromorphone, levorphenol, morphine, methadone, mycophenolate, tramadol, hydromorphine, topiramate, diacetyl morphine, codeine, olanzapine, hydrocortisone, prednisone, sufentanyl, alfentanyl, carbamazapine, lamotrigine, doxepin, or haloperidol;
(ii) an effective amount of a phosphonated polyphenol that is phosphonated quercetin, phosphonated isoquercetin, phosphonated flavon, phosphonated chrysin, phosphonated apigenin, phosphonated rhoifolin, phosphonated diosmin, phosphonated galangin, phosphonated fisetin, phosphonated morin, phosphonated rutin, phosphonated kaempferol, phosphonated myricetin, phosphonated taxifolin, phosphonated naringenin, phosphonated naringin, phosphonated hesperetin, phosphonated hesperidin, phosphonated chalcone, phosphonated phloretin, phosphonated phlorizdin, phosphonated genistein, phosphonated 5,7-dideoxyquercetin, phosphonated biochanin A, phosphonated catechin, or phosphonated epicatechin; and
(iii) a pharmaceutical excipient.

In some embodiments, the composition further contains, (iv) an effective amount of a second therapeutic agent. Exemplary second therapeutic agents include aspirin, acetaminophen, and ibuprofen.

In some embodiments, the pharmaceutical composition may be a solid pharmaceutical composition suitable for oral consumption.

In some embodiments, the invention provides a pharmaceutical composition comprising:
(i) an effective amount of a therapeutic agent that is tacrolimus, sirolimus, mycophenolate, methadone, cyclosporin, prednisone, or voclosporin;
(ii) an effective amount of a phosphonated polyphenol that is phosphonated quercetin, phosphonated fisetin, phosphonated 5,7-dideoxyquercetin, phosphonated galangin, or phosphonated kaempferol; and
(iii) a pharmaceutical excipient.

In some embodiments, the composition further contains (iv) an effective amount of a second therapeutic agent. Exemplary second therapeutic agents include aspirin, acetaminophen, and ibuprofen.

In some embodiments, the pharmaceutical composition may be a solid pharmaceutical composition suitable for oral consumption. In some embodiments, the pharmaceutical composition may be a liquid pharmaceutical composition suitable for oral consumption.

In some embodiments, the invention provides a solid pharmaceutical composition for oral administration containing an effective amount of sirolimus, an amount of phosphonated quercetin, phosphonated fisetin, or phosphonated 5,7-dideoxyquercetin that is effective in reducing or eliminating a side effect of sirolimus, and a pharmaceutically acceptable excipient. In some embodiments, the composition further includes an effective amount of acetaminophen. In some embodiments, the invention provides a liquid pharmaceutical composition for oral administration containing an effective amount of sirolimus, an amount of phosphonated quercetin, phosphonated fisetin, or phosphonated 5,7-dideoxyquercetin that is effective in reducing or eliminating a side effect of sirolimus, and a pharmaceutically acceptable excipient. In some embodiments, the composition further includes an effective amount of acetaminophen.

In some embodiments, the invention provides a solid pharmaceutical composition for oral administration containing sirolimus at about 1-160 mg, phosphonated quercetin, phosphonated fisetin, or phosphonated 5,7-dideoxyquercetin at about 10-1000 mg and a pharmaceutically acceptable excipient. In some embodiments, the invention provides a liquid pharmaceutical composition for oral administration containing sirolimus at about 1-200 mg/ml, phosphonated quercetin, phosphonated fisetin, or phosphonated 5,7-dideoxyquercetin at about 10-1000 mg/ml and a pharmaceutically acceptable excipient. In some embodiments, the composition further includes acetaminophen at about 10-750 mg/ml.

In some embodiments, the invention provides a solid pharmaceutical composition for oral administration containing an effective amount of tacrolimus, an amount of phosphonated quercetin, phosphonated fisetin, or phosphonated 5,7-dideoxyquercetin that is effective in reducing or eliminating a side effect of tacrolimus, and a pharmaceutically acceptable excipient. In some embodiments, the invention provides a liquid pharmaceutical composition for oral administration containing an effective amount of tacrolimus, an amount of phosphonated quercetin, phosphonated fisetin, or phosphonated 5,7-dideoxyquercetin that is effective in reducing or eliminating a side effect of tacrolimus, and a pharmaceutically acceptable excipient

In some embodiments, the invention provides a solid pharmaceutical composition for oral administration containing tacrolimus at about 1-160 mg, phosphonated quercetin, phosphonated fisetin, or phosphonated 5,7-dideoxyquercetin at about 10-1000 mg and a pharmaceutically acceptable excipient. In some embodiments, the composition further includes acetaminophen at about 200-750 mg. In some embodiments, the invention provides a liquid pharmaceutical composition for oral administration containing tacrolimus at about 1-200 mg/ml, phosphonated quercetin, phosphonated fisetin, or phosphonated 5,7-dideoxyquercetin at about 10-1000 mg/ml and a pharmaceutically acceptable excipient.

In some embodiments, the invention provides a solid pharmaceutical composition for oral administration containing an effective amount of cyclosporin, an amount of phosphonated quercetin, phosphonated fisetin, or phosphonated 5,7-dideoxyquercetin that is effective in reducing or eliminating a side effect of cyclosporin, and a pharmaceutically acceptable excipient. In some embodiments, the invention provides a liquid pharmaceutical composition for oral administration containing an effective amount of cyclosporin, an amount of phosphonated quercetin, phosphonated fisetin, or phosphonated 5,7-dideoxyquercetin that is effective in reducing or eliminating a side effect of cyclosporin, and a pharmaceutically acceptable excipient.

In some embodiments, the invention provides a solid pharmaceutical composition for oral administration containing cyclosporin at about 100-800 mg, phosphonated quercetin, phosphonated fisetin, or phosphonated 5,7-dideoxyquercetin at about 10-1000 mg and a pharmaceutically acceptable excipient. In some embodiments, the invention provides a liquid pharmaceutical composition for oral administration containing cyclosporin at about 5-500 mg/ml, phosphonated quercetin, phosphonated fisetin, or phosphonated 5,7-dideoxyquercetin at about 10-1000 mg/ml and a pharmaceutically acceptable excipient.

### III. Methods Of Treatment

A. Conditions Treated by a Pyrone Analog

Described herein are compounds, pharmaceutical compositions and methods for regulating, preventing, and treating one or more of: cholesterol, chylomicrons, very low density lipoprotein (VLDL), intermediate density lipoprotein (IDL), low density lipoprotein (LDL), high density lipoprotein (HDL), hyperlipidemia, hypercholesterolemia, triglycerides, hypertriglyceridemia, lipid transport, glucose intolerance, hyperglycemia, diabetes mellitus, atherosclerosis, hypertension, liver diseases (e.g., NAFLD (Non-alcoholic fatty liver disease) or NASH (nonalcoholic steatohepatitis)), pancreatitis, obesity, kidney diseases, Niemann-Pick disease, cardiovascular disease, hypoinsulinemia, insulin resistance, vascular sentosis, inflammation, or development of atherosclerotic plaques by administering an effective amount of a pyrone analog (or a derivative thereof) or a phosphorylated or phosphonated pyrone analog (or a derivative thereof) as described herein, alone or in combination with one or more additional agents (e.g., lipid-lowering agents or glucose lowering agents).

Provided herein is a method of maintaining cellular physiological conditions for cell survival, comprising administering to a subject in an effective amount of a pyrone analog that modulates activity of a cellular transporter. Cellular transporters include, but are not limited to, ABCA1, ABCA2, ABCA7, ALDP, ALDR, ABCG1, ABCG4, ABCG5, ABCG6 or ABCG8. Provided herein is a method of treating a disease, comprising administering to a subject an effective amount of a pyrone analog, wherein the pyrone analog modulates activity of a cell surface transporter. Provided herein is a method of treating a metabolic disease and promoting pancreatic function (e.g., increase islet cell function, increase islet cell survival, protection against hyperglycemia, protection against insulin insufficiency during nutrient stimulated insulin release and synthesis, protection against triglyceride elevation, protection against cholesterol elevation, protection against weight gain, protection against stress of glucose loads, etc.), comprising administering to a subject an effective amount of a pyrone analog, wherein the pyrone analog modulates activity of a cell surface transporter. In one embodiment, a cell surface transporter is ABCA1, ABCA2, ABCA7, ALDP, ALDR, ABCG1, ABCG4, ABCG5, ABCG6 or ABCG8. Diseases or metabolic diseases being treated include, but are not limited to, amyloidosis, diabetes, disorders of myelin formation, hyperglycemia, impaired wound healing, neuropathy, insulin resistance, hyperinsulinemia, hypoinsulinemia, hypertension, hyperlipidemia, hypertriglyceridemia, hyperchlesterolemia, malignancy, microvascular retinopathy, surfactant abnormalities, vascular stenosis, inflammation, and hydronephrosis.

Provided herein is a method of maintaining cellular physiological conditions for pancreatic islet cell survival, comprising administering to a subject an effective amount of a pyrone analog.

Provided herein is a method of treating pancreatic cell stress or injury comprising administering to a subject an effective amount of at least one pyrone analog, wherein at least one effect of stress or injury is improved in one or more cell types of the subject.

In one embodiment, a pyrone analog modulates insulin levels in the subject. In another embodiment, a pyrone analog modulates glucose levels in the subject. In another embodiment, a pyrone analog modulates triglyceride levels in the subject. In another embodiment, a pyrone analog modulates body weight in the subject. In another embodiment, a pyrone analog modulates fat weight in the subject. In another embodiment, a pyrone analog modulates adiponectin levels in the subject. In another embodiment, a pyrone analog modulates cholesterol in the subject. In another embodiment, a pyrone analog modulates high density lipoprotein levels in the subject. In another embodiment, a pyrone analog modulates medium density lipoprotein levels in the subject. In another embodiment, a pyrone analog modulates low density lipoprotein levels in the subject. In another embodiment, a pyrone analog modulates very low density lipoprotein levels in the subject. In another embodiment, a pyrone analog modulates prostaglandin levels in the subject. In another embodiment, a pyrone analog modulates development of cancer in the subject. In another embodiment, a pyrone analog modulates inflammation mediator levels in the subject. In another embodiment, a pyrone analog modulates cytokine levels in the subject. In another embodiment, a pyrone analog modulates foam cell levels in the subject. In another embodiment, a pyrone analog modulates development of atherosclerotic streaks in the subject. In another embodiment, a pyrone analog modulates development of atherosclerotic plaques in the subject. In yet another embodiment, a pyrone analog modulates development of vascular stenosis in the subject. In another embodiment, a pyrone analog modulates HbA1C levels in the subject. In another embodiment, a pyrone analog modulates phospholipid levels in the subject. In another embodiment, a pyrone analog modulates surfactant levels in the subject.

Glycated hemoglobin (HbA1C) is a form of hemoglobin used primarily to identify the average plasma glucose concentration over prolonged periods of time. It is formed in a non-enzymatic pathway by hemoglobin's normal exposure to high plasma levels of glucose. A high HbA1c represents poor glucose control. Higher levels of HbA1c are found in people with persistently elevated blood sugar, as in diabetes mellitus.

Adiponectin (also referred to as Acrp30, apM1) is a protein hormone that modulates a number of metabolic processes, including glucose regulation and fatty acid catabolism. Adiponectin is secreted from adipose tissue into the bloodstream and is abundant in plasma relative to many hormones. Levels of the hormone are inversely correlated with body fat percentage in adults, while the association in infants and young children is more unclear. The hormone plays a role in the suppression of the metabolic derangements that may result in type 2 diabetes, obesity, atherosclerosis and non-alcoholic fatty liver disease (NAFLD).

Somatostatin (also known as growth hormone inhibiting hormone (GHIH) or somatotropin release-inhibiting factor (SRIF)) is a peptide hormone that regulates the endocrine system and affects neurotransmission and cell proliferation via interaction with G-protein-coupled somatostatin receptors and inhibition of the release of numerous secondary hormones. Somatostatin has two active forms produced by alternative cleavage of a single preproprotein: one of 14 amino acids, the other of 28 amino acids. Somatostatin suppresses the release of pancreatic hormones (i.e., inhibits the release of insulin and glucagon).

Glucagon helps maintain the level of glucose in the blood by binding to glucagon receptors on hepatocytes, causing the liver to release glucose—stored in the form of glycogen—through a process known as glycogenolysis. As these stores become depleted, glucagon then encourages the liver to synthesize additional glucose by gluconeogenesis. This glucose is released into the bloodstream. Both of these mechanisms lead to glucose release by the liver, preventing the development of hypoglycemia. Glucagon also regulates the rate of glucose production through lipolysis.

Ghrelin is a hormone that signals appetite and stimulates food intake. Ghrelin is known to exist in at least two forms: 1) n-octanoyl ghrelin in which the third serine residue is n-octanoylated and 2) des-n-octanoyl ghrelin in which the n-octanoyl group is removed. Ghrelin is the first identified peripheral hormone signaling appetite. People who were given ghrelin increased their appetite resulting in up to one third more food intake than control subjects. In addition to stimulating food intake, ghrelin levels drop once an individual starts eating. Consequently, ghrelin may act as a trigger to start food intake; ghrelin levels do not fall after eating in obese individuals which suggests that this trigger is not reset in such individuals.

Vasoactive intestinal peptide (VIP) is a 28 amino acid peptide. This peptide belongs to a family of structurally related, small polypeptides that includes helodermin, secretin, the somatostatins, and glucagon. The biological effects of VIP are mediated by the activation of membrane-bound receptor proteins that are coupled to the intracellular cAMP signaling system. Pituitary adenylate cyclase-activating polypeptide (PACAP) is a neuropeptide belonging to the secretin/glucagon/vasoactive intestinal polypeptide (VIP) family. The physiological function of the peptide is responsible for diverse roles such as the regulating actions on hormonal synthesis and secretion in pituitary and adrenal medulla, and the differentiation and growth-promoting actions of nerve cells and germ cells. PACAP immuno-positive nerve projects into islets; the expressions of a PAC1 receptor displaying high affinity to PACAP among PACAP receptor subtypes and a VPAC2 receptor displaying nearly equal affinities to both of PACAP and VIP are observed in pancreatic beta cells; and (c) PACAP promotes the glucose-inducible insulin secretion by the isolated islet at a low level.

Prostaglandins are a family of substances showing a wide diversity of biological effects. Prostaglandins of the 1-, 2-, and 3-series, respectively, incorporate one, two, or three double bonds in their basic 20-carbon carboxylic fatty acid structure which incorporates a 5-member cyclopentene ring. The 1-series of prostaglandins are strong vasodilators, and inhibit cholesterol and collagen biosynthesis, as well as platelet aggregation. On the other hand, the 2-series prostaglandins are known to enhance platelet aggregation, cholesterol, and collagen biosynthesis, and also to enhance endothelial cell proliferation. The main effect of the 3-series prostaglandins, particularly PGE3, is the suppression of the 2-series prostaglandins. The precursor of the 2-series prostaglandins is arachidonic acid (A11-Z-5,8,11,14-eicosatetraenoic acid). DHLA is the precursor for the 1-series prostaglandins, and, as indicated hereinabove, EPA and DHA are precursors for the 3-series prostaglandins. EPA and DHA are effective precursors for prostaglandin PGE3, which suppresses the 2-series prostaglandins. Additionally, EPA and/or DHA itself competes with arachidonic acid on the same enzymatic system and thus inhibits the biosynthesis of 2-series prostaglandins. This inhibition of the 2-series prostaglandins results in an increase of the ratio of PGE1:PGE2.

In the methods disclosed herein, cells can be pancreatic islet cells. Pancreatic islet cells may be damaged or subject to destruction such as, for example, by apoptosis, necrosis and/or autophagy.

Provided herein is a method of assessing cellular protective effects in pancreatic islet cells, comprising: i) selecting a patient for treatment based on one or more biomolecule levels in a sample compared to a control sample; ii) administering an effective amount of a pyrone analog to a subject; and iii) monitoring said one or more biomolecule levels in a subject. Biomolecules include, but are not limited to, insulin, somatostatin, glucagon, grehlin, VIP, glucose, and adiponectin. In one embodiment, insulin levels are stable and do not decrease.

Certain biomarkers (biomolecules) can be expressed at increased or decreased levels in response to administration of a pyrone analog to a patient.

As used herein, the term “expression,” when used in connection with detecting the expression of a gene, can refer to detecting transcription of the gene and/or to detecting translation of the gene. To detect expression of a gene refers to the act of actively determining whether a gene is expressed or not. This can include determining whether the gene expression is upregulated as compared to a control, downregulated as compared to a control, or unchanged as compared to a control. Therefore, the step of detecting expression does not require that expression of the gene actually is upregulated or downregulated, but rather, can also include detecting that the expression of the gene has not changed (i.e., detecting no expression of the gene or no change in expression of the gene).

Biomarkers (biomolecules) to be assessed in connection with the present invention include, but are not limited to, insulin, somatostatin, glucagon, grehlin, VIP, glucose, amylin, GP-1 and adiponectin.

For assessment of biomarker (biomolecule) expression, patient samples can be used in methods described herein and further known in the art. Briefly, the level of expression of the biomarker (biomolecule) can be assessed by assessing the amount (e.g., absolute amount or concentration) of the marker in a sample, obtained from a patient, or other patient sample containing material derived from a patient (e.g., blood, serum, urine, or other bodily fluids or excretions as described herein above). A cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample.

One can detect expression of biomarker proteins having at least one portion which is displayed on the surface of cells which express it. One can determine whether a marker protein, or a portion thereof, is exposed on the cell surface. For example, immunological methods can be used to detect such proteins on whole cells, or well known computer-based sequence analysis methods can be used to predict the presence of at least one extracellular domain (i.e., including both secreted proteins and proteins having at least one cell-surface domain). Expression of a marker protein having at least one portion which is displayed on the surface of a cell which expresses it can be detected without necessarily lysing the cell (e.g., using a labeled antibody which binds specifically with a cell-surface domain of the protein).

Expression of biomarkers can be assessed by any of a wide variety of well known methods for detecting expression of a transcribed nucleic acid or protein. Non-limiting examples of such methods include, for example, immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods or any other method known in the art.

A mixture of transcribed polynucleotides obtained from the sample can be contacted with a substrate having fixed thereto a polynucleotide complementary to or homologous with at least a portion (e.g., at least 7, 10, 15, 20, 25, 30, 40, 50, 100, 500, or more nucleotide residues) of a biomarker nucleic acid. If polynucleotides complementary to, or homologous with, are differentially detectable on the substrate (e.g., detectable using different chromophores or fluorophores, or fixed to different selected positions), then the levels of expression of a plurality of biomarkers can be assessed simultaneously using a single substrate (e.g., a “gene chip” microarray of polynucleotides fixed at selected positions). When a method of assessing biomarker expression is used which involves hybridization of one nucleic acid with another, hybridization can be performed under stringent hybridization conditions.

An exemplary method for detecting the presence or absence of a biomarker protein or nucleic acid in a biological sample involves obtaining a biological sample from a test subject and contacting the biological sample with a compound or an agent capable of detecting the polypeptide or nucleic acid (e.g., mRNA, genomic DNA, or cDNA). The detection methods can, thus, be used to detect mRNA, protein, cDNA, or genomic DNA, for example, in a biological sample in vitro as well as in vivo. In vitro techniques for detection of mRNA include, for example, reverse transcriptase-polymerase chain reaction (RT-PCR; e.g., the experimental embodiment set forth in Mullis, 1987, U.S. Pat. No. 4,683,202), Northern hybridizations and in situ hybridizations. In vitro techniques for detection of a biomarker protein include, but are not limited to, enzyme linked immunosorbent assays (ELISAs), Western blots, immunoprecipitations and immunofluorescence. In vitro techniques for detection of genomic DNA include, for example, Southern hybridizations. In vivo techniques for detection of mRNA include, for example, polymerase chain reaction (PCR), quantitative PCR, Northern hybridizations and in situ hybridizations. Furthermore, in vivo techniques for detection of a biomarker protein include introducing into a subject a labeled antibody directed against the protein or fragment thereof. For example, the antibody can be labeled with a radioactive marker whose presence and location in a subject can be detected by standard imaging techniques.

A general principle of such diagnostic and prognostic assays involves preparing a sample or reaction mixture that may contain a biomarker, and a probe, under appropriate conditions and for a time sufficient to allow the biomarker and probe to interact and bind, thus forming a complex that can be removed and/or detected in the reaction mixture. These assays can be conducted in a variety of ways using a variety of methods.

It is also possible to directly detect biomarker/probe complex formation without further manipulation or labeling of either component (biomarker or probe), for example by utilizing the technique of fluorescence energy transfer (i.e., FET, see for example, Lakowicz et al., U.S. Pat. No. 5,631,169; and Stavrianopoulos, et al., U.S. Pat. No. 4,868,103).

In another embodiment, determination of the ability of a probe to recognize a biomarker can be accomplished without labeling either assay component (probe or biomarker) by utilizing a technology such as real-time Biomolecular Interaction Analysis (BIA; see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” refer to a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

As an alternative to making determinations based on the absolute expression level of the biomarker, determinations can be based on the normalized expression level of the biomarker. Expression levels are normalized by correcting the absolute expression level of a biomarker by comparing its expression to the expression of a gene that is not a biomarker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-tumor sample, or between samples from different sources.

Alternatively, the expression level can be provided as a relative expression level. To determine a relative expression level of a biomarker, the level of expression of the biomarker is determined for 10 or more, 20 or more, 30 or more, 40 or more, or 50 or more samples of normal versus cell isolates prior to the determination of the expression level for the sample in question. The mean expression level assayed in the larger number of samples is determined and this is used as a baseline expression level for the biomarker. The expression level of the biomarker determined for the test sample (absolute level of expression) is then divided by the mean expression value obtained for that biomarker. This provides a relative expression level.

In another embodiment, a biomarker protein is detected. One type of agent for detecting biomarker protein is an antibody capable of binding to such a protein or a fragment thereof such as, for example, a detectably labeled antibody. Antibodies can be polyclonal or monoclonal. An intact antibody, or an antigen binding fragment thereof (e.g., Fab, F(ab′)₂, Fv, scFv, single binding chain polypeptide) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunosorbant assay (ELISA). A skilled artisan can readily adapt known protein/antibody detection methods for use in determining whether tumor cells express a biomarker of the present invention. A combination of two or more of the assays for the detection of biomarkers (non-limiting examples include those described above) can also be used to assess one or more biomarkers.

The endocrine pancreas consists primarily of islet cells that synthesize and secrete the peptide hormone glucagon, insulin, somatostatin and pancreatic polypeptide. Insulin gene expression is restricted to pancreatic islet beta-cells of the mammalian pancreas through control mechanisms mediated, in part, by transcription factors.

Provided herein is a method of assessing pancreatic islet gene expression profile in a subject or a cell. By “pancreatic gene expression profile” is meant to include one or more genes that are normally transcriptionally silent in non-endocrine tissues, e.g., a pancreatic transcription factor an endocrine gene, or an exocrine gene, for example, expression of PC1/3, insulin, glucagon, somatostatin or endogenous PDX-1. The method includes administering to a subject a pyrone analog and assessing gene expression in a sample obtained from said subject.

Induction of a pancreatic gene expression profile can be detected using techniques well known to one of ordinary skill in the art. For example, pancreatic hormone RNA sequences can be detected in, e.g., northern blot hybridization analyses, amplification-based detection methods such as reverse-transcription based polymerase chain reaction or systemic detection by microarray chip analysis. Alternatively, expression can be also measured at the protein level, i.e., by measuring the levels of polypeptides encoded by the gene. Such methods are well known in the art and include, e.g., immunoassays based on antibodies to proteins encoded by the genes, or HPLC.

A sample can be taken from any tissue such as, for example, pancreas, liver, spleen, or kidney. When alterations in gene expression are associated with gene amplification or deletion, sequence comparisons in test and reference populations can be made by comparing relative amounts of the examined DNA sequences in the test and reference samples.

B. Lipid Synthesis and Transport

### Cholesterol Regulation

Cholesterol is a lipid found in the cell membranes and transported in the blood plasma of all animals. It is an essential component of mammalian cell membranes where it is required to establish proper membrane permeability and fluidity. Cholesterol is the principal sterol synthesized by animals while smaller quantities are synthesized in other eukaryotes such as plants and fungi. In contrast cholesterol is almost completely absent among prokaryotes. Most cholesterol is synthesized by the body but significant quantities can also be absorbed from the diet. While minimum level of cholesterol is essential for life, excess can contribute to diseases such as atherosclerosis.

Since cholesterol is insoluble in blood, it is transported in the circulatory system within lipoproteins, complex spherical particles which have an exterior composed mainly of water-soluble proteins; fats and cholesterol are carried internally. There is a large range of lipoproteins within blood, generally called, from larger to smaller size: chylomicrons, very low density lipoprotein (VLDL), intermediate density lipoprotein (IDL), low density lipoprotein (LDL) and high density lipoprotein (HDL). The cholesterol within all the various lipoproteins is identical. Cholesterol is minimally soluble in water; it cannot dissolve and travel in the water-based bloodstream. Instead, it is transported in the bloodstream by lipoproteins that are water-soluble and carry cholesterol and triglycerides internally. The apolipoproteins forming the surface of the given lipoprotein particle determine from what cells cholesterol will be removed and to where it will be supplied.

Cholesterol is transported towards peripheral tissues by the lipoproteins chylomicrons, very low density lipoproteins (VLDL) and low-density lipoproteins (LDL). Large numbers of small dense LDL (sdLDL) particles are strongly associated with the presence of atheromatous disease within the arteries. For this reason, LDL is referred to as “bad cholesterol”. On the other hand, high-density lipoprotein (HDL) particles transport cholesterol back to the liver for excretion. In contrast, having small numbers of large HDL particles is independently associated with atheromatous disease progression within the arteries.

Chylomicrons

Chylomicrons are the largest (1000 nm) and least dense (<0.95) of the lipoproteins. They contain only 1-2% protein, 85-88% triglycerides, ˜8% phospholipids, ˜3% cholesteryl esters and ˜1% cholesterol. Chylomicrons contain several types of apolipoproteins including apo-AI, II & IV, apo-B48, apo-CI, II & III, apo-E and apo-H. Chylomicrons are produced for the purpose of transporting dietary triglycerides and cholesterol absorbed by intestinal epithelia. Chylomicron assembly originates in the intestinal mucosa. Excretion into the plasma is facilitated through the lymphatic system. In the plasma, chylomicrons acquire apo-CII and apo-E from HDL. Once transported to tissues, triglycerides contained in chylomicrons are hydrolyzed by apo-CII-dependent activation of lipoprotein lipase contained on the endothelial cell walls. The chylomicron remnant, including residual cholesterol, is taken up by the liver via receptor-mediated endocytosis by recognition of its apo-E component.

Very Low Density Lipoproteins (VLDL)

Very low density lipoproteins are the next step down from chylomicrons in terms of size and lipid content. They are approximately 25-90 nm in size (MW 6-27 million), with a density of ˜0.98. They contain 5-12% protein, 50-55% triglycerides, 18-20% phospholipids, 12-15% cholesteryl esters and 8-10% cholesterol. VLDL also contains several types of apolipoproteins including apo-B100, apo-CI, II & III and apo-E. VLDL also obtains apo-CII and apo-E from plasma HDL. VLDL assembly in the liver involves the early association of lipids with apo-B100 mediated by microsomal triglyceride transfer protein while apo-B100 is translocated to the lumen of the ER. Lipoprotein lipase also removes triglycerides from VLDL in the same way as from chylomicrons.

Intermediate Density Lipoproteins (IDL)

Intermediate density lipoproteins are smaller than VLDL (40 nm) and more dense (˜1.0). They contain the same apolipoproteins as VLDL. They are composed of 10-12% protein, 24-30% triglycerides, 25-27% phospholipids, 32-35% cholesteryl esters and 8-10% cholesterol. IDLs are derived from triglyceride depletion of VLDL. IDLs can be taken up by the liver for reprocessing, or upon further triglyceride depletion, become LDL.

Low Density Lipoproteins (LDL) and Lipoprotein (a)

Low density lipoproteins are smaller than IDL (26 nm) (MW approximately 3.5 million) and more dense (˜1.04). They contain the apolipoprotein apo-B100. LDL contains 20-22% protein, 10-15% triglycerides, 20-28% phospholipids, 37-48% cholesteryl esters and 8-10% cholesterol. LDL and HDL transport both dietary and endogenous cholesterol in the plasma. LDL is the main transporter of cholesterol and cholesteryl esters and makes up more than half of the total lipoprotein in plasma. LDL is absorbed by the liver and other tissues via receptor mediated endocytosis. The cytoplasmic domain of the LDL receptor facilitates the formation of coated pits; receptor-rich regions of the membrane. The ligand binding domain of the receptor recognizes apo-B100 on LDL, resulting in the formation of a clathrin-coated vesicle. ATP-dependent proton pumps lower the pH inside the vesicle resulting dissociation of LDL from its receptor. After loss of the clathrin coat the vesicles fuse with lysozomes, resulting in peptide and cholesteryl ester enzymatic hydrolysis. The LDL receptor can be recycled to the cell membrane. Insulin, tri-iodothyronine and dexamethasome have shown to be involved with the regulation of LDL receptor mediated uptake.

High Density Lipoproteins

High density lipoproteins are the smallest of the lipoproteins (6-12.5 nm) (MW 175-500KD) and most dense (˜1.12). HDL contains several types of apolipoproteins including apo-AI, II & IV, apo-CI, II & III, apo-D and apo-E. HDL contains approximately 55% protein, 3-15% triglycerides, 26-46% phospholipids, 15-30% cholesteryl esters and 2-10% cholesterol. HDL is produced as a protein rich particle in the liver and intestine, and serves as a circulating source of Apo-CI & II and Apo-E proteins. The HDL protein particle accumulates cholesteryl esters by the esterification of cholesterol by lecithin-cholesterol acyl-transferase (LCAT). LCAT is activated by apo-AI on HDL. HDL can acquire cholesterol from cell membranes and can transfer cholesteryl esters to VLDL and LDL via transferase activity in apo-D. HDL can return to the liver where cholesterol is removed by reverse cholesterol transport, thus serving as a scavenger to free cholesterol. The liver can then excrete excess cholesterol in the form of bile acids. In a normal fasting individual, HDL concentrations range from 1.0-2.0 g/L.

Hyperlipidemia

Hyperlipidemia is an elevation of lipids in the bloodstream. These lipids include cholesterol, cholesterol esters, estersphospholipids and triglycerides. Lipid and lipoprotein abnormalities are considered as a highly modifiable risk factor for cardiovascular disease due to the influence of cholesterol, one of the most clinically relevant lipid substances, on atherosclerosis. In addition, some forms may predispose to acute pancreatitis.

Hypercholesterolemia

Hyperchlesterolemia refers to an abnormally high cholesterol level. Higher concentrations of LDL and lower concentrations of functional HDL are strongly associated with cardiovascular disease because these promote atheroma development in arteries (atherosclerosis). This disease process leads to myocardial infarction (heart attack), stroke and peripheral vascular disease. Since higher blood LDL, especially higher LDL particle concentrations and smaller LDL particle size, contribute to this process more than the cholesterol content of the LDL particles, LDL particles are often termed “bad cholesterol” because they have been linked to atheroma formation. On the other hand, high concentrations of functional HDL, which can remove cholesterol from cells and atheroma, offer protection and are sometimes referred to colloquially as “good cholesterol”.

Conditions with elevated concentrations of oxidized LDL particles, especially “small dense LDL” (sdLDL) particles, are associated with atherosclerosis, which is the principal cause of coronary heart disease and other forms of cardiovascular disease. In contrast, HDL particles (especially large HDL) have been identified as a mechanism by which cholesterol and inflammatory mediators can be removed from atheroma. Increased concentrations of HDL correlate with lower rates of atheroma progressions and even regression.

Elevated levels of the lipoprotein fractions, LDL, IDL and VLDL are regarded as atherogenic (prone to cause atherosclerosis). Levels of these fractions, rather than the total cholesterol level, correlate with the extent and progress of atherosclerosis. Conversely, the total cholesterol can be within normal limits, yet be made up primarily of small LDL and small HDL particles, under which conditions atheroma growth rates would still be high. In contrast, however, if LDL particle number is low (mostly large particles) and a large percentage of the HDL particles are large, then atheroma growth rates are usually low, even negative, for any given total cholesterol concentration.

Multiple human trials utilizing HMG-CoA reductase inhibitors, known as statins, have repeatedly confirmed that changing lipoprotein transport patterns from unhealthy to healthier patterns significantly lowers cardiovascular disease event rates, even for people with cholesterol values currently considered low for adults. As a result, people with a history of cardiovascular disease may derive benefit from statins irrespective of their cholesterol levels.

The 1987 report of National Cholesterol Education Program, Adult Treatment Panels suggest the total blood cholesterol level should be: <200 mg/dL normal blood cholesterol, 200-239 mg/dL borderline-high, >240 mg/dL high cholesterol. The American Heart Association provides a similar set of guidelines for total (fasting) blood cholesterol levels and risk for heart disease as listed in Table 1.

TABLE 1

| Level | Level | |
|---|---|---|
| (mg/dL) | (mmol/L) | Interpretation |
| <200 | <5.2 | Desirable level corresponding to |
| | | lower risk for heart disease |
| 200-240 | 5.2-6.2 | Borderline high risk |
| >240 | >6.2 | High risk |

The desirable LDL level is considered to be less than 100 mg/dL (2.6 mmol/L), although a newer target of <70 mg/dL can be considered in higher risk individuals based on some of the above-mentioned trials. A ratio of total cholesterol to HDL, another useful measure, of far less than 5:1 is thought to be healthier.

Triglyceride

Triglyceride also known as triacylglycerol, TAG or triacylglyceride is glyceride in which the glycerol is esterified with three fatty acids. Triglycerides, as major components of VLDL and chylomicrons, play an important role in metabolism as energy sources and transporters of dietary fat. In the intestine, triglycerides are split into glycerol and fatty acids via lipolysis, which are then moved into the cells lining the intestines (absorptive enterocytes). The triglycerides are rebuilt in the enterocytes from their fragments and packaged together with cholesterol and proteins to form chylomicrons. These are excreted from the cells and collected by the lymph system and transported to the large vessels near the heart before being mixed into the blood. Various tissues can capture the chylomicrons, releasing the triglycerides to be used as a source of energy. Fat and liver cells can synthesize and store triglycerides. When the body requires fatty acids as an energy source, the hormone glucagon signals the breakdown of the triglycerides by hormone-sensitive lipase to release free fatty acids. As the brain cannot utilize fatty acids as an energy source (unless converted to a ketone), the glycerol component of triglycerides can be converted into glucose, via gluconeogenesis, for brain fuel when it is broken down. Triglycerides cannot pass through cell membranes freely. Lipoprotein lipases must break down triglycerides into fatty acids and glycerol. Fatty acids can then be taken up by cells via the fatty acid transporter (FAT).

Hypertriglyceridemia

In the human body, high levels of triglycerides in the bloodstream have been linked to atherosclerosis, and, by extension, the risk of heart disease and stroke. However, the relative negative impact of raised levels of triglycerides compared to that of LDL: HDL ratios is as yet unknown. The risk can be partly accounted for by a strong inverse relationship between triglyceride level and HDL-cholesterol level. Another disease caused by high triglycerides is pancreatitis. When some fatty acids are converted to ketone bodies, overproduction can result in ketoacidosis in diabetics. The American Heart Association has set guidelines for triglyceride levels as listed in Table 2.

TABLE 2

| Level (mg/dL) | Level (mmol/L) | Interpretation |
|---|---|---|
| <150 | <1.69 | Normal range, low risk |
| 150-199 | 1.70-2.25 | Borderline high |
| 200-499 | 2.26-5.65 | High |
| >500 | >5.65 | Very high: high risk |

Triglyceride levels as tested after fasting 8 to 12 hours.

Provided herein is a method of treating acute hypertriglyceridemia during acute lymphoblastic leukemia by administering to a patient an effective amount of a pyrone analog, such as phosphorylated or phosphonated fisetin or phosphorylated or phosphonated quercetin, which reduces or eliminates hypertriglyceridemia and/or one or more symptoms of hypertriglyceridemia.

Moderating the consumption of fats, alcohol and carbohydrates and partaking of aerobic exercise are considered essential to reducing triglyceride levels. Omega-3 fatty acids from fish, flax seed oil or other sources, Omega-6 fatty acids, one or more grams of niacin per day and some statins reduce triglyceride levels. In some cases, fibrates have been used as they can bring down triglycerides substantially. However they are not used as a first line measure as they can have unpleasant or dangerous side effects.

### Lipid Transport—ATP Mediated Transporter

ATP-binding cassette transporters (ABC-transporter) are members of a superfamily, i.e., ATP-mediated transporter family that is one of the largest and most ancient families with representatives in all extant phyla from prokaryotes to humans. These are transmembrane proteins that function in the transport of a wide variety of substrates across extra- and intracellular membranes, including metabolic products, lipids and sterols, and drugs. Proteins are classified as ABC transporters based on the sequence and organization of their ATP-binding domain(s), also known as nucleotide-binding folds (NBFs). ABC transporters are involved in tumor resistance, cystic fibrosis, bacterial multidrug resistance, and a range of other inherited human diseases.

ABC-transporters utilize the energy of ATP hydrolysis to transport various substrates across cellular membranes. Within eukaryotes, ABC-transporters mainly transport molecules to the outside of the plasma membrane or into membrane-bound organelles such as the endoplasmic reticulum, mitochondria, etc. The transported compounds include but are not limited to lipids and sterols; ions and small molecules; drugs and large polypeptides. In some embodiments, the lipid transport protein is an ABC transport protein. In some embodiments, the lipid transport protein modulator is a lipid transport protein activator. In some embodiments, the lipid transport protein modulator is a modulator of ABCA1, ABCA2, ABCA7, ALDP, ALDR, ABCG1, ABCG4, ABCG5, ABCG6 or ABCG8. In other embodiments, the lipid transport protein modulator is a modulator of ABCA1. In other embodiments, the lipid transport protein modulator is a modulator of ABCG1. In other embodiments, the lipid transport protein modulator is a modulator of ABCG4. In other embodiments, the lipid transport protein modulator is a modulator of ABCG8.

Provided herein are methods for treating or preventing hyperlipidemia, hypercholesterolemia, hypertriglyceridemia, hyperglycemia, or a disease associated with hyperlipidemia, hypercholesterolemia, hypertriglyceridemia, or hyperglycemia by administering a pyrone analog alone or in combination with one or more compounds that lower the level of lipid or glucose in a subject. In some embodiment, the pyrone analog modulates a cholesterol transporter. In some embodiments, the cholesterol transporter is ATP-binding cassette, sub-family A member 1 (ABCA1). The ABCA1 gene belongs to a group of genes called the ATP-binding cassette family, which provides instructions for making proteins that transport molecules across cell membranes. This transporter is a major regulator of cellular cholesterol and phospholipid homeostasis. With cholesterol and phospholipids as its substrate, this protein functions as a cholesterol and phospholipids efflux pump in the cellular lipid removal pathway. Mutations in this gene have been associated with Tangier's disease and familial high-density lipoprotein deficiency. The ABCA1 protein is produced in many tissues, but especially in the liver and in immune system cells called macrophages. Macrophages are phagocytes, acting in both innate immunity as well as cell-mediated immunity of vertebrate animals. ABCA1 transfers cholesterol and phospholipids across the cell membrane to the outside of the cell. These substances are then taken up by a protein called apolipoprotein A-1 (apoA1) that circulates in the bloodstream. More specifically, ABCA1 exports excess cellular cholesterol to apoA1 associated with nascent-high-density lipoprotein (HDL) discs, which are assembled in hepatocytes and released into circulation. ApoA1 is used to make HDL. HDL particles carry cholesterol from the body's tissues to the liver for elimination through bile, a yellow substance made by the liver that aids in the digestion of fats. Mature HDL particles are internalized by hepatocytes and free cholesterol is released concomitantly. Free oxysterol and cholesterol levels in hepatocytes provide feedback regulation to cholesterol and fatty acid biosynthesis. The process of removing excess cholesterol from peripheral cells and transporting it to the liver for removal is extremely important for the homeostasis of cholesterol and the cardiovascular health. There is a wide consensus that cholesterol and/or cholesteryl ester accumulation in macrophages plays a role in atherogenesis and that this process occurs through an inflammatory process. A corollary to this premise is that factors that affect the balance between cholesterol retention and cholesterol efflux in macrophages will be pro- or antiatherogenic. With ABCA1 deficiency, apoA-I is rapidly cleared before it is able to acquire cholesterol. Thus, the loss of HDL in ABCA1 deficiency may account for the severe cholesteryl ester storage phenotype seen in tissue macrophages and in hepatocytes of Tangier patients and WHAM chickens.

ABCA1 is well documented as the gate keeper for reverse cholesterol transport. Extrahepatic tissues synthesize cholesterol and also derive cholesterol through the uptake of lipoproteins via the LDL receptor and scavenger receptors. The cholesteryl ester is in a dynamic equilibrium with free cholesterol, through the opposing actions of acylCoA:cholesterol acyltransferase (ACAT) and neutral cholesterol esterase. Free cholesterol effluxes to extracellular acceptors, most notably phospholipid/apoA-I disks (pre-β-HDL). This process is directly (or indirectly through phospholipid efflux) dependent on functional ABCA1. Proper lipidation is essential for the stability of HDL. In the absence of sufficient cholesterol efflux, apoA-I is rapidly cleared from the circulation by the kidneys. Cholesterol that associates with apoA-I/phospholipid disks is a substrate for lecithin:cholesterol acyltransferase (LCAT). LCAT transfers a fatty acyl chain from phosphatidylcholine to cholesterol, forming cholesteryl ester. The cholesteryl ester partitions into the hydrophobic core of the lipoprotein, thus forming spherical HDL particles. These particles can then deliver cholesteryl ester to the liver and steroidogenic tissues. B: Selective uptake of cholesteryl esters from HDL. The interaction of spherical HDL particles with the scavenger receptor class B type I (SR-BI) leads to selective delivery of cholesteryl esters. SR-BI interacts with spherical HDL particles but not with apoA-I or poorly lipidated HDL disks. The cholesteryl esters are hydrolyzed by a neutral cholesterol esterase, providing free cholesterol for secretion across the apical (bile canalicular) membrane of the hepatocyte and for bile acid synthesis. Growing evidence suggests that a major source of cholesterol for ABCA1-mediated transport to HDL is the liver.

Peroxisome proliferator-activated receptors (PPARs) are a group of nuclear receptor proteins that function as transcription factors regulating the expression of genes. All PPARs heterodimerize with the retinoid X receptor (RXR) and bind to specific regions on the DNA of target genes. The orphan nuclear receptor peroxisome proliferator-activated receptor gamma (PPARγ) is considered as a regulator of adipocyte development and has become a potential therapeutic target for the treatment of a diverse array of disorders, including but not limited to type 2 diabetes, dyslipidaemia, inflammation and malignancy. Thiazolidinediones (TZDs, e.g. rosiglitazone and pioglitazone) are high-affinity PPARγ ligands, and are used as a novel class of antidiabetic agent, licensed for use in the management of type 2 diabetes mellitus.

PPARγ has been implicated in the regulation of CD36 expression and macrophage uptake of oxidized LDL (oxLDL). In addition to lipid uptake, PPARγregulates a pathway of cholesterol efflux. PPARγinduces ABCA1 expression and cholesterol removal from macrophages through a transcriptional cascade mediated by the nuclear receptor LXR alpha. Ligand activation of PPARγleads to primary induction of LXR alpha and to coupled induction of ABCA1. Transplantation of PPARγnull bone marrow into LDLR −/− mice results in a significant increase in atherosclerosis, consistent with the hypothesis that regulation of LXR alpha and ABCA1 expression is protective in vivo. Thus, PPARγcoordinates a complex physiologic response to oxLDL that involves particle uptake, processing, and cholesterol removal through ABCA1.

ATP-binding cassette, sub-family G member 1 (ABCG1) is another cholesterol transporter. Studies indicate a synergistic relationship between ABCA1 and ABCG1 in peripheral tissues, where ABCA1 lipidates any lipid-poor/free apoA-I to generate nascent or pre-β-HDL. These particles in turn may serve as substrates for ABCG1-mediated cholesterol export.

### Glucose Intolerance, Hyperglycemia and Hypoinsulinemia

Hyperglycemia or high blood sugar is a condition in which an excessive amount of glucose circulates in the blood plasma. This is generally a blood glucose level of 100+ mmol/L, but symptoms and effects may not start to become noticeable until later numbers such as 150-200+ mmol/L.

Hypoinsulinemia is a condition wherein lower than normal amounts of insulin circulate throughout the body and wherein obesity is generally not involved. This condition includes Type I diabetes.

Diabetes Mellitus

Provided herein are methods that can be used to prevent or treat diabetes mellitus.

Diabetes mellitus is encompassed within insulin resistance and hypoinsulinemia and refers to a state of chronic hyperglycemia, i.e., excess sugar in the blood, consequent upon a relative or absolute lack of insulin action. There are three basic types of diabetes mellitus, Type I or insulin-dependent diabetes mellitus (IDDM), Type 2 or non-insulin-dependent diabetes mellitus (NIDDM), and Type A insulin resistance, although Type A is relatively rare. Patients with either Type I or Type 2 diabetes can become insensitive to the effects of exogenous insulin through a variety of mechanisms. Type A insulin resistance results from either mutations in the insulin receptor gene or defects in post-receptor sites of action critical for glucose metabolism. Diabetic subjects can be easily recognized by the physician, and are characterized by fasting hyperglycemia, impaired glucose tolerance, glycosylated hemoglobin, and, in some instances, ketoacidosis associated with trauma or illness. “Non-insulin dependent diabetes mellitus” or “NIDDM” refers to Type 2 diabetes. NIDDM patients have an abnormally high blood glucose concentration when fasting and delayed cellular uptake of glucose following meals or after a diagnostic test known as the glucose tolerance test. Diabetes mellitus is a syndrome of disordered metabolism, usually due to a combination of hereditary and environmental causes, resulting in hyperglycemia. Blood glucose levels are controlled by insulin made in the beta cells of the pancreas. The two most common forms of diabetes are due to either a diminished production of insulin, or diminished response by the body to insulin. Both lead to hyperglycemia, which largely causes the acute signs of diabetes: excessive urine production, resulting compensatory thirst and increased fluid intake, blurred vision, unexplained weight loss, lethargy, and changes in energy metabolism.

Chronic hyperglycemia that persists even in fasting states is most commonly caused by diabetes mellitus, and in fact chronic hyperglycemia is the defining characteristic of the disease. Type 2 diabetes mellitus is characterized by insulin resistance or reduced insulin sensitivity, combined with reduced insulin secretion. Insulin causes cellular uptake of glucose from the blood (including liver, muscle, and fat tissue cells), storing it as glycogen in the liver and muscle. When insulin is absent (or low) or when tissues fail to response to the presense of insulin, glucose is not taken up by cells, resulting in hyperglycemia.

ABCA1 and ABCG1 are highly expressed in pancreatic islet cells. Mice with specific inactivation of ABCA1 in pancreatic β-cells had markedly impaired glucose tolerance and defective insulin secretion but normal insulin sensitivity. Islets isolated from these mice showed altered cholesterol homeostasis and impaired insulin secretion in vitro. Modulating the activities of pancreatic ABCA1 and ABCG1 is expected to improve pancreatic islet function and normalize glucose stimulated insulin secretion.

ABCA1 and ABCG1 are expressed in skeletal muscles. Excess fatty acid stored in skeletal muscle cells interferes with insulin signaling and desensitize insulin induced glucose uptake. Modulating the activities of skeletal muscle ABCA1 and ABCG1 is expected to improve muscle glucose uptake and reduce insulin resistance.

Provided herein is a method of treating diabetes mellitus by administering to a patient, e.g. a diabetic patient an effective amount of a pyrone analog, such as phosphorylated or phosphonated fisetin or phosphorylated or phosphonated quercetin, which reduces or eliminates hyperglycemia and/or one or more symptoms of hyperglycemia. Modulation of insulin regulation, glucose tolerance, and glucose transport can be evaluated with a variety of imaging and assessment techniques known in the art. Assessment criteria known in the art include, but are not limited to: assessment of insulin levels, assessment of blood glucose levels and glucose uptake studies by oral glucose challenge, assessment of cytokine profiles, blood-gas analysis, extent of blood-perfusion of tissues, and angiogenesis within tissues. Additional criteria for assessing the treatment of diabetes will be employed to assess the beneficial effects of treatment with pyrone analogs.

Provided herein is a method of treating hyperlipidemia, hypercholesterolemia, hypertriglyceridemia, or hyperglycemia by administering one or more pyrone analogs, which modulate and activate ABCA1 and ABCG1, thereby increasing cholesterol and phospholipid efflux from cells containing excess lipids to ApoA1 and HDL particles in circulating blood. The reduced cellular levels of cholesterol and fatty acids restore or normalize glucose-stimulated insulin-induced glucose uptake and β-cell energy metabolism, and also restore glucose sensing through increased insulin synthesis and release as well as β-cell expansion.

In one aspect, provided herein is a method of treating hyperlipidemia, the method comprising administering a therapeutically effective amount of a pyrone analog to a subject in need thereof, wherein the pyrone analog reduces hyperlipidemia and/or one or more symptoms associated with hyperlipidemia in the subject. In another aspect, provided herein is a method of treating hypercholesterolemia, the method comprising administering a therapeutically effective amount of a pyrone analog to a subject in need thereof, wherein the pyrone analog reduces hypercholesterolemia and/or one or more symptoms associated with hypercholesterolemia in the subject.

In another aspect, provided herein is a method of treating hypertriglyceridemia, the method comprising administering a therapeutically effective amount of a pyrone analog to a subject in need thereof, wherein the pyrone analog reduces hypertriglyceridemia and/or one or more symptoms associated with hypertriglyceridemia in the subject.

In yet another aspect, provided herein is a method of treating or preventing a disease associated with hyperlipidemia, hypercholesterolemia, or hypertriglyceridemia, the method comprising administering a therapeutically effective amount of a pyrone analog to a subject in need thereof, wherein the pyrone analog prevents or alleviates at least one symptom of the disease.

Inflammatory mediator responses (e.g., PGE2, IL-1 beta, and TNF-alpha) represent a risk marker for periodontal diseases in insulin-dependent diabetes mellitus patients. Tumor necrosis factor (TNF) is a cytokine produced primarily by monocytes and macrophages. TNF is found in higher amounts within the plasma of patients with diabetes.

In one embodiment, provided herein is a method of lowering levels of TNF in a diabetic patient. Also provided herein are methods for facilitating metabolic control in a subject. In one aspect, the method for facilitating metabolic control in a subject decreases the level of IL-1 beta in the subject.

The methods described herein generally involve the administration of one or more drugs for the treatment of one or more diseases. Combinations of agents can be used to treat one disease or multiple diseases or to modulate the side-effects of one or more agents in the combination. When a pyrone analog and a lipid or glucose-lowering compound as described herein are used in combination for treatment of a condition such as diabetes mellitus, any suitable ratio of the two agents, e.g., molar ratio, wt/wt ratio, wt/volume ratio, or volume/volume ratio, as described herein, may be used.

In one aspect, provided herein are methods for treating hyperlipidemia associated diseases by administering to a subject in need a pyrone analog or a derivative thereof that modulates a lipid transporter. In another aspect, provided herein are methods for treating hyperglycemia associated diseases by administering to a subject in need a pyrone analog or a derivative thereof that modulates a lipid transporter.

Cardiovascular disease refers to the class of diseases that involve the heart or blood vessels (arteries and veins). While the term technically refers to any disease that affects the cardiovascular system, it is usually used to refer to those related to atherosclerosis (arterial disease). These conditions have similar causes, mechanisms, and treatments.

Atherosclerosis, the most prevalent of cardiovascular diseases, is the principal cause of heart attack, stroke, and gangrene of the extremities, and thereby a principle cause of death. Atherosclerosis is a complex disease involving many cell types and molecular factors. The process, in normal circumstances a protective response to insults to the endothelium and smooth muscle cells (SMCs) of the wall of the artery, consists of the formation of fibrofatty and fibrous lesions or plaques, preceded and accompanied by inflammation. The advanced lesions of atherosclerosis may occlude the artery concerned, and result from an excessive inflammatory-fibroproliferative response to numerous different forms of insult. For example, shear stresses are thought to be responsible for the frequent occurrence of atherosclerotic plaques in regions of the circulatory system where turbulent blood flow occurs, such as branch points and irregular structures.

One observable event in the formation of an atherosclerotic plaque occurs when blood-borne monocytes adhere to the vascular endothelial layer and transmigrate through to the sub-endothelial space. Adjacent endothelial cells at the same time produce oxidized low density lipoprotein (LDL). These oxidized LDL's are then taken up in large amounts by the monocytes through scavenger receptors expressed on their surfaces. In contrast to the regulated pathway by which native LDL (nLDL) is taken up by nLDL specific receptors, the scavenger pathway of uptake is not regulated by the monocytes.

These lipid-filled monocytes are called foam cells, and are the major constituent of the fatty streak. Interactions between foam cells and the endothelial and SMCs which surround them lead to a state of chronic local inflammation which can eventually lead to smooth muscle cell proliferation and migration, and the formation of a fibrous plaque. Such plaques occlude the blood vessel concerned and thus restrict the flow of blood, resulting in ischemia.

Foam cells are cells in an atheroma derived from both macrophages and smooth muscle cells which have accumulated low density lipoproteins, LDLs, by endocytosis. The LDL has crossed the endothelial barrier and has been oxidized by reactive oxygen species produced by the endothelial cells. Foam cells can also be known as fatty like streaks and typically line the intima media of the vasculature.

Foam cells can become a health problem when they accumulate at a particular foci, thus creating a necrotic center of the atherosclerosis. If the fibrous cap that prevents the necrotic center from spilling into the lumen of a vessel ruptures, a thrombus can form which can lead to emboli occluding smaller vessels. The occlusion of small vessels results in ischemia, and contributes to stroke and myocardial infarction, two of the leading causes of cardiovascular-related death.

Vascular Stenosis

Provided herein are methods that can be used to prevent or treat vascular stenosis. Vascular stenosis (and restenosis) is a pathological condition which often results from vascular trauma or damage to blood vessel walls. Vascular trauma or damage is relatively common when a patient undergoes vascular surgery or other therapeutic techniques such as angioplasty. The term “vascular stenosis” is used in a broad sense and refers to a pathological process in which the cavity of a blood vessel is narrowed and which usually results in a pathological condition characterized by impaired flow through the vessel. Following administration of a compound described herein to a patient, the patient's physiological condition can be monitored in various ways well known to the skilled practitioner.

Atherosclerosis

Provided herein are methods that can be used to prevent or treat atherosclerosis. Atherosclerosis is a disease affecting arterial blood vessels. It is a chronic inflammatory response in the walls of arteries, in large part due to the accumulation of foam cells derived from macrophage white blood cells promoted by oxidized low density lipoproteins (oxLDL) and without adequate removal of fats and cholesterol from the macrophages by high density lipoproteins (HDL). Increased activity of ABCA1 and ABCG1 are expected to increase removal of cholesterol and lipids from macrophages and prevent the development of foam cells.

Provided herein is a method of treating atherosclerosis by administering a pyrone analog or a derivative thereof to a subject. Pyrone analogs or derivatives thereof may also be administered in combination with other agents to treat atherosclerosis. Thus, a pyrone analog or a derivative thereof may be co-administered with a statin, niacin, low dose aspirin, intestinal cholesterol absorption-inhibiting supplements (ezetimibe and others, and to a much lesser extent fibrates), or a combination thereof.

Hypertension

Provided herein are methods that can be used to prevent or treat hypertension by administering a pyrone analog or a derivative thereof to a subjec. Hypertension, also referred to as high blood pressure, is a medical condition in which the blood pressure is chronically elevated. It normally refers to arterial hypertension. Hypertension is related to hyperglycemia and hyperlipidemia. In normotensive individuals, insulin may stimulate sympathetic activity without elevating mean arterial pressure. However, in more extreme conditions such as that of the metabolic syndrome, the increased sympathetic neural activity may over-ride the vasodilatory effects of insulin. Insulin resistance and/or hyperinsulinemia have been suggested as being responsible for the increased arterial pressure in some patients with hypertension.

There are many classes of medications for treating hypertension, together called antihypertensives, which, by varying means, act by lowering blood pressure. Evidence suggests that reduction of the blood pressure by 5-6 mmHg can decrease the risk of stroke by 40%, of coronary heart disease by 15-20%, and reduces the likelihood of dementia, heart failure, and mortality from cardiovascular disease. Common drugs for treating hypertension include but are not limited to ACE inhibitors, angiotensin II receptor antagonists, alpha blockers, beta blockers, calcium channel blockers, direct renin inhibitors, and diuretics.

Liver Diseases

Provided herein are methods that can be used to prevent or treat liver diseases by administering a pyrone analog or a derivative thereof to a subject. Hypercholesterolemia is a common feature of primary biliary cirrhosis (PBC) and other forms of cholestatic liver disease. Primary biliary cirrhosis is an autoimmune disease of the liver marked by the slow progressive destruction of the small bile ducts (bile canaliculi) within the liver. When these ducts are damaged, bile builds up in the liver (cholestasis) and over time damages the tissue. This can lead to scarring, fibrosis, cirrhosis, and ultimately liver failure. Hyperlipidemia with a marked increase of low-density lipoprotein (LDL) and high density lipoprotein (HDL) cholesterol levels is a common feature in patients with chronic cholestatic liver disease (Matteo Longo Current Treatment Options in Gastroenterology, 2007).

Pancreatitis

Provided herein are methods that can be used to prevent or treat pancreatitis. Pancreatitis is the inflammation of the pancreas. One of the causes of pancreatitis is hypertriglyceridemia (but not hypercholesterolemia) and only when triglyceride values exceed 1500 mg/dl (16 mmol/L). Development of pancreatitis in pregnant women could be a reflection of the hypertriglyceridemia because estrogen may raise blood triglyceride levels.

Provided herein is a method of treating acute hyperlipidemic pancreatitis in pregnancy by administering to a patient an effective amount of a pyrone analog, such as phosphorylated or phosphonated fisetin or phosphorylated or phosphonated quercetin, which reduces or eliminates hyperlipidemia and/or one or more symptoms of hyperlipidemia.

Obesity

Provided herein are methods that can be used to prevent or treat obesity. Central obesity, characterized by its high waist to hip ratio, is an important risk for metabolic syndrome. Metabolic syndrome is a combination of medical disorders which often includes diabetes mellitus type 2, high blood pressure, high blood cholesterol, and triglyceride levels (Grundy S M (2004), J. Clin. Endocrinol. Metab. 89(6): 2595-600). There are two commonly prescribed medications for obesity. One is orlistat, which reduces intestinal fat absorption by inhibiting pancreatic lipase; the other is sibutramine, which is a specific inhibitor of the neurotransmitters norepinephrine, serotonin, and dopamine in the brain. Orlistat and rimonabant lead to a reduced incidence of diabetes, and all drugs have some effect on cholesterol.

Kidney Diseases

Provided herein are methods that can be used to prevent or treat kidney diseases. Diabetes is the most common cause of chronic kidney disease and kidney failure, accounting for nearly 44 percent of new cases. Even when diabetes is controlled, the disease can lead to chronic kidney disease and kidney failure. Most people with diabetes do not develop chronic kidney disease that is severe enough to progress to kidney failure. Nearly 24 million people in the United States have diabetes, and nearly 180,000 people are living with kidney failure as a result of diabetes. High blood pressure, or hypertension, is a major factor in the development of kidney problems in people with diabetes.

Niemann-Pick Disease

Provided herein are methods that can be used to prevent or treat Niemann-Pick disease. Niemann-Pick disease is one of a group of lysosome storage diseases that affect metabolism and that are caused by genetic mutations. The three most commonly recognized forms are Niemann-Pick Types A, B and C. Niemann-Pick Type C(NPC) patients are not able to metabolize cholesterol and other lipids properly within the cell. In Niemann Pick Type C, cholesterol and glycolipids are the materials being stored rather than sphingomyelin. These fats have varied roles in the cell. Cholesterol is normally used to either build the cell, or forms an ester. In the case of an individual with NPC, there are large amounts of cholesterol that are not used as a building material and also do not form esters. This cholesterol accumulates within the cells throughout the body, but especially in the spleen, the liver and the bone marrow. Currently, there is no known cure for NPC. There is also no standard treatment that has proven to be effective. Provided herein are methods for potential treatment of NPC.

Other Disorders

Provided herein are methods that can be used to prevent or treat other disorders including but not limited to eating disorders that result in hyperlipidemia and/or hyperglycemia. A high proportion of patients suffering an acute stress such as stroke or myocardial infarction may develop hyperglycemia. In addition, hyperglycemia occurs naturally during times of infection and inflammation. When the body is stressed, endogenous catecholamines are released that serve to raise the blood glucose levels. The amount of increase varies from person to person and from inflammatory response to response.

It should be noted that although exemplary diseases are provided herein, compounds described herein may be used to treat or prevent any disease that is associated with hyperlipidemia, hypercholesterolemia, hypertriglyceridemia, or hyperglycemia.

In another aspect, compounds of the present invention may be administered in combination with lipid-lowering compounds.

Atorvastatin (marketed under the name Lipitor, Lipidra, Aztor, Torvatin, Sortis, Torvast, Torvacard, Totalip, Tulip, Xarator, Atorpic, Liprimar, Atorlip and other names), is a member of the drug class known as statins, used for lowering blood cholesterol. Atorvastatin inhibits the rate-determining enzyme located in hepatic tissue that produces mevalonate, a small molecule used in the synthesis of cholesterol and other mevalonate derivatives. This lowers the amount of cholesterol produced which in turn lowers the total amount of LDL cholesterol. As with other statins, atorvastatin is a competitive inhibitor of HMG-CoA reductase. It is a completely synthetic compound. HMG-CoA reductase catalyzes the reduction of 3-hydroxy-3-methylglutaryl-coenzyme A (HMG-CoA) to mevalonate, which is the rate-limiting step in hepatic cholesterol biosynthesis. Inhibition of the enzyme decreases de novo cholesterol synthesis, increasing expression of low-density lipoprotein receptors (LDL receptors) on hepatocytes. This increases LDL uptake by the hepatocytes, decreasing the amount of LDL-cholesterol in the blood. Like other statins, atorvastatin also reduces blood levels of triglycerides and slightly increases levels of HDL-cholesterol. In clinical trials, adding ezetimibe (Zetia) to Lipitor lowered cholesterol more effectively than Vytorin (ezetimibe+simvastatin). Atorvastatin is indicated as an adjunct to diet for the treatment of dyslipidemia, specifically hypercholesterolaemia. It has also been used in the treatment of combined hyperlipidemia (Rossi S, editor. Australian Medicines Handbook 2006).

Atorvastatin calcium tablets are currently marketed by Pfizer under the trade name Lipitor®, in tablets (10, 20, 40 or 80 mg) for oral administration. Tablets are white, elliptical, and film coated. Pfizer also packages the drug in combination with other drugs, such as is the case with its Caduet. Lipitor In most cases, the recommended Lipitor dosage for patients who are just starting the medication is Lipitor 10 mg to 20 mg once a day; however, some people may start on Lipitor 40 mg once a day if their cholesterol is extremely high. The recommended Lipitor dosage for children ages 10 to 17 is begins at Lipitor 10 mg once a day; the maximum recommended dose for children is Lipitor 20 mg.

Drugs that decrease triglyceride level include but are not limited to ascorbic acid, asparaginase, clofibrate, colestipol, fenofibrate mevastatin, pravastatin, simvastatin, fluvastatin, or omega-3 fatty acid. Drugs that decrease LDL cholesterol level include but are not limited to clofibrate, gemfibrozil, and fenofibrate, nicotinic acid, mevinolin, mevastatin, pravastatin, simvastatin, fluvastatin, lovastatin, cholestyrine, colestipol or probucol.

In another aspect, compounds of the present invention may be administered in combination with glucose-lowering compounds.

The medication class of thiazolidinedione (also called glitazones) has been used as an adjunctive therapy for hyperglycemia and diabetes mellitus (type 2) and related diseases. Thiazolidinediones or TZDs act by binding to PPARs (peroxisome proliferator-activated receptors), specifically PPARγ(gamma). The normal ligands for these receptors are free fatty acids (FFAs) and eicosanoids. When activated, the receptor migrates to the DNA, activating transcription of a number of specific genes. Chemically, the members of this class are derivatives of the parent compound thiazolidinedione, and include but are not limited to Rosiglitazone (Avandia) and Pioglitazone (Actos). For pioglitazone, the oral dosage for monotherapy is 15-30 mg once daily; if response is inadequate, the dosage may be increased in increments up to 45 mg once daily. The maximum recommended dose is 45 mg once daily. For combination therapy, the maximum recommended dose is 45 mg/day.

Drugs that decrease glucose level include but are not limited to glipizide, exenatide, incretins, sitagliptin, pioglitizone, glimepiride, rosiglitazone, metformin, exantide, vildagliptin, sulfonylurea, glucosidase inhibitor, biguanide, repaglinide, acarbose, troglitazone, and nateglinide.

In some embodiments, provided herein is a method of treating a condition by administering to an animal suffering from the condition an effective amount a lipid transport protein activator sufficient to reduce or eliminate hyperlipidemia, hypercholesterolemia, hypertriglyceridemia, or hyperglycemia and/or one or more symptoms of hyperlipidemia, hypercholesterolemia, hypertriglyceridemia, or hyperglycemia.

In some embodiments, provided herein is a method of treating a condition by administering to an animal suffering from the condition an effective amount a lipid transport protein activator in combination with a lipid-lowering compound sufficient to reduce or eliminate hyperlipidemia, hypercholesterolemia, hypertriglyceridemia, or hyperglycemia and/or one or more symptoms of hyperlipidemia, hypercholesterolemia, hypertriglyceridemia, or hyperglycemia. In some embodiments, provided herein is a method of treating a condition by administering to an animal suffering from the condition an effective amount a lipid transport protein activator in combination with a glucose-lowering compound sufficient to reduce or eliminate hyperlipidemia, hypercholesterolemia, hypertriglyceridemia, or hyperglycemia and/or one or more symptoms of hyperlipidemia, hypercholesterolemia, hypertriglyceridemia, or hyperglycemia.

In some embodiments, provided herein is a method of treating a condition by administering to an animal suffering from the condition an effective amount a lipid transport protein activator, e.g. a pyrone analog, sufficient to reduce lipid level, cholesterol level, triglyceride level or glucose level in a physiological compartment. In some embodiments, the physiological compartment is a lipid accumulating cell. In some embodiments, the physiological compartment is a macrophage. In some embodiments, the physiological compartment is a muscle cell. In some embodiments, the physiological compartment is an adipocyte. In some embodiments, the physiological compartment is a pancreatic islet cell. In some embodiments, the physiological compartment is a pancreatic beta-cell. In some embodiments, the physiological compartment is a hepatocyte.

In some embodiments the subject is an animal. In some embodiments, the animal is a mammal. Non-limiting examples of mammals are primates (e.g. lemurs, Aye-aye, lorids, galagos, tarsiers, monkeys, chimpanzees, gorillas, orangutans, and humans), cetaceans (e.g. whales, dolphins and porpoises), chiropterans (e.g. bats), perrisodactyls (e.g. horses and rhinoceroses), rodents (e.g. mice, rats, squirrels, chipmunks, gophers, porcupines, beavers, hamsters, gerbils, guinea pigs, degus, chinchillas, prairie dogs, and groundhogs), and certain kinds of insectivores such as shrews, moles and hedgehogs. In some embodiments, the mammal is a human. In some embodiments the subject is a patient.

In some embodiments, the pyrone analog and the lipid-lowering compound are co-administered. Co-administration includes simultaneous administration in separate compositions, administration at different times in separate compositions, or administration in a composition in which both agents are present. Typically, the pyrone analog is present in the composition in an amount sufficient to reduce hyperlipidemia, hypercholesterolemia, hypertriglyceridemia, or hyperglycemia and/or one or more symptoms of hyperlipidemia, hypercholesterolemia, hypertriglyceridemia, or hyperglycemia. In some embodiments, the pyrone analog is present in the composition in an amount sufficient to substantially eliminate or reduce hyperlipidemia, hypercholesterolemia, hypertriglyceridemia, or hyperglycemia and/or one or more symptoms of hyperlipidemia, hypercholesterolemia, hypertriglyceridemia, or hyperglycemia by an average of at least about 5, 10, 15, 20, 25, 30, 40, 50, 60, 70, 80, 90, more than 90%, compared to the effect without the pyrone analog.

Administration of the compounds described herein may be by any suitable means. In some embodiments, the pyrone analog is administered by oral administration, transdermal administration, or by injection (e.g., intravenous).

Administration of a pyrone analog and a second compound (e.g., a lipid-lowering compound or a glucose-lowering compound) may be by any suitable means. If the pyrone analog and a second compound (e.g., a lipid-lowering compound or a glucose-lowering compound) are administered as separate compositions, they may be administered by the same route or by different routes. If the pyrone analog and the second compound are administered in a single composition, they may be administered by any suitable route such as, for example, oral administration, transdermal administration, or by injection.

In some embodiments, dosages for pyrone analogs may be determined based on patient weight; for example, a dosage may be about 0.5-100 mg/kg of body weight, between 0.1-50 mg/kg of body weight, between 0.1-10 mg/kg of body weight, between 0.1-50 mg/kg of body weight, or between 0.1-3 mg/kg of body weight.

The compounds described herein may be used for treatment of any suitable condition including but not limited to chronic hyperlipidemia, acute hyperlipidemia, acute hypercholesterolemia, chronic hypercholesterolemia, acute hypertriglyceridemia, chronic hypertriglyceridemia, chronic hyperglycemia, acute hyperglycemia, diabetes mellitus, non-diabetic hyperglycemia, stress-induced hyperglycemia, inflammation-induced hyperglycemia, organ transplant, an autoimmune disease, cardiovascular disease, heart attack, stroke, coronary artery disease, hypertension, liver disease, primary bile cirrhosis, pancreatitis, Niemann-Pick disease, obesity, cataracts, Wilson's disease, kidney disease and an inflammatory disease.

Cardiovascular Disease

Provided herein is a method of treating cardiovascular disease in a patient by administering to the patient an effective amount of a pyrone analog, such as phosphorylated or phosphonated fisetin or phosphorylated or phosphonated quercetin, which reduces or eliminates hyperlipidemia and/or hyperglycemia and/or one or more symptoms of hyperlipidemia or hyperglycemia. Examples of cardiovascular diseases include but are not limited to atherosclerosis, Ischemic heart disease, acute myocardial infarction, congestive heart failure and stroke.

Hyperlipidemia, Hypercholesterolemia, Hypertriglyceridemia, and Hyperglycemia

In some embodiments, provided herein is a method of treating non-diabetic hyperglycemia by administering to a patient in need of treatment an effective amount of a pyrone analog, such as phosphorylated or phosphonated fisetin or phosphorylated or phosphonated quercetin, which reduces or eliminates hyperglycemia and/or one or more symptoms of hyperglycemia. Certain eating disorders can produce acute non-diabetic hyperglycemia, as in the binge phase of bulimia nervosa, when the subject consumes a large amount of calories at once, frequently from foods that are high in simple and complex carbohydrates. Certain medications increase the risk of hyperglycemia, including beta blockers, thiazide diuretics, corticosteroids, niacin, pentamidine, protease inhibitors, L-asparaginase, and some antipsychotic agents.

In some embodiments, provided herein is a method of treating stress-induced hyperglycemia by administering to a patient in need of treatment an effective amount of a pyrone analog, such as phosphorylated or phosphonated fisetin or phosphorylated or phosphonated quercetin, which reduces or eliminates hyperglycemia and/or one or more symptoms of hyperglycemia. A high proportion of patients suffering an acute stress such as stroke or myocardial infarction may develop hyperglycemia, even in the absence of a diagnosis of diabetes. Human and animal studies suggest that this is not benign, and that stress-induced hyperglycemia is associated with a high risk of mortality after both stroke and myocardial infarction.

In some embodiments, provided herein is a method of treating inflammation-induced hyperglycemia by administering to a patient in need of treatment an effective amount of a pyrone analog, such as phosphorylated or phosphonated fisetin or phosphorylated or phosphonated quercetin, which reduces or eliminates hyperglycemia and/or one or more symptoms of hyperglycemia.

In some embodiments, provided herein is a method of preventing, decreasing and/or reversing hyperlipidemia, hypercholesterolemia, hypertriglyceridemia, or hyperglycemia and/or one or more symptoms of hyperlipidemia, hypercholesterolemia, hypertriglyceridemia, or hyperglycemia by administering a lipid transport protein activator to a patient with a known or suspected symptom of hyperlipidemia, hypercholesterolemia, hypertriglyceridemia, or hyperglycemia. In some embodiments, the patient has tested positive for hyperglycemia (e.g. after a fasting glucose test) prior to administering the lipid transport protein activator, i.e. pyrone analog. In some embodiments, the patient, e.g. human, has tested positive for hyperlipidemia (e.g. after a fasting cholesterol test) prior to administering the lipid transport protein activator, i.e. pyrone analog. In some embodiments, the patient has displayed one or more symptoms of hyperglycemia as described herein prior to administering the lipid transport protein activator. In some embodiments, the patient has displayed one or more symptoms of hyperlipidemia, hypercholesterolemia, or hypertriglyceridemia as described herein prior to administering the lipid transport protein activator. In some embodiments, the patient possesses a trait (e.g. genetic trait or physical trait such as obesity) that makes the patient predisposed to hyperlipidemia, hypercholesterolemia, or hypertriglyceridemia and/or one or more symptoms of hyperlipidemia, hypercholesterolemia, or hypertriglyceridemia; and a lipid transport protein activator, i.e. a pyrone analog is administered to the patient alone or in combination with a lipid-lowering compound to prevent hyperlipidemia, hypercholesterolemia, hypertriglyceridemia and/or one more symptoms of hyperlipidemia, hypercholesterolemia, hypertriglyceridemia. In some embodiments, the patient possesses a trait (e.g. genetic trait or physical trait such as obesity) that makes the patient predisposed to hyperglycemia and/or one or more symptoms of hyperglycemia; and a lipid transport protein activator, i.e. a pyrone analog, is administered to the patient alone or in combination with a glucose-lowering compound to prevent hyperglycemia and/or one more symptoms of hyperglycemia. For example, a diabetic patient can be prescribed treatment with one or more of the pyrone analogs described herein after testing positive for hyperglycemia from a glucose blood level test such as the fasting glucose test. In another example, a patient suffering from atherosclerosis can be prescribed treatment with one or more of the pyrone analogs described herein after testing positive for hyperlipidemia from a cholesterol or triglyceride blood level test such as the fasting cholesterol or triglyceride test. Alternatively, a patient that possesses a trait (e.g. genetic trait or physical trait such as obesity) that makes the patient predisposed to hyperglycemia or hyperlipidemia and/or one or more symptoms of hyperglycemia or hyperlipidemia can be prescribed treatment with one or more of pyrone analogs described herein to prevent hyperglycemia or hyperlipidemia and/or one more symptoms of hyperglycemia or hyperlipidemia, even when the patient is not experiencing hyperglycemia or hyperlipidemia and/or one or more symptoms of hyperglycemia or hyperlipidemia.

In some embodiments, provided herein is a method for reversing hyperglycemia or hyperlipidemia and/or one or more symptoms of hyperglycemia or hyperlipidemia in a human by administering to the human an amount of a pyrone analog e.g. phosphorylated or phosphonated fisetin or phosphorylated or phosphonated quercetin, sufficient to partially or completely reverse hyperglycemia or hyperlipidemia and/or one or more symptoms of hyperglycemia or hyperlipidemia in that human. In some embodiments, the lipid transport protein modulator is a pyrone analog.

The pyrone analog can be administered by any suitable route such as orally or by injection, e.g., intravenously or intraperitoneally, in a dose sufficient to partially or completely reverse hyperglycemia, hyperlipidemia, and/or one or more symptoms of hyperglycemia or hyperlipidemia. Such a dose in a human can be, e.g., about 0.1-100 mg, or about 0.5-50 mg, or about 1-40 mg, or 1, 2, 3, 4, 5, 6, 7, 8, 9, 10, 12, 14, 16, 18, or 20 mg. In general, the dose can be in the range of 0.1-3 mg/kg of body weight.

In addition to the compounds referred to herein, other compounds that activate a lipid transporter are also anticipated to lower the level of lipid, preferably cholesterol and triglycerol, and thus be useful in treating hyperlipidemia.

For therapeutic applications, the lipid transporter activator, i.e. pyrone analog, may be incorporated into pharmaceutical compositions, such as tablets, pills, capsules, solutions, suspensions, creams, ointments, gels, salves, lotions and the like, using such pharmaceutically acceptable excipients and vehicles which per se are well known in the art. For example, preparation of topical formulations are well described in Remington's Pharmaceutical Science, Edition 17, Mack Publishing Company, Easton, Pa.; for topical application, the pyrone analog could also be administered as a powder or spray, particularly in aerosol form. If the pyrone analog is to be administered systemically, it may be prepared as a powder, pill, tablet or the like or as a syrup or elixir suitable for oral administration. For intravenous or intraperitoneal administration, the pyrone analog may be prepared as a solution or suspension capable of being administered by injection. In certain cases, it may be useful to formulate the pyrone analog in a solution for injection. In other cases, it may be useful to formulate the pyrone analog in suppository form or as extended release formulation for deposit under the skin or intramuscular injection.

A pyrone analog may be administered in a therapeutically effective dose. In some embodiments, a therapeutic concentration will be that concentration which is effective to lower the concentration of lipids, for example triglycerol and cholesterol, in a patient. In other embodiments, a therapeutic concentration will be that concentration which is effective to lower the concentration of glucose in a patient. For example, a formulation comprising between about 0.1 and about 3 mg of a pyrone analog/kg of body weight, between about 0.3 mg/kg and 2 mg/kg, about 0.7 mg/kg, or about 1.5 mg/kg will constitute a therapeutically effective concentration for oral application, with routine experimentation providing adjustments to these concentrations for other routes of administration if necessary.

In one embodiment, a pharmaceutical composition comprising the pyrone analog is administered orally. Such composition may be in the form of a liquid, syrup, suspension, tablet, capsule, or gelatin-coated formulation. In another embodiment, a pharmaceutical composition comprising a pyrone analog is topically administered. Such composition may be in the form of a patch, cream, lotion, emulsion, or gel. In yet another embodiment, a pharmaceutical composition comprising the pyrone analog may be inhaled. Such composition may be formulated as an inhalant, suppository or nasal spray.

In some embodiments, a pyrone analog, such as phosphorylated or phosphonated fisetin or phosphorylated or phosphonated quercetin, is administered alone or with a pharmaceutically acceptable carrier. In some embodiments, a pyrone analog is administered in combination with a lipid-lowering compound that reduces hyperlipidemia and/or one or more symptoms of hyperlipidemia. In some embodiments, a pyrone analog is administered in combination with a glucose-lowering compound that reduces hyperglycemia and/or one or more symptoms of hyperglycemia.

In some embodiments, more than one pyrone analogs and/or lipid or glucose-lowering compounds or other agents are also administered. When two or more agents are co-administered, they may be co-administered in any suitable manner, e.g., as separate compositions, in the same composition, by the same or by different routes of administration.

In some embodiments, a pyrone analog is administered in a single dose. In some embodiments, a pyrone analog or a combination (mixture) of compounds is administered in multiple doses.

Dosing may be about once, twice, three times, four times, five times, six times, or more than six times per day. In some embodiments, dosing may be about once a month, once every two weeks, once a week, once every other day or any other suitable interval. In some embodiments, the administration continues for more than about 6, 10, 14, 28 days, two months, six months, or one year. In some cases, continuous dosing is achieved and maintained as long as necessary, e.g., in a diabetic patient, which may require dosing for the rest of his or her life.

Administration of the one or more agents may continue as long as necessary. In some embodiments, a pyrone analog is administered for more than about 1, 2, 3, 4, 5, 6, 7, 14, or 28 days. In some embodiments, a pyrone analog is administered for less than about 28, 14, 7, 6, 5, 4, 3, 2, or 1 day. In some embodiments, a pyrone analog is administered chronically on an ongoing basis, e.g., for the treatment of chronic effects.

An effective amount of a lipid transport protein modulator may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

The lipid transport protein modulator i.e. pyrone analog may be administered in dosages as described herein. Dosing ranges for lipid-lowering or glucose-lowering compounds are known in the art and are contemplated herein. Individualization of dosing regimen may be utilized for optimal therapy due to inter-subject variability and pharmacokinetics. Dosing for the lipid transport modulator may be determined empirically.

For a flavonoid, e.g., phosphorylated or phosphonated fisetin or phosphorylated or phosphonated quercetin, typical daily dose ranges include, for example, about 1-5000 mg, about 1-3000 mg, about 1-2000 mg, about 1-1000 mg, about 1-500 mg, about 1-100 mg, about 10-5000 mg, about 10-3000 mg, about 10-2000 mg, about 10-1000 mg, about 10-500 mg, about 10-200 mg, about 10-100 mg, about 20-2000 mg, about 20-1500 mg, about 20-1000 mg, about 20-500 mg, about 20-100 mg, about 50-5000 mg, about 50-4000 mg, about 50-3000 mg, about 50-2000 mg, about 50-1000 mg, about 50-500 mg, about 50-100 mg, about 100-5000 mg, about 100-4000 mg, about 100-3000 mg, about 100-2000 mg, about 100-1000 mg, or about 100-500 mg. In some embodiments, the daily dose of phosphorylated or phosphonated fisetin or a phosphorylated or phosphonated fisetin derivative is about 10 mg, about 20 mg, about 40 mg, about 80 mg, about 100, about 200, about 300, about 400, about 500, about 600, about 700, about 800, about 900, or about 1000 mg. In some embodiments, the daily dose of phosphorylated or phosphonated quercetin or a phosphorylated or phosphonated quercetin derivative is about 10 mg, about 20 mg, about 40 mg, about 80 mg, about 100, about 200, about 300, about 400, about 500, about 600, about 700, about 800, about 900, or about 1000 mg.

Daily doses may be administered in single or multiple doses. For instance, in some embodiments the lipid transport modulator is administered 3 times per day of an oral dose of 500 mg. In other embodiments the lipid transport modulator is administered 3 times per day of an i.v. dose of 150 mg. Daily doses of fisetin, a fisetin derivative, a phosphorylated or phosphonated fisetin, or a phosphorylated or phosphonated fisetin derivative may be administered in the same or separate composition as other pyrone analogs, lipid-lowering compound or glucose-lowering compound. Daily dose range may depend on the form of flavonoid, e.g., the carbohydrate moieties attached to the flavonoid, and/or factors with which the flavonoid is administered, as described herein.

When a lipid transport protein, which is the target of the pyrone analog, is present on the cells, unit dose forms of the pyrone analog may be adjusted such that hyperglycemia, hyperlipidemia, and/or one or more symptoms of hyperglycemia or hyperlipidemia, are reduced to have the maximum therapeutic effect.

C. Additional Methods of Using Phosphonated Pyrone Analogs

In another aspect, the invention provides methods, including methods of treatment, methods of decreasing the concentration of a substance in a physiological compartment (e.g., methods of delaying the onset or preventing chronic neurodegenerative diseases), methods of enhancing a therapeutic effect of a substance, methods of delaying, preventing, reducing or eliminating tolerance or dependence in an animal that is administered a substance, methods of drug wash-out, and methods for identifying modulators of bloodtissue barrier transport proteins.

For simplicity, some methods will be described in terms of reduction of a side effect of a substance. It is understood that the methods apply equally to exclusion of a substance from the fetal compartment, or reduction of fetal effects of a substance.

The term “animal” or “animal subject” as used herein includes humans as well as other mammals. The methods generally involve the administration of one or more drugs for the treatment of one or more diseases. Combinations of agents can be used to treat one disease or multiple diseases or to modulate the side-effects of one or more agents in the combination.

The term “treating” and its grammatical equivalents as used herein include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

In some embodiments, the invention provides a method of treating a condition by administering to an animal in need of treatment an effective amount of a phosphonated polyphenol e.g. phosphonated pyrone analog such as a phosphonated flavonoid, such as a phosphonated quercetin, phosphonated fisetin, or phosphonated 5,7-dideoxyquercetin sufficient to reduce or eliminate a side effect of the therapeutic agent. In some embodiments, the activator reduces or eliminates a plurality of side effects of the therapeutic agent. In some embodiments the animal is a mammal, e.g., a human.

The therapeutic agent and the phosphonated polyphenol e.g. phosphonated pyrone analog such as a phosphonated flavonoid, such as a phosphonated quercetin, phosphonated fisetin, or phosphonated 5,7-dideoxyquercetin are co-administered. “Co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present. Thus, in some embodiments, the phosphonated polyphenol e.g. phosphonated pyrone analog such as a phosphonated flavonoid, such as a phosphonated quercetin, phosphonated fisetin, or phosphonated 5,7-dideoxyquercetin are administered in a single composition. In some embodiments, the therapeutic agent and the phosphonated polyphenol e.g. phosphonated pyrone analog such as a phosphonated flavonoid, such as a phosphonated quercetin, phosphonated fisetin, or phosphonated 5,7-dideoxyquercetin are admixed in the composition. Typically, the therapeutic agent is present in the composition in an amount sufficient to produce a therapeutic effect, and the phosphonated polyphenol e.g. phosphonated pyrone analog such as a phosphonated flavonoid, such as a phosphonated quercetin, phosphonated fisetin, or phosphonated 5,7-dideoxyquercetin is present in the composition in an amount sufficient to reduce a side effect of the therapeutic agent. In some embodiments, the therapeutic agent is present in an amount sufficient to exert a therapeutic effect and the phosphonated polyphenol e.g. phosphonated pyrone analog such as a phosphonated flavonoid, such as a phosphonated quercetin, phosphonated fisetin, or phosphonated 5,7-dideoxyquercetin is present in an amount sufficient to decrease a side effect of the therapeutic agent by an average of at least about 5, 10, 15, 20, 25, 30, 40, 50, 60, 70, 80, 90, more than 90%, or substantially eliminate a side effect compared to the effect without the phosphonated polyphenol e.g. phosphonated pyrone analog such as a phosphonated flavonoid, such as a phosphonated quercetin, phosphonated fisetin, or phosphonated 5,7-dideoxyquercetin.

In some embodiments the methods of the invention are used to reduce the side effect and/or increase the effectiventss of an immunosuppressant. The immunosuppressant can be a cyclosporin (Neoral, Sandimmune, SangCya), an azathioprine (Imuran), a corticosteroid such as prednisolone (Deltasone, Orasone), basiliximab (Simulect), daclizumab (Zenapax), muromonab CD3 (Orthoclone OKT3), tacrolimus (Prograf®), ascomycin, pimecrolimus (Elidel), azathioprine (Imuran), cyclosporin (Sandimmune, Neoral), glatiramer acetate (Copaxone), mycopehnolate (CellCept), sirolimus (Rapamune), or voclosporin.

In some embodiments methods of the invention are used to reduce the side effect and/or increase the effectiventss of a calcineurin inhibitor such as tacrolimus (Prograf®),

The methods of the invention can be used to reduce the side effect and/or increase the effectiventss of a selective estrogen receptor modulator (SERM), such as tamoxifen.

The methods of the invention can be used to reduce the side effect and/or increase the effectiventss of an antilipedimic agent such as an HMG-CoA inhibitor such as lovastatin, simvastatin, pravastatin, fluvastatin, or atorvastatin

The methods of the invention can be used to reduce the side effect and/or increase the effectiventss of an antihyperglycemic agent (antiglycemics, hypoglycemic agents) such as glyburide, glipizide, gliclazide, or glimepride; a meglitinide such as repaglinide or netaglinide, a biguanide such as metformin, a thiazolidinedione, an α-glucosidase inhibitor such as acarbose or miglitol, glucagon, somatostatin, or diazoxide.

The methods of the invention can be used to reduce the side effect and/or increase the effectiventss of a cannabinoid.

The methods of the invention can be used to reduce the side effect and/or increase the effectiventss of an antidepressant. In some embodiments, antidepressants cause the side effects of high blood sugar and diabetes. The methods of the invention can be used, for example to reduce these side effects. In some embodiments the therapeutic agent is an antidepressant selected from the group of aripiprazone (Abilify), nefazodone (Serzone), escitalopram oxalate (Lexapro), sertraline (Zoloft), escitalopram (Lexapro), fluoxetine (Prozac), bupropion (Wellbutrin, Zyban), paroxetine (Paxil), venlafaxine (Effexor), trazodone (Desyrel), amitriptyline (Elavil), citalopram (Celexa), duloxetine (Cymbalta), mirtazapine (Remeron), nortriptyline (Pamelor), imipramine (Tofranil), amitriptyline (Elavil), clomipramine (Anafranil), doxepin (Adapin), trimipramine (Surmontil), amoxapine (Asenidin), desipramine (Norpramin), maprotiline (Ludiomil), protryptiline (Vivactil), citalopram (Celexa), fluvoxamine (Luvox), phenelzine (Nardil), trancylpromine (Parnate), and selegiline (Eldepryl).

The methods of the invention can be used to reduce the side effect and/or increase the effectiventss of an antineuropathic agent such as gabapentin.

The methods of the invention can be used to reduce the side effect and/or increase the effectiventss of an anticonvulsant. In some cases, it can be an anticonvulsant that also has efficacy in the treatment of pain. The therapeutic agent can be, for example, acetazolamide (Diamox), carbamazepine (Tegretol), clobazam (Frisium), clonazepam (Klonopin/Rivotril), clorazepate (Tranxene-SD), diazepam (Valium), divalproex sodium (Depakote), ethosuximide (Zarontin), ethotoin (Peganone), felbamate (Felbatol), fosphenyloin (Cerebyx), gabapentin (Neurontin), lamotrigine (Lamictal), levetiracetam (Keppra), lorezepam (Ativan), mephenyloin (Mesantoin), metharbital (Gemonil), methsuximide (Celontin). Methazolamide (Neptazane), oxcarbazepine (Trileptal), phenobarbital, phenyloin (Dilantin/Epanutin), phensuximide (Milontin), pregabalin (Lyrica), primidone (Mysoline), sodium valproate (Epilim), stiripentol (Diacomit), tiagabine (Gabitril), topiramate (Topamax), trimethadione (Tridione), valproic acid (Depakene/Convulex), vigabatrin (Sabril), zonisamide (Zonegran), or cefepime hydrochloride (Maxipime).

In some embodiments, the phosphonated polyphenol will have higher water solubility than the non-phosphonated polyphenol. In some embodiments the phosphonated polyphenol will have multiple phosphonates and will have higher water solubility than the polyphenol with fewer phosphonate groups. For example, quercetin aglycone has relatively low solubility in water, and relatively low solubility in the blood. The addition of a phosphonate to quercetin will tend to improve the solubility of the quercetin in water and in the blood and thus increase its bioavailability. The addition of the phosphonate group can increase water solubility by adding polarity, by adding an ionic substituent, and in some cases due to geometrical (molecular shape) factors. In some embodiments of the invention, the phosphonated polyphenol is at least about 10%, 20%, 25%, 30%, 40%, 50%, 60%, 75%, 90%, or 100% or at least about 2, 3, 4 5, 10, 20, 50, 100, 1,000, or 10,000 times more water soluble than the corresponding non-phosphonated polyphenol. In some embodiments of the invention, the phosphonated polyphenol is at least about 10%, 20%, 25%, 30%, 40%, 50%, 60%, 75%, 90%, or 100% or at least about 2, 3, 4 5, 10, 20, 50, 100, 1,000, or 10,000 times more soluble in a bodily fluid than the corresponding non-phosphonated polyphenol. Methods for determining solubility are well known in the art. Where the fluid is clear, optical methods may be used for determining solubility. It is also possible to determine solubility by a direct measurement of the dissolved component, for example by HPLC. The solubility may be dependent on pH. In some embodiments the pH of the solution is neutral pH. In some embodiments the pH is between 6.8 and 7.2, in some embodiments the pH is between 6.5 and 7.5, in some embodiments the pH is between 6.0 and 7.0, in some embodiments the pH is between 5 and 9, in some embodiments the pH is between 4 and 10, in some embodiments the pH is between 3 and 11, in some embodiments the pH is between 2 and 12. The biological fluids of the present invention can be any fluid in an animal. Non-limiting examples of biological fluids are: blood, lymph, saliva, mucus, gastric juice, urine, aqueous humor, and semen.

One embodiment of the invention is a method for the treatment of an animal by oral administration of a therapeutic agent and a phosphonated polyphenol, e.g. phosphonated pyrone analog such as a phosphonated flavonoid, such as a phosphonated quercetin, phosphonated fisetin, or phosphonated 5,7-dideoxyquercetin that is greater than about 10%, 20%, 25%, 30%, 40%, 50%, 60%, 75%, 90%, or 100% or about 2, 3, 4 5, 10, 20, 50, 100, 1,000, or 10,000 times more soluble in water than the corresponding non-phosphonated polyphenol. One embodiment of the invention is a method for the treatment of an animal by oral administration of a therapeutic agent and a phosphonated polyphenol, e.g. phosphonated pyrone analog such as a phosphonated flavonoid, such as a phosphonated quercetin, phosphonated fisetin, or phosphonated 5,7-dideoxyquercetin that is greater than about 10%, 20%, 25%, 30%, 40%, 50%, 60%, 75%, 90%, or 100% or greater than about 2, 3, 4 5, 10, 20, 50, 100, 1,000, or 10,000 times more soluble in a bodily fluid than the corresponding non-phosphonated polyphenol. In some embodiments the therapeutic agent is an immunosuppressive agent, e.g. a calcineurin inhibitor such as tacrolimus or sirolimus.

In some embodiments, the increased water solubility will result in increased solubility of the polyphenol in a bodily fluid. In some embodiments, the increased solubility in a bodily fluid will result in greater bioavailability of the phosphonated polyphenol than for the corresponding non-phosphonated polyphenol.

In some embodiments, the phosphonated polyphenol will provide a longer half-life of drug effect than for a non-phosphonated polyphenol. For example, and without being limited by mechanism, where a phosphonated polyphenol is not an active BTB transport protein modulator, and its de-phosphonated form is active as a BTB transport protein modulator, the amount of active form can depend on the rate of de-phosphonation. If the rate of de-phosphonation is relatively slow, the de-phosphonation process can act to delay the delivery of the active form. Under these conditions, the phosphonated form acts as a kind of reservoir for the active form of the drug, thus extending the half life of drug effect. It will be understood by those of skill in the art that the relative rates of de-phosphonation and the relative rates of absorption, clearance, and volume of distribution of the phosphonated and de-phosphonated forms can influence the half life of drug effect for the drug. In some embodiments, the de-phosphonation of the phosphonated form can be used as a tool to control the timing and the area to which the active compound is delivered, allowing the control of the target concentration and of the maintenance dose.

In some embodiments, the phosphonated form is also an active form, i.e., dephosphonation is not necessary in order to achieve the desired modulation of side effects of a therapeutic agent. The phosphonated form may be more active, equally active, or less active than the dephosphonated form, and the effects of the phosphonated form may be due to a combination of its own effect and the effect and timing of appearance of the dephosphonated from. However, it will be understood that the modulation of one or more side effects and/or therapeutic effects of a therapeutic agent by the phosphonated pyrone analogs, as described herein, is not limited by the mechanism by which it is achieved.

In some embodiments, the therapeutic agent and the phosphonated polyphenol are administered, at least in part, as an ionic complex between an opiate or an immunomodulator and a phosphonated polyphenol. In some cases, the administration of the ionic complex results in higher solubility and greater bioavailability than where the compounds are administered without comprising an ionic complex.

Administration of the therapeutic agent and the phosphonated polyphenol e.g. phosphonated pyrone analog such as a phosphonated flavonoid, such as a phosphonated quercetin, phosphonated fisetin, or phosphonated 5,7-dideoxyquercetin may be any suitable means. If the agents are administered as separate compositions, they may be administered by the same route or by different routes. If the agents are administered in a single composition, they may be administered by any suitable route. In some embodiments, the agents are administered as a single composition by oral administration. In some embodiments, the agents are administered as a single composition by transdermal administration. In some embodiments, the agents are administered as a single composition by injection.

In some embodiments, the phosphonated polyphenol e.g. phosphonated pyrone analog such as a phosphonated flavonoid, such as a phosphonated quercetin, phosphonated fisetin, or phosphonated 5,7-dideoxyquercetin is a side effect modulator, e.g. BTB transport protein modulator. BTB transport protein modulators are as described herein. In some embodiments, a phosphonated polyphenol is used. In some embodiments, a phosphonated pyrone analog such as a phosphonated flavonoid is used. In some embodiments, the phosphonated pyrone analog such as a phosphonated flavonoid is phosphonated quercetin, phosphonated isoquercetin, phosphonated flavon, phosphonated chrysin, phosphonated apigenin, phosphonated rhoifolin, phosphonated diosmin, phosphonated galangin, phosphonated fisetin, phosphonated morin, phosphonated rutin, phosphonated kaempferol, phosphonated myricetin, phosphonated taxifolin, phosphonated naringenin, phosphonated naringin, phosphonated hesperetin, phosphonated hesperidin, phosphonated chalcone, phosphonated phloretin, phosphonated phlorizdin, phosphonated genistein, phosphonated 5,7-dideoxyquercetin, phosphonated biochanin A, phosphonated catechin, or phosphonated epicatechin. In some embodiments, the phosphonated pyrone analog such as a phosphonated flavonoid is phosphonated quercetin, phosphonated fisetin, phosphonated 5,7-dideoxyquercetin, phosphonated kaempferol, or phosphonated galangin. In some embodiments, the phosphonated pyrone analog such as a phosphonated flavonoid is phosphonated quercetin. In some embodiments, the phosphonated pyrone analog such as a phosphonated flavonoid is phosphonated fisetin. In some embodiments, the phosphonated pyrone analog such as a phosphonated flavonoid is phosphonated 5,7-dideoxyquercetin. In some embodiments, the phosphonated pyrone analog such as a phosphonated flavonoid is quercetin-3′-O-methylphosphonate. Dosages are as provided for compositions. Typically, the daily dosage of the side effect modulator, e.g. BTB transport protein modulator will be about 0.5-100 mg/kg.

The therapeutic agent may be any therapeutic agent described herein. In some embodiments, the therapeutic agent is an immunosuppressant, antineoplastic, amphetamine, antihypertensive, vasodilator, barbiturate, membrane stabilizer, cardiac stabilizer, glucocorticoid, chemotherapeutic agent, or antiinfective, immunomodulator, tolerogen, immunostimulants, drug acting on the blood and the blood-forming organs, hematopoietic agent, growth factor, mineral, and vitamin, anticoagulant, thrombolytic, antiplatelet drug, hormone, hormone antagonist, pituitary hormone, thyroid and antithyroid drug, estrogen and progestin, androgen, adrenocorticotropic hormone; adrenocortical steroid and synthetic analogs, insulin, oral hypoglycemic agents, calcium, phosphonate, parathyroid hormone, vitamin D, calcitonin, and other compounds.

The methods of the invention may be used for treatment of any suitable condition, e.g., diseases of the heart, circulation, lipoprotein metabolism, hemostasis and thrombosis, respiratory system, kidney, gastrointestinal tract, endocrine system, reproductive system, or hemopoeitic system, where one or more therapeutic agents are used that have side effect. For example, in some embodiments, the methods of the invention include the treatment of hypertension in an animal by administering to an animal in need of treatment an effective amount of an antihypertensive and an effective amount of a phosphonated polyphenol e.g. phosphonated pyrone analog such as a phosphonated flavonoid, such as a phosphonated quercetin, phosphonated fisetin, or phosphonated 5,7-dideoxyquercetin that reduces or eliminates a side effect of the hypertensive. Another exemplary embodiment is the treatment or prevention of infection in an animal by administering to an animal in need of treatment or prevention of infection an effective amount of an antiinfective agent and an effective amount of a phosphonated polyphenol e.g. phosphonated pyrone analog such as a phosphonated flavonoid, such as a phosphonated quercetin, phosphonated fisetin, or phosphonated 5,7-dideoxyquercetin that reduces or eliminates a side effect of the antiinfective agent.

Another exemplary embodiment is the treatment or prevention of cancer in an animal by administering to an animal in need of treatment or prevention of cancer an effective amount of an chemotherapeutic agent such as tamoxifen and an effective amount of a phosphonated polyphenol e.g. phosphonated pyrone analog such as a phosphonated flavonoid, such as a phosphonated quercetin, phosphonated fisetin, or phosphonated 5,7-dideoxyquercetin that reduces or eliminates a side effect of the chemotherapeutic agent.

Another exemplary embodiment is the treatment of graft rejection in an animal by administering to an animal in need of prevention or treatment an effective amount of an immunosuppressive agent, e.g., an calcineurin inhibitor such as sirolimus or tacrolimus, and an effective amount of a phosphonated polyphenol e.g. phosphonated pyrone analog such as a phosphonated flavonoid, such as a phosphonated quercetin, phosphonated fisetin, or phosphonated 5,7-dideoxyquercetin that reduces or eliminates a side effect or endocrine effect of the immunosuppressive agent.

Another exemplary embodiment is the prevention of organ rejection in an animal by administering to an animal that has received or will receive an organ transplant an effective amount of a calcineurin inhibitor such as tacrolimus or a tacrolimus analog and an effective amount of a phosphonated polyphenol e.g. phosphonated pyrone analog such as a phosphonated flavonoid, such as a phosphonated quercetin, phosphonated fisetin, or phosphonated 5,7-dideoxyquercetin that reduces or eliminates a side effect, e.g., a hyperglycemic effect or a side effect of the calcineurin inhibitor.

When a therapeutic agent and a phosphonated polyphenol e.g. phosphonated pyrone analog such as a phosphonated flavonoid, such as a phosphonated quercetin, phosphonated fisetin, or phosphonated 5,7-dideoxyquercetin that reduces or eliminates a side effect of the therapeutic agent are used in combination, any suitable ratio of the two agents, e.g., molar ratio, wt/wt ration, wt/volume ratio, or volume/volume ratio, as described herein, may be used.

In some embodiments of the methods of the invention, the invention provides a method of treating a condition by administering to an animal suffering from the condition an effective amount of tacrolimus and an amount of a BTB transport protein modulator sufficient to change the concentration of tacrolimus in a physiological compartment. In some embodiments of the methods of the invention the physiological compartment is selected from the group consisting of blood, lymph nodes, spleen, peyer's patches, lungs, heart kidney, pancreas liver, and gull bladder. In some embodiments of the methods of the invention the BTB transport modulator decrease the clearance of tacrolimus from a compartment where the drug is exerting therapeutic effect.

1. Methods of Modulating the Concentration of a Substance in a Physiological Compartment

The invention provides methods for reducing the concentration of a substance in a physiological compartment by selectively increasing efflux of the substance from the physiological compartment to an external environment. The physiological compartment preferably is a central nervous system or a fetal compartment.

In some embodiments, compositions of the invention may be administered chronically to an individual in order to prevent, delay the appearance, or slow or halt the progression of a chronic neurodegenerative condition. In some embodiments, compositions of the invention may be administered chronically to an individual in order to remove from the CNS one or more substances associated with a chronic neurodegenerative condition. In some embodiments, the neurodegenerative condition is prion disease, Alzheimer's disease (AD), Parkinson's disease (PD), Huntington's disease (HD), ALS, multiple sclerosis, transverse myelitis, motor neuron disease, Pick's disease, tuberous sclerosis, lysosomal storage disorders, Canavan's disease, Rett's syndrome, spinocerebellar ataxias, Friedreich's ataxia, optic atrophy, or retinal degeneration. In some embodiments, the neurodegenerative disease is AD. In some embodiments, the substance associated with a neurodegenerative disease is amyloid beta. In some embodiments, a phosphonated pyrone analog such as a phosphonated flavonoid is administered to the individual, such as phosphonated quercetin, phosphonated isoquercetin, phosphonated flavon, phosphonated chrysin, phosphonated apigenin, phosphonated rhoifolin, phosphonated diosmin, phosphonated galangin, phosphonated fisetin, phosphonated morin, phosphonated rutin, phosphonated kaempferol, phosphonated myricetin, phosphonated taxifolin, phosphonated naringenin, phosphonated naringin, phosphonated hesperetin, phosphonated hesperidin, phosphonated chalcone, phosphonated phloretin, phosphonated phlorizdin, phosphonated genistein, phosphonated 5,7-dideoxyquercetin, phosphonated biochanin A, phosphonated catechin, or phosphonated epicatechin. In some embodiments, the individual is a human and is chronically administered an amount of phosphonated quercetin, phosphonated fisetin, or phosphonated 5,7-dideoxyquercetin effective in removing amyloid beta from the CNS. In some embodiments, the phosphonated quercetin, phosphonated fisetin, or phosphonated 5,7-dideoxyquercetin is administered in a pharmaceutical composition with a pharmaceutically acceptable excipient at a dose of 100 mg-10,000 mg per day. Other dosages of phosphonated quercetin, phosphonated fisetin, or phosphonated 5,7-dideoxyquercetin, as described herein, may also be used.

In some embodiments, the invention provides a method of increasing the concentration of a therapeutic agent in a non-CNS compartment by the administration of a phosphonated polyphenols, e.g. phosphonated pyrone analog such as a phosphonated flavonoid such as phosphonated quercetin, phosphonated fisetin, or phosphonated 5,7-dideoxyquercetin. While not being bound by theory, a BTB transport protein activator can result in the exclusion of a compound or removal of compound from the CNS compartment. Because the compartments of the body are interconnected, where the compound, such as a therapeutic agent, is excluded from the CNS compartment, there can be more of the compound available to the periphery than where the compound is distributed into the periphery as well. In some embodiments, the concentration of therapeutic agent in a non-CNS compartment is at least about 1, 5, 10, 15, 20, 25, 30, 35, 40, 45, 50, 55, 60, 65, 70, 75, 80, 85, 90, 95, or more than 95% higher than without the administration of a phosphonated polyphenol, e.g. phosphonated pyrone analog such as a phosphonated flavonoid such as phosphonated quercetin, phosphonated fisetin, or phosphonated 5,7-dideoxyquercetin.

In some embodiments of the methods of the invention, the invention provides a method of treating a condition by administering to an animal suffering from the condition an effective amount of tacrolimus and an amount of a BTB transport protein modulator sufficient to change the concentration of tacrolimus in a physiological compartment. In some embodiments of the methods of the invention the physiological compartment is selected from the group consisting of blood, lymph nodes, spleen, peyer's patches, lungs, heart kidney, pancreas liver, and gull bladder. In some embodiments of the methods of the invention the BTB transport modulator decrease the clearance of tacrolimus from a compartment where the drug is exerting therapeutic effect.

2. Methods of Treating Pain.

The invention provides methods of treating pain such as acute or chronic pain, using therapeutic agents and the phosphonated compositions of the invention. Any suitable type of pain, whether acute or chronic, may be treated by the methods of the invention. Thus, in some embodiments, the invention provides a method of treating an animal for pain by administering to an animal in pain an effective amount of an opioid analgesic agent, e.g. an opioid receptor agonist such as oxycodone or morphine and an amount of a polyphenol e.g. phosphonated pyrone analog such as a phosphonated flavonoid, such as a phosphonated quercetin, phosphonated fisetin, or phosphonated 5,7-dideoxyquercetin sufficient to reduce a side effect of the opioid agent. Further description of types of pain, opioid agents and treatment of pain may be found in U.S. Patent Publication No. US2006/0111308 and PCT Publication No. WO/06055672, incorporated by reference herein in their entirety.

3. Wash-Out Methods

The invention further provides methods of reversing one or more side effects of a substance by administering a phosphonated polyphenol e.g. phosphonated pyrone analog such as a phosphonated flavonoid, such as a phosphonated quercetin, phosphonated fisetin, or phosphonated 5,7-dideoxyquercetin to an animal that has received an amount of the substance sufficient to produce one or more side effects. The methods are especially useful in a situation where it is desired to rapidly reverse one or more side effects of a substance, e.g., in an overdose situation or to enhance recovery from general anesthesia. Any suitable phosphonated polyphenol e.g. phosphonated pyrone analog such as a phosphonated flavonoid, such as a phosphonated quercetin, phosphonated fisetin, or phosphonated 5,7-dideoxyquercetin described herein may be used.

In some embodiments, the invention provides a method for reversing a side effect of an agent in a human by administering to the human an amount of a phosphonated polyphenol e.g. phosphonated pyrone analog such as a phosphonated flavonoid, such as a phosphonated quercetin, phosphonated fisetin, or phosphonated 5,7-dideoxyquercetin sufficient to partially or completely reverse a central nervous system effect of the agent, where the human has received an amount of said agent sufficient to produce a central nervous system effect. In some embodiments, the agent is a general anesthetic. Examples of general anesthetics include, but not limited to, desflurane, dexmedetomidine, diazepam, droperidol, enflurane, etomidate, halothane, isoflurane, ketamine, lorazepam, methohexital, methoxyflurane, midazolam, nitrous oxide propofol, sevoflurane, and thiopental. In some embodiments, the human has received an overdose of the agent producing the side effect. In some embodiments, the individual continues to experience peripheral effects of the agent. In some embodiments, the phosphonated polyphenol e.g. phosphonated pyrone analog such as a phosphonated flavonoid, such as a phosphonated quercetin, phosphonated fisetin, or phosphonated 5,7-dideoxyquercetin is a side effect modulator, e.g. BTB transport protein modulator. In some embodiments, the phosphonated pyrone analog such as a phosphonated flavonoid is phosphonated quercetin, phosphonated isoquercetin, phosphonated flavon, phosphonated chrysin, phosphonated apigenin, phosphonated rhoifolin, phosphonated diosmin, phosphonated galangin, phosphonated fisetin, phosphonated morin, phosphonated rutin, phosphonated kaempferol, phosphonated myricetin, phosphonated taxifolin, phosphonated naringenin, phosphonated naringin, phosphonated hesperetin, phosphonated hesperidin, phosphonated chalcone, phosphonated phloretin, phosphonated phlorizdin, phosphonated genistein, phosphonated 5,7-dideoxyquercetin, phosphonated biochanin A, phosphonated catechin, or phosphonated epicatechin. In some embodiments, the phosphonated pyrone analog such as a phosphonated flavonoid is phosphonated quercetin. In some embodiments, the phosphonated pyrone analog such as a phosphonated flavonoid is phosphonated fisetin. In some embodiments, the phosphonated pyrone analog such as a phosphonated flavonoid is phosphonated 5,7-dideoxyquercetin. In some embodiments, the phosphonated pyrone analog such as a phosphonated flavonoid is quercetin-3′-O-methylphosphonate. Typically, the phosphonated pyrone analog such as a phosphonated flavonoid, such as a phosphonated quercetin will be administered by injection, e.g., intravenously or intraperitoneally, in a dose sufficient to partially or completely reverse a side effect of the substance. Such a dose in a human can be, e.g., about 0.1-100 gm, or about 0.5-50 gm, or about 1-20 gm, or 1, 2, 3, 4, 5, 6, 7, 8, 9, 10, 12, 14, 16, 18, or 20 gm. In general, the dose can be 0.01-1.5 gm/kg.

4. Administration

The methods of the invention involve the administration of a phosphonated polyphenol e.g. phosphonated pyrone analog such as a phosphonated flavonoid, such as a phosphonated quercetin, phosphonated fisetin, or phosphonated 5,7-dideoxyquercetin. In some embodiments, a therapeutic agent that produces a side effect is administered in combination with a phosphonated polyphenol e.g. phosphonated pyrone analog such as a phosphonated flavonoid, such as a phosphonated quercetin, phosphonated fisetin, or phosphonated 5,7-dideoxyquercetin that reduces a side effect of the therapeutic agent. In some embodiments, other agents are also administered, e.g., other therapeutic agents. When two or more agents are co-administered, they may be co-administered in any suitable manner, e.g., as separate compositions, in the same composition, by the same or by different routes of administration.

In some embodiments, the phosphonated polyphenol e.g. phosphonated pyrone analog such as a phosphonated flavonoid, such as a phosphonated quercetin, phosphonated fisetin, or phosphonated 5,7-dideoxyquercetin is administered in a single dose. This may be the case, e.g., in wash-out methods where the agent is introduced into an animal to quickly, for example to lower the side effect of a substance already present in the body. Typically, such administration will be by injection, e.g., intravenous injection, in order to introduce the agent quickly. However, other routes may be used as appropriate. A single dose of a phosphonated polyphenol e.g. phosphonated pyrone analog such as a phosphonated flavonoid, such as a phosphonated quercetin, phosphonated fisetin, or phosphonated 5,7-dideoxyquercetin may also be used when it is administered with the substance (e.g., a therapeutic agent that produces a side effect) for treatment of an acute condition.

In some embodiments, the phosphonated polyphenol e.g. phosphonated pyrone analog such as a phosphonated flavonoid, such as a phosphonated quercetin, phosphonated fisetin, or phosphonated 5,7-dideoxyquercetin is administered in multiple doses. Dosing may be about once, twice, three times, four times, five times, six times, or more than six times per day. Dosing may be about once a month, once every two weeks, once a week, or once every other day. In one embodiment the drug is an immunosuppressive. In another embodiment the immunosuppressive compound and the transport protein activator are administered together about once per day to about 6 times per day. In another embodiment the administration of the immunosuppressive compound and the transport protein activator continues for less than about 7 days. In yet another embodiment the administration continues for more than about 6, 10, 14, 28 days, two months, six months, or one year. In some cases, continuous dosing is achieved and maintained as long as necessary, e.g., intravenous administration of immunosuppressive in a post-operative situation.

Administration of the agents of the invention may continue as long as necessary. In some embodiments, an agent of the invention is administered for more than 1, 2, 3, 4, 5, 6, 7, 14, or 28 days. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic pain.

An effective amount of a phosphonated polyphenol and an effective amount of a drug may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

The phosphonated polyphenol e.g. phosphonated pyrone analog such as a phosphonated flavonoid, such as a phosphonated quercetin, phosphonated fisetin, or phosphonated 5,7-dideoxyquercetin and the therapeutic agent may be administered in dosages as described herein (see, e.g., Compositions). Dosing ranges for therapeutic agents are known in the art. Dosing for the phosphonated polyphenol e.g. phosphonated pyrone analog such as a phosphonated flavonoid, such as a phosphonated quercetin, phosphonated fisetin, or phosphonated 5,7-dideoxyquercetin may be found by routine experimentation. For a phosphonated pyrone analog such as a phosphonated flavonoid, e.g., phosphonated quercetin, phosphonated fisetin, or phosphonated 5,7-dideoxyquercetin, typical daily dose ranges are, e.g. about 1-5000 mg, or about 1-3000 mg, or about 1-2000 mg, or about 1-1000 mg, or about 1-500 mg, or about 1-100 mg, or about 10-5000 mg, or about 10-3000 mg, or about 10-2000 mg, or about 10-1000 mg, or about 10-500 mg, or about 10-200 mg, or about 10-100 mg, or about 20-2000 mg or about 20-1500 mg or about 20-1000 mg or about 20-500 mg, or about 20-100 mg, or about 50-5000 mg, or about 50-4000 mg, or about 50-3000 mg, or about 50-2000 mg, or about 50-1000 mg, or about 50-500 mg, or about 50-100 mg, about 100-5000 mg, or about 100-4000 mg, or about 100-3000 mg, or about 100-2000 mg, or about 100-1000 mg, or about 100-500 mg. In some embodiments, the daily dose of phosphonated quercetin is about 100, 200, 300, 400, 500, 600, 700, 800, 900, 1000, 1100, 1200, 1300, 1400, 1500, 1600, 1700, 1800, 1900 or 2000 mg. In some embodiments, the daily dose of phosphonated quercetin is 100 mg. In some embodiments, the daily dose of phosphonated quercetin is 500 mg. In some embodiments, the daily dose of phosphonated quercetin is 1000 mg. In some embodiments, the daily dose of phosphonated quercetin is 1500 mg. In some embodiments, the daily dose of phosphonated quercetin is 2000 mg. Daily dose range may depend on the form of phosphonated pyrone analog such as a phosphonated flavonoid, e.g., the carbohydrate moieties attached to the phosphonated pyrone analog such as a phosphonated flavonoid, and/or factors with which the phosphonated pyrone analog such as a phosphonated flavonoid is administered, as described herein.

In some embodiments, the phosphonated polyphenol e.g. phosphonated pyrone analog such as a phosphonated flavonoid, such as a phosphonated quercetin, phosphonated fisetin, or phosphonated 5,7-dideoxyquercetin is administered two to three times a day with an oral dose of about 500 mg or an intravenous dose of about 150 mg. In some embodiments the phosphonated polyphenol e.g. phosphonated pyrone analog such as a phosphonated flavonoid, such as a phosphonated quercetin, phosphonated fisetin, or phosphonated 5,7-dideoxyquercetin is administered about one hour or about 30 minutes prior to administration of the therapeutic agent. In some embodiments the phosphonated polyphenol e.g. phosphonated pyrone analog such as a phosphonated flavonoid, such as a phosphonated quercetin, phosphonated fisetin, or phosphonated 5,7-dideoxyquercetin is administered such that it is in the bloodstream 30 minutes prior to administration of the therapeutic agent. This timing may be accomplished by administering the phosphonated polyphenol and the therapeutic agent separately, or by administering the quercetin and agent in the same composition that is formulated such that quercetin reaches the bloodstream before the therapeutic agent.

The serum half-life for, e.g., quercetin aglycone, is known to be about 19-25 hours. Where a phosphonated polyphenol of the invention has a serum half life in the same range, single dose accuracy is not crucial.

When a phosphonated polyphenol e.g. phosphonated pyrone analog such as a phosphonated flavonoid such as phosphonated quercetin, phosphonated fisetin, or phosphonated 5,7-dideoxyquercetin, is administered as a BTB transport modulator in a composition that comprises one or more therapeutic agents, and the therapeutic agent has a shorter half-life than BTB transport modulator, unit dose forms of the therapeutic agent and the BTB transport modulator may be adjusted accordingly. Thus, for example, if phosphonated polyphenol with a serum half life similar to that of quercetin is given in a composition also containing, e.g., tramadol, a typical unit dose form is, e.g., 50 mg tramadol/100 mg phosphonated polyphenol, or 50 mg tramadol/500 mg phosphonated polyphenol. See, e.g., Compositions.

Table 3 below, provides exemplary dosing schemes for selected immunosuppressive agents and quercetin phosphonate. These dosages are provided by way of example only and do not limit the invention.

| Therapeutic Agent (A) + | Per Dose (A:QP)* | | Per Day (A:QP) | |
|---|---|---|---|---|
| Phosphonated Quercetin (QP) | ~mole:mole | ~mg:mg | ~mole:mole | ~mg:mg |
| Tacrolimus | 0.006:1 | 10:1000 | 0.01:1 | 30:2000 |
| Sirolimus | 0.1:1 | 100:1000 | 0.2:1-0.3:1 | 400-600:2000 |
| Cyclosporin | 0.07:1 | 80:1000 | 0.1:1 | 240:2000 |
| Mycophenolate | 0.04:1 | 40:1000 | 0.2:1 | 400:2000 |
| Prednisone | 0.6:1 | 300:1000 | 0.8:1 | 900:2000 |
| | 1.75:1 | 900:1000 | 2.6:1 | 2700:2000 |

*2000 mg phosphonated quercetin daily, given in two divided doses, e.g., with two doses of the immunosuppressive.
Some doses of immunosuppressive are given without phosphonated quercetin.

### V. Packages and Kits

In still further embodiments, the present application concerns a kit for use with the compounds described above. Pyrone analogs or derivatives thereof (e.g., phosphorylated or phosphonated pyrone analogs) can be provided in a kit. The kits will comprise, in suitable container means, a composition of one or more pyrone analogs or derivatives thereof (e.g., phosphorylated or phosphonated pyrone analogs). The kit may comprise one or more compounds in suitable container means. Additionally, the packages or kits provided herein can further include any of the other moieties provided herein such as, for example, one or more therapeutic agents that have a side effect, lipid-lowering agents and/or glucose-lowering agents.

The container means of the kits will generally include at least one vial, test tube, flask, bottle, syringe and/or other container means, into which the at least one compound can be placed, and/or preferably, suitably aliquoted. The kits can include a means for containing at least one compound, and/or any other reagent containers in close confinement for commercial sale. Such containers may include injection and/or blow-molded plastic containers in which the desired vials are stored. Kits can also include printed material for use of the materials in the kit.

Packages and kits can additionally include, for example, pharmaceutically acceptable carriers, excipients, diluents, buffering agents, preservatives, stabilizing agents, etc., in a pharmaceutical formulation. Each component of the kit can be enclosed within an individual container and all of the various containers can be within a single package. Invention kits can be designed for cold storage or room temperature storage.

Additionally, the preparations can contain stabilizers (such as bovine serum albumin (BSA)) to increase the shelf-life of the kits. Where the compositions are lyophilized, the kit can contain further preparations of solutions to reconstitute the lyophilized preparations. Acceptable reconstitution solutions are well known in the art and include, for example, pharmaceutically acceptable phosphate buffered saline (PBS).

Packages and kits can further include one or more components for an assay. Samples to be tested in this application include, for example, blood, plasma, and tissue sections and secretions, urine, lymph, and products thereof. Packages and kits can further include one or more components for collection of a sample (e.g., a syringe, a cup, a swab, etc.).

Packages and kits can further include a label specifying, for example, a product description, mode of administration, discussion of clinical studies, listing of side effects, and/or indication of treatment. Packages provided herein can include any of the compositions as described herein. The package can further include a label for treating a condition described herein.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions. Kits, therefore, can additionally include labels or instructions for using the kit components in any method described herein. A kit can include a compound in a pack, or dispenser together with instructions for administering the compound in a method described herein. Where more than one compound is included in a kit, the package can include more than one pack, or dispenser together with instructions for administering the compounds in a method described herein.

Instructions can include instructions for practicing any of the methods described herein including treatment methods. Instructions can additionally include indications of a satisfactory clinical endpoint or any adverse symptoms that may occur, or additional information required by regulatory agencies such as the Food and Drug Administration for use on a human subject.

The instructions may be on “printed matter,” e.g., on paper or cardboard within or affixed to the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, magnetic tape, electrical storage media such as RAM and ROM, IC tip and hybrids of these such as magnetic/optical storage media.

Provided herein is a kit comprising a pyrone analog effective for generating a cellular protective effect and printed instructions for using the pyrone analog. In one embodiment, the kit further comprises one or more additional agents including, but not limited to, a lipid-lowering agent, a glucose-lowering agent, or both. Such additional agents may be packaged in individual containers or combined in a single container. Kits may further comprise a label for treating a condition including, but not limited to, amyloidosis, diabetes, disorders of myelin formation, hyperglycemia, impaired wound healing, neuropathy, insulin resistance, hyperinsulinemia, hypoinsulinemia, hypertension, hyperlipidemia, hypertriglyceridemia, hyperchlesterolemia, malignancy, microvascular retinopathy, surfactant abnormalities, vascular stenosis, inflammation, and hydronephrosis.

It will be apparent to those of skill in the art that variations may be applied without departing from the concept, teachings described herein. More specifically, it will be apparent that certain agents that both chemically and physiologically related may be substituted for the agents described herein while the same or similar results would be achieved. All such similar substitutes and modifications apparent to those skilled in the art are deemed to be within the teachings and concepts as defined by the appended claims.

### EXAMPLES

### Example 1

### Synthesis of Phosphorylated Quercetin and Phosphorylated Fisetin (Cyclic and Ring-Opened)

A suspension of quercetin dihydrate (1 g, 3.31 mmol) and triethylamine (2.3 mL, 16.5 mmol) in dichloromethane (100 mL) at room temperature is treated dropwise with a 10% solution of phosphorus oxychloride in dichloromethane (3.6 mL, 3.97 mmol). The resulting mixture is stirred overnight to afford a heterogeneous mixture along will a brown sticky precipitate. The LCMS of the solution showed clean conversion to a single species with the correct mass for the cyclic phosphate. The solution is separated and the solvent is removed in vacuo to give a yellow solid (presumably the TEA salt of cyclic phosphate). Some of the solid is taken and dissolved in water and a few drops of acetonitrile. Allowing this solution to sit overnight results in the hydrolytic ring opening of the cyclic phosphate to give acyclic phosphate as a yellow solid.

Using fisetin as the starting material in place of quercetin, phosphorylated fisetin is obtained.

### Example 2

### Synthesis of Quercetin-3′-O-phosphate

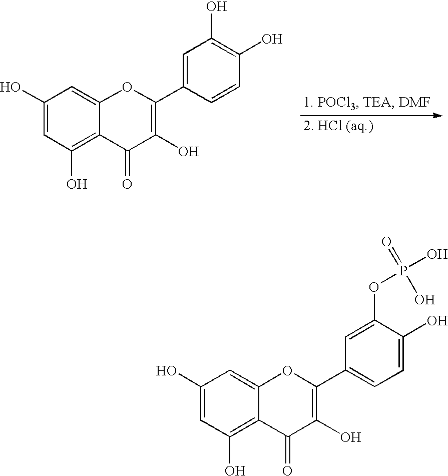

Quercetin dihydrate (90 g. 266 mmol, 1.0 eq.) was added to DMF (900 mL), followed by TEA (210 mL, 1463 mmol, 5.5 eq.) in one portion. The mixture was cooled to −1° C. by an acetone/dry ice bath while stirring. POCl₃ (30 mL, 319 mmol, 1.2 eq.) was slowly added through an addition funnel keeping the internal temperature below 5° C. The mixture was carefully kept between −1° C. and 5° C. until the addition of POCl₃ was complete. The acetone/dry ice bath was then removed and replaced by an ice/water bath.

The mixture was slowly warmed to room temperature over 18 h. To the solution was added 10% HCl (approx. 140 mL) until pH 5. The solution was concentrated in vacuo and the solid was dissolved in water (approx. 160 mL). The residue was purified over a 600 g, C-18 reverse phase column with 60 mL injections in a gradient. 100% D.I.U.F. water (3 L), 10% MeOH in water (1 L), 20% MeOH in water (1 L), 30% MeOH in water (1 L), and 1:1 water:MeOH (1 L). The desired product elutes in the 1 L fraction of 1:1-water:MeOH. This fraction is concentrated in vacuo. The residue was suspended in 500 volumes of water and Na₂CO₃ (s) was added until pH 9. To the solution, 50% H₂SO₄ (v/v) was added until pH 1. The mixture was kept at 4° C. for 24 h. The yellow solid was collected by vacuum filtration. The base/acid precipitation was repeated until no triethylamine remained (NMR). The pasty yellow solid was suspended in 100 volumes of water and centrifuged and the water was decanted off. The suspension and centrifugation process was repeated two more times. The paste was collected, frozen and lyophilized, giving quercetin-3′-O-phosphate as a yellow solid. The procedure was repeated until 5 kg of quercetin was processed with a combined yield of 280 g (4.4%). ¹H NMR (500 MHz/DMSO-d₆): δ 7.75 (s, 1H), 7.70 (d, 1H), 6.88 (s, 1H), 6.37 (s, 1H), 6.15 (s, 1H); ¹³C NMR (75.4 MHz/DMSO-d₆): δ 176.3, 164.9, 161.1, 156.7, 152.9, 146.8, 142.1, 136.3, 124.5, 122.8, 122.4, 119.1, 103.5, 99.0, 94.2.

### Example 3

### Synthesis of fisetin-3′-O-phosphate and fusetin-3′-O-phosphate monosodium salt hydrate

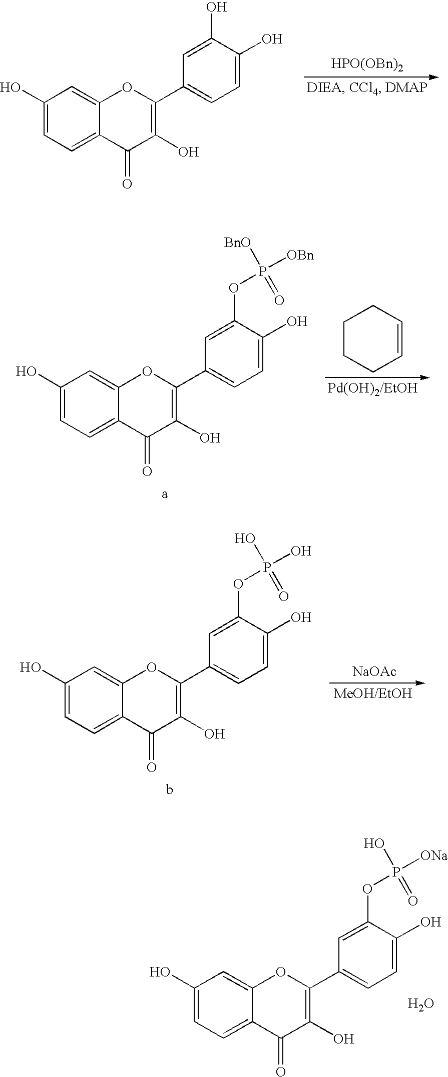

Dibenzyl 5-(3,7-dihydroxy-4-oxo-4H-chromen-2-yl)-2-hydroxyphenyl phosphate (a): Fisetin (8.2 g, 28.5 mmol, 1 equiv), dibenzylphosphite (11.2 g, 42.7 mmol, 1.5 equiv), N,N-diisopropylethylamine (18.9 mL, 114.0 mmol, 4 equiv), carbon tetrachloride (27.6 mL, 285.0 mmol, 10 equiv) and 4-(dimethylamino)-pyridine (3.5 g, 28.5 mmol, 1 equiv) were stirred in tetrahydrofuran at −10° C. for 2 hours. The mixture was allowed to warm to room temperature and stirred for 16 hr. The mixture was added to saturated potassium dihydrogenphosphate solution (500 mL) and extracted with ethyl acetate (100 mL×3). The combined organic solution was washed with brine, dried over sodium sulfate and concentrated in vacuo. The crude product was purified by chromatography on an Analogix system (SF 65-400 g) using 0-50% ethyl acetate (with 10% methanol)/heptane as the eluent. The product was obtained as yellow solid (2.72 g, 4.98 mmol, 17% yield).

Fisetin-3′-O-phosphate (b): Dibenzyl 5-(3,7-dihydroxy-4-oxo-4H-chromen-2-yl)-2-hydroxyphenyl phosphate (a) (5.8 g, 10.6 mmol) and palladium hydroxide (20% wt, 2.1 g) were stirred in cyclohexene (200 mL) and ethanol (200 mL). The reaction was heated at reflux for 16 hr. The reaction mixture was cooled to room temperature, filtered through Celite and concentrated in vacuo. The residual solid was triturated with water to provide the product as orange solid (3.17 g, 8.66 mmol, 81% yield).

Fisetin-3′-O-phosphate monosodium salt hydrate: Fisetin-3′-O-phosphate (b) (2.52 g, 6.89 mmol, 1 equiv) was added to a mixture of methanol (130 mL) and ethanol (200 mL). The solid completely dissolved upon heating at −50° C. for 2 min. Sodium acetate (0.56 g, 6.89 mmol, 1 equiv) was then added to the solution. The mixture was stirred at room temperature for 3 hr., with formation of an off-white precipitate. The solid was filtered, washed with ethanol and dried in a vacuum oven at room temperature to give the product as light yellow solid (1.94 g, 5.0 mmol, 72% yield). ¹H NMR (300 MHz/D₂O): δ 7.65 (d, 1H), 7.22-7.19 (m, 2H), 7.04 (s, 1H), 7.01 (d, 1H), 6.51 (d, 1H). Anal. Calcd for C₁₅H₁₂NaO₁₀P: C, 44.35; H, 2.98; Na, 5.66; P, 7.62. Found: C, 44.86; H, 2.67; Na, 5.78; P, 7.45.

### Example 4

### Stability of quercetin-3′-O-phosphate and fisetin-3′-O-phosphate in water

Quercetin-3′-O-phosphate is dissolved in water at about pH 8. After 24 hours in water at pH 8, no degradation is seen by NMR and HPLC after 24 hours at ambient temperature.

Fisetin-3′-O-phosphate is dissolved in water at about pH 8. After 24 hours in water at pH 8, no degradation is seen by NMR and HPLC after 24 hours at ambient temperature.

### Example 5

### Somatostatin Release

Rat hippocampal slices (thickness 350 μm, round slice) are prepared by a standard method. Twenty rat hippocampal slices are placed in a perfusion chamber, incubated at 37° C. and perfused by a batch method while exchanging the incubation buffer every 10 minutes. The incubation buffer has the composition: NaCl, 124 mM; KCl, 5 mM; KH₂ PO₄, 1.24 mM; MgSO₄, 1.3 mM; CaCl₂, 2.4 mM; NaHCO₃, 26 mM; D-glucose, and 10 mM. A mixed gas of oxygen (95%) and carbon dioxide (5%) is used to saturate the buffer.

Perfusion for 150 minutes provides fractions 1-15. To fraction 9 is applied a high K+ (50 mM) stimulation. A pyrone analog is added to fractions 7-15 to the concentration of 10⁻⁹ M, 10⁻⁷ M, 10⁻⁷ M, 10⁻⁶ M, respectively. Examples of a pyrone analog include phosphorylated quercetin and phosphorylated fisetin. Nothing is added to control group. The respective fractions thus obtained are concentrated by lyophilization and somatostatin in the perfusate is quantified by radioimmunoassay (RIA). After the completion of the experiment, somatostatin remaining in the slices is extracted by a conventional method and quantified by radioimmunoassay. The somatostatin amount released by high K⁺ (50 mM) stimulation is calculated and the amount of somatostatin released due to the property of the pyrone analog is measured.

Somatostatin release (%) by the pyrone analog at each concentration is calculated as in the following. The somatostatin amount of each fraction is expressed by the percentage (%) relative to the somatostatin residual amount at the time the fraction is obtained. The value of fraction 8 immediately before high K⁺ (50 mM) stimulation is taken as the base and the values exceeding the base value are added with regard to fraction 9 and the subsequent peak fractions exceeding the base value to give somatostatin release (%). The number of the test samples measured is 10 or 11. Each value (%) is expressed by mean±S.E.M. The property of the pyrone analog is subjected to Dunnett's multiple comparison test relative to control group.

### Example 6

### Glucagon Screening

Glugacon may be assessed using standard techniques such as, for example, a random blood glucose test, a fasting blood glucose test, a blood glucose test two hours after 75 g of glucose, or an even more formal oral glucose tolerance test (OGTT).

People with a confirmed diagnosis of diabetes are tested routinely for complications. This includes, for example, yearly urine testing for microalbuminuria and examination of the retina of the eye for retinopathy.

### Example 7

### Oral Glucose Tolerance Test (OGTT)

A patient fasts for 8-14 hours (water is allowed). Usually the OGTT is scheduled to begin in the morning (0700-0800) as glucose tolerance exhibits a diurnal rhythm with a significant decrease in the afternoon. A zero time (baseline) blood sample is drawn.

The patient is then given a glucose solution to drink within 5 minutes. The standard dose is 1.75 grams of glucose per kilogram of body weight, to a maximum dose of 75 g.

Blood is drawn at intervals for measurement of glucose (blood sugar), and sometimes insulin levels. The intervals and number of samples vary according to the purpose of the test. For simple diabetes screening, the most important sample is the 2 hour sample and the 0 and 2 hour samples may be the only ones collected. In research settings, samples may be taken on many different time schedules.

If renal glycosuria (sugar excreted in the urine despite normal levels in the blood), then urine samples may also be collected for testing along with the fasting and 2 hour blood tests.

Fasting plasma glucose should be below 6.1 mmol/l (110 mg/dl). Fasting levels between 6.1 and 7.0 mmol/l (110 and 126 mg/dl) are borderline (“impaired fasting glycaemia”), and fasting levels repeatedly at or above 7.0 mmol/l (126 mg/dl) are diagnostic of diabetes.

The 2 hour glucose level should be below 7.8 mmol/l (140 mg/dl). Levels between this and 11.1 mmol/l (200 mg/dl) indicate “impaired glucose tolerance.” Glucose levels above 11.1 mmol/l (200 mg/dl) at 2 hours confirm a diagnosis of diabetes.

1999 WHO Diabetes criteria - Interpretation of Oral Glucose Tolerance Test

| | Glucose levels | | | | | | | |
|---|---|---|---|---|---|---|---|---|
| | | | Impaired | | Impaired | | | |
| | | | Fasting | | Glucose | | Diabetes | |
| | | | Glycaemia | | Tolerance | | Mellitus | |
| Venous | Normal | | (IFG) | | (IGT) | | (DM) | |
| Plasma | Fasting | 2 hrs | Fasting | 2 hrs | Fasting | 2 hrs | Fasting | 2 hrs |
| (mmol/l) | <6.1 | <7.8 | ≧6.1 & | <7.8 | <7.0 | ≧7.8 | ≧7.0 | ≧11.1 |
| | | | <7.0 | | | | | |
| (mg/dl) | <100 | <140 | ≧100 & | <140 | <125 | ≧140 | ≧126 | ≧200 |
| | | | <125 | | | | | |

### Example 8

### Grehlin Screening

Pyrone analogs or derivatives thereof can be tested with regard to their ability to stimulate ghrelin release using conventional means in the art. Examples of a pyrone analog include phosphorylated quercetin and phosphorylated fisetin.

Briefly, in one method, pyrone analogs are made as 100× stock solutions by dissolving them in pure ethanol, as a vehicle. The pyrone analogs are then diluted 1/100 in the Leibovitz L-15 medium containing 0.5% fetal bovine serum (FBS). RF-48 cells are grown during incubation at 37° C. in Leibovitz's L15 medium with 2 mM L-glutamine and containing 10% (vol/vol) FBS in the absence of CO₂.

After cell confluence is obtained, the cells are plated in 24-well cultures plates (1×10⁵ cells/well). Several wells are subsequently exposed to one of the pyrone analogs as prepared above. For each type and concentration of pyrone analog tested, a series of three tests is carried out.

After 1 hour of incubation of the thus-filled wells containing both the cells and the pyrone analog at the same conditions as those applied during growing of the RF-48 cells, a sample is taken from each well to measure ghrelin release.

Each sample is centrifuged at 3000 rpm to remove the cells from the sample and the supernatant (containing the ghrelin formed as well as the medium and the pyrone analog) is transferred to a separate tube. Ghrelin release is measured using a commercial enzyme immunoassay kit (from Phoenix Pharmaceuticals, Belmont, Calif., USA).

### Example 9

### Screening Foam Cells

Screening (assessing) of the effect of the pyrone analogs with respect to foam cells described herein may be assessed using conventional techniques. Examples of a pyrone analog include phosphorylated quercetin and phosphorylated fisetin.

Briefly, in one non-limiting example, human blood is drawn and peripheral monocytes are isolated by methods routinely practiced in the art. These human monocytes can then be used immediately or cultured in vitro, using methods routinely practiced in the art, for 5 to 9 days where they develop more macrophage-like characteristics such as the upregulation of scavenger receptors. These cells are then treated for various lengths of time with pyrone analogs. Control monocytes that are untreated or treated with native LDL are grown in parallel. At a certain time after addition of the pyrone analogs or controls, the cells are harvested and analyzed for differential expression as described in U.S. Pat. No. 6,124,433 which is incorporated herein by reference in its entirety.

Cells treated with pyrone analogs can be examined for phenotypes associated with cardiovascular disease. In the case of monocytes, such phenotypes include but are not limited to increases in rates of LDL uptake, adhesion to endothelial cells, transmigration, foam cell formation, fatty streak formation, and production by foam cells of growth factors such as bFGF, IGF-I, VEGF, IL-1, M-CSF, TGF-beta, TGF-alpha, TNF-alpha, HB-EGF, PDGF, IFN-gamma, and GM-CSF.

### Example 10

### Expression Analysis

RNA Isolation and RT-PCR Analysis

Total RNA is isolated from frozen tissues using standard techniques and kits such as, for example, Tri-Reagent (Molecular Research Center, Ohio).

Real Time PCR

RT-PCR is performed, for example, on a LightCycler (Roche Applied Science, Mannheim, Germany), using SYBR-Green I dye.

Amplification conditions include initial denaturation at 95° C. for 10 minutes, followed by 55 cycles for both specific genes, or 30 cycles for beta-actin. The fluorescent signal is monitored. A melting curve program is carried out according to standard techniques to analyze the specificity of the generated products. Gene expression levels are normalized to the respective beta-actin mRNA levels, in the same samples.

Alternatively, quantitative real-time RT-PCR is performed using, for example, ABI Prism 7000 sequence Detection system (Applied Biosystems).

Fluorogenic probes such as from Assay-On-Demand (Applied Biosystems) and amplification conditions may be applied according to standard techniques. The mRNA levels are corrected for human beta-actin mRNA.

### Example 11

### Pancreatic Hormones Immunohistochemistry

Slides of 4 μm paraffin-embedded sections are deparaffinized, incubated in 3% H₂O₂, and are incubated in blocking solution (for both Insulin and Glucagon detection), using a commercially available Histomouse™-SP Kit (Zymed laboratories, South San Francisco, Calif.). Sections are then incubated for 1 h at 37° C. with monoclonal antibodies against human insulin and against human glucagon (Sigma), both at a dilution of 1:200. Slides are exposed to the secondary biotinylated IgG for 30 minutes at room temperature and then incubated in strepavidin-peroxidase followed by a chromogen peroxide solution. A control using only secondary without primary antibodies followed by strepavidin-peroxidase and a chromogen peroxide solution is performed to rule out possible background of the system.

### Example 12

### Radioimmunoassay (RIA) for Pancreatic Hormones

Pancreas and livers are isolated, immediately frozen in liquid nitrogen, and stored at −70° C. Frozen tissues are homogenized in 0.18N HCl/35% ethanol. The homogenates are extracted overnight at 4° C. with continuous stirring, and the supernatants are lyophilized. Samples are dissolved in 0.8 ml RIA Assay Buffer, supplemented by a cocktail of protease inhibitors (Sigma). Hepatic insulin and glucagon levels are determined using rat radioimmunoassay (RIA, catalog no. SRI-13K and GL-32K, Linco, Mo., USA, and Coat-A-Count, DPC; Calif., USA). Somatostatin concentrations are determined by RIA (Euro-diagnostica, Sweden). Hepatic content of pancreatic hormones is normalized to the weight of the extracted tissue.

### Example 13

### Determination of Hepatic Function

Serum biochemistry profile consisting of albumin, AST (Aspartate aminotransferase), ALT (Alanine aminotransferase) and total billirubin may be determined using standard techniques and kits provided by, for example, Olympus AU 2700 Apparatus (Olympus, Germany) in serum samples.

### Example 14

### Insulin and C-Peptide Detection

Insulin and C-peptide secretion and content from primary adult liver cells are measured by static incubation of 48 hours after 3 days of treatment. Insulin secretion into the media is measured by RIA using the Ultra Sensitive Human Insulin RIA kit (Linco Research) and C-peptide secretion is measured by Human C-Peptide RIA kit (Linco Research).

Insulin content is measured after homogenizing the cell pellet in 0.18 N HCl, 35% ethanol. The homogenates are extracted overnight at 4° C. with continuous stirring, and the supernatants are lyophilized. Samples are dissolved in 0.5 ml PBS containing 0.2% BSA and Protease Inhibitory cocktail (Sigma). One hundred (100) μl sample are used for the RIA. Insulin content is normalized to total cellular protein, measured by the Bio-Rad Protein Assay kit.

### Example 15

### Glucose Challenge Assay

Adult liver cells are treated with pyrone analogs or controls for 5 days. The cells are plated in 6-well plates at 10⁵ cells per well.

For time course analysis, the cells are preincubated for 2 hours in Krebs-Ringer buffer (KRB) containing 0.1% BSA, followed by incubation for the indicated period in media containing 2 mM or 25 mM glucose. At each time point media samples are analyzed for insulin (Ultra Sensitive Human Insulin RIA kit—Linco Research) and C-peptide secretion (Human C-Peptide RIA kit—Linco Research).

To measure glucose dose response, cells are preincubated for 2 hours with KRB containing 0.1% BSA, washed and challenged thereafter with increasing concentrations of D-Glucose or 2-deoxy-Glucose (0-25 mM) for 2 hours. At the end of the incubation period at 25 mM glucose, the cells are washed with KRB and incubated for additional 2 hours in 2 mM glucose containing media.

### Example 16

### Electron Microscopy

Liver cells are fixed in 2.5% gluteraldehyde, osmificated, dehydrated with a graded series of ethanol and propylene oxide, and embedded in Araladite solution (Polyscience Inc.). Ultra-thin sections are cut in an ultramicrotome, stained with 2% uranyl acetate and Reynolds' lead citrate solution. For post-embedding immunogold reactions, 50-90 nm liver sections are put on nickel grids. The grids are incubated with antibody against insulin (guinea-pig polyclonal; 7.8 μg/ml, Dako) at room temperature overnight and then incubated with immunogold-conjugated antibody against guinea-pig IgG (15-nm gold; diluted 1:40, Dako) for 1.5 hours at room temperature. The sections are observed under an electron microscope (Jeol 1200EX2).

### Example 17

### Hyperglycemia Test

Blood glucose is measured twice weekly using, for example, an Accutrend® GC Glucose Analyzer (Boehringer Mannheim, Mannheim, Germany).

### Example 18

### In vitro toxicity screening of fisetin-3′-O-phosphate or quercetin-3′-O-phosphate

A secondary pharmacological screening of molecules of interest at a fixed concentration is often practiced in the pharmaceutical industry in order to evaluate the effect of the compound on secondary targets that could result in untoward toxicity in vivo. These secondary screens are well known in the art and can be carried out by labs which specialize in these tests such as MDS-Panlabs and CEREP. A secondary toxicity screen is performed with quercetin-3% O-phosphate or fisetin-3′-O-phosphate at a concentration of 10 uM against 122 targets in enzyme, radioligand binding, and cellular assays by MDS Pharma Services by methods well known in the art. Inhibition is found in only the following targets (percent inhibition at 10 μM in parentheses): ATPase, Na+/K+, Heart, Pig (65%), Nitric Oxide Synthase, Endothelial (eNOS) (72%), Protein Tyrosine Kinase, FGFR2 (94%), Protein Tyrosine Kinase, FGFR1 (96%), Protein Tyrosine Kinase, Insulin Receptor (91%), Protein Tyrosine Kinase, (82%), Protein Tyrosine Kinase, ZA70 (ZAP-70) (74%), UDP Glucuronosyltransferase, UGT1A1 (52%), Adenosine A₁ (50%), Adrenergic α_{2A} (57%), Dopamine D₄₇ (51%), Peripheral Benzodiazepine Receptor (PBR) (53%), Transporter, Monoamine rabbit (68%), Serotonin (5-Hydroxytryptamine) 5-HT_{1A} (62%).

The compound is additionally tested in Adenosine_{A1}, Adrenergic_{A2A}, DopamineD₂₅, Histamine H₁-, and μ-Opiate GTPγS functional assays using a concentration of 10 μM. The compound demonstrated 48% antagonist activity in the Adenosine_{A1} assay, and marked negative inhibition in the Adrenergic_{A2A} assay, potentially indicating PAF-5 could be acting as an inverse agonist in this assay.

The findings of this toxicology screen indicate that quercetin-3′-O-phosphate or fisetin-3′-O-phosphate has low toxicity properties, especially in light of the fact that the concentration tested, 10 μM, is high as compared to a therapeutic dose (e.g. greater than ˜100 times).

### Example 19

### Pyrone Analog Decreases Cholesterol and Triglyceride Levels in Human

A 32-year-old, obese, Caucasian male has a cholesterol level of 299 mg/dL, a triglyceride level of 440 mg/dL, an LDL level of 199 mg/dL, and an HDL level of 25 mg/dL. He does not have diabetes, kidney, or liver disease. He has a family history of coronary artery disease—his father suffers a heart attack at age 50. Because this patient is a male, obese, and has a positive family history of heart disease, he is advised to immediately start using the composition described herein on a daily basis. Preferably, the composition is a tablet containing 20 mg of phosphorylated quercetin or phosphorylated fisetin. Additionally, he must strictly adhere to a low fat diet, and regularly exercise 30 minutes daily or 45 minutes every other day.

The patient follows up with his doctor in 3 months with a repeat lipid profile. The blood test result shows an improvement of decreased cholesterol and triglycerides to 250 mg/dL and 280 mg/dL, respectively. The follow up plan also includes maintaining the same dosage of composition at 20 mg for two months, since the patient tolerates the medication well.

### Example 20

### Pyrone Analog Decreases Triglyceride Level in Human

A 45-year-old Hispanic male with a history of gout and gastritis has a triglyceride level of 950 mg/dL, and a cholesterol level of 300 mg/dL. The patient begins using a composition described herein, for example a tablet containing 50 mg of phosphorylated quercetin or phosphorylated fisetin, twice daily with no side effects. The patient is very compliant with respect to taking the medication everyday, along with consuming a low fat diet and regularly exercising. As a result, the patient's triglyceride level decreases to 450 mg/dL. His gout and gastritis conditions also improve as a direct result of lowering his triglycerides levels and his low fat diet. He is to maintain the dosage of a composition described herein at 50 mg twice daily for the best results.

### Example 21

### Pyrone Analog Decreases LDL Level and Increases HDL Level in Human

A 55-year-old Asian female has menopause, hypertension, and hyperlipidemia. She is currently taking Prampro™ hormone replacement therapy for menopause, and Atenolol™ for hypertension, which is controlled at this time. Her lipid profiles show an elevated LDL level of 180 mg/dL (normal <130), a low HDL level of 28 mg/dL (normal >40), a normal triglyceride level of 170 mg/dL (normal <160), and a cholesterol level of 210 mg/dL (normal< or =200). Since the patient does not like to take medication, her doctor agrees to wait six to twelve months to monitor her lipid profiles without the lipid-lowering medication, counting on the hormone replacement therapy and a low fat diet to help reduce the LDL cholesterol level. However, after one year, the LDL and HDL levels are not adequately reduced. Her doctor decides to start administering a composition described herein at a dose of 10 mg daily for 6 months. Subsequently, the LDL level decreased to 130 mg/dL and the HDL level increased to 60 mg/dL. Even though the patient's lipid profile improved to normal range, it is recommended that she continues to take a composition described herein, for example a tablet containing 10 mg of phosphorylated quercetin or phosphorylated fisetin daily, to prevent future accumulation of LDL, which causes cholesterol plague in coronary vessels. Also, she is recommended to take 81 mg of aspirin daily to prevent stroke and heart disease.

### Example 22

### Pyrone Analog in Combination with Other Drugs Prevent Myocardial Infarction in Diabetic Patient

A 34-year-old Hispanic female with diabetes mellitus type 2 has high cholesterol levels and high LDL levels. During an office visit, she experiences a silent heart attack without congestive heart failure. She is then admitted to the hospital for further cardiac evaluation and subsequently discharged after three days. She is currently taking Glucotrol™ XL 5 mg daily, Glucophage™ 500 mg twice a day (diabetes medications), Tenormin™ 25 mg/day, Zestril™ 10 mg/day (to prevent chest pain, and high blood pressure), and aspirin 81 mg/day. She is also taking a composition described herein at the dosage of 10 mg-20 mg phosphorylated quercetin or phosphorylated fisetin daily to prevent a second myocardial infarction in the future.

### Example 23

### Pyrone Analog Treats Hypercholesterolemia in Human

A 42-year-old Asian male has strong a familial hypercholesterolemia. Hypercholesterolemia is a condition in which cholesterol is overly produced by the liver for unknown reasons. Furthermore, hypercholesterolemia is a strong risk factor for myocardial infarction (MI), diabetes, obesity, and other illnesses. The patient is not overweight, but is very thin. He has a very high level of cholesterol, over 300 mg/dL, and a triglyceride level of over 600 mg/dL. His diet consists of very low fat, high protein foods, and no alcohol. He has a very active lifestyle, but one which is not stressful. However, he still has to take medication to lower his cholesterol and triglyceride levels. The medications he takes include a composition described herein. He is advised to continue taking a composition described herein, for example a tablet containing 40 mg of phosphorylated quercetin or phosphorylated fisetin, daily for the remainder of his life in order to control his unusual familial hypercholesterolemia condition.

### Example 24

### Pyrone Analog Decreases Triglyceride Level in Human

A 22-year-old male patient presents with triglyceride level of 250 mg/dL. The patient is given oral tablets containing about 20 mg to about 100 mg of a pyrone analog, for example phosphorylated quercetin or phosphorylated fisetin. The patient's level of triglyceride is measured 24 hours after ingesting the tablets. The measurement shows a decrease of about 20% to 50% of triglycerides as compared to the initial level.

### Example 25

### Pyrone Analog Decreases Blood Glucose Level in Human

A 46-year-old African American female with diabetes mellitus type 2 has hyperglycemia with a blood glucose level of 20 mmol/L, i.e. approximately 360 mg/dL. She is taking tablets described herein at the dosage of 10 mg-20 mg phosphorylated quercetin or phosphorylated fisetin once daily. The patient's level of blood glucose is measured 24 hours after ingesting the tablets. The measurement shows that the patient's blood glucose level returns to 6 mmol/L (i.e. 108 mg/dL) after fasting, which is within the normal range of about 80 to 120 mg/dL or 4 to 7 mmol/L.

### Example 26

### Effect of Pyrone Anolog on Serum Triglyerides in Cynomologus Monkeys

Five male cynomologus monkeys are employed in the animal study. Three of the five monkeys are treated with phosphorylated quercetin at a daily dosage of 1.25 mg/kg (orally) for a period of 25 days. Phosphorylated quercetin is a lipid transporter activator. The remaining two are similarly treated with a vehicle to serve as control. Serum samples are collected on days 1, 8, 15, 22 and 25 for triglyceride determination. Serum samples from days 8, 15, 22 and 25 are also assayed for the concentration of phosphorylated quercetin. All monkeys appear healthy throughout the study period with no change in body weight or rate of food consumption. A highly significant decrease of serum triglycerides is observed in each of the three monkeys receiving phosphorylated quercetin treatment (Table 3). When compared to day 1 (baseline), the average decrease is 58%, 55% and 51% for the three monkeys treated with phosphorylated quercetin, while the two control monkeys have an average increase of 91% and 80%. The triglyceride lowering effect and the relatively high blood concentration of phosphorylated quercetin (Table 4) indicate that phosphorylated quercetin is well absorbed by monkeys when given orally. From the data presented, it is concluded that phosphorylated quercetin lowers serum triglycerides in monkeys at a daily dose of 1.25 mg/kg without any noticeable abnormal clinical signs.

TABLE 3
Serum triglycerides (mg/dl) of male cynomolgus monkeys treated
with phosphorylated quercetin by gastric intubation

| phosphorylated | | | | | Day | Day |
|---|---|---|---|---|---|---|
| quercetin | Animal # | Day 1 | Day 8 | Day 15 | 22 | 25 |
| 0.0 mg/0.4 mL/kg | 1 | 43.2 | 82.2 | 94.7 | 85.0 | 82.3 |
| | 2 | 41.7 | 53.6 | 78.9 | 83.4 | 75.1 |
| | Mean | 42.5 | 67.9 | 86.8 | 84.2 | 78.7 |
| 1.0 mg/0.4 mL/kg | 3 | 47.9 | 22.1 | 19.3 | 25.2 | 19.8 |
| | 4 | 51.5 | 24.6 | 33.1 | 22.4 | 23.2 |
| | 5 | 58.5 | 29.2 | 36.7 | 31.9 | 28.3 |
| | Mean | 52.6 | 25.3 | 29.7 | 26.5 | 23.8 |

TABLE 4
Serum concentration (ng/mL) of phosphorylated quercetin
in male cynomolgus monkeys treated with
phosphorylated quercetin by gastric intubation

| phosphorylated | Animal | | | | |
|---|---|---|---|---|---|
| quercetin | # | Day 8 | Day 15 | Day 22 | Day 25 |
| 0.0 mg/0.4 | 1 | BLQ | 0.635 | 0.247 | 1.21 |
| mL/kg | | | | | |
| | 2 | 0.584 | 1.2 | 0.137 | 1.29 |
| 1.0 mg/0.4 | 3 | >200 | 1308 | 498 | >2900 |
| mL/kg | | | | | |
| | 4 | 397 | 160 | 782 | 437 |
| | 5 | >150 | >180 | >120 | >2000 |

### Example 27

### Effect of Pyrone Anologs on Serum Triglyerides and Hepatic Triglyceride Output in Male SJL Mice

Male SJL mice are dosed orally with vehicle, phosphorylated quercetin, or phosphorylated fisetin, for 4 consecutive days. The test compounds are dissolved in corn oil and given at a dosage/volume of 20 mg/5 mL/kg. On day 3, serum triglycerides (STG) are determined from samples collected at 7 a.m. On day 4, animals are fasted after dosing, starting at 8 am. Following 6 hours of fasting, blood samples are collected prior to intravenous injection of WR-1339 at 100 mg/5 ml/kg. Additional serum samples are collected at 1 and 2 hours after WR-1339 injection. WR-1339, also known as Triton WR 1339 or 4-(2,4,4-trimethylpentan-2-yl)phenol, is a detergent which inactivates lipoprotein lipase and thus prevents the removal of triglycerides from circulation. By measuring the increase of STG after WR-1339 administration in fasted animals, one can estimate the hepatic triglyceride (HTG) output during fasting. Results are listed in Table 5.

Phosphorylated quercetin appears to lower non-fasting STG (Day 3,8 a.m.) but not fasting STG (Day 4, 2 p.m.). A reduction of HTG output after WR-1339 injection is observed with phosphorylated quercetin. These effects are not observed with phosphorylated fisetin given orally.

The result also indicates that male SJL mouse is a suitable model for in vivo screening of retinoid effect on serum triglycerides. The effect could be detected after 2 days of dosing.

Due to the lack of effect of phosphorylated fisetin at 20 mg/kg, the dose is increased to 100 mg/kg in the same set of mice. STG is determined on day 3 prior to dosing (Day 3, 8 am.). Again, no lowering of STG is observed (Table 5). To ensure that phosphorylated fisetin would be bioavailable, phosphorylated fisetin is dissolved in DMSO and given by intraperitoneal injections, once at 4 p.m. on day 3 and once at 8 a.m. on day 4, at a dosage of 100 mg/kg/injection. Administration of WR-1339 and blood collections on day 4 are similarly conducted as described above. Results (Table 6) indicate that a clear lowering of STG is observed 16 hours after a single intraperitoneal 100 mg/kg dose (Day 4, 8 a.m.). Similar to phosphorylated quercetin, this effect disappears after fasting (Day 4, 2 p.m.). HTG output is also reduced with intraperitoneal injection of phosphorylated fisetin. It is likely that phosphorylated fisetin may not be bioavailable when given orally to mice.

Without wishing to limit the embodiments to any theory or mechanism of operation, it is believed that pyrone analogs are capable of lowering serum triglycerides in mice when they are made bioavailable by proper route of administration. Furthermore, this lowering of triglycerides of pyrone analogs may be due, at least partially, to a reduced HTG output.

TABLE 5
Serum triglycerides in mice treated with phosphorylated quercetin
and phosphorylated fisetin by oral gavages

| | | | Day 4 post-WR-1339 | | |
|---|---|---|---|---|---|
| | Animal | Day 3 | 0 hr | 1 hr | 2 hr |
| Group/Treatment | # | 8 am | (2 pm) | (3 pm) | (4 pm) |
| 1 (Males) | 1 | 111.8 | 81.3 | 431.2 | 763.1 |
| Vehicle (corn oil) | 2 | 199.7 | 95.4 | 432.4 | 956.2 |
| 100 mg/kg tyloxapol IV | 3 | 154.4 | 75.3 | 468 | 890.3 |
| | 4 | 104.4 | 85.7 | 287.1 | 497 |
| | 5 | 127.4 | 77.6 | 307.8 | 579 |
| | 6 | 133.4 | 73.4 | 226.4 | 391.8 |
| | 7 | 90.8 | 72.7 | 245.2 | 498.3 |
| | 8 | 111.8 | 85 | 289.7 | 523.5 |
| | 9 | 70.6 | 35.9 | 277.5 | 531.2 |
| | 10 | 99.6 | 79.9 | 333 | 679.8 |
| Group 1 Mean | | 120.4 | 76.2 | 329.8 | 631.0 |
| Group 1 SD | | 36.3 | 15.7 | 84.6 | 185.5 |
| 2 (males) | 11 | 128.7 | 63.1 | 360.1 | 726.9 |
| 20 mg/kg phosphorylated | 12 | | | | |
| fisetin | | | | | |
| 100 mg/kg tyloxapol IV | 13 | 124 | 91.7 | 380.1 | 723.7 |
| | 14 | 150.3 | 43 | 464.1 | 770.2 |
| | 15 | 110.5 | 72.1 | 241.9 | 590 |
| | 16 | 118.6 | 90.8 | 331.7 | 575.2 |
| | 17 | 124.7 | 76 | 329.8 | 700.4 |
| | 18 | 112.5 | 68.2 | 262.6 | 462.8 |
| | 19 | 106.4 | 73.4 | 311 | 659.1 |
| | 20 | 131.4 | 73.4 | 326.5 | 612.6 |
| Group 2 Mean | | 123.0 | 72.4 | 334.2 | 646.8 |
| Group 2 SD | | 13.3 | 14.6 | 65.1 | 96.2 |
| 3 (Males) | 21 | 71.2 | 76.6 | 216.8 | 328.5 |
| 20 mg/kg phosphorylated | 22 | 105.7 | 76 | | |
| quercetin | | | | | |
| 100 mg/kg tyloxapol IV | 23 | 67.9 | 57.3 | 307.2 | 548 |
| | 24 | 113.2 | 74.7 | 294.9 | 562.9 |
| | 25 | 134.8 | 80.5 | 311.7 | 577.1 |
| | 26 | 76.6 | 71.5 | 238.7 | 493.8 |
| | 27 | 63.1 | 73.4 | 303.9 | 508 |
| | 28 | 84.1 | 61.1 | 260 | 550 |
| | 29 | 95.6 | 67.6 | 252.3 | 542.9 |
| | 30 | 115.2 | 76 | 210.9 | 259.1 |
| Group 3 Mean | | 92.7 | 71.5 | 266.3 | 485.6 |
| Group 3 SD | | 24.0 | 7.4 | 39.5 | 113.0 |

TABLE 6
Serum triglycerides in mice treated with phosphorylated fisetin by oral
gavages (day 1 to 3) and subcutaneous injections (day 3 to 4)

| | | | Day 4 | Day 4 post-WR-1339 | | |
|---|---|---|---|---|---|---|
| Group/ | | Day 3 | 0 hr | 0 hr | 1 hr | 2 hr |
| Treatment | I.D. | 0 Hour | (8 am) | (2 pm) | (3 pm) | (4 pm) |
| 1 | 1 | 167 | 121 | 58 | 527 | 857 |
| Vehicle | 2 | 91 | 112 | 45 | 403 | 695 |
| | 3 | 95 | 140 | 50 | 279 | 544 |
| | 4 | 67 | 51 | 45 | 222 | 415 |
| | 5 | 127 | 160 | 58 | 354 | 585 |
| Group 1 | | 109 | 117 | 51 | 357 | 619 |
| Mean | | | | | | |
| Group 1 SD | | 39 | 41 | 7 | 118 | 166 |
| 2 | 6 | 81 | 58 | 42 | 220 | 285 |
| phosphorylated | 7 | 104 | 79 | 36 | 195 | 272 |
| fisetin | | | | | | |
| Day 1-3, 100 | 8 | 103 | 51 | 42 | 248 | 396 |
| mg/kg, oral | | | | | | |
| Day 3-4, 100 | 9 | 139 | 114 | 73 | 345 | 531 |
| mg/kg, I.P. | | | | | | |
| | 10 | 107 | 50 | 59 | 126 | 200 |
| | 11 | 171 | 125 | 50 | 197 | 387 |
| Group 2 | | 118 | 79 | 50 | 222 | 345 |
| Mean | | | | | | |
| Group 2 SD | | 32 | 33 | 14 | 72 | 118 |

### Example 28

### LIM-0705 and LIM-0741 Protect Against Onset of Type 2 Diabetes and Attendant Complications in Diabetic Rat Model

Animals: Seven (7) week old male Zucker Diabetic Fatty (ZDF) rats are used. The ZDF rat is a model for Type 2 diabetes based on impaired glucose tolerance caused by the inherited obesity gene mutation that leads to insulin resistance. Between 7 and 10 weeks of age, a male ZDF rat has high blood insulin levels when fed with Purina 5008 chow that subsequently drop as pancreatic beta cells cease to respond to glucose. By 12 weeks of age, a male ZDF rat on a diet consisting of Purina 5008 chow is fully diabetic.

General procedures for animal care and housing are in accordance with the National Research Council (NRC) Guide for the Care and Use of Laboratory Animals (1996) and the Animal Welfare Standards incorporated in 9 CFR Part 3, 1991.

Experimental Design: This study is a 6 week study. Forty-eight (48) 7-week old male ZDF rats are chosen and divided into 6 treatment arms (8 rats/arm). The rats are kept on a diet of Purina 5008 to induce the onset of diabetes. The animals are treated intraperitoneally (i.p.) daily with the following compounds:

| Group | Treatment (I.P.) |
|---|---|
| 1 | Bicarbonate Vehicle |
| 2 | Captisol ® Vehicle |
| 3 | Rosiglitazone 6 mg/kg |
| 4 | [LIM-0705] 114 mg/kg |
| 5 | [LIM-0705] 11.4 mg/kg |
| 6 | [LIM-0741] 85 mg/kg |

Blood is collected from the rats at day 1, 4, 7, 11, 14, 21, 28, 35, 42 and assayed for levels of cholesterol, serum glucose, insulin, and triglycerides. Body weight is also measured on the same days. Animals are sacrificed at the end of the 6-week study to obtain liver and kidney weights, aspartate transaminase (AST) and alanine aminotransferase (ALT) levels for toxicity analysis, mesenteric and epididmyal fat weight, and glucagon, glycated hemoglobin (% HbA1c) and adiponectin levels.

### Results:

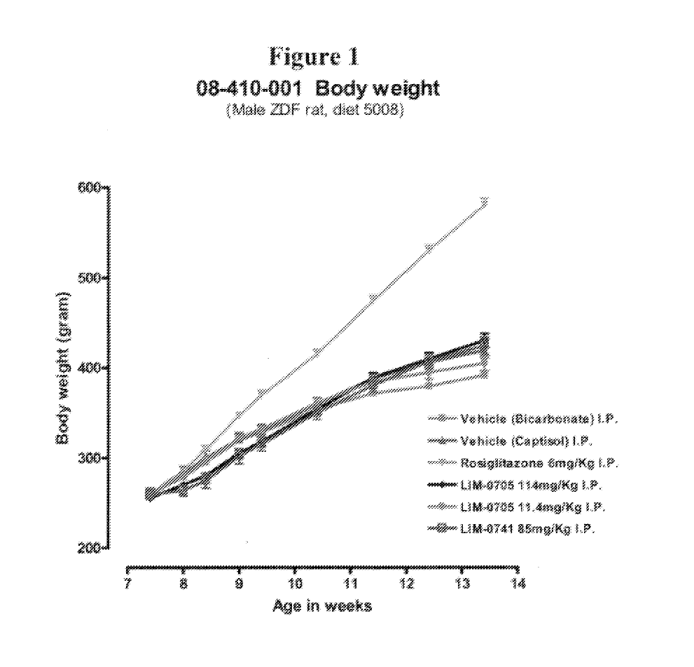

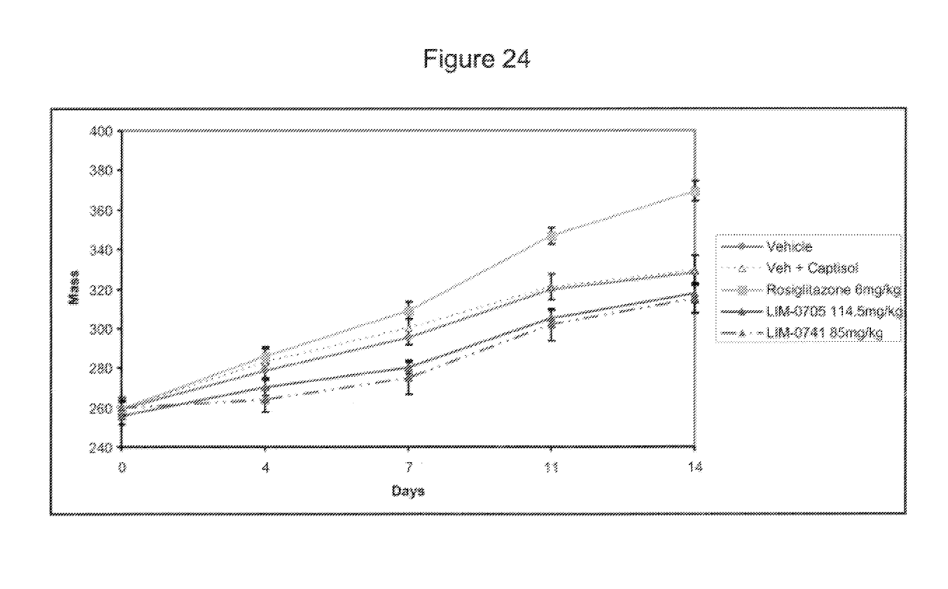

Body weight: Treatment with 6 mg/kg/day of the anti-diabetic drug, rosiglitazone causes marked increases in body weight over vehicle controls and treatment with the pyrone analogs. At the end of the 6 week of the study, ZDF rats treated with rosiglitazone have a mass of over 550 grams whereas rats with vehicle, [LIM-0705] and [LIM-0741] treatment have a mass of 400 grams. See FIG. 1. This increase in body weight by rosiglitazone can be attributed directly to the increase in mesenteric and epididymal fat. FIG. 24 shows that pyrone analogs LIM-0705 and LIM-0741 have little impact on weight gain of ZDF rats over 2 weeks of daily treatment.

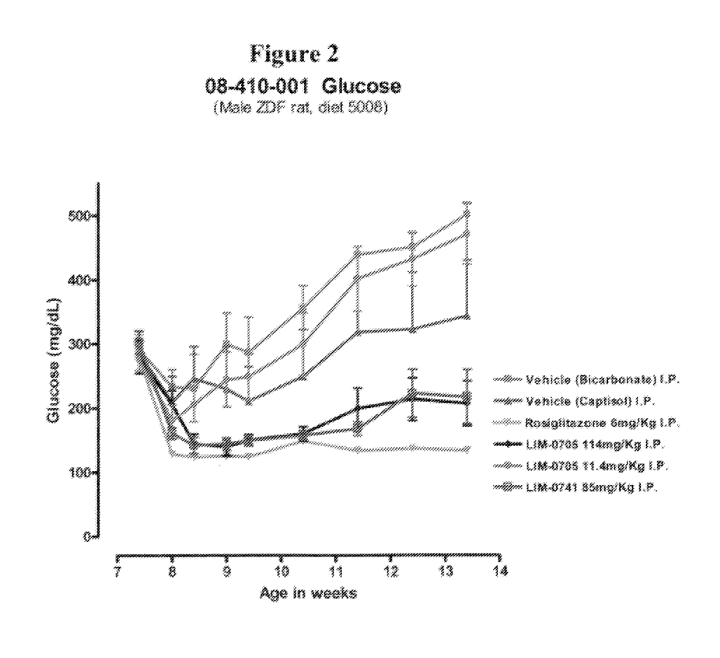

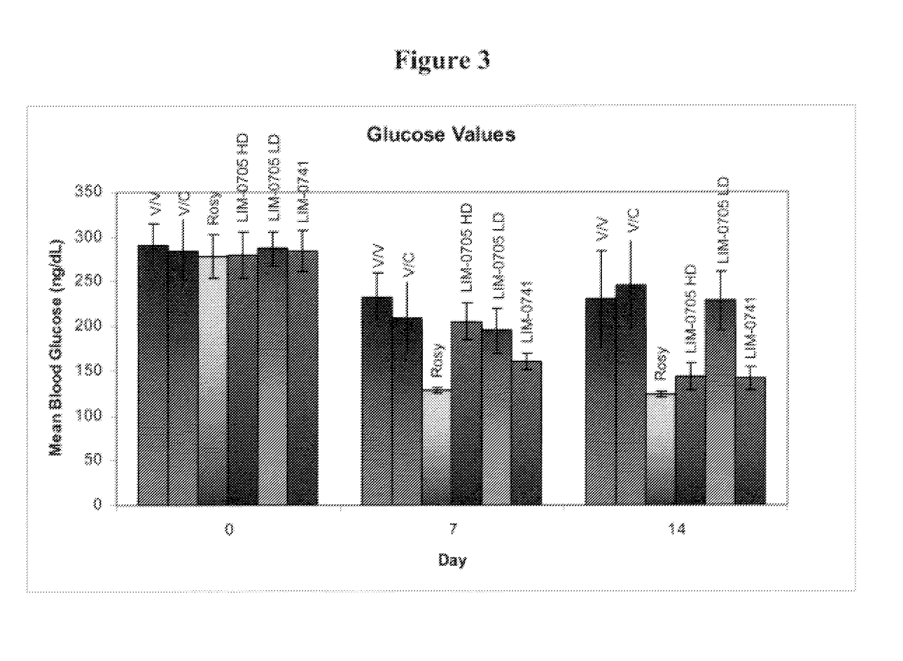

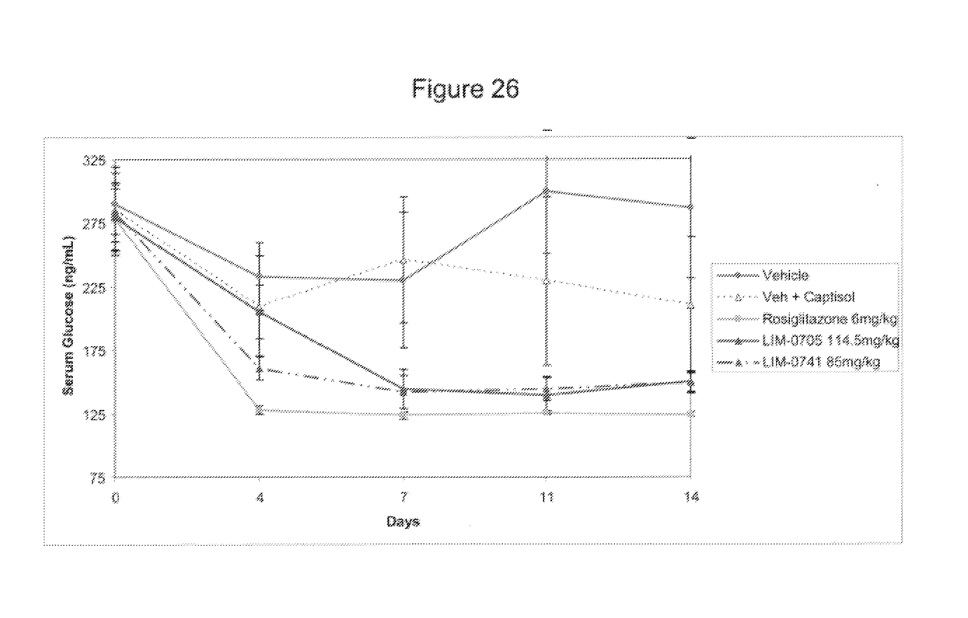

Serum glucose levels: The serum glucose levels show that the high dose (114 mg/kg) of [LIM-0705] and (85 mg/kg) of [LIM-0741] treatment maintains steady glucose levels similar to rosiglitazone treatment while vehicle and the low dose (11.4 mg/kg) of [LIM-0705] treatments cause increase in blood glucose levels over 6 weeks of daily treatment. These stable glucose levels indicate that both [LIM-0705] and [LIM-0741] treatments maintain the pancreatic beta cell response to glucose uptake. See FIGS. 2 and 3. FIG. 26 shows that pyrone analogs LIM-0705 (high dose) and LIM-0741 impact glucose levels in ZDF rats over 2 weeks of daily treatment.

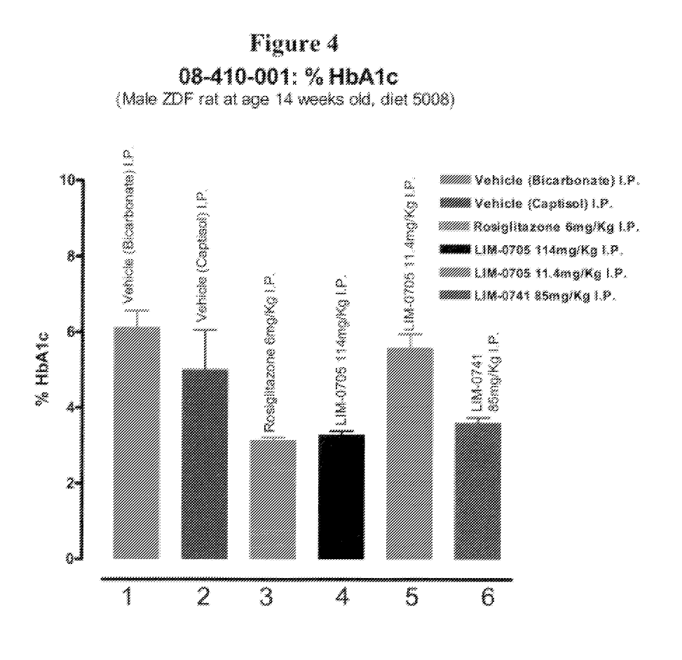

These results are also correlated by measuring glycated hemoglobin levels (% HbA1c). See FIG. 4.

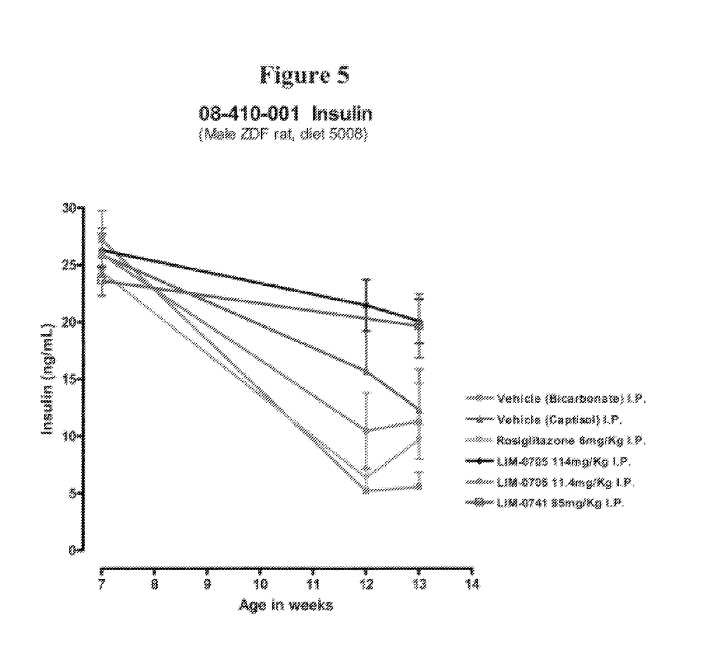

Insulin levels: The high dose (114 mg/kg) of [LIM-0705] and [LIM-0741] treatment also reduce decreases in insulin levels in comparison with vehicle controls, the low dose (11.4 mg/kg) of [LIM-0705] and rosiglitazone treatment suggesting that [LIM-0705] and [LIM-0741] treatment maintain beta cell function in secreting insulin during diabetes disease progression. See FIG. 5.

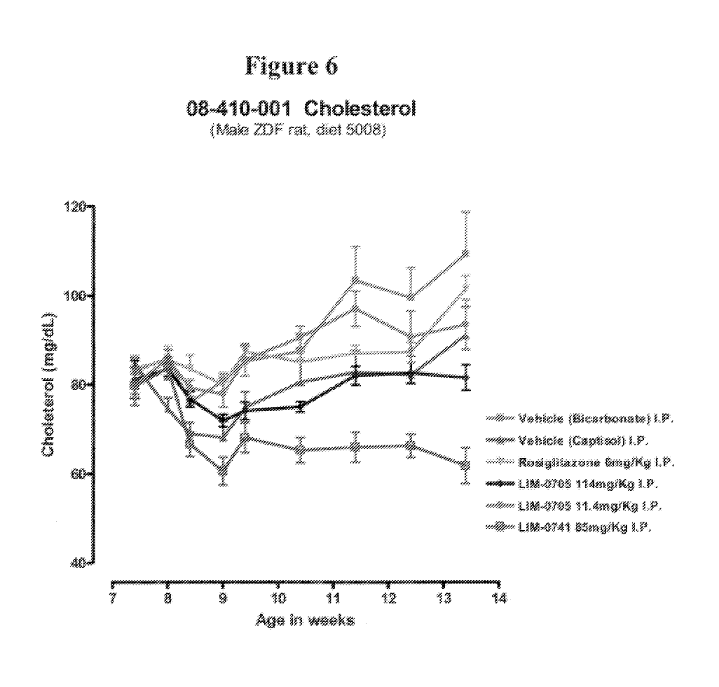

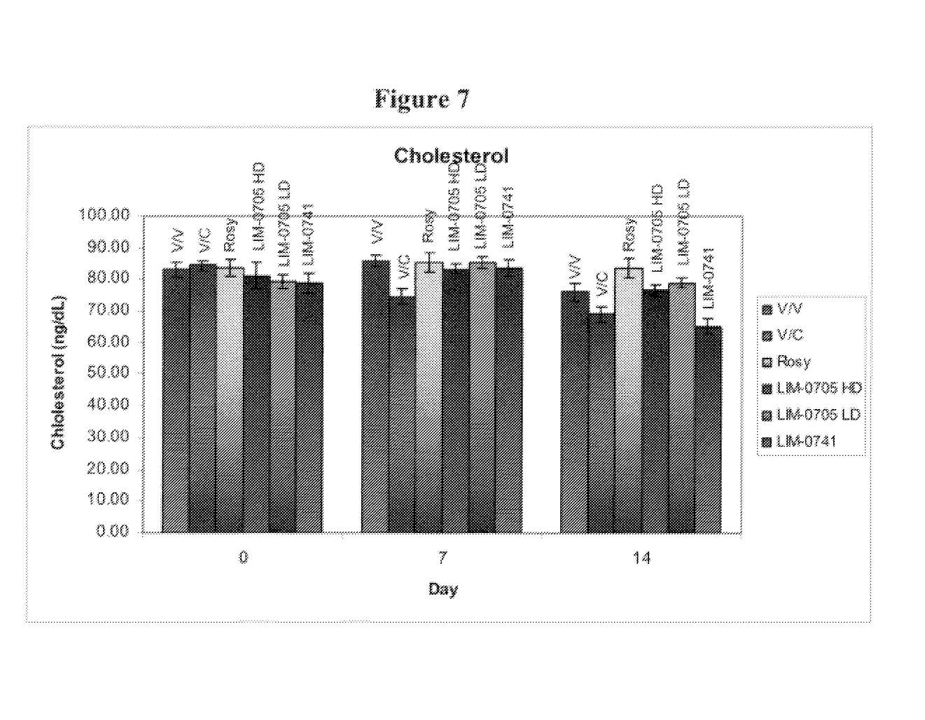

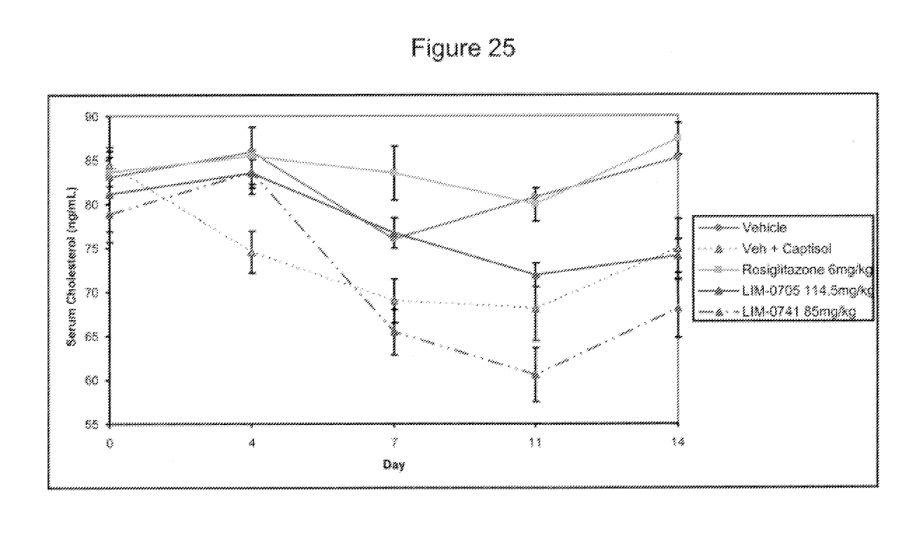

Cholesterol levels: Cholesterol levels show that the high dose (114 mg/kg) of [LIM-0705] and [LIM-0741] treatment lower cholesterol levels with respect to baseline vehicle control and rosiglitazone treatment. See FIG. 6. FIG. 7 illustrates cholesterol levels at days 1, 7 and 14 of treatment in animals treated with controls, Rosiglitazone, LIM-0705 or LIM-0741. FIG. 25 shows the effect of pyrone analogs LIM-0705 and LIM-0741 on cholesterol levels in ZDF rats over 2 weeks of daily treatment.

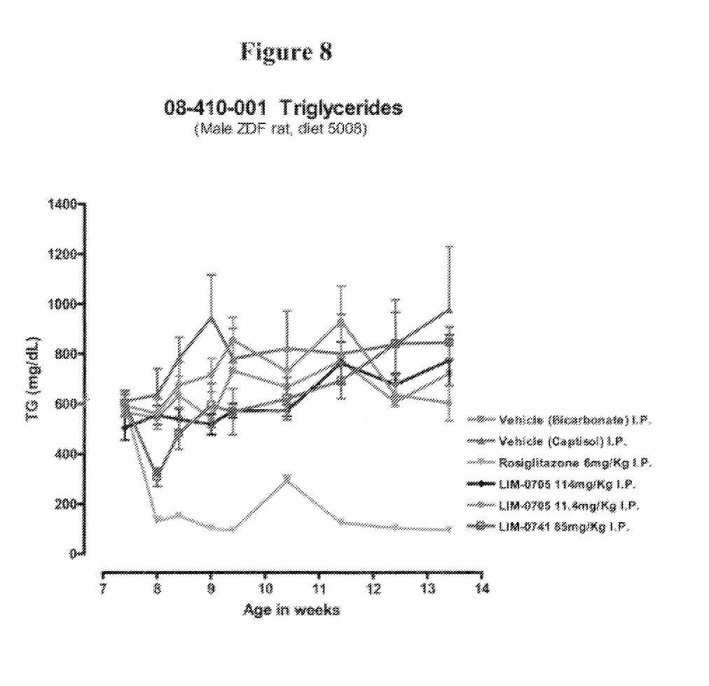

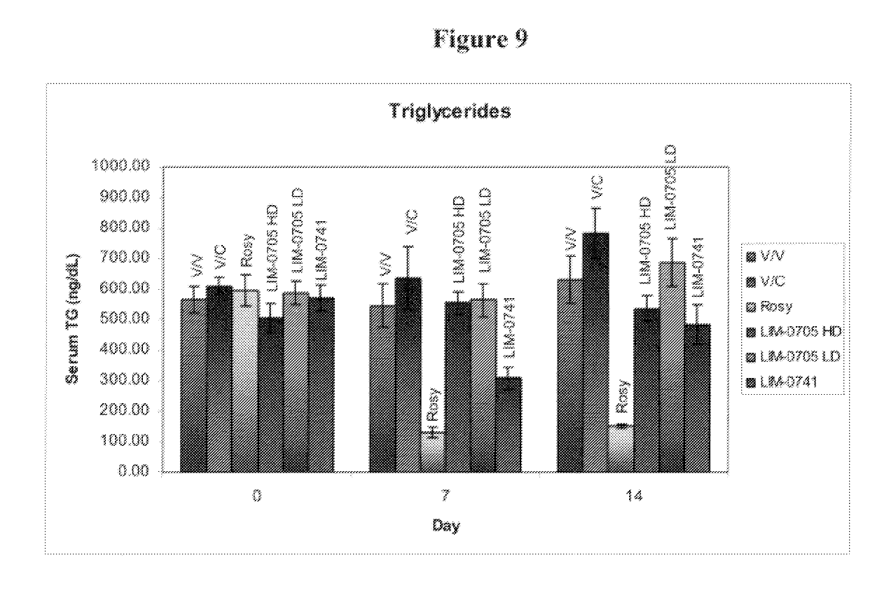

Triglycerides: Treatment with 6 mg/kg/day of the anti-diabetic drug, rosiglitazone causes marked decreases in triglycerides (mg/dL) over vehicle controls and treatment with the pyrone analogs, [LIM-0705] and [LIM-0741]. See FIG. 8. FIG. 9 illustrates triglyceride levels at days 1, 7 and 14 of treatment.

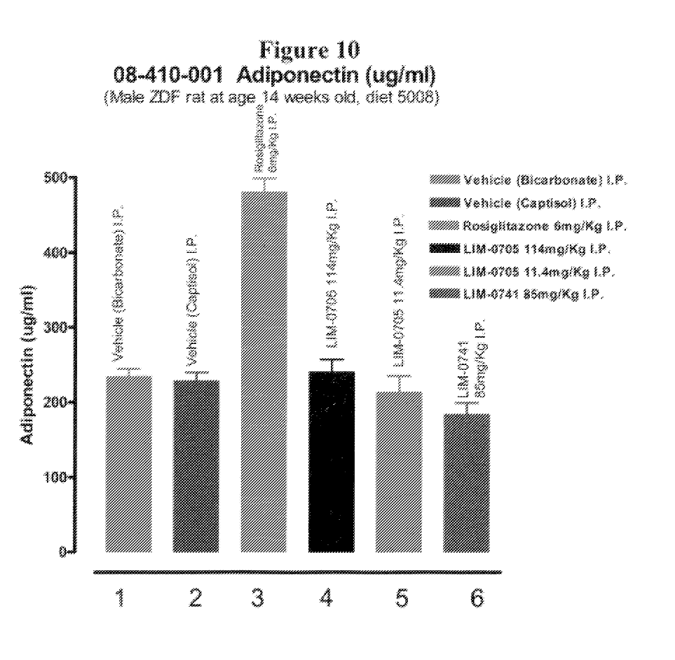

Adiponectin: Treatment with 6 mg/kg/day of the anti-diabetic drug, rosiglitazone causes marked increases in adiponectin (μg/mL) over vehicle controls and treatment with the pyrone analogs, [LIM-0705] and [LIM-0741]. See FIG. 10.

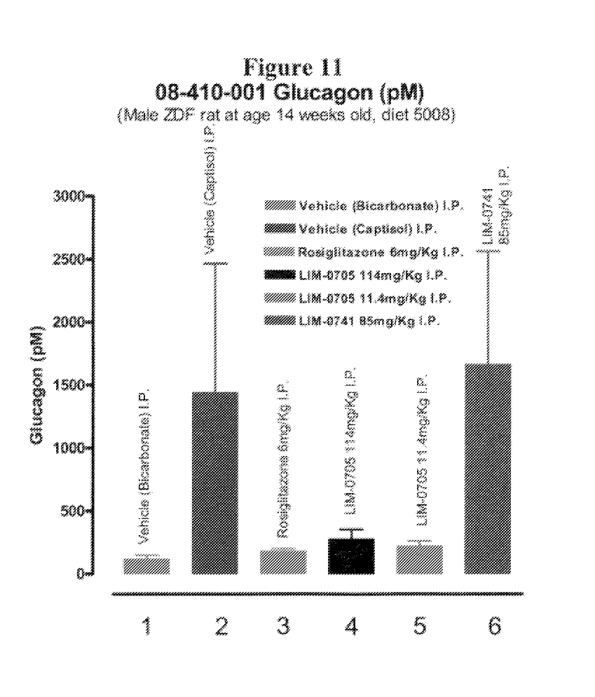

Glucagon: Treatment with 6 mg/kg/day of the anti-diabetic drug, rosiglitazone causes similar effects to the low and high doses of LIM-0705, whereas treatment with LIM-0741 caused effects similar to vehicle control with Captisol®. See FIG. 11.

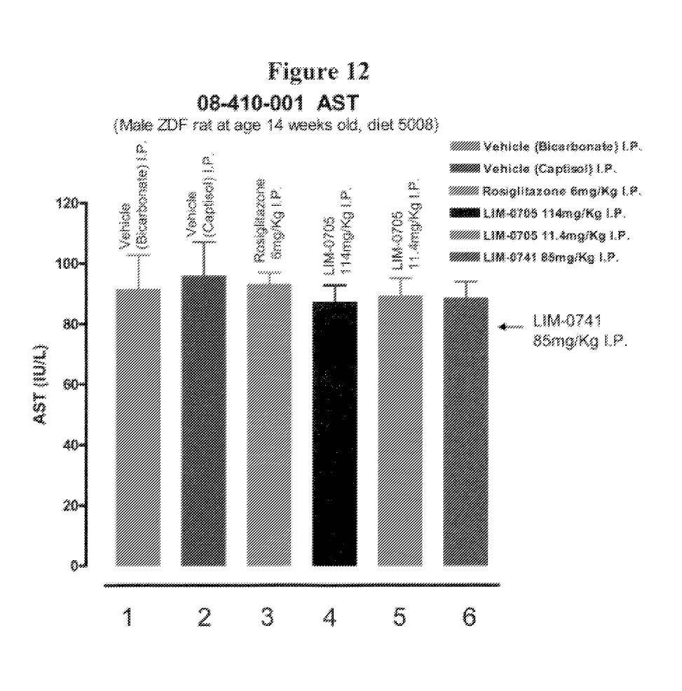

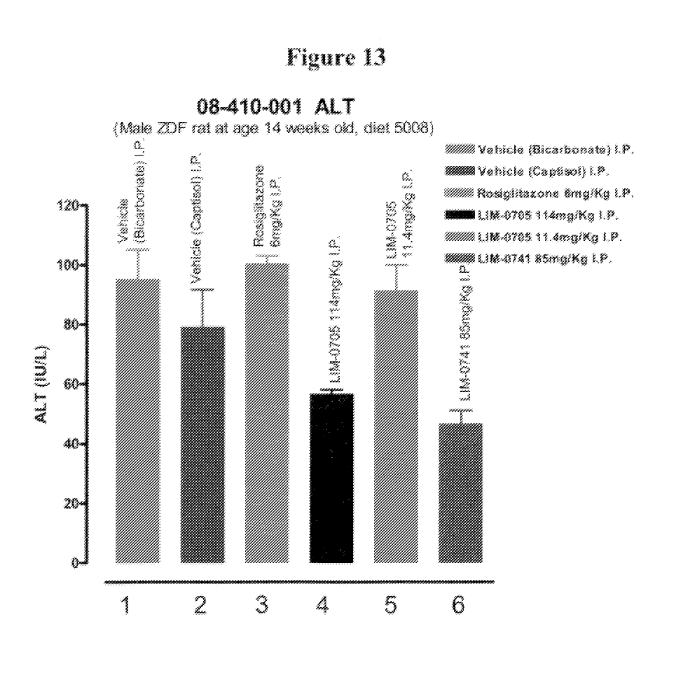

AST and ALT levels: AST levels also show no differences (see FIG. 12), while ALT levels are down over vehicle control when [LIM-0705] and [LIM-0741] are used for treatment (see FIG. 13). These results indicate that [LIM-0705] and [LIM-0741] have little effect on liver and kidney injury and toxicity.

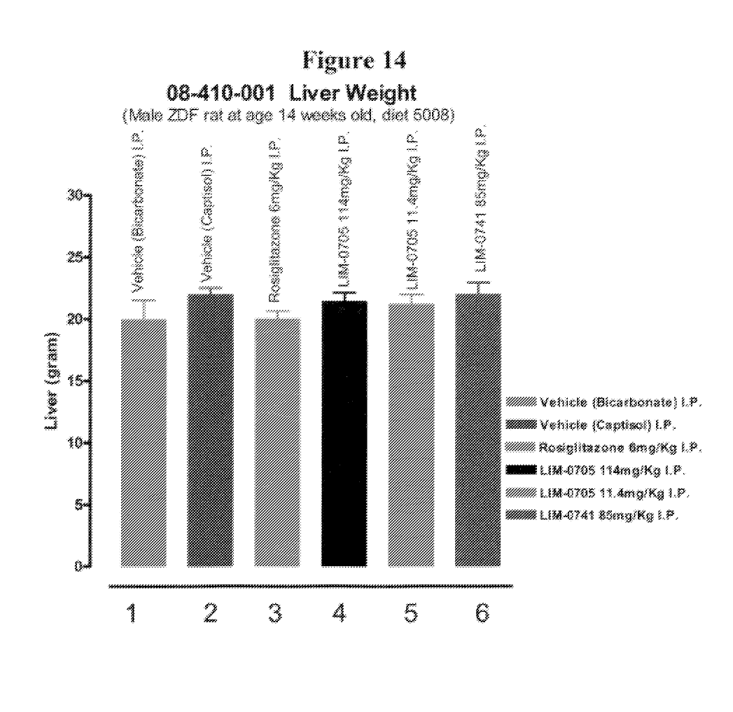

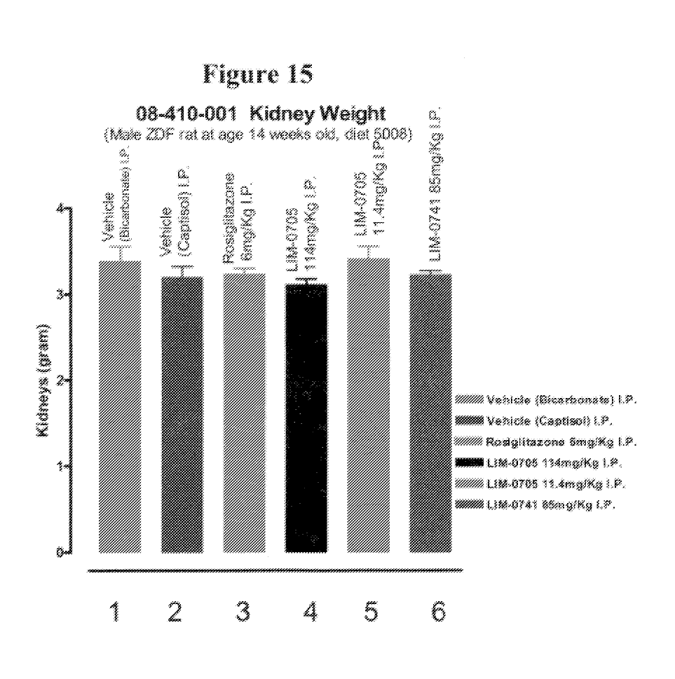

Liver and kidney weight: Treatment of either the pyrone analogs, [LIM-0705] and [LIM-0741], rosiglitazone or vehicles show similar liver and kidney weight at the end of week 6 (see FIGS. 14 and 15, respectively).

### Example 29

### LIM-0742 Protect Against Onset of Type 2 Diabetes and Attendant Complications in Diabetic Rat Model

Animals: Seven (7) week old male Zucker Diabetic Fatty (ZDF) rats are used. The ZDF rat is a model for Type 2 diabetes based on impaired glucose tolerance caused by the inherited obesity gene mutation that leads to insulin resistance. Between 7 and 10 weeks of age, a male ZDF rat has high blood insulin levels when fed with Purina 5008 chow that subsequently drop as pancreatic beta cells cease to respond to glucose. By 12 weeks of age, a male ZDF rat on a diet consisting of Purina 5008 chow is fully diabetic.

General procedures for animal care and housing are in accordance with the National Research Council (NRC) Guide for the Care and Use of Laboratory Animals (1996) and the Animal Welfare Standards incorporated in 9 CFR Part 3, 1991.

Experimental Design: This study is a 6 week study. Forty-eight (48) 7-week old male ZDF rats are chosen and divided into 6 treatment arms (8 rats/arm). The rats are kept on a diet of Purina 5008 to induce the onset of diabetes. The animals are treated daily with the following compounds:

| Group | Treatment |
|---|---|
| 1 | Water Vehicle (IP) |
| 2 | Rosiglitazone 6 mg/kg (PO) |
| 3 | Atorvastatin 10 mg/kg (PO) |
| 4 | LIM-0742 100 mg/kg |

Blood is collected from the rats at day 1, 4, 7, 11, 14, 21, 28, 35, 42 and assayed for levels of cholesterol, serum glucose, insulin, and triglycerides. Body weight is also measured on the same days. Animals are sacrificed at the end of the 6-week study to obtain liver and kidney weights, aspartate transaminase (AST) and alanine aminotransferase (ALT) levels for toxicity analysis, mesenteric and epididmyal fat weight, and glucagon, glycated hemoglobin (% HbA1c) and adiponectin levels.

### Results:

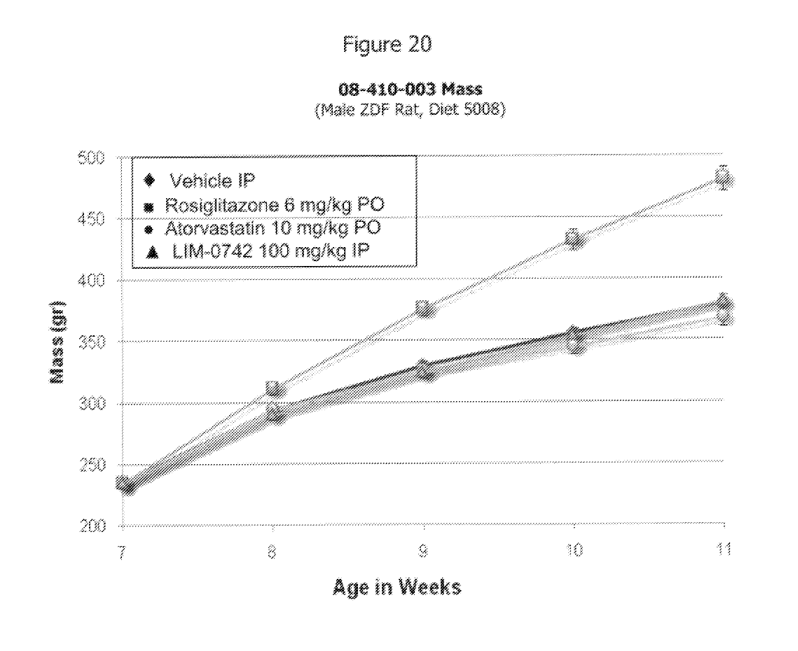

Body weight: Treatment with 6 mg/kg/day of the anti-diabetic drug, rosiglitazone causes marked increases in body weight over vehicle controls and treatment with the pyrone analogs. FIG. 20 shows that pyrone analog LIM-0742 has little impact on weight gain in ZDF rats. Rosiglitazone treated animals gain excessive weight compared to control, LIM-0742 and Atorvastatin treated animals. This increase in body weight by rosiglitazone can be attributed directly to the increase in mesenteric and epididymal fat.

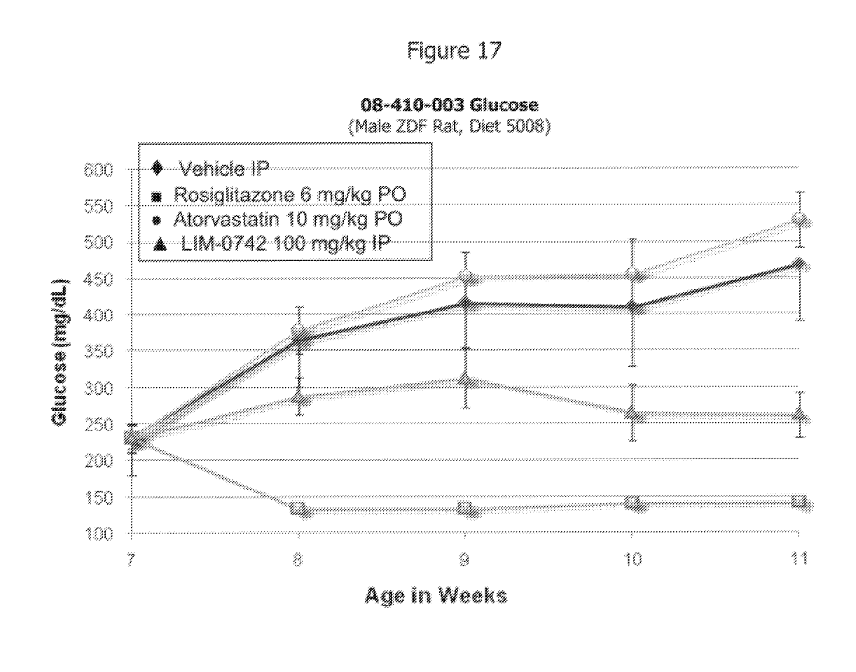

Serum glucose levels: FIG. 17 shows the effect of pyrone analog LIM 0742 on glucose levels in ZDF rats during 6 weeks of daily treatment. Rosiglitazone treated animals show optimal glucose control. LIM-0742 treated animals show glucose control that is superior to vehicle control.

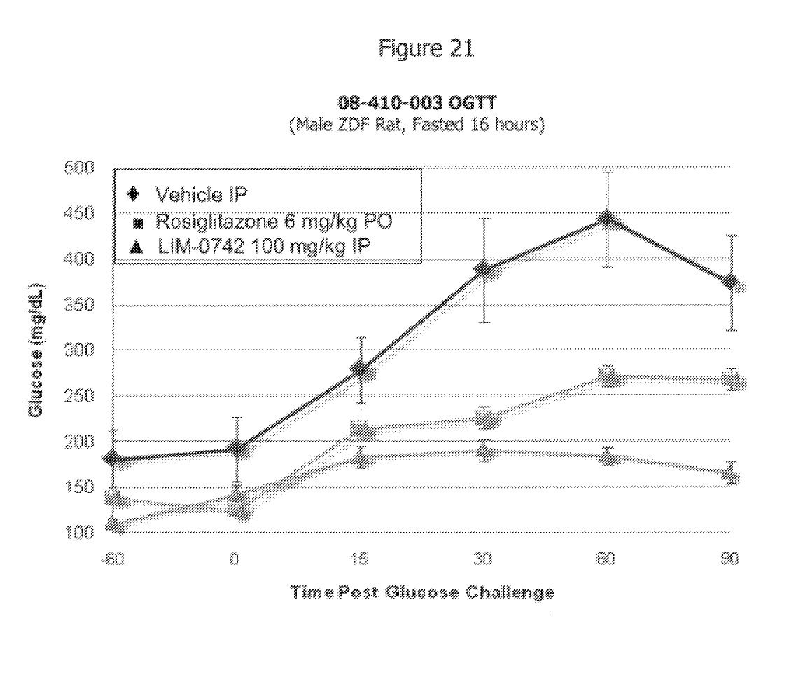

FIG. 21 shows that pyrone analog LIM 0742 protects against hyperglycemia after a glucose load (2 mg/kg) in fasted and aging ZDF rats. Glucose level stays in physiologic range in LIM-0742 arm treated animals compared to the elevated level observed in Rosiglitazone treated animals.

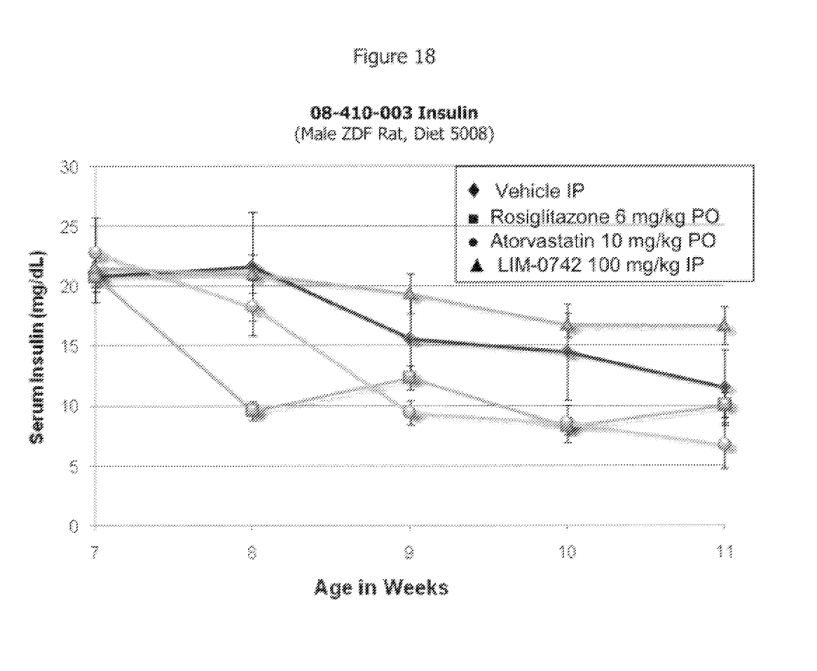

Insulin levels: FIG. 18 shows that pyrone analog LIM 0742 produces elevated insulin levels in ZDF rats during 6 weeks of daily treatment. Rosiglitazone treated animals are insulin sensitized. LIM-0742 treated animals maintain insulin output throughout the study.

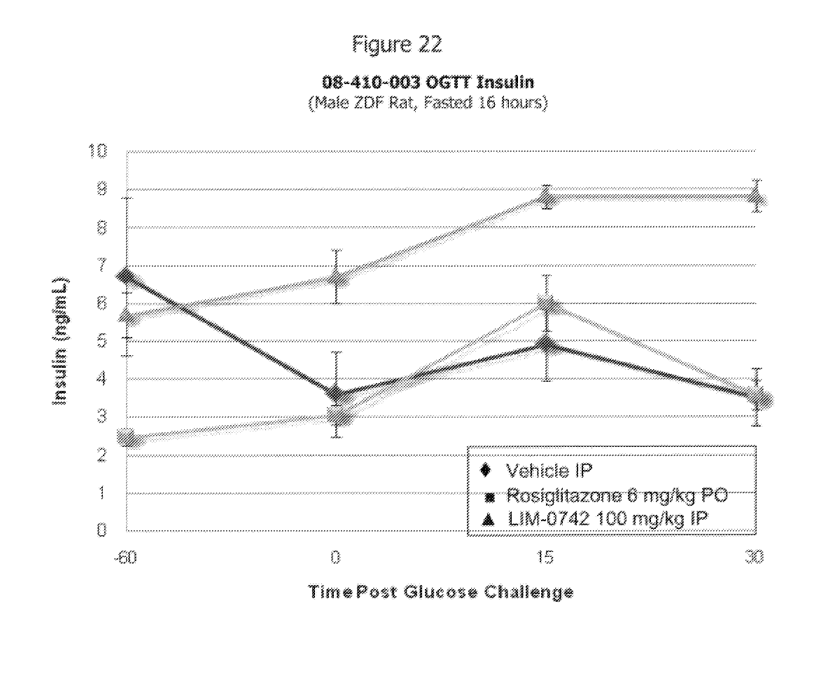

FIG. 22 shows that pyrone analog LIM 0742 produces an insulin response after a glucose load (2 gr/kg) in fasted and aging ZDF rats. Rosiglitazone treated animals cannot maintain sufficient insulin output to handle glucose load. LIM-0742 arm treated animals maintain an effective insulin response.

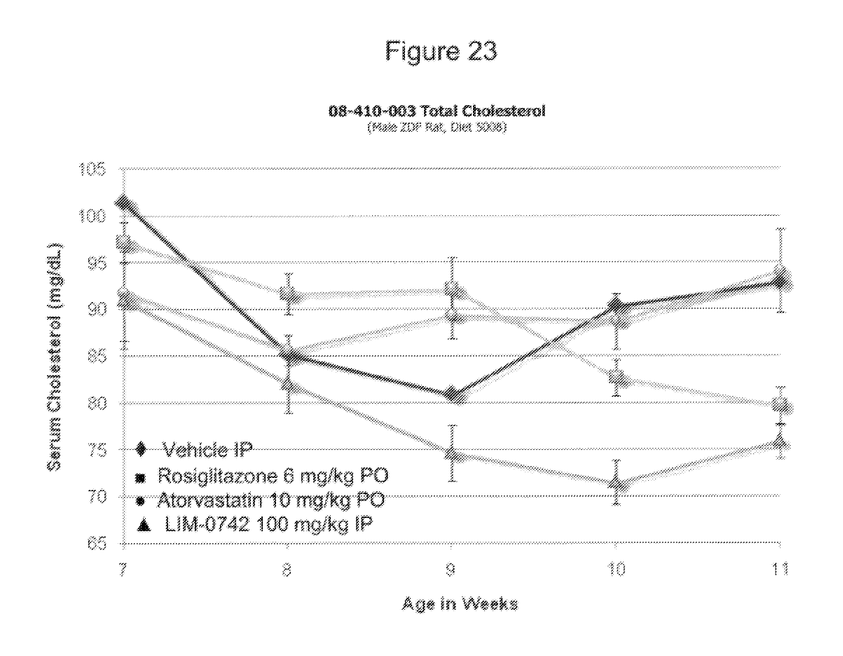

Cholesterol levels: FIG. 23 demonstrates that Rosiglitazone treated animals and LIM-0742 treated animals have similar benefits with respect to total cholesterol reduction compared to vehicle control.

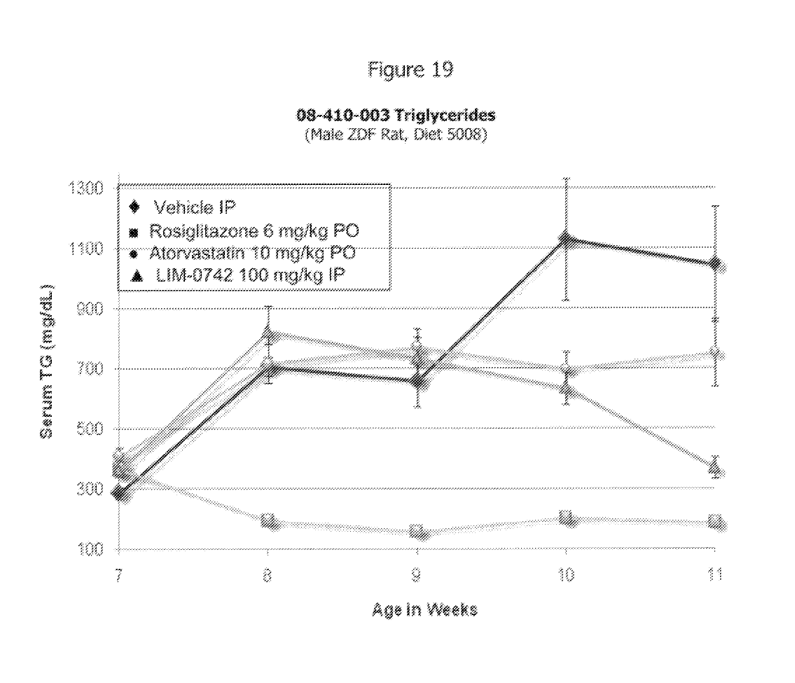

Triglycerides: FIG. 19 shows the effect of pyrone analogs on circulating triglyceride levels in aging ZDF rats. Rosiglitazone treated animals and LIM-0742 animals see similar benefits at triglyceride reduction.

### Example 30

### Synthesis of Quercetin-3-O-methylphosphonic acid (Compound 1)

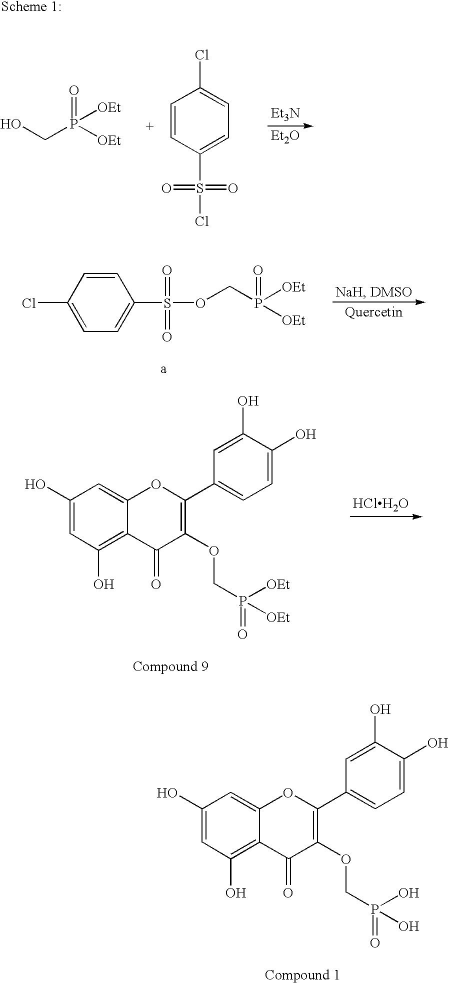

(Diethoxyphosphoryl)methyl 4-chlorobenzenesulfonate (a): Diethyl(hydroxymethyl)phosphonate (10.0 g, 59.5 mmol, 1 equiv) is dissolved in dry ether (80 mL), and triethylamine (902 mL, 65.5 mmol, 1.1 equiv) is added. The reaction mixture is cooled to −10° C. and a solution of 4-chlorobenzenesulfonyl chloride (13.8 g, 65.5 mmol, 1.1 equiv) in dry ether (30 mL) is added dropwise via a syringe. The reaction mixture is stirred at 0° C. for 2 hr then allowed to warm to room temperature and stirred for 17 hr. The reaction mixture is filtered. Water (150 mL) and ethyl acetate (150 mL) are added to the filtrate and the phases separated. The organic phase is washed with brine, dried over sodium sulfate, and concentrated in vacuo. The product is purified by chromatography on an Analogix system (SF40-115 g column) using 40-100% heptane/dichloromethane as the eluent. The product is obtained as a white solid (14.2 g, 41.4 mmol, 70% yield).

Diethyl (2-(3,4-dihydroxyphenyl)-5,7-dihydroxy-4-oxo-4H-chromen-3-yl)methylphosphonate (Compound 9): Quercetin dihydrate (4.48 g, 13.25 mmol, 1.0 equiv) is stirred in DMSO (45 mL), while 60% sodium hydride in mineral oil (1.59 g, 66.25 mmol, 4.0 equiv) is added slowly portion-wise. The reaction mixture is allowed to stir at room temperature for 5 min. A solution of (diethoxyphosphoryl)methyl 4-chlorobenzenesulfonate (a) (5.00 g, 14.58 mmol, 1.1 equiv) in dimethylsulfoxide (15 mL) is added and the reaction mixture is stirred at room temperature for 16 hr, at which time LC-MS indicated quercetin is still present. Additional 60% sodium hydride in mineral oil (0.3 g, 13.25 mmol, 1.0 equiv) is added and the reaction mixture is stirred at room temperature for an additional 4 hr, at which time LC-MS indicated the reaction is complete. The reaction mixture is diluted with water (300 mL), and 10% hydrochloric acid is added to adjust the pH to 2. The mixture is extracted with ethyl acetate (100 mL×3). The combined organic phases are washed with brine, dried over sodium sulfate and concentrated in vacuo. The product is purified by chromatography on an Analogix system (SF25-60 g column) using 0-10% methanol/dichloromethane as the eluent. The product is obtained as an orange oil (3.56 g, 7.88 mmol, 60% yield).

Quercetin-3-O-methylphosphonic acid (Compound 1): A mixture of diethyl (2-(3,4-dihydroxyphenyl)-5,7-dihydroxy-4-oxo-4H-chromen-3-yl)methylphosphonate (Compound 9) (3.56 g, 7.88 mmol) and concentrated hydrochloric acid (20 mL) is refluxed for 16 hr. Orange precipitate is formed. After cooling to room temperature, the reaction mixture is filtered. The residual orange solid is dissolved in water (20 mL) and purified by C-18 reverse-phase column chromatography on an Analogix systems (SF40-150 g column) (10 mL per injection) using 0-10% methanol/water as the eluent. Compound 1 (also called quercetin-3-O-methylphosphonate) is obtained as yellow solid (0.054 g, 0.136 mmol, 1.7% yield, >99.5 purity). ¹H NMR (300 MHz/DMSO-d₆): δ 7.68-7.64 (m, 2H), 6.90 (d, 1H), 6.41 (s, 1H), 6.20 (s, 1H), 4.26 (d, 2H).

### Example 31

### Synthesis of Quercetin-3′-O-methylphosphonic Acid (Compound 2)

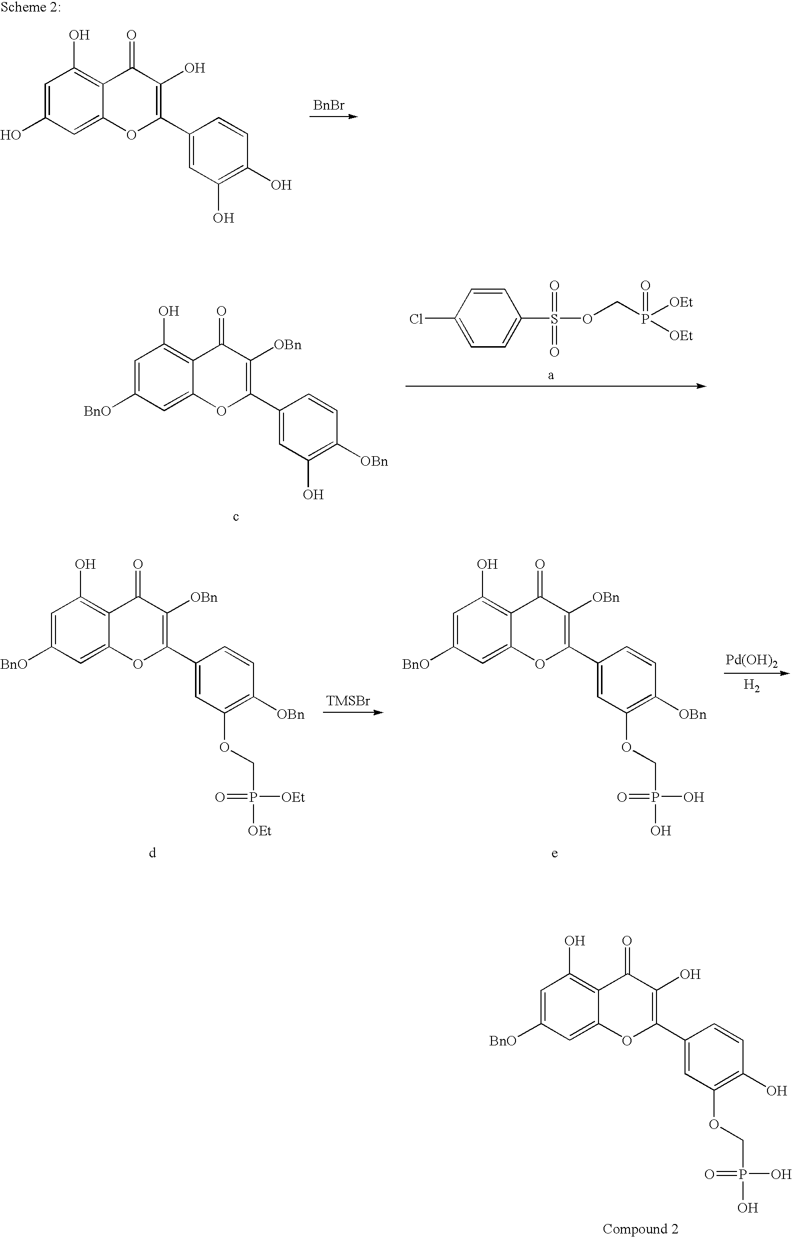

3,7-Bis(benzyloxy)-2-(4-(benzyloxy)-3-hydroxyphenyl)-5-hydroxy-4H-chromen-4-one (c): Quercetin dihydrate (300.0 g, 887.6 mmol, 1.00 equiv) is stirred in DMF (6 L) at 0° C., while potassium carbonate (361.3 g, 2618.3 mmol, 2.95 equiv) is added. Benzyl bromide (311 mL, 2618.3 mmol, 2.95 equiv) is added slowly via an additional funnel. The reaction is stirred vigorously for 2 hr at 0° C. and then slowly warmed to room temperature in 2 hr. The reaction is allowed to stir at room temperature for 16 hr and poured into water (20 L). The mixture is acidified with 1N hydrochloric acid until pH=3 and extracted with ethyl acetate (3×8 L). The organic layer is washed with saturated brine (4 L), dried over sodium sulfate, filtered and concentrated under reduced pressure. The product is purified by flash chromatography using 1:1 toluene/dichloromethane (16 L), dichloromethane (12 L) and 3% ethyl acetate/dichloromethane (20 L) as eluents. The resulting solid is triturated from ten-butyl methyl ether and then 1:1 toluene/dichloromethane. The product is obtained as yellow solid (159 g, 31% yield).

(Diethoxyphosphoryl)methyl 4-chlorobenzenesulfonate (a): Diethyl (hydroxymethyl) phosphonate (386.0 g, 2297.6 mmol, 1.0 equiv) is dissolved in tert-butyl methyl ether (3.5 L), and triethylamine (355 mL, 2527.4 mmol, 1.1 equiv) is added. The reaction mixture is cooled to −10° C. by acetone-dry ice bath and a solution of 4-chloro-benzenesulfonyl chloride (533.5 g, 2527.4 mmol, 1.1 equiv) in tert-butyl methyl ether (1.5 L) is added slowly via an additional funnel. The acetone-dry ice bath is replaced with an ice-water bath. The reaction mixture is stirred at 0° C. for 2 h, warmed to room temperature and stirred for 17 hr. The reaction mixture is poured into water (4 L) and ethyl acetate (4 L) and the phases separated. The organic phase is washed with saturated brine, dried over sodium sulfate, and concentrated under reduced pressure to give amber oil. The product crystallized overnight at room temperature. The crystals are filtered and washed with heptane to give product as a white solid (296 g). The mother liquor is concentrated under reduced pressure and purified by flash chromatography using 0-100% ethyl acetate/heptanes as the eluent. The product is obtained as white solid (432 g, 55% yield).

Diethyl (2-(benzyloxy)-5-(3,7-bis(benzyloxy)-5-hydroxy-4-oxo-4H-chromen-2-yl)phenoxy)methyl-phosphonate (d): 3,7-Bis(benzyloxy)-2-(4-(benzyloxy)-3-hydroxyphenyl)-5-hydroxy-4H-chromen-4-one (c) (158 g, 276.2 mmol, 1.0 equiv) is stirred in DMF (2800 mL) at room temperature, while potassium tert-butoxide (74 g, 662.9 mmol, 2.4 equiv) is added portion-wise. The reaction mixture is allowed to stir at room temperature for 15 min. (Diethoxyphosphoryl)methyl 4-chlorobenzenesulfonate (a) (109 g, 317.6 mmol, 1.15 equiv) is added and the reaction mixture is stirred at room temperature for 16 hr, at which time LC-MS indicated the reaction is completed. The reaction mixture is diluted with water (3 L) and 1N hydrochloric acid (800 mL) is added to adjust the pH to 4. Yellow precipitates formed and the mixture is filtered. The solid is crystallized from ethanol to give product 151 g of product, Compound d. The filtrate is extracted with ethyl acetate (2×2 L), washed with water, saturated brine, dried over sodium sulfate, concentrated under reduced pressure. The residue is crystallized from ethanol to give an additional 17 g of product. The solids are combined to give the product as yellow solid (168 g, 84% yield, >97% purity by LC-MS).

(2-(Benzyloxy)-5-(3,7-bis(benzyloxy)-5-hydroxy-4-oxo-4H-chromen-2-yl)phenoxy)methylphosphonic acid (e): Diethyl (2-(benzyloxy)-5-(3,7-bis(benzyloxy)-5-hydroxy-4-oxo-4H-chromen-2-yl)-phenoxy)methylphosphonate (d) (84 g, 116 mmol, 1 equiv) is stirred in chloroform (1.8 L) in a 3 L round bottom flask charged with nitrogen at room temperature, while bromotrimethylsilane (215 mL, 1627 mmol, 14 equiv) is added. The reaction mixture is allowed to stir at room temperature for 16 hr and concentrated under reduced pressure to give orange wax. Methanol (2.8 L) is added to the residue and the mixture is heated to reflux for 2 hr. Yellow precipitate formed upon cooling and is collected by filtration. The yellow solid is washed with cold methanol and ethyl acetate, and dried in the vacuum oven at 30° C. for 16 hr. Compound e is obtained as yellow solid (73 g, 95% yield).

Quercetin-3′-O-methylphosphonic acid (Compound 2): (2-(Benzyloxy)-5-(3,7-bis(benzyloxy)-5-hydroxy-4-oxo-4H-chromen-2-yl)phenoxy)methylphosphonic acid (e) (20.0 g, 30.0 mmol, 1 equiv) and palladium hydroxide (20% wt) (1.2 g) are suspended in ethanol (1.2 L). The reaction is hydrogenated for 7 hr @ 50 psi. The mixture is filtered through celite, concentrated under reduced pressure and dried in vacuum oven for 16 hr to give yellow solid, at which time ¹H NMR indicated significant amount of ethanol. The yellow solid is then stirred in D.I.U.F. water (deionized ultra-filtered water, 300 mL) at 35° C. for 0.5 hr and lyophilized for 2 days. Compound 2 is obtained as yellow solid (12 g, 100% yield, >98% purity by LC-MS, structure is confirmed by 2-D NMR). ¹H NMR (500 MHz/CD₃OD): δ 7.89 (d, 1H), 7.75 (dd, 1H), 6.93 (d, 1H), 6.37 (s, 1H), 6.15 (s, 1H), 4.32 (d, 2H); ¹³C NMR (125.8 MHz/CD₃OD): δ 177.3, 165.6, 162.5, 158.2, 150.0, 148.0, 147.3, 137.4, 124.2, 124.0, 117.0, 114.1, 104.5, 99.3, 94.5, 65.7.

### Example 32

### Synthesis of Fisetin-3-O-methylphosphonic Acid (Compound 3)

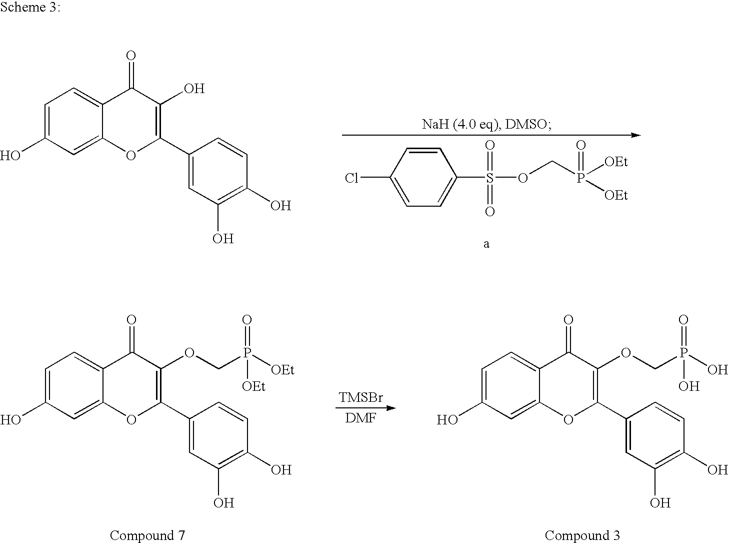

Diethyl (2-(3,4-dihydroxyphenyl)-7-hydroxy-4-oxo-4H-chromen-3-yloxy)methylphosphonate (Compound 7): To a solution of fisetin (60.74 g, 212.20 mmol, 1.0 equiv) under nitrogen in anhydrous DMSO (600 mL) is added sodium hydride (20.88 g, 870.02 mmol, 4.1 equiv) as a 60% suspension in oil in 4 equal portions over 1 hour. To this dark-red heterogeneous mixture is added (diethoxyphosphoryl)methyl 4-chlorobenzenesulfonate (Compound a) (80.00 g, 233.42 mmol, 1.1 equiv) in anhydrous DMSO (200 mL) dropwise over 2 hours. The mixture is allowed to stir at room temperature for 67 hours. Water (2 L) is added to the mixture while cooling with an ice-bath to maintain the temperature below 30° C. The mixture is acidified to pH ˜3 by adding 1N HCl. Multiple portions (1 L each) of the mixture are extracted with 10% isopropanol in dichloromethane (4×300 mL). The combined organic layers are concentrated under reduced pressure. The resultant dark oil is chromatographed through a column of silica gel eluting with 0-10% methanol in dichloromethane. The fractions containing product are combined and concentrated. The resultant oil is chromatographed using an Analogix C-18 40-300 g reverse phase column eluting with 0-100% methanol (with 0.1% formic acid) in water (with 0.1% formic acid). The fractions containing product are combined and concentrated under vacuum. The resultant yellow solid is dried in a vacuum oven at 50° C. overnight to provide Compound 7 (6.62 g, 7.1% yield) as a yellow solid with >98% purity.

Fisetin-3-O-methylphosphonic acid (Compound 3): To a solution of diethyl (2-(3,4-dihydroxyphenyl)-7-hydroxy-4-oxo-4H-chromen-3-yloxy)methylphosphonate (Compound 7) (3.40 g, 7.79 mmol, 1.0 equiv) in anhydrous DMF (3.0 mL) under nitrogen is added trimethylsilyl bromide (15.42 mL, 17.89 g, 116.85 mmol, 15 equiv) dropwise over 5 minutes. The temperature is kept below 30° C. during addition by using ice/water bath. The mixture is stirred at room temperature for 15 hours. The solvent is then removed under reduced pressure and the resultant orange oil is dissolved in methanol (20 mL). Water (5 mL) is added and the mixture is allowed to stir for 1 hour at room temperature. The solvents are removed under reduced pressure. Deionized water (50 mL) is added to the residue and 24% sodium hydroxide (w/w) is added dropwise until all the material had dissolved. The aqueous solution is washed with dichloromethane (20 mL) and ethyl acetate (40 mL). The aqueous solution is acidified until pH˜1 using concentrated HCl. The mixture is allowed to stir for 15 minutes as a fine orange precipitate formed. The solid is filtered and rinsed with deionized water. The resultant solid is dried under a stream of nitrogen and then in a vacuum oven at 40° C. overnight (19 hours) to provide (Compound 3) (3.0 g) as an orange solid (quantitative yield, 99.1% purity by LC/MS). ¹H NMR (300 MHz/CD₃OD) δ 4.22 (d, 2H, J_{P}=9.4 Hz), 6.92 (m, 3H), 7.65 (s, 1H), 7.66 (dd, 1H, J=2.0, 9.9 Hz), 8.00 (dd, 1H, J=2.0, 7.2 Hz); ¹³C NMR (75 MHz/CD₃OD) δ 66.79 (d, J_{P}=157.8 Hz), 101.91, 115.27, 115.34, 116.13, 121.50, 121.83, 126.77, 139.15, 139.32, 145.34, 149.03, 157.30, 157.45, 163.75, 175.03.

### Example 33

### Synthesis of Fisetin-3′-O-methylphosphonic Acid (Compound 4)

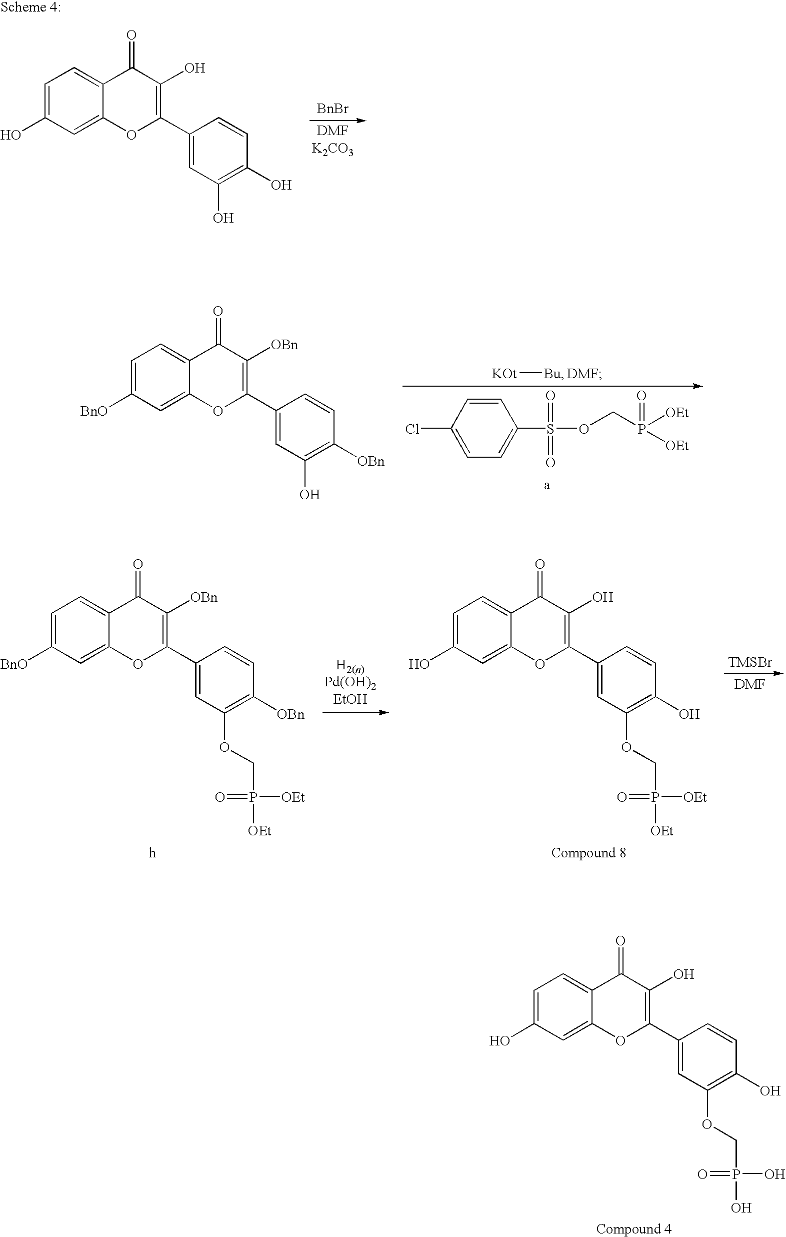

3,7-Bis(benzyloxy)-2-(4-(benzyloxy)-3-hydroxyphenyl)-4H-chromen-4-one (g): To a solution of Fisetin (180.0 g, 0.63 mol, 1 equiv.) in anhydrous DMF (4 L) under nitrogen at 0° C. is added potassium carbonate (260.3 g, 1.89 mmol, 3 equiv), portionwise over 30 minutes. Benzyl bromide (224 mL, 322.6 g, 1.89 mmol, 3 equiv) is added dropwise over 1 hour at 0° C. The mixture is allowed to warm to room temperature and stirred over the weekend (63 hours). The mixture is poured into ethyl acetate (20 L) and washed with water (3×12 L). The organic layer is dried over sodium sulfate and concentrated under reduced pressure to obtain a yellow solid. The resultant yellow solid is triturated with dichloromethane (700 mL) at 38° C. for 30 minutes to break up the solid. The mixture is then stirred at 0° C. for 30 minutes, filtered, and rinsed to provide 71 g (21%) of product (>95% purity by LC/MS) as a white solid. The filtrate contained ˜1:1 mixture of (g) and the tetrabenzylated product.

Diethyl (2-(benzyloxy)-5-(3,7-bis(benzyloxy)-4-oxo-4H-chromen-2-yl)phenoxy)methylphosphonate (h): To a solution of 3,7-bis(benzyloxy)-2-(4-(benzyloxy)-3-hydroxyphenyl)-4H-chromen-4-one (g) (50.0 g, 89.93 mmol, 1 equiv.) in anhydrous DMF (400 mL) is added potassium tert-butoxide (12.1 g, 107.80 mmol, 1.2 equiv) in 2 equal portions. (Note: The solution went from yellow to dark red.) The mixture is allowed to stir at room temperature for 30 minutes. (Diethoxyphosphoryl)methyl 4-chlorobenzenesulfonate (a) (40.0 g, 116.78 mmol, 1.3 equiv) in anhydrous DMF (150 mL) is added dropwise over 40 minutes with cooling to maintain temperature below 25° C. during the addition. The mixture is allowed to stir at room temperature for 18 hours. The mixture is diluted with water (400 mL) and acidified with 1N HCl until pH˜3. The mixture is extracted with ethyl acetate (600 mL). After sitting over weekend, a white, crystalline solid formed in the ethyl acetate layer which is filtered and rinsed with additional ethyl acetate. The solid is dried in a vacuum oven at 50° C. for 18 hours to provide Compound h (20.20 g) as a white, crystalline solid. The filtrate is concentrated under vacuum to provide a solid which is triturated with ethanol, filtered, and dried under vacuum at 50° C. to provide a second crop of (h) (10.64 g). The aforementioned aqueous layer contained a suspended solid which is subsequently filtered and dried under vacuum. This solid is then triturated with ethanol, filtered, and dried under vacuum at 50° C. to yield a third crop of (h) (23.86 g) The combined crops provided 54.7 g of (h) (86% yield, >95% purity).

Diethyl (5-(3,7-dihydroxy-4-oxo-4H-chromen-2-yl)-2-hydroxyphenoxy)-methylphosphonate (Compound 8): A suspension of diethyl (2-(benzyloxy)-5-(3,7-bis(benzyloxy)-4-oxo-4H-chromen-2-yl)phenoxy)methyl-phosphonate (h) (49.70 g, 70.32 mmol, 1 equiv) and palladium hydroxide (7.0 g) is hydrogenated in ethanol (1 L) @ 50 psi. After 18 hours LC/MS indicated that all the starting material had been consumed. The hydrogenation vessel is depressurized and placed under a blanket of nitrogen. The mixture is filtered through celite while rinsing with methanol until the filtrate is colorless. The filtrate is concentrated under reduced pressure and dried in a vacuum oven for 6 hours at 50° C. to yield Compound 8 (30.2 g, 98% yield) as a yellow solid with >95% purity.

Fisetin-3′-O-methylphosphonic acid (Compound 4): To a solution of diethyl (5-(3,7-dihydroxy-4-oxo-4H-chromen-2-yl)-2-hydroxyphenoxy)methylphosphonate (Compound 8) (27.22 g, 62.38 mmol) in anhydrous DMF (150 mL) is added TMSBr (123.49 mL, 143.25 g, 935.72 mmol, 15 equiv) dropwise over 30 minutes. The temperature is kept below 30° C. during addition by using ice/water bath. The mixture is stirred at room temperature for 4.5 hours. The solvent is then removed under reduced pressure and the resultant dark oil is dried in a vacuum oven at 50° C. for 17 hours. The resultant oily solid is placed under nitrogen and methanol (300 mL) is added to dissolve the material. After fully dissolved, the solution is allowed to stir at room temperature for 1 hour. To this mixture is added deionized water (1200 mL). After stirring for 3 hours a thick, beige precipitate had formed. The solid is filtered and the solid is dried in a vacuum oven at 50° C. overnight. To remove residual traces of DMF, the solid is suspended in deionized water (−75 mL) and sonicated for 30 minutes. The suspension is then placed into a centrifuge and the aqueous layer is decanted. The resultant solid is dried in a vacuum oven at 50° C. overnight (19 hours) to provide Compound 4 (20.54 g) as an orange solid (87% yield, 98.3% purity by LC/MS). ¹H NMR (300 MHz/DMSO-d₆) δ 4.20 (d, 2H, J_{P}=9.3 Hz), 6.92 (dd, 1H, J=2.3, 8.8 Hz), 6.99 (d, 1H, J=6.1 Hz), 7.00 (s, 1H), 7.77 (dd, 1H, J=2.0, 8.5 Hz), 7.87 (d, 1H, J=2.0 Hz), 7.95 (d, 1H, J=8.8 Hz), 9.18 (br, 1H), 10.76 (br, 1H); ¹³C NMR (75 MHz/DMSO-d₆) δ 66.9 (d, J_{P}=160.0 Hz), 102.8, 114.7, 114.9, 115.4, 116.5, 123.2, 123.3, 127.1, 138.0, 145.3, 147.6 (d, J_{P}=12.0 Hz), 149.4, 157.0, 162.9, 172.7.

### Example 34

### Somatostatin Release

Rat hippocampal slices (thickness 350 μm, round slice) are prepared by a standard method. Twenty rat hippocampal slices are placed in a perfusion chamber, incubated at 37° C. and perfused by a batch method while exchanging the incubation buffer every 10 minutes. The incubation buffer has the composition: NaCl, 124 mM; KCl, 5 mM; KH₂ PO₄, 1.24 mM; MgSO₄, 1.3 mM; CaCl₂, 2.4 mM; NaHCO₃, 26 mM; D-glucose, and 10 mM. A mixed gas of oxygen (95%) and carbon dioxide (5%) is used to saturate the buffer.

Perfusion for 150 minutes provides fractions 1-15. To fraction 9 is applied a high K⁺ (50 mM) stimulation. A phosphonated pyrone analog is added to fractions 7-15 to the concentration of 10⁻⁹ M, 10⁻⁸ M, 10⁻⁷ M, and 10⁻⁶M, respectively. Examples of a phosphonated pyrone analog include quercetin-3-O-methylphosphonate (compound 1), quercetin-3′-O-methylphosphonate (compound 2), fisetin-3-O-methylphosphonate (compound 3), fisetin-3′-O-methylphosphonate (compound 4), quercetin-4′-O-methylphosphonate (compound 5), and fisetin-4′-O-methylphosphonate (comopund 6). Nothing is added to control group. The respective fractions thus obtained are concentrated by lyophilization and somatostatin in the perfusate is quantified by radioimmunoassay (RIA). After the completion of the experiment, somatostatin remaining in the slices is extracted by a conventional method and quantified by radioimmunoassay. The somatostatin amount released by high K⁺ (50 mM) stimulation is calculated and the amount of somatostatin released due to the property of the phosphonated pyrone analog is measured.

Somatostatin release (%) by the phosphonated pyrone analog at each concentration is calculated as in the following. The somatostatin amount of each fraction is expressed by the percentage (%) relative to the somatostatin residual amount at the time the fraction is obtained. The value of fraction 8 immediately before high K⁺ (50 mM) stimulation is taken as the base and the values exceeding the base value are added with regard to fraction 9 and the subsequent peak fractions exceeding the base value to give somatostatin release (%). The number of the test samples measured is 10 or 11. Each value (%) is expressed by mean±S.E.M. The property of the phosphonated pyrone analog is subjected to Dunnett's multiple comparison test relative to control group.

### Example 35

### In Vitro Toxicity Screening of Fisetin-3′-O-Phosphonate

A secondary pharmacological screening of molecules of interest at a fixed concentration is often practiced in the pharmaceutical industry in order to evaluate the effect of the compound on secondary targets that could result in untoward toxicity in vivo. These secondary screens are well known in the art and can be carried out by labs which specialize in these tests such as MDS-Panlabs and CEREP. A secondary toxicity screen is performed with fisetin-3′-O-phosphonate at a concentration of 10 uM against 122 targets in enzyme, radioligand binding, and cellular assays by MDS Pharma Services by methods well known in the art. Inhibition may be found in some targets (percent inhibition at 10 μM in parentheses): ATPase, Na+/K+, Heart, Pig (65%), Nitric Oxide Synthase, Endothelial (eNOS) (72%), Protein Tyrosine Kinase, FGFR2 (94%), Protein Tyrosine Kinase, FGFR1 (96%), Protein Tyrosine Kinase, Insulin Receptor (91%), Protein Tyrosine Kinase, (82%), Protein Tyrosine Kinase, ZA70 (ZAP-70) (74%), UDP Glucuronosyltransferase, UGT1A1 (52%), Adenosine A₁ (50%), Adrenergic α_{2A} (57%), Dopamine D₄₇ (51%), Peripheral Benzodiazepine Receptor (PBR) (53%), Transporter, Monoamine rabbit (68%), Serotonin (5-Hydroxytryptamine) 5-HT_{1A} (62%).

The compound may be additionally tested in Adenosine_{A1}, Adrenergic_{A2A}, DopamineD₂₅, Histamine H₁-, and μ-Opiate GTPγS functional assays using a concentration of 10 μM. The compound may demonstrate 48% antagonist activity in the Adenosine_{A1} assay, and marked negative inhibition in the Adrenergic_{A2A} assay, potentially indicating PAF-5 could be acting as an inverse agonist in this assay.

The findings of this toxicology screen may indicate that fisetin-3′-O-phosphonate has low toxicity properties, especially in light of the fact that the concentration at 10 μM, is high as compared to a therapeutic dose (e.g. greater than ˜100 times).

### Example 36

### Phosphonated Pyrone Analog Decreases Cholesterol and Triglyceride Levels in Human

A 32-year-old, obese, Caucasian male has a cholesterol level of 299 mg/dL, a triglyceride level of 440 mg/dL, an LDL level of 199 mg/dL, and an HDL level of 25 mg/dL. He does not have diabetes, kidney, or liver disease. He has a family history of coronary artery disease—his father suffers a heart attack at age 50. Because this patient is a male, obese, and has a positive family history of heart disease, he is advised to immediately start using the composition described herein on a daily basis. Preferably, the composition is a tablet containing 20 mg of a phosphonated pyrone analog. Additionally, he must strictly adhere to a low fat diet, and regularly exercise 30 minutes daily or 45 minutes every other day. Examples of a phosphonated pyrone analog include quercetin-3-O-methylphosphonate (compound 1), quercetin-3′-O-methylphosphonate (compound 2), fisetin-3-O-methylphosphonate (compound 3), fisetin-3′-O-methylphosphonate (compound 4), quercetin-4′-O-methylphosphonate (compound 5), and fisetin-4′-O-methylphosphonate (comopund 6).

The patient follows up with his doctor in 3 months with a repeat lipid profile. The blood test result shows an improvement of decreased cholesterol and triglycerides to 250 mg/dL and 280 mg/dL, respectively. The follow up plan also includes maintaining the same dosage of composition at 20 mg for two months, since the patient tolerates the medication well.

### Example 37

### Phosphonated Pyrone Analog Decreases Triglyceride Level in Human

A 45-year-old Hispanic male with a history of gout and gastritis has a triglyceride level of 950 mg/dL, and a cholesterol level of 300 mg/dL. The patient begins using a composition described herein, for example a tablet containing 50 mg of a phosphonated pyrone analog, twice daily with no side effects. Examples of a phosphonated pyrone analog include quercetin-3-O-methylphosphonate (compound 1), quercetin-3′-O-methylphosphonate (compound 2), fisetin-3-O-methylphosphonate (compound 3), fisetin-3′-O-methylphosphonate (compound 4), quercetin-4′-O-methylphosphonate (compound 5), and fisetin-4′-O-methylphosphonate (comopund 6). The patient is very compliant with respect to taking the medication everyday, along with consuming a low fat diet and regularly exercising. As a result, the patient's triglyceride level decreases to 450 mg/dL. His gout and gastritis conditions also improve as a direct result of lowering his triglycerides levels and his low fat diet. He is to maintain the dosage of a composition described herein at 50 mg twice daily for the best results.

### Example 38

### Phosphonated Pyrone Analog Decreases LDL Level and Increases HDL Level in Human

A 55-year-old Asian female has menopause, hypertension, and hyperlipidemia. She is currently taking Prampro™ hormone replacement therapy for menopause, and Atenolol™ for hypertension, which is controlled at this time. Her lipid profiles show an elevated LDL level of 180 mg/dL (normal <130), a low HDL level of 28 mg/dL (normal >40), a normal triglyceride level of 170 mg/dL (normal <160), and a cholesterol level of 210 mg/dL (normal <or =200). Since the patient does not like to take medication, her doctor agrees to wait six to twelve months to monitor her lipid profiles without the lipid-lowering medication, counting on the hormone replacement therapy and a low fat diet to help reduce the LDL cholesterol level. However, after one year, the LDL and HDL levels are not adequately reduced. Her doctor decides to start administering a composition described herein at a dose of 10 mg daily for 6 months. Subsequently, the LDL level decreased to 130 mg/dL and the HDL level increased to 60 mg/dL. Even though the patient's lipid profile improved to normal range, it is recommended that she continues to take a composition described herein, for example a tablet containing 10 mg of a phosphonated pyrone analog daily, to prevent future accumulation of LDL, which causes cholesterol plague in coronary vessels. Also, she is recommended to take 81 mg of aspirin daily to prevent stroke and heart disease. Examples of a phosphonated pyrone analog include quercetin-3-O-methylphosphonate (compound 1), quercetin-3′-O-methylphosphonate (compound 2), fisetin-3-O-methylphosphonate (compound 3), fisetin-3′-O-methylphosphonate (compound 4), quercetin-4′-O-methylphosphonate (compound 5), and fisetin-4′-O-methylphosphonate (comopund 6).

### Example 39

### Phosphonated Pyrone Analog in Combination with Other Drugs Prevent Myocardial Infarction in Diabetic Patient

A 34-year-old Hispanic female with diabetes mellitus type 2 has high cholesterol levels and high LDL levels. During an office visit, she experiences a silent heart attack without congestive heart failure. She is then admitted to the hospital for further cardiac evaluation and subsequently discharged after three days. She is currently taking Glucotrol™ XL 5 mg daily, Glucophage™ 500 mg twice a day (diabetes medications), Tenormin™ 25 mg/day, Zestril™ 10 mg/day (to prevent chest pain, and high blood pressure), and aspirin 81 mg/day. She is also taking a composition described herein at the dosage of 10 mg-20 mg of a phosphonated pyrone analog daily to prevent a second myocardial infarction in the future. Examples of a phosphonated pyrone analog include quercetin-3-O-methylphosphonate (compound 1), quercetin-3′-O-methylphosphonate (compound 2), fisetin-3-O-methylphosphonate (compound 3), fisetin-3′-O-methylphosphonate (compound 4), quercetin-4′-O-methylphosphonate (compound 5), and fisetin-4′-O-methylphosphonate (comopund 6).

### Example 40

### Phosphonated Pyrone Analog Treats Hypercholesterolemia in Human

A 42-year-old Asian male has strong a familial hypercholesterolemia. Hypercholesterolemia is a condition in which cholesterol is overly produced by the liver for unknown reasons. Furthermore, hypercholesterolemia is a strong risk factor for myocardial infarction (MI), diabetes, obesity, and other illnesses. The patient is not overweight, but is very thin. He has a very high level of cholesterol, over 300 mg/dL, and a triglyceride level of over 600 mg/dL. His diet consists of very low fat, high protein foods, and no alcohol. He has a very active lifestyle, but one which is not stressful. However, he still has to take medication to lower his cholesterol and triglyceride levels. The medications he takes include a composition described herein. He is advised to continue taking a composition described herein, for example a tablet containing 40 mg of a phosphonated pyrone analog, daily for the remainder of his life in order to control his unusual familial hypercholesterolemia condition. Examples of a phosphonated pyrone analog include quercetin-3-O-methylphosphonate (compound 1), quercetin-3′-O-methylphosphonate (compound 2), fisetin-3-O-methylphosphonate (compound 3), fisetin-3′-O-methylphosphonate (compound 4), quercetin-4′-O-methylphosphonate (compound 5), and fisetin-4′-O-methylphosphonate (compound 6).

### Example 41

### Phosphonated Pyrone Analog Decreases Triglyceride Level in Human

A 22-year-old male patient presents with triglyceride level of 250 mg/dL. The patient is given oral tablets containing about 20 mg to about 100 mg of a phosphonated pyrone analog. Examples of a phosphonated pyrone analog include quercetin-3-O-methylphosphonate (compound 1), quercetin-3′-O-methylphosphonate (compound 2), fisetin-3-O-methylphosphonate (compound 3), fisetin-3′-O-methylphosphonate (compound 4), quercetin-4′-O-methylphosphonate (compound 5), and fisetin-4′-O-methylphosphonate (comopund 6). The patient's level of triglyceride is measured 24 hours after ingesting the tablets. The measurement shows a decrease of about 20% to 50% of triglycerides as compared to the initial level.

### Example 42

### Phosphonated Pyrone Analog Decreases Blood Glucose Level in Human

A 46-year-old African American female with diabetes mellitus type 2 has hyperglycemia with a blood glucose level of 20 mmol/L, i.e. approximately 360 mg/dL. She is taking tablets described herein at the dosage of 10 mg-20 mg of a phosphonated pyrone analog once daily. Examples of a phosphonated pyrone analog include quercetin-3-O-methylphosphonate (compound 1), quercetin-3′-O-methylphosphonate (compound 2), fisetin-3-O-methylphosphonate (compound 3), fisetin-3′-O-methylphosphonate (compound 4), quercetin-4′-O-methylphosphonate (compound 5), and fisetin-4′-O-methylphosphonate (compound 6). The patient's level of blood glucose is measured 24 hours after ingesting the tablets. The measurement shows that the patient's blood glucose level returns to 6 mmol/L (i.e. 108 mg/dL) after fasting, which is within the normal range of about 80 to 120 mg/dL or 4 to 7 mmol/L.

### Example 43

### Phosphonated Pyrone Analogs Protect Against Onset of Type 2 Diabetes and Attendant Complications in Diabetic Rat Model

Animals: The Zucker Diabetic Fatty (ZDF) rat is a model for Type 2 diabetes based on impaired glucose tolerance caused by the inherited obesity gene mutation that leads to insulin resistance and subsequently diabetes. When fed with Purina 5008 chow, plasma insulin of male ZDF rats increase steadly between 7 and 10 weeks of age, and subsequently drop as pancreatic islets fail to respond to glucose. By 12 weeks of age, a male ZDF rat on a diet consisting of Purina 5008 chow is fully diabetic.

Seven week old male ZDF rats are used for the study. General procedures for animal care and housing are in accordance with the National Research Council (NRC) Guide for the Care and Use of Laboratory Animals (1996) and the Animal Welfare Standards incorporated in 9 CFR Part 3, 1991.

Experimental Design: This study is a 4 week study Using 32 7-week old male ZDF rats, divided into 4 treatment arms (8 rats/arm). The rats are kept on a diet of Purina 5008 to induce the onset of hyperglycemia, hyper insulinmia, and diabetes. The animals are treated intraperitoneally (i.p.) daily with the following compounds unless specified in the following table otherwise:

| Group | Treatment (I.P.) |
|---|---|
| 1 | Vehicle-saline |
| 2 | Rosiglitazone 6 mg/kg |
| 3 | compound 2 100 mg/kg |
| 4 | compound 3 100 mg/kg for day 4 and 5 |
| | and 50 mg/kg for day 6-28 |

Blood is collected from the rats at day 0, 7, 14, 21 and 28, and assayed for levels of plasma glucose, insulin, and triglycerides. Body weight is also measured on the same days. Animals are sacrificed at the end of the 4 week study after the OGTT study to obtain pancreas, liver, and heart puncture blood.

### Results:

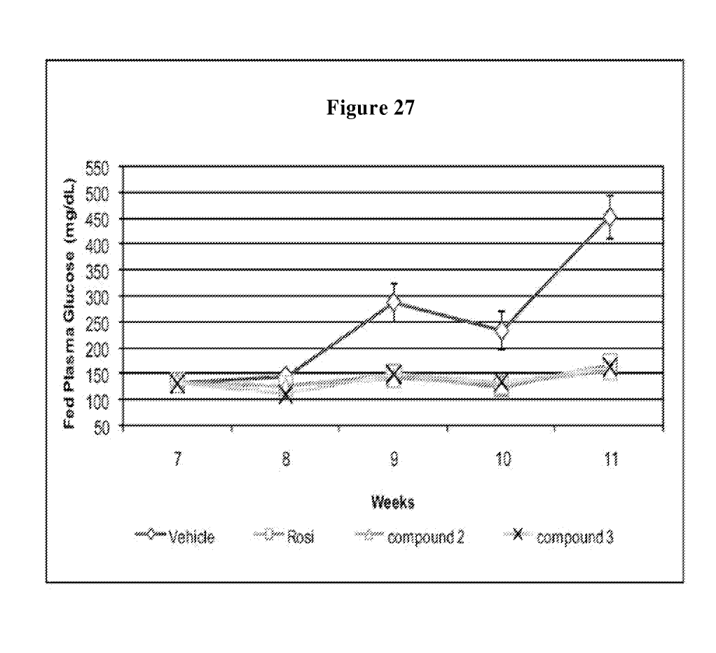

Plasma glucose levels: The plasma glucose levels show that both compound 2 and compound 3 treatment maintains steady glucose levels similar to rosiglitazone treatment while vehicle treatments cause increase in blood glucose levels. These stable glucose levels indicate that both compound 2 and compound 3 protect against hyperglycemia. See FIG. 27.

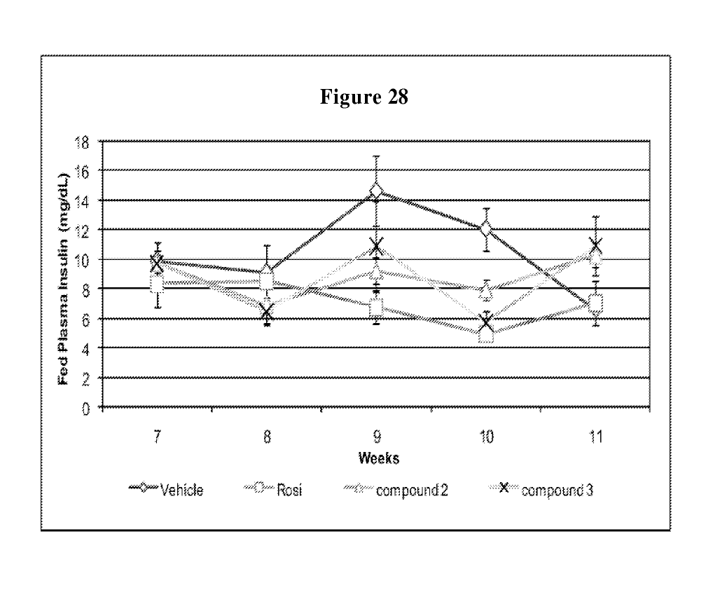

Insulin levels: The plasma insulin levels show that compound 2 or compound 3 treated animals require lower insulin levels to maintain nomal glucose levels compared to vehicle treated animals. See FIG. 28. As expected, by week-11 the vehicle treated animals are fully diabetic with very high plasma glucose (FIG. 27) and very low plasma insulin (FIG. 28).

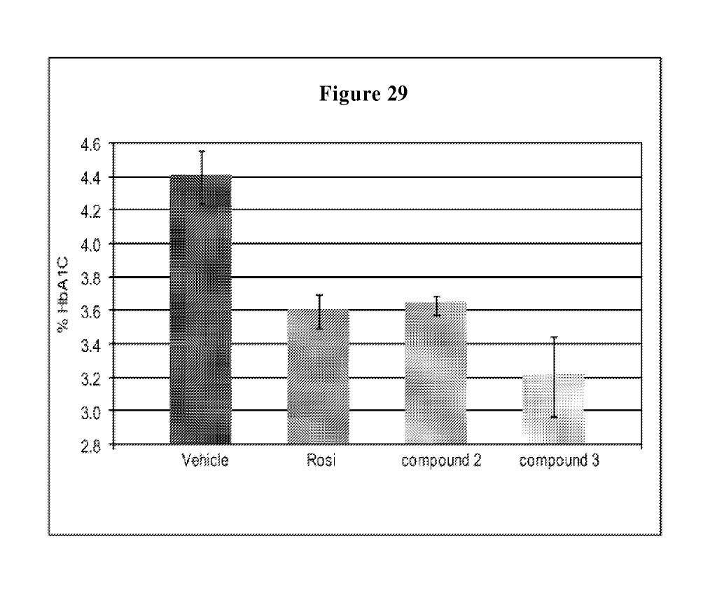

HbA1c levels: Similar to rosiglitazone, both compound 2 and compound 3 treated animals show lower glycated hemoglobin levels (% HbA1c) at termination compared to vehicle treated animals. See FIG. 29.

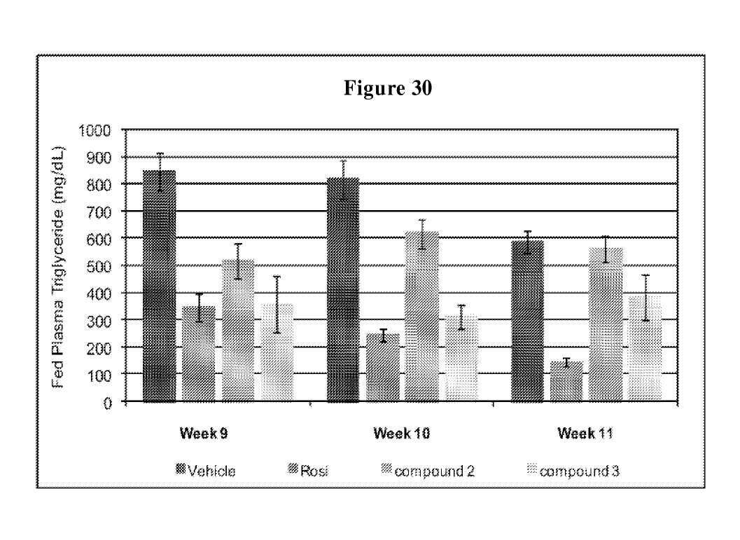

Triglyceride levels: Rosiglitazone, compound 2 and compound 3 cause marked decreases in triglycerides (mg/dL) over vehicle controls, as demonstrated by triglyceride levels at day 14 and day 21. See FIG. 30.

### Example 44

### Phosphonated Pyrone Analogs Protect Against Hyperglycemia During Oral Glucose Tolerance Test (OGTT)

Following the 4 week study described in Example 26 above, the animals were fasted for 12 hours and on day-29, pre-drug blood samples are collected and glucose level and insulin level are measured. The animals are treated intraperitoneally (i.p.) with the following compounds:

| Group | Treatment (I.P.) |
|---|---|
| 1 | Vehicle-saline |
| 2 | Rosiglitazone 6 mg/kg |
| 3 | compound 2 100 mg/kg |
| 4 | compound 3 50 mg/kg |

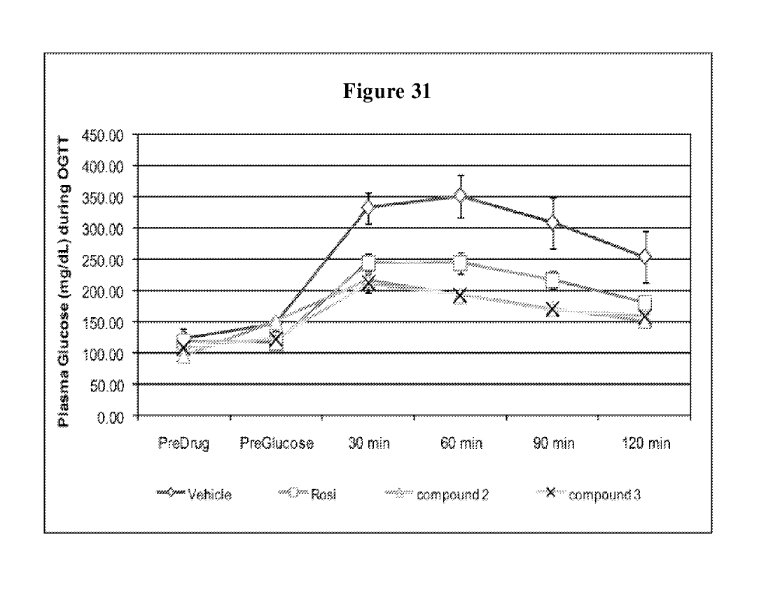

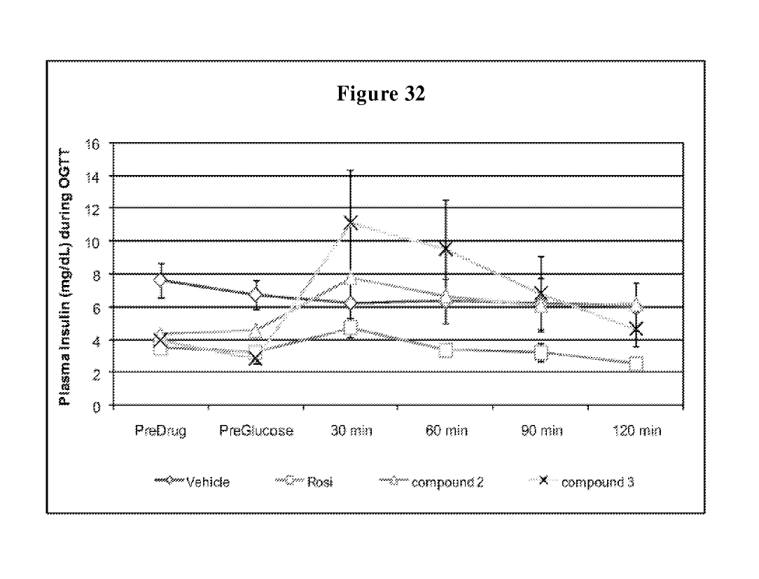

Thirty (30) minutes after the treatment, pre-glucose blood samples are collected and the animals are then dosed with glucose (2 g/Kg, PO). Tail vein blood samples are collected at 30, 60, 90 and 120 minutes. Glucose levela and insulin leveal are measured. FIG. 31 show the plasma glucose level measured during OGTT. Phosphonated pyrone analogs protect against hyperglycemia during OGTT. FIG. 32 show the plasma insulin level measured during OGTT. Phosphonated pyrone analogs raise insulin output in response to OGTT.

### Example 45

### The Histopathological Effects of Phosphonated Pyrone Analogs on Insulitis and Beta Cell Mass in Diabetic Rat Model

### Experimental Design:

Pancreas samples from Example 26 are stored in 10% NBF and used in this study. All tissue samples were processed through graded alcohols, cleared in xylene and infiltrated and embedded into paraffin. For the pancreas four 4 micron sections were cut, one was stained with Hematoxylin and Eosin for histopathology, two were immunohistochemically stained with an antibody to insulin to determine beta cell mass and one was stained for apoptosis with the tunel technique.

The insulin staining procedure was performed as follows: Endogenous peroxidase activity was inhibited by incubation in 3% hydrogen peroxide. Non-specific staining was blocked with DAKO Protein Block Serum-Free (Dako, Carpinteria, Calif.). Insulin positive cells were immunohistochemically stained with a polyclonal guinea pig anti-swine insulin antibody at room temperature for 30 minutes (Dako, Carpinteria, Calif.). After incubation with primary antibody, the tissue sections were sequentially incubated with Envision+ HRP Rabbit (Dako, Carpinteria, Calif.). Staining was developed with Liquid DAB+ (Dako, Carpinteria, Calif.) and counterstained with hematoxylin.

Pancreas samples were stained for apoptosis as follows: 4 micron sections were cut and stained for TUNEL utilizing the following kit In Situ Cell Death Detection Kit, AP (Roche Applied Science, catalog 11 684 809 910).

### Results:

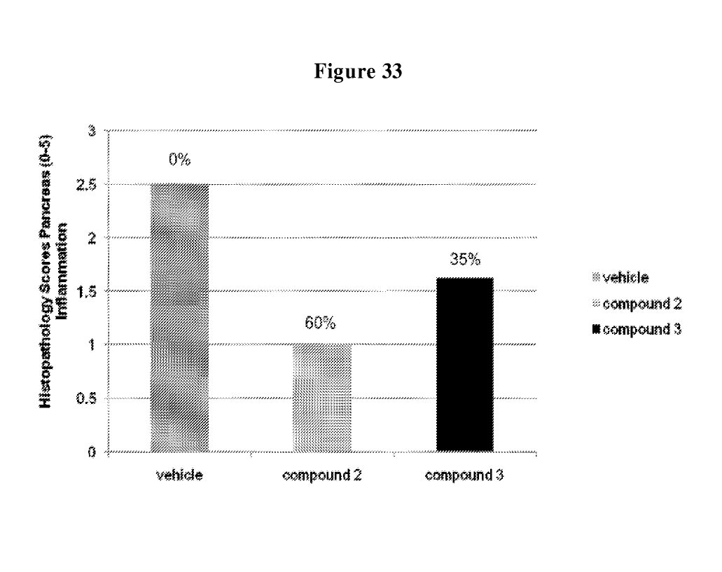

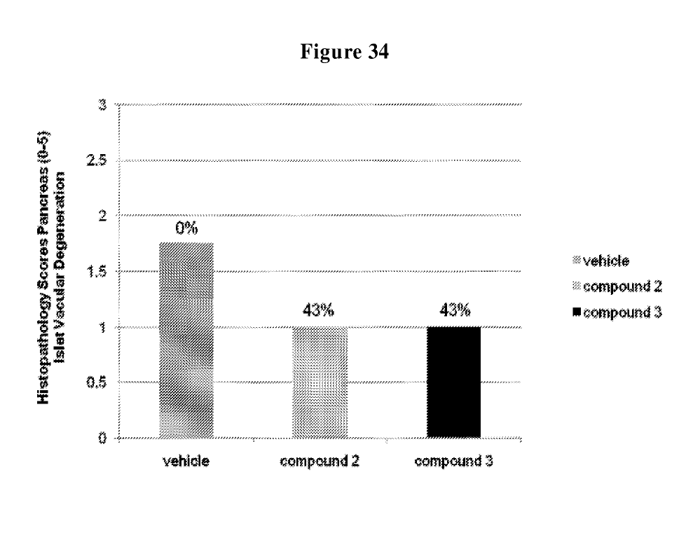

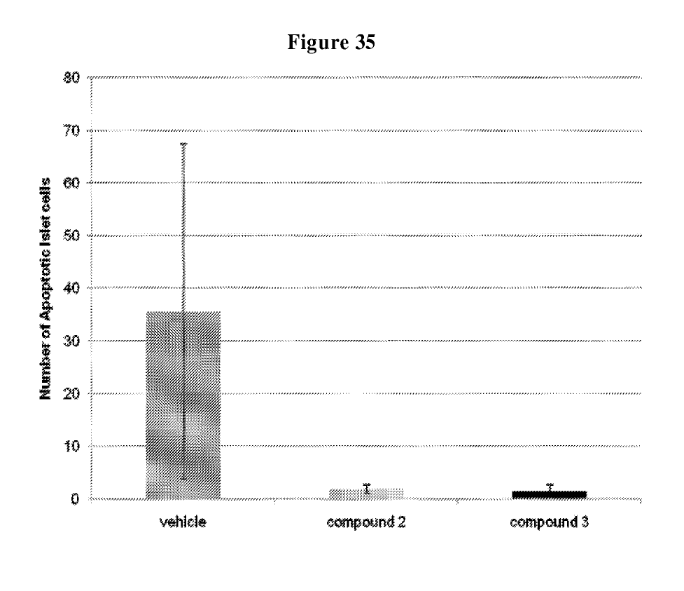

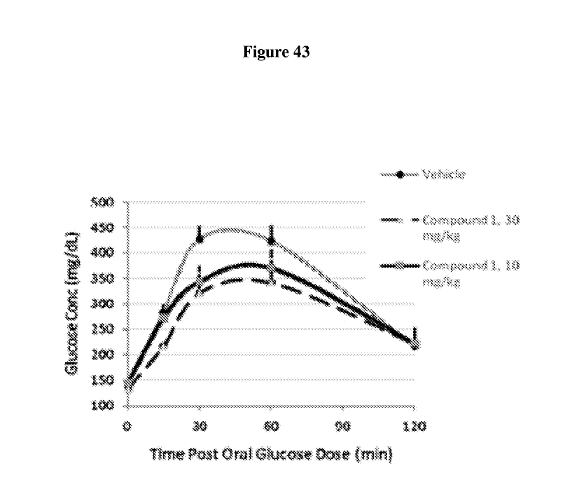

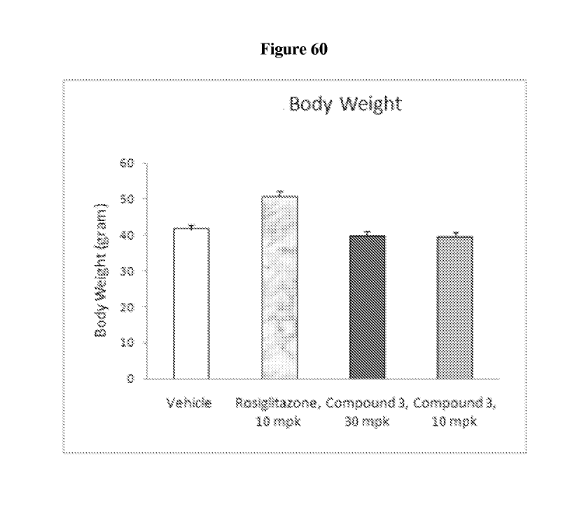

Treatment with compound 2 and compound 3 result in significant decreases in pancreatic inflammation (see FIGS. 33, 60% and 35% inhibitions, respectively). Treatment with compound 2 and compound 3 result in significant decreases in islet vacuolar degeneration (see FIGS. 34, 43% and 43% inhibitions, respectively). Treatment with compound 2 and compound 3 result in decreases in islet cell apoptosis (see FIG. 35). The ratio of insulin positive islet cells to pancreas cells is significantly increased in the animals treated with compound 2.

### Example 46

### Effect of Phosphonated Pyrone Analogs on Diet Induced Obese Mice

### Animals and Experimental Design:

Twenty-three (23) week old C57Bl/6 mice are placed on 60% high fat diet (HFD). After 9 weeks of HFD, mice are injected daily with either vehicle (PBS/NaCO₃) or fisetin-3-O-methylphosphonate (compound 3) at a dosage of 10 mg/kg delivered via I.P. injection. HFD is continued throughout the injection period. After 2 weeks (week 10 and 11) of injection, mice are tested for basal blood (non-fasted) glucose levels by tail blood withdrawal. After 3 weeks (week 12) of injection, IP glucose tolerance test (IP-GTT) is performed at a dose of 1 gm dextrose per kilogram body weight. After 4 weeks (week 13) of injection, cannulation surgery is performed on anesthetized mice. Mice are allowed to recover 6 days. After 6 days, hyperinsulinemic euglycemic clamp studies are performed on the mice. After 7 weeks (week 16) of injections, a second IP-GTT is performed on the mice. Three days after the second IP-GTT, mice are sacrificed and blood and tissue samples are collected for further analysis.

### Results:

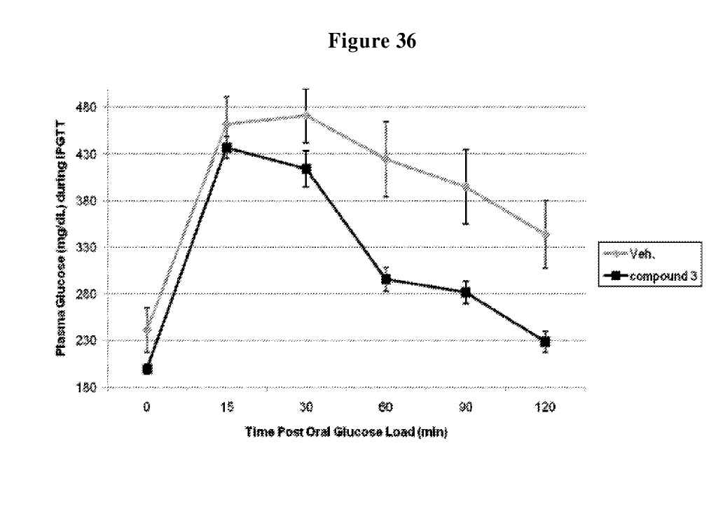

FIG. 36 demonstrates that phosphonated pyrone analog compound 3 improves response to glucose challenge in diet-induced obese mice.

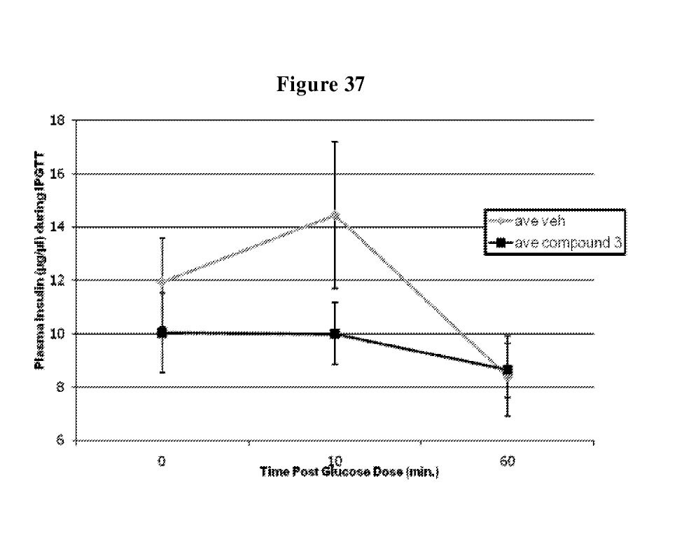

FIG. 37 demonstrates that less insulin output is required in response to glucose challenge in compound 3 treated diet-induced obese mice, indicating reduced insulin resistance compared to vehicle treated obese mice.

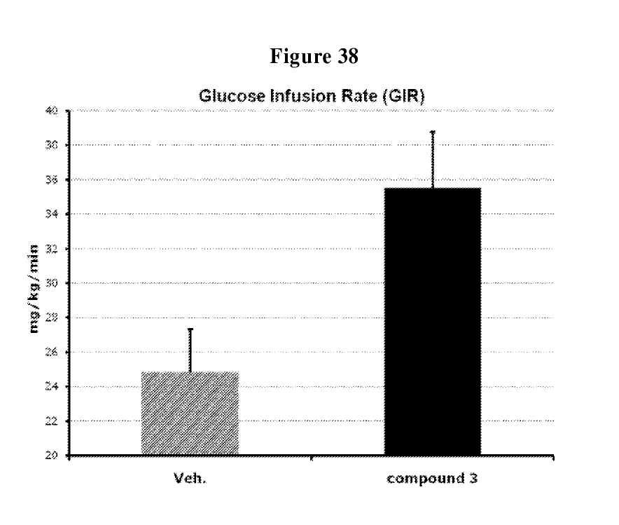

FIG. 38 demonstrates that phosphonated pyrone analog compound 3 enhances tissue glucose uptake during hyperinsulinemic euglycemic clamp study in diet-induced obese mice.

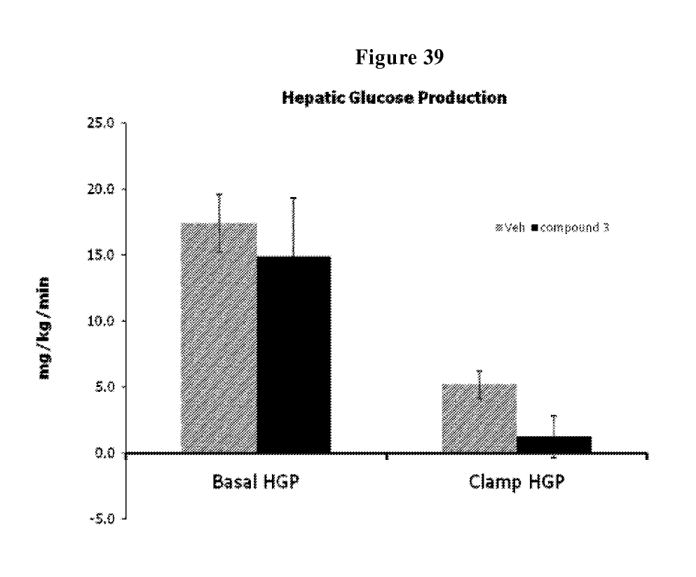

FIG. 39 demonstrates that phosphonated pyrone analog compound 3 impacts hepatic glucose output in diet-induced obese mice.

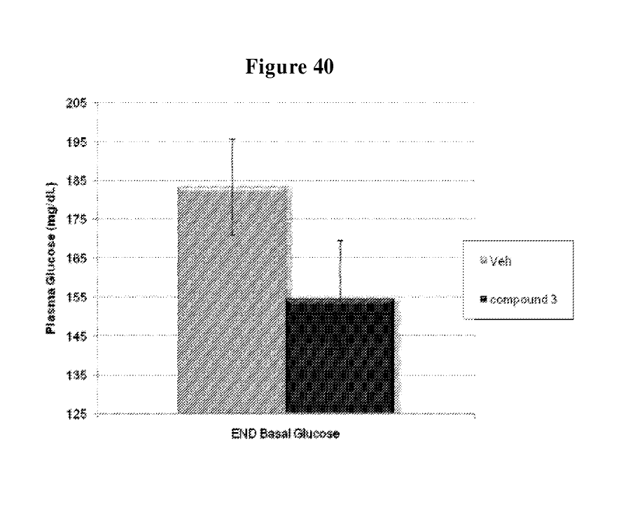

FIG. 40 demonstrates that phosphonated pyrone analog compound 3 reduces basal glucose in diet-induced obese mice.

### Example 47

### Stability of quercetin-3′-O-methylphosphonate in Water

Quercetin-3′-O-methylphosphonate (Compound 2) is dissolved in water at about pH 8. After 24 hours in water at pH 8, no degradation is seen by NMR after 24 hours at ambient temperature.

### Example 48

### Blood Glucose Levels in Rats Co-Administered with Quercetin-3′-O-methylphosphonate and Tacrolimus

One set of 5 rats is treated from day 1 to day 25 with inert vehicle 2 intraperitoneally and treated from day 11 to day 25 with inert vehicle 1 intraperitoneally. A second set of 5 rats is treated from day 1 to day 25 with tacrolimus (Prograf®) at 0.5 mg/kg, and treated from day 11 to day 25 with inert vehicle 2. A third set 5 of rats is treated from day 1 to day 25 with tacrolimus (Prograf®) intraperitoneally at 0.5 mg/kg, and treated from day 11 to day 25 intraperitoneally with quercetin-3′-O-methylphosphonate (Compound 2) at 114 mg/kg. The blood glucose level in the rats is measured on days 1, 10, 15, 20, and 25. The results show that phosphonated pyrone analogs such as Compound 2 can attenuate tacrolimus induced hyperglycemia.

### Example 49

### Renal Pathology in Rats Co-Administered with Quercetin-3′-O-methylphosphonate and Tacrolimus

Tissue is removed from the kidney of rats treated with tacrolimus (Prograf®) at 0.5 mg/kg and inert vehicle, and from rats treated with tacrolimus (Prograf®) at 0.5 mg/kg and quercetin-3′-O-methylphosphonate (Compound 2) at 11 mg/kg, 28 mg/kg, and at 114 mg/kg for 25 days. The tissue from rats treated with tacrolimus and vehicle show significant vacuolation. The tissue from rats treated with Compound 2 and tacrolimus show no vacuoles. The results indicate that Compound 2 exerts a significant protective effect with respect to the kidneys when co-administered with tacrolimus.

### Example 50

### In-Vitro Toxicity Screening of Quercetin-3′-O-methylphosphonate

A secondary pharmacological screening of molecules of interest at a fixed concentration is often practiced in the pharmaceutical industry in order to evaluate the effect of the compound on secondary targets that could result in untoward toxicity in-vivo. These secondary screens are well known in the art and can be carried out by labs which specialize in these tests such as MDS-Panlabs and CEREP. A secondary toxicity screen can be performed with Quercetin-3′-O-methylphosphonate (Compound 2) at a concentration of 10 μM against 122 targets in enzyme, radioligand binding, and cellular assays by MDS Pharma Services by methods well known in the art. Inhibition can be found in the following targets (percent inhibition at 10 μM in parentheses): ATPase, Na+/K+, Heart, Pig (65%), Nitric Oxide Synthase, Endothelial (eNOS) (72%), Protein Tyrosine Kinase, FGFR2 (94%), Protein Tyrosine Kinase, FGFR1 (96%), Protein Tyrosine Kinase, Insulin Receptor (91%), Protein Tyrosine Kinase, (82%), Protein Tyrosine Kinase, ZA70 (ZAP-70) (74%), UDP Glucuronosyltransferase, UGT1A1 (52%), Adenosine A₁ (50%), Adrenergic α_{2A} (57%), Dopamine D₄₇ (51%), Peripheral Benzodiazepine Receptor (PBR) (53%), Transporter, Monoamine rabbit (68%), and Serotonin (5-Hydroxytryptamine) 5-HT_{1A} (62%).

The compound can be additionally tested in Adenosine_{A1}, Adrenergic_{A2A}, DopamineD₂₅, Histamine H₁-, and μ-Opiate GTPγS functional assays using a concentration of 10 μM. The compound which demonstrates 48% antagonist activity in the Adenosine_{A1} assay, and marked negative inhibition in the Adrenergic_{A2A} assay, potentially indicating PAF-5 can be acting as an inverse agonist in this assay.

The findings of this toxicology screen can be an indication that Quercetin-3′-O-methylphosphonate has low toxicity properties, especially in light of the fact that the concentration tested, 10 μM, is high as compared to a therapeutic dose (e.g. greater than ˜100 times).

All of the methods disclosed and claimed herein can be made and executed without undue experimentation in light of the present disclosure. It will be apparent to those of skill in the art that variations may be applied without departing from the concept, spirit and scope of the invention. More specifically, it will be apparent that certain agents that both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 51

### Phosphonated Pyrone Analog Compound 1 Protects Against Onset of Type 2 Diabetes and Attendant Complications in Diabetic Rat Model

Experimental Design: This study is a 4-week study conducted with male ZDF rats (24 animals, 4 treatment arms, 6 rats/arm) 6-week old at the start of compound treatment. The animals are maintained (starting at 5-week old) on a 12-hr light and 12-hr dark cycle, and provided with commercial rodent chow Purina 5008 and water ad libitum. The animals are treated intraperitoneally (i.p.) daily (at 8 hr after start of light cycle) with the following compounds:

| Group | Treatment (I.P.) |
|---|---|
| 1 | Vehicle-saline |
| 2 | Rosiglitazone 6 mg/kg |
| 3 | compound 1 30 mg/kg |
| 4 | compound 1 10 mg/kg |

Predose tail-vein-blood samples are collected from each rat at day 0, 7, 14, 21 and 28, and plasma glucose concentrations are determined. Body weights are also measured on the same days. Insulin and glycated hemoglobin (HbA1c) concentrations are also determined on day-28 blood. Oral glucose (2 g/kg, 5 ml/kg) tolerance test (OGTT) including fasting insulin are performed on all animals on day-29 after 12-hr fast. Animals are sacrificed after OGTT to obtain pancreas for insulin and liver for triglyceride determination.

### Results:

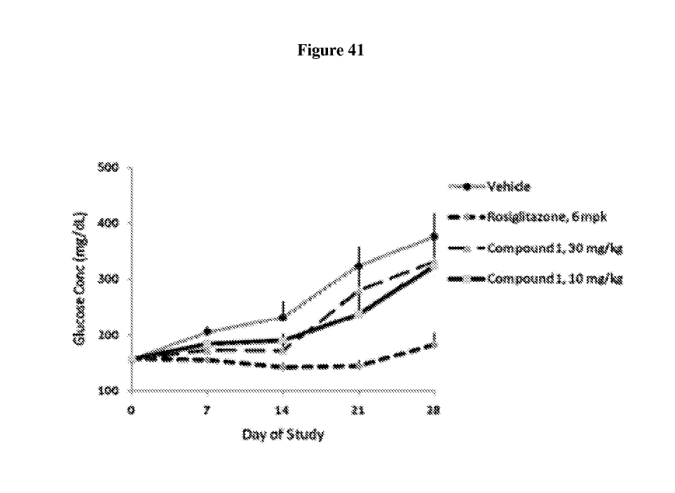

Plasma glucose levels: The plasma glucose levels show that compound 1 treatment maintains a lower plasma glucose level than vehicle treatment. Compound 1 treatment, however, did not stabilize plasma glucose levels as rosiglitazone treatment did (FIG. 41). Compound 1 appeas to slow down the development of diabetes and have a different mode of action from that of rosiglitazone.

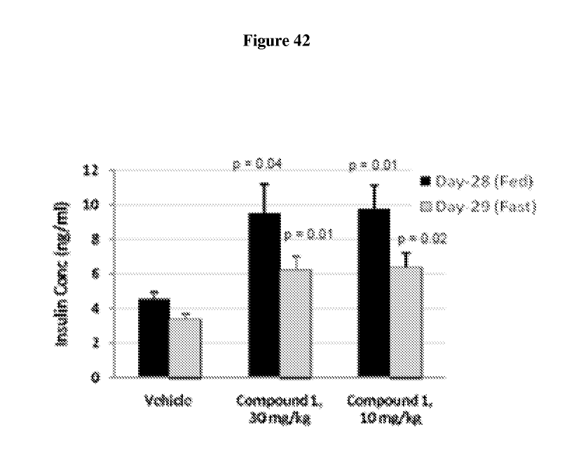

Insulin levels: Plasma insulin measurements (FIG. 42) showed that compound 1 treated animals maintain significantly higher plasma insulin levels under both fed and fasting conditions. Untreated male ZDF rats under experimental conditions described above develop diabetes between the age of 8 and 10 weeks old (day-14 to day-28 of this example). As shown in FIG. 102, vehicle treated animals have low insulin levels corresponding to the full diabetic state. The significantly higher insulin levels of compound 1 treated animals provides further evidence that compound 1 treatment slows down the development of diabetes in male ZDF rats.

OGTT: Plasma glucose concentrations at 15, 30, 60, and 90 minutes post oral glucose challenge (FIG. 43) showed that compound 1 treated animals are able to maintain lower plasma glucose level during OGTT. Thus, consistent with slowing down diabetes development, compound 1 treatment improves oral glucose tolerance of aging male ZDF rats.

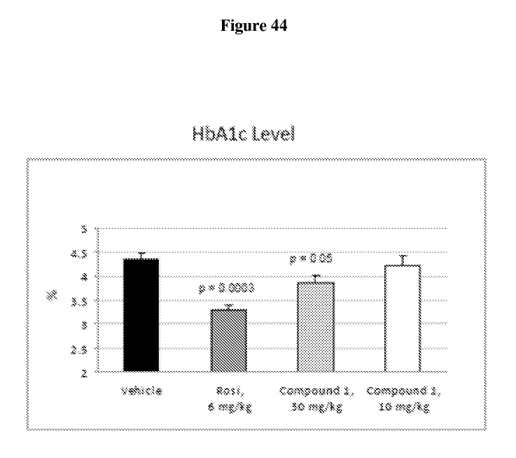

HbA1c levels: Similar to rosiglitazone, 30 mg/kg compound 1 treated animals show significantly lower glycated hemoglobin levels (% HbA1c) at termination compared to vehicle treated animals (FIG. 44)

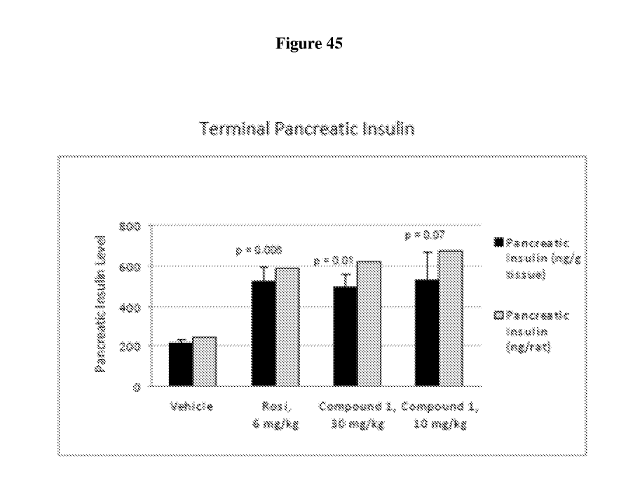

Pancreatic Insulin: Similar to rosiglitazone treated animals, the pancreatic insulin levels of 30 mg/kg compound 1 treated animals are significantly higher than that of the vehicle treated animals (FIG. 45). This is consistent with compound 1 treatment slows down the development of diabetes.

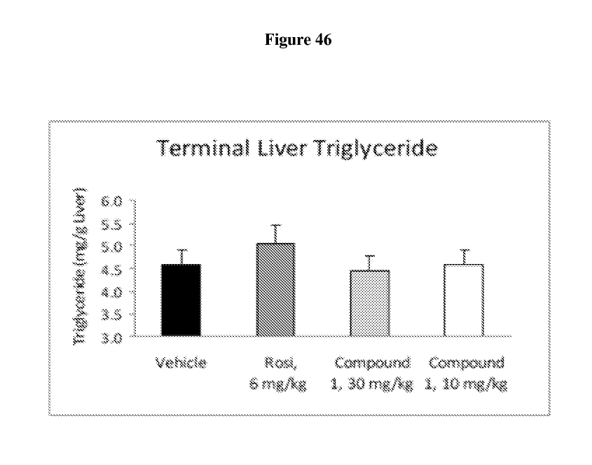

Liver Triglyceride: In contrast of rosiglitazone treatment, compound 1 treated animals did not increase liver triglyceride (FIG. 46).

### Example 52

### Compound 1 Protects Against Cyclosporine Induced Islet Injury in Rat

Experimental Design: This study is a 2-week study conducted with male Wistar rats (25 animals, 5 treatment groups, 5 rats/group) 10.5-week old at the start of compound treatment. The animals are maintained (starting at 9-week old) on a 12-hr light and 12-hr dark cycle, and provided with commercial rodent chow Purina 5008 and water ad libitum. The animals are treated daily intraperitoneally (IP) with Treatment-1 (at 6 hr, 45 minutes after the start of light cycle) followed by oral dose (PO) of Treatment-2 (at 7 hr after the start of the light cycle) as shown in the following table. Neoral® oral solution (containing 100 mg/ml of cyclosporine is used to prepare the cyclosporine dose.

| Group | Treatment-1 (IP) | Treatment-2 (PO) |
|---|---|---|
| 1 | Vehicle #1 (saline) | Vehicle #2 (skim milk) |
| 2 | Vehicle #1 (saline) | Cyclosporine, 10 mg/kg |
| 3 | Compound 1, 30 mg/kg | Cyclosporine, 10 mg/kg |
| 4 | Compound 1, 10 mg/kg | Cyclosporine, 10 mg/kg |
| 5 | Compound 1, 3 mg/kg | Cyclosporine, 10 mg/kg |

Predose tail-vein-blood samples are collected from each rat at day 0, 7, and 14, and plasma glucose concentrations are determined. Body weights are also measured on the same days. Oral glucose (2 g/kg, 5 ml/kg) tolerance test (OGTT) are performed on all animals on day-15 after 12-hr fast. Animals are sacrificed after OGTT to obtain pancreas for histopathological evaluation.

### Results:

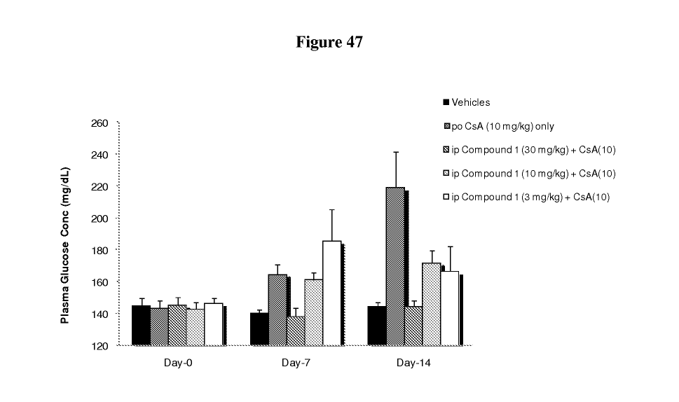

Plasma glucose levels: Cyclosporine induced hyperglycemia is clearly demonstrated by day-14 of cyclosporine (Group-2) treated rats (FIG. 47). Co-administration of compound 1 at all three dose levels reduced cyclosporine induced hyperglycemia. At 30 mg/kg, compound 1 completely prevented cyclosporine induced hyperglycemia.

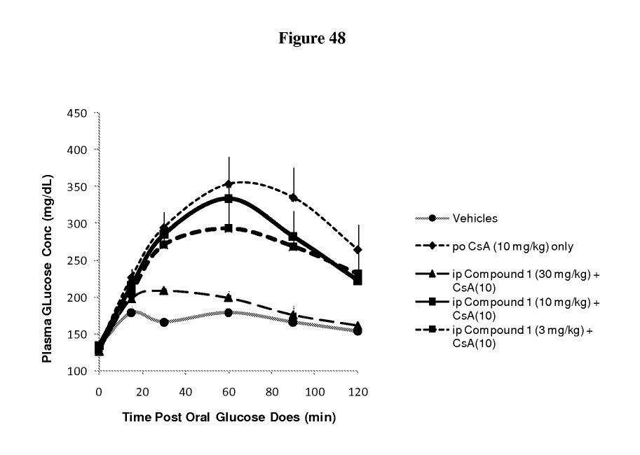

OGTT: Plasma glucose concentrations at 0, 15, 30, 60, 90, and 120 minutes post oral glucose challenge (FIG. 48) showed that 30 mg/kg compound 1 treated animals are able to maintain plasma glucose level during OGTT at a level similar to that of vehicle treated animals (Group-1). Thus, co-administration of compound 1 prevented cyclosporine induced glucose intolerance.

### Example 53

### Compound 1 Protects Against Cyclosporine Induced Islet Apoptosis

### Experimental Method:

Pancreases from animals in Groups 1-3 in Example-52 above are fixed in 10% neutral buffered formalin, processed and stained with hematoxylin and eosin for histopathology, and with the tunnel method for apoptosis. Islet cell hyperplasia and degeneration/necrosis are evaluated using the following scoring system (one slide of each animal is scored): 0=no significant lesions; 1=minimal lesions; 2=mild lesions; 3=moderate lesions; and 4=marked lesions. Number of apoptotic cells stained by the Tunnel method in one entire slide from each animal is manually counted

### Results:

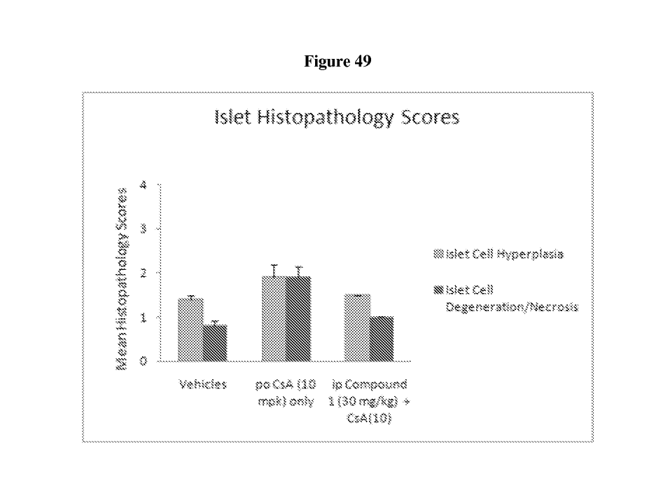

Islet Cell Histopathology: As shown in FIG. 49, pancreatic islets from cyclosporine treated animals (Group-2) showed increased hyperplasia and degeneration/necrosis compared to vehicle treated animals (Group-1), while co-administration with compound 1 (Group-3) normalized the scores to that of vehicle treated animals. Thus, Compound 1 treatment prevented cyclosporine induced islet injury.

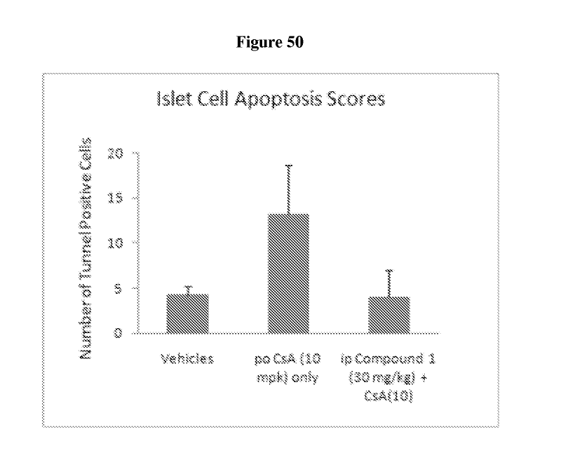

Islet Apoptosis: As shown in FIG. 50, pancreatic islets from cyclosporine treated animals (Group-2) showed increased apoptosis compared to vehicle treated animals (Group-1), while co-administration with compound 1 (Group-3) reduced the number of apoptotic cells to that of vehicle treated animals. Thus, Compound 1 treatment prevented cyclosporine induced islet cell apoptosis.

### Example 54

### Compound 1 Protects Against Tacrolimus Induced Hyperglycemia in Rat

### Experimental Design:

This study is a 2-week study conducted with male Wistar rats (25 animals, 5 treatment groups, 5 rats/group) 11-week old at the start of compound treatment. The animals are maintained (starting at 9-week old) on a 12-hr light and 12-hr dark cycle, and provided with commercial rodent chow Purina 5008 and water ad libitum. The animals are treated daily orally (PO) with Treatment-1 (at 7 hr after the start of light cycle) followed immediately with intraperitoneal dose (IP) of Treatment-2 as shown in the following table. Stock solution (5 mg/ml) of tacrolimus is prepared in HCO-60 in 80% alcohol. Dose solution of tacrolimus is prepared from the stock solution by 1:10 dilution with skim milk.

| Group | Treatment-1 (PO) | Treatment-2 (IP) |
|---|---|---|
| 1 | Vehicle #1 (HCO-60 + skim milk) | Vehicle #2 (saline) |
| 2 | Tacrolimus, 2 mg/kg | Vehicle #2 (saline) |
| 3 | Tacrolimus, 2 mg/kg | Compound 1, 30 mg/kg |
| 4 | Tacrolimus, 2 mg/kg | Compound 1, 10 mg/kg |
| 5 | Tacrolimus, 2 mg/kg | Compound 1, 3 mg/kg |

Predose tail-vein-blood samples are collected from each rat at day 0, 7, and 14, and plasma glucose concentrations are determined. Body weights are also measured on the same days. Oral glucose (2 g/kg, 5 ml/kg) tolerance test (OGTT) are performed on all animals on day-15 after 12-hr fast. Teil vein blood samples are collected pre-glucose challenge and at 15, 30, 60, 90 and 120 min post-glucose challenge.

### Results:

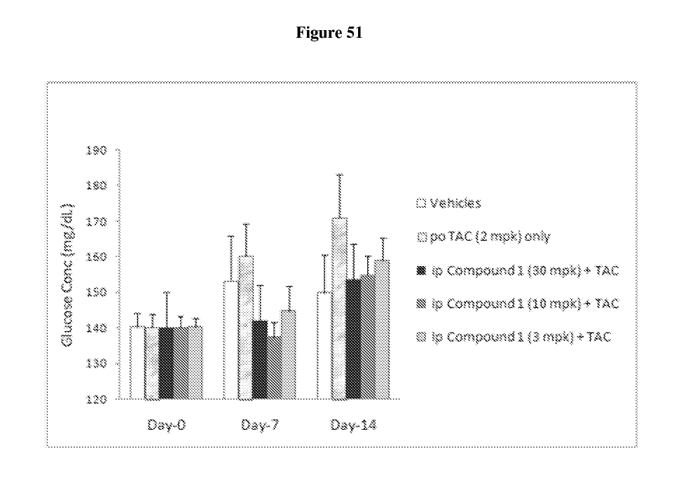

Plasma glucose levels: Tacrolimus induced hyperglycemia is clearly demonstrated by day-14 of tacrolimus (Group-2) treated rats (FIG. 51). Co-administration of compound 1 at all three dose levels reduced tacrolimus induced hyperglycemia.

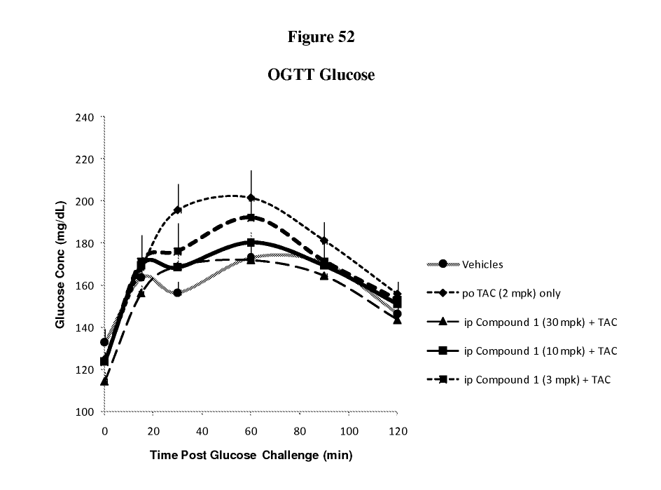

Plasma glucose level during OGTT: Plasma glucose concentrations at 0, 15, 30, 60, 90, and 120 minutes post oral glucose challenge (FIG. 52) showed that 30 mg/kg and 10 mg/kg compound 1 treated animals are able to maintain plasma glucose level during OGTT at a level similar to that of vehicle treated animals (Group-1). Thus, co-administration of compound 1 prevented tacrolimus induced glucose intolerance.

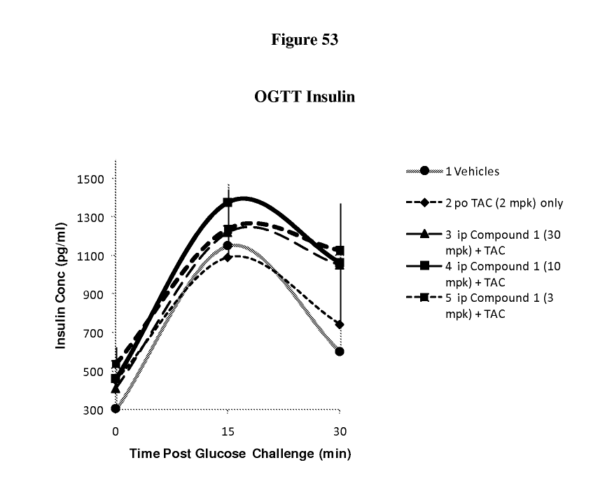

Plasma insulin level during OGTT: Plasma insulin concentrations at 0, 15 and 30 minutes post oral glucose challenge (FIG. 53) showed that the amount of insulin released by compound 1 treated animals, at all three dose levels, are higher than tacrolimus treated (group-2) or vehicle treated (Group-1) animals. Thus co-administration of compound 1 prevented tacrolimus induced glucose intolerance during glucose challenge by increasing insulin release.

### Example 55

### Tacrolimus Uptake Assay

### Cell Culture:

BB19 cells, human BCECs (brain capillary endothelial cells) immortalized with the E6E7 genes of human papilloma virus, were kindly provided from Jacques G. Prudhomme, Department of Biology, University of California, Riverside, U.S.A. The cells were cultured as monolayer culture in Amniomax™-C100 basal medium (catalog number 17001-157) with Amniomax™-C100 supplement (catalog number 12556-049) (Invitrogen, Basel, CH).

Cells were seeded onto culture surfaces precoated with 10 μg/cm² rat tail collagen type I (BD Bioscience, Allschwil, CH) at a density of approximately 50′000 cells/cm² and were cultured in an incubator at 37° C. with 5% CO₂, 95% fresh air and saturated humidity. For uptake assays, cells were seeded on precoated 96-well plates. Assays were performed at confluency, after 6 days in culture.

### Uptake Assay:

BB19 cells were seeded 50′000 cells/cm² on 96-well plates (Falcon) and incubated under condition described above for 6 days. Before starting the assay, cells were washed 2 times with pre-warmed Dulbecco's MEM with Glutamax-I (catalog number 61965) (Invitrogen AG, Basel, CH), supplemented with 1% non essential amino acids, 1% sodium pyruvate, 50 μg/ml gentamycin (Invitrogen AG) (DMEM). Cells were then incubated for 15 min at 37° C. and 120 rpm in DMEM with or without the specified concentrations of test compounds under investigation. After this pre-incubation, cells were incubated in DMEM containing [propyl-3H]-dihydro-FK506 (3H-tacrolimus, 1 μCi/ml, 34 nM) and with or without the specified concentrations of test compounds for 1 hour at 37° C. and 120 rpm. During this 1-hour incubation period, influx and efflux of the substrate, ³H-tacrolimus, occur. After the 1-hour incubation period, assay was terminated by placing the assay plate on ice and washing the cells twice with ice-cold DMEM. Determination of cell associated ³H-tacrolimus was performed by addition of Insta Gel and scintillation liquid to trypsinized cells followed by analysis on a scintillation counter (Packard TriCarb2000, Can berra Packard S.A.). The results are expressed as: % of control=100×[³H-tacrolimus conc. with test compound]/[³H-tacrolimus conc. with vehicle]

All compounds tested showed a reduction of cell associated ³H-tacrolimus uptake after 1 hour incubation when compared to the vehicle. The table below illustrates the reduction of cell associated ³H-tacrolimus uptake in the presence of 0.1 μM test compounds.

Reduction of cell associated ³H-tacrolimus uptake in the presence of 0.1 μM test compounds

| Compound Number | % of control | Compound Number | % of control |
|---|---|---|---|
| 1 | 60 | 2 | 49 |
| 1 | 51 | 18 | 66 |
| 1 | 21 | 18 | 60 |
| 1 | 48 | 20 | 62 |
| 1 | 61 | 20 | 67 |
| 1 | 60 | 4 | 58 |
| 1 | 46 | 4 | 57 |
| 2 | 51 | 3 | 33 |
| 2 | 51 | 3 | 44 |
| 2 | 62 | 10 | 51 |
| 2 | 62 | 16 | 54 |
| 2 | 49 | 11 | 49 |
| 2 | 49 | 11 | 29 |

### Example 56

### Cyclosporin Uptake Assay

The cyclosporin uptake assay was performed similarly as described above for tacrolimus uptake assay except that ³H-cyclosporin was used in place of ³H-tacrolimus.

All compounds tested showed a reduction of cell associated ³H-cyclosporin uptake after 1 hour incubation when compared to the vehicle. The table below illustrates the reduction of cell associated ³H-cyclosporin uptake in the presence of 0.1 μM test compounds.

Reduction of cell associated ³H-cyclosporin uptake in the presence of 0.1 μM test compounds

| Compound Number | % of control |
|---|---|
| 1 | 24 |
| 1 | 61 |
| 1 | 60 |
| 2 | 54 |
| 18 | 77 |
| 20 | 77 |
| 4 | 71 |
| 3 | 71 |
| 11 | 78 |

### Example 57

### Phosphonated Pyrone Analog Compound 3 Reduce Plasma and Liver Triglyceride in C57BL/6 Mice

Experimental Design: This study is a 2-week study conducted with male C57BL/6 mice (24 animals, 3 treatment arms, 8 rats/arm) 9-week old at the start of compound treatment. The animals are maintained (starting at 8-week old) on a 12-hr light and 12-hr dark cycle, and provided with commercial rodent chow Purina 5001 and water ad libitum. The animals are treated intraperitoneally (i.p.) daily (at 8 hr after start of light cycle) for 13 days with vehicle and compound 3 as shown below.

| Group | Treatment (i.p.) |
|---|---|
| 1 | Vehicle (HCO-60) |
| 2 | compound 3, 30 mg/kg |
| 3 | compound 3, 3 mg/kg |

The stock solution of vehicle (HCO-60) is composed of 200 mg of HCO-60 per mL of 80% alcohol. Vehicle is prepared by diluting the HCO-60 stock solution 1:10 with saline. Compound 3 stock solutions are prepared in HCO-60 stock solution and diluted with saline or vehicle to the appropriate dose concentration.

Tail-vein-blood samples are collected from each rat 24 hour after the last treatment (day-14) for plasma triglyceride determination. Animals are sacrificed to obtain liver for triglyceride determination.

### Results:

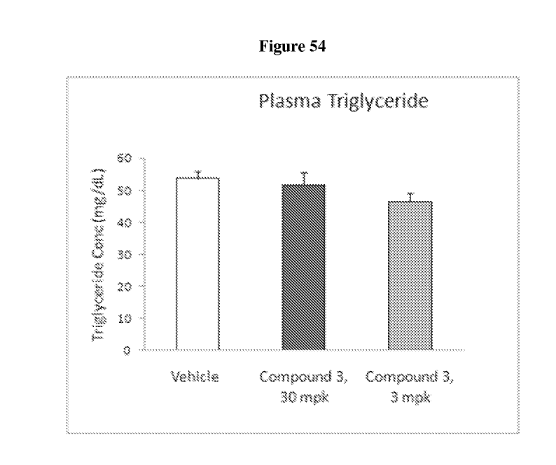

Plasma triglyceride levels: The plasma triglyceride concentrations of animals treated with vehicle or compound 3 are shown in FIG. 54. Daily treatment of compound 3 for 13 days reduces plasma triglyceride in C57BL/6 mice.

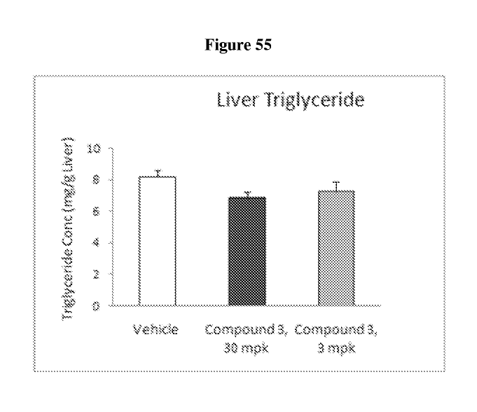

Liver triglyceride levels: The liver triglyceride concentrations of animals treated with vehicle or compound 3 are shown in FIG. 55. Compared to vehicle treated controls (Group-1), daily treatment with 30 mg/kg of compound 3 for 13 days significantly reduces liver triglyceride of C57BL/6 mice.

### Example 58

### Phosphonated Pyrone Analog Compound 3 Reduce Plasma Glucose, Plasma Triglyceride, Plasma Cholesterol and Liver Triglyceride in db/db Mice without Excessive Body Weight Gain

Experimental Design: This study is a 4-week study conducted with male db/db mice (Jackson Stock #000642, 24 animals, 4 treatment arms, 6 rats/arm) 8-week old at the start of compound treatment. The animals are maintained (starting at 7-week old) on a 12-hr light and 12-hr dark cycle, and provided with commercial rodent chow, LabDiet® 5K52, and water ad libitum. The animals are treated intraperitoneally (i.p.) daily (at 8 hr after start of light cycle) for 28 days with vehicle and compound 3 as shown below.

| Group | Treatment (i.p.) |
|---|---|
| 1 | Vehicle (Saline) |
| 2 | Rosiglitazone, 10 mg/kg |
| 3 | compound 3, 30 mg/kg |
| 4 | compound 3, 10 mg/kg |

Pre-dose tail-vein-blood samples are collected from each rat at day 0, 7, 14, 21 and 28, and plasma glucose, triglyceride and cholesterol concentrations are determined. Body weights are also measured on the same days. Animals are sacrificed on day-29 to obtain liver for triglyceride determination.

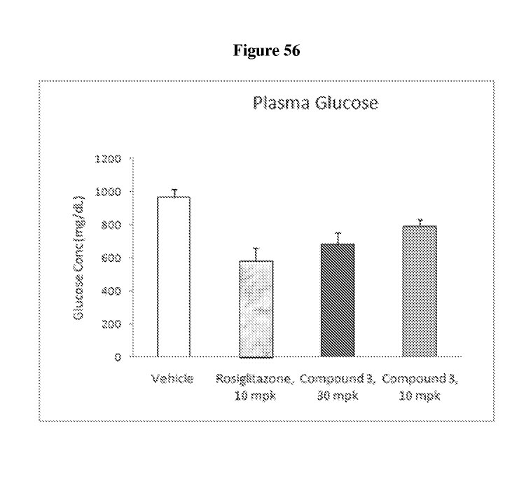

Plasma glucose levels: Plasma glucose level on Day-28 of compound 3 treated mice, similar to those treated with rosiglitazone, are lower than vehicle treated control mice (FIG. 56). Thus, compound 3 significantly reduced diabetes-associated hyperglycemia in db/db mice after 4 weeks of treatment.

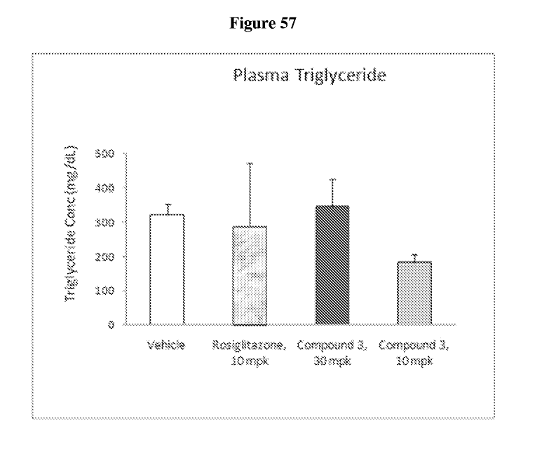

Plasma triglyceride levels: Plasma triglyceride levels on Day-28 of 10 mg/kg compound 3 treated mice are significantly lower than vehicle or rosiglitazone treated mice (FIG. 57). Thus, compound 3 reduced diabetes-associated hypertriglyceridemia in db/db mice after 4 weeks of treatment.

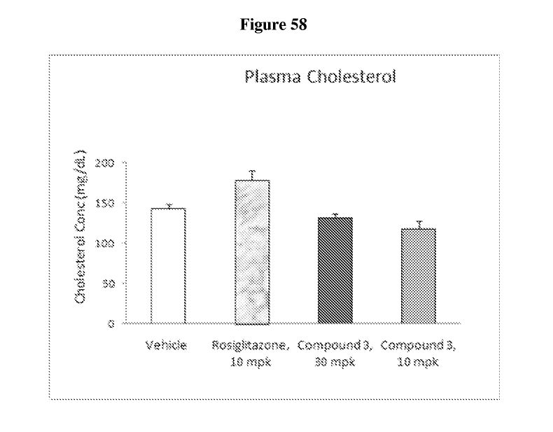

Plasma cholesterol levels: Plasma cholesterol levels on Day-28 of 10 mg/kg compound 3 treated mice are significantly lower than vehicle or rosiglitazone treated mice (FIG. 58). Thus, compound 3 reduced diabetes-associated hypercholesterolemia in db/db mice after 4 weeks of treatment.

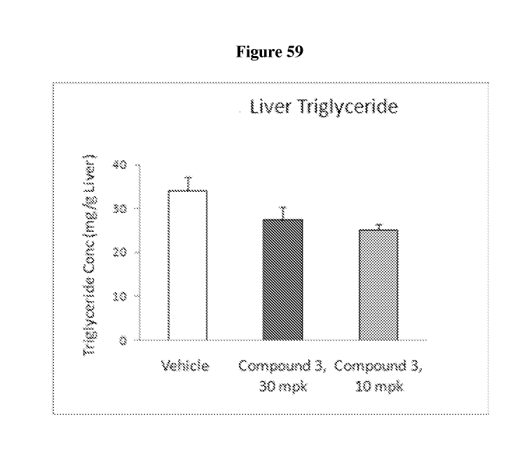

Liver Triglyceride: Liver triglyceride at sacrifice (day-29) of 10 mg/kg compound 3 treated mice are significantly lower than vehicle treated mice (FIG. 59). Thus, compound 3 reduced diabetes-associated fatty liver in db/db mice after 4 weeks of treatment.

Body Weight gain: In contrast to rosiglitazone treatment, body weights of compound 3 treated animals are essentially identical that of vehicle treated animals (FIG. 60). Thus, 4 weeks of daily compound 3 treatments reduce plasma glucose, plasma triglyceride, plasma cholesterol and liver triglyceride in db/db mice without excessive body weight gain.

### Example 59

### Synthesis of Diethyl (2-(3,4-dihydroxyphenyl)-5,7-dihydroxy-4-oxo-4H-chromen-3-yloxy)methylphos-phonate (Compound 9) and Ethyl hydrogen (2-(3,4-dihydroxyphenyl)-5,7-dihydroxy-4-oxo-4H-chromen-3-yloxy)-methylphosphonate (Compound 19)

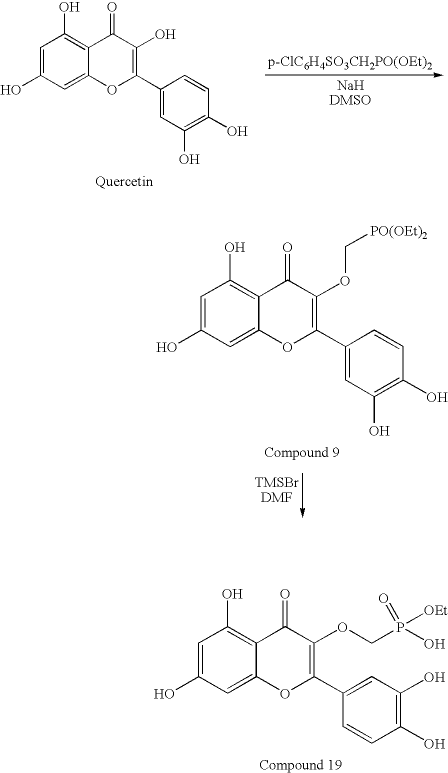

Diethyl (2-(3,4-dihydroxyphenyl)-5,7-dihydroxy-4-oxo-4H-chromen-3-yloxy)methylphos-phonate (Compound 9): A solution of quercetin dihydrate (10.0 g, 29.6 mmol) in DMSO (100 mL) was treated portion-wise with sodium hydride (60% dispersion in mineral oil) (3.6 g, 88.8 mmol). The flask was surrounded by a heat sink of water. The reaction mixture was stirred at room temperature for 5 min, then a solution of (diethoxyphosphoryl)methyl 4-chlorobenzenesulfonate (11.2 g, 32.5 mmol) in DMSO (50 mL) was added drop-wise and the reaction mixture stirred at room temperature for 16 hours. A quenching solution of ice-water (600 mL) and 1N hydrochloric acid (100 mL) was sparged with nitrogen, and the reaction was poured into this mixture slowly while stirring. The mixture was extracted with EtOAc (3×250 mL). The combined organic phases were washed with saturated brine, dried over sodium sulfate and concentrated in vacuo giving a brown oil. The crude product oil was purified twice by Analogix flash chromatography using an SF25-120 g column and gradient of 0-10% methanol in DCM as the eluent. The cleanest fractions from the second column were concentrated to yield a yellow oil that was triturated with DCM to yield 650 mg (5% yield) of diethyl (2-(3,4-dihydroxyphenyl)-5,7-dihydroxy-4-oxo-4H-chromen-3-yloxy)methylphosphonate (Compound 9) as yellow solid: ¹H NMR (300 MHz/DMSO-d₆) δ 12.56 (s, 1H), 10.85 (br, 1H), 9.25 (br, 1H), 7.52 (dd, 1H), 7.46 (d, 1H), 6.88 (d, 1H), 6.41 (d, 1H), 6.21 (d, 1H), 4.45 (d, 2H), 4.03 (m, 4H), 1.20 (t, 6H); MS m/z 453.3 [M+H⁺].

Ethyl hydrogen (2-(3,4-dihydroxyphenyl)-5,7-dihydroxy-4-oxo-4H-chromen-3-yloxy)-methylphosphonate (Compound 19): Diethyl (2-(3,4-dihydroxyphenyl)-5,7-dihydroxy-4-oxo-4H-chromen-3-yloxy)methylphosphonate (Compound 9) (0.20 g, 0.44 mmol, 1 equiv) was stirred in DMF (10 mL) at room temperature, while TMSBr (0.29 mL, 2.21 mmol, 5 equiv) was added. The reaction was stirred at room temperature for 16 hr, quenched with methanol (10 mL), and concentrated under reduced pressure. The mixture was purified by reverse phase chromagraphy and eluting with gradient of 0-8% acetonitrile in water (AnaLogix SF25-100 g, RP C-18), concentrated under reduced pressure and lyophilized for 2 days to give 61 mg (32% yield) of ethyl hydrogen (2-(3,4-dihydroxyphenyl)-5,7-dihydroxy-4-oxo-4H-chromen-3-yloxy)methyl-phosphonate (Compound 19) as yellow solid (>98% purity by LC/MS): ¹H NMR (300 MHz/CD₃OD) δ 7.59 (d, 1H), 7.50 (dd, 1H), 6.81 (d, 1H), 6.30 (d, 1H), 6.10 (d, 1H), 4.26 (d, 2H), 3.99 (m, 2H), 1.18 (t, 3H); MS m/z 423.0 [M−H⁺].

### Example 60

### Synthesis of Diethyl (2-(3,4-dihydroxyphenyl)-7-hydroxy-4-oxo-4H-chromen-3-yloxy)-methylphosphonate (Compound 7)

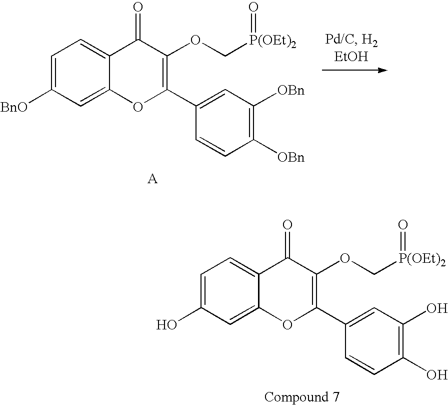

Diethyl (2-(3,4-dihydroxyphenyl)-7-hydroxy-4-oxo-4H-chromen-3-yloxy)methylphosphonate (Compound 7): A mixture of diethyl (7-(benzyloxy)-2-(3,4-bis(benzyloxy)phenyl)-4-oxo-4H-chromen-3-yloxy)methylphosphonate (A) (0.20 g, 0.28 mmol, 1 equiv), 10% palladium-on-carbon (50% wt) (0.05 g) and ethanol (10 mL) was hydrogenated for 6 hr @ 30 psi at room temperature. The mixture was filtered through Celite, concentrated under reduced pressure, purified by reverse phase chromagraphy and eluting with gradient of 0-50% methanol in water (AnaLogix SF15-30 g, RP C-18) and dried in the vacuum oven to yield 120 mg (99%) of diethyl (2-(3,4-dihydroxy-phenyl)-7-hydroxy-4-oxo-4H-chromen-3-yloxy)methylphosphonate (Compound 7) as yellow solid (>99% purity by LC/MS): ¹H NMR (300 MHz/DMSO-d₆) δ 7.91 (d, 1H), 7.50 (dd, 1H), 7.46 (d, 1H), 6.86-6.94 (m, 3H), 4.47 (d, 2H), 4.05 (m, 4H), 1.20 (t, 6H); MS m/z 435.3 [M−H⁺].

### Example 61

### Synthesis of (2-Hydroxy-5-(3-hydroxy-4-oxo-4H-chromen-2-yl)phenoxy)methylphosphonic acid (Compound 18)

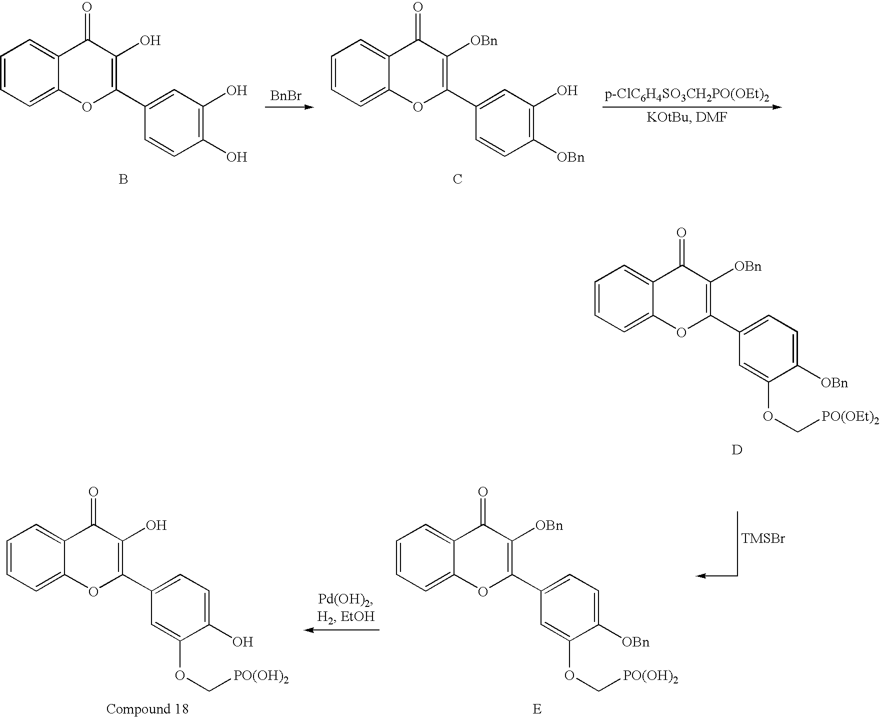

3-(Benzyloxy)-2-(4-(benzyloxy)-3-hydroxyphenyl)-4H-chromen-4-one (C): Suspension of 2-(3,4-dihydroxyphenyl)-3-hydroxy-4H-chromen-4-one (B) (10.0 g, 37.0 mmol), potassium carbonate (10.2 g, 74.1 mmol) and DMF (100 mL) was stirred at 0° C., while benzyl bromide (8.8 mL, 74.1 mmol) was added dropwise. The reaction was slowly warmed to room temperature and allowed to stir for 20 hr and poured into water (200 mL). Mixture was acidified with 1N HCl until pH 3 and extracted with ethyl acetate (3×300 mL). The organic layer was washed with saturated brine (300 mL), dried over sodium sulfate, filtered and concentrated under reduced pressure. The product was purified by an AnaLogix (SF 40-240 g) column using gradient of 0-30% ethyl aceate in heptane as the eluent. 3-(benzyloxy)-2-(4-(benzyloxy)-3-hydroxyphenyl)-4H-chromen-4-one (C) was obtained as yellow solid (13.0 g, 78% yield).

Diethyl (2-(benzyloxy)-5-(3-(benzyloxy)-4-oxo-4H-chromen-2-yl)phenoxy)methylphospho-nate (D): A solution of 3-(benzyloxy)-2-(4-(benzyloxy)-3-hydroxyphenyl)-4H-chromen-4-one (C) (13.0 g, 28.9 mmol) and DMF (260 mL) was stirred at room temperature, while potassium tert-butoxide (3.9 g, 34.7 mmol) was added portion-wise. After stirring at room temperature for 15 min, (diethoxyphosphoryl)methyl 4-chlorobenzenesulfonate (11.9 g, 34.7 mmol) was added. After stirring at room temperature for 16 hr, the reaction mixture was diluted with water (500 mL) and extracted with ethyl acetate (3×300 mL). The organic layer was washed with saturated brine (300 mL), dried over sodium sulfate, filtered and concentrated under reduced pressure. The product was purified by an AnaLogix (SF 40-150 g) column using gradient of 0-50% ethyl acetate in heptane solution as the eluent. Diethyl (2-(benzyloxy)-5-(3-(benzyloxy)-4-oxo-4H-chromen-2-yl)phenoxy)methylphosphonate (D) (12.8 g, 74% yield) was obtained as white solid.

(2-(Benzyloxy)-5-(3-(benzyloxy)-4-oxo-4H-chromen-2-yl)phenoxy)methylphosphonic acid (E): Diethyl (2-(benzyloxy)-5-(3-(benzyloxy)-4-oxo-4H-chromen-2-yl)phenoxy)methylphospho-nate (D) (12.8 g, 21.3 mmol) was stirred in chloroform (200 mL) at room temperature, while TMSBr (42.1 mL, 319.5 mmol) was added. The reaction mixture was allowed to stir at room temperature for 20 hr and concentrated under reduced pressure to give orange wax. Methanol (100 mL) was added to the residue, upon which a light yellow precipitate formed. The solid was filtered and dried in the vacuum oven at 30° C. for 16 hr. (2-(Benzyloxy)-5-(3-(benzyloxy)-4-oxo-4H-chromen-2-yl)phenoxy)methylphosphonic acid (E) was obtained as salmon-colored solid (10.3 g, 89% yield).

(2-Hydroxy-5-(3-hydroxy-4-oxo-4H-chromen-2-yl)phenoxy)methylphosphonic acid (Compound 18): A mixture of (2-(benzyloxy)-5-(3-(benzyloxy)-4-oxo-4H-chromen-2-yl)phenoxy)methyl-phosphonic acid (E) (10.0 g, 18.4 mmol), 10% by weight palladium-on-carbon (50% wet) (2.0 g) and ethanol (500 mL) was hydrogenated for 7 hr at 50 psi. The mixture was filtered through Celite and the Celite was washed with methanol. The filtrate was concentrated under reduced pressure, dried in vacuum oven and lyophilized to afford 6.1 g (91% yield) of (2-hydroxy-5-(3-hydroxy-4-oxo-4H-chromen-2-yl)phenoxy)methylphosphonic acid (Compound 18) as an orange solid (>97% purity by LC-MS): ¹H NMR (500 MHz/CD₃OD) δ 8.15 (d, 1H), 8.02 (d, 1H), 7.89 (d, 1H), 7.72 (m, 2H), 7.00 (t, 1H), 6.97 (d, 1H), 4.35 (d, 2H); MS m/z 363.0 [M−H⁺].

### Example 62

### Synthesis of (2-(3,4-Dihydroxyphenyl)-4-oxo-4H-chromen-3-yloxy)methylphosphonic acid (Compound 20), Diethyl (2-(3,4-dihydroxyphenyl)-4-oxo-4H-chromen-3-yloxy)methylphosphonate (Compound 13), and Ethyl hydrogen (2-(3,4-dihydroxyphenyl)-4-oxo-4H-chromen-3-yloxy)methylphosphonate (Compound 21)

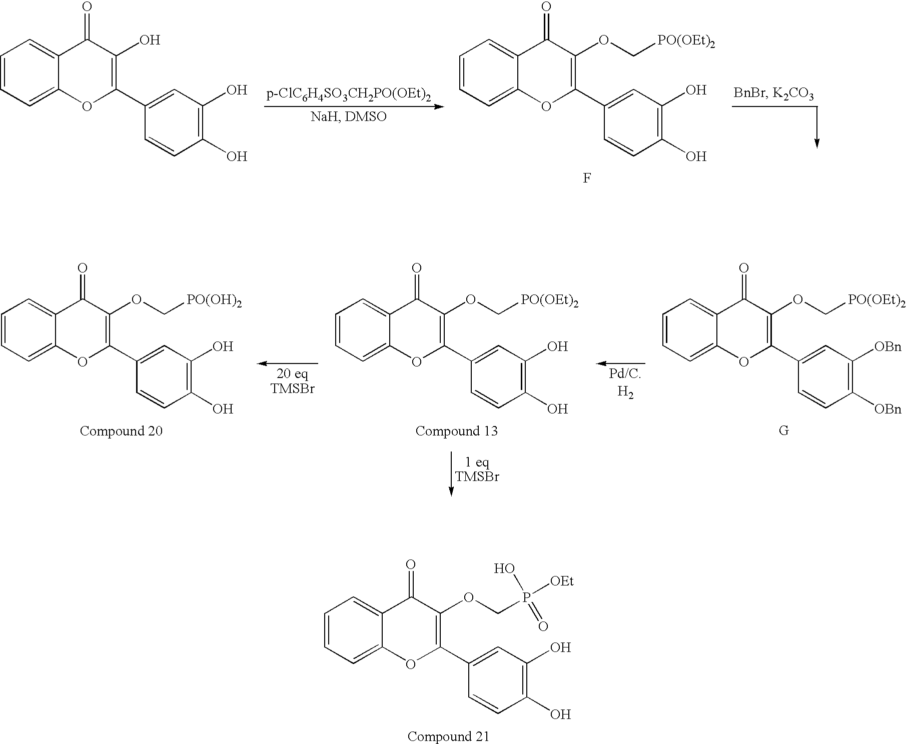

Diethyl (2-(3,4-dihydroxyphenyl)-4-oxo-4H-chromen-3-yloxy)methylphosphonate (F): A solution of 2-(3,4-dihydroxyphenyl)-3-hydroxy-4H-chromen-4-one (22.7 g, 84.1 mmol) and DMSO (200 mL) was stirred at room temperature, while 60% sodium hydride in mineral oil (5.0 g, 126.2 mmol) was added portion-wise. The reaction was stirred at room temperature for 10 min, then (diethoxyphosphoryl)methyl 4-chlorobenzenesulfonate (31.7 g, 92.5 mmol) in DMSO (100 mL) was added. The reaction was further stirred at room temperature for 16 hr, diluted with water (1 L) and acidified with 1N hydrochloric acid until pH 2. The mixture was extracted with ethyl acetate (3×600 mL). The organic layer was washed with saturated brine (500 mL), dried over sodium sulfate, filtered and concentrated under reduced pressure. The product was purified by an AnaLogix (SF 65-100 g) column using gradient of 0-50% methanol in methylene chloride as the eluent. Diethyl (2-(3,4-dihydroxyphenyl)-4-oxo-4H-chromen-3-yloxy)methylphosphonate (F) was obtained as brown oil (27 g, 77% yield) with 50% product by LC/MS.

Diethyl (2-(3,4-bis(benzyloxy)phenyl)-4-oxo-4H-chromen-3-yloxy)methylphosphonate (G): A mixture of diethyl (2-(3,4-dihydroxyphenyl)-4-oxo-4H-chromen-3-yloxy)methylphosphonate (F) (26.0 g, 61.9 mmol), potassium carbonate (25.6 g, 185.7 mmol) and DMF (260 mL) was stirred at 0° C., while benzyl bromide (22.1 mL, 185.7 mmol) was added slowly via an additional funnel. The reaction was allowed to slowly warm to room temperature and stir for 16 hr. The reaction mixture was diluted by water (0.5 L), acidified with 1N hydrochloric acid until pH 2 and extracted with ethyl acetate (3×0.5 L). The organic layer was washed with brine, dried over sodium sulfate, filtered and concentrated. Product was purified by Analogix (SF 65-400 g) using gradient of 0-80% ethyl acetate in heptanes as eluent. Diethyl (2-(3,4-bis(benzyloxy)phenyl)-4-oxo-4H-chromen-3-yloxy)methylphosphonate (G) was obtained as light brown oil (10.0 g, contained DMF by ¹H NMR).

Diethyl (2-(3,4-dihydroxyphenyl)-4-oxo-4H-chromen-3-yloxy)methylphosphonate (Compound 13): A mixture of diethyl (2-(3,4-bis(benzyloxy)phenyl)-4-oxo-4H-chromen-3-yloxy)methyl-phosphonate (G) (10.0 g), 10% palladium-on-carbon (50% wt) (2.0 g) and ethanol (0.5 L) was hydrogenated for 7 hr under 50 PSI of pressure of hydrogen. The mixture was filtered through Celite, concentrated and dried in vacuum oven for 4 hr to give dark green oil (6.7 g, 96% yield). A 700 mg sample of the crude product was purified by Analogix (SF 25-100 g, RP C18) using a gradient of 0-70% methanol (Compound 13) as yellow solid (0.46 g, >99% purity by LC-MS): ¹H NMR (300 MHz/DMSO-d₆) δ 9.81 (br, 1H), 9.22 (br, 1H), 8.08 (dd, 1H), 7.79-7.84 (m, 1H), 7.64-7.73 (m, 2H), 7.56 (d, 1H), 7.45 (t, 1H), 6.89 (d, 1H), 4.38 (d, 2H), 3.98 (q, 4H), 1.19 (t, 6H); MS m/z 419.1 [M−H⁺].

(2-(3,4-Dihydroxyphenyl)-4-oxo-4H-chromen-3-yloxy)methylphosphonic acid (Compound 20): A solution of diethyl (2-(3,4-dihydroxyphenyl)-4-oxo-4H-chromen-3-yloxy)methylphosphonate (Compound 13) (5.0 g, 11.9 mmol), chloroform (60 mL) and TMSBr (31.4 mL, 238.1 mmol) was stirred at room temperature for 16 hr and quenched by water (50 mL). The mixture was concentrated under reduced pressure, triturated with ethyl acetate to afford 3.2 g (74% yield) of (2-(3,4-dihydroxyphenyl)-4-oxo-4H-chromen-3-yloxy)methylphosphonic acid (Compound 20) as brown solid (>97% purity by LC/MS): ¹H NMR (300 MHz/DMSO-d₆) δ 8.18 (dd, 1H), 7.66-7.83 (m, 4H), 7.48 (t, 1H), 6.93 (d, 1H), 4.29 (d, 2H); MS m/z 363.0 [M−H⁺].

Ethyl hydrogen (2-(3,4-dihydroxyphenyl)-4-oxo-4H-chromen-3-yloxy)methylphosphonate (Compound 21): A solution of ethyl (2-(3,4-dihydroxyphenyl)-4-oxo-4H-chromen-3-yloxy)methyl-phosphonate (Compound 13) (1.0 g, 2.38 mmol) and chloroform (12 mL) was stirred at room temperature, while TMSBr (0.31 mL, 2.38 mmol) was added. The reaction mixture was allowed to stir at room temperature for 16 hr and quenched by water (5 mL). Mixture was concentrated under reduced pressure and purified by Analogix (SF 25-100 g, RP C18) using gradient of 0-40% methanol in water as eluent to give 140 mg, (15% yield) of ethyl hydrogen (2-(3,4-dihydroxy-phenyl)-4-oxo-4H-chromen-3-yloxy)methylphosphonate (Compound 21) as orange solid (>99% purity by LC-MS): ¹H NMR (300 MHz/DMSO-d₆) δ 9.81 (br, 1H), 9.25 (br, 1H), 8.10 (dd, 1H), 7.79-7.85 (m, 1H), 7.73 (m, 1H), 7.47-7.70 (m, 3H), 6.90 (d, 1H), 4.51 (d, 2H), 4.06 (q, 2H), 1.20 (t, 3H); MS m/z 391.2 [M−H⁺].

### Example 63

### Synthesis of (3,7-Dihydroxy-4-oxo-2-phenyl-4H-chromen-5-yloxy)methylphosphonic acid (Compound 16) and (3,5-Dihydroxy-4-oxo-2-phenyl-4H-chromen-7-yloxy)methylphosphonic acid (Compound 10)

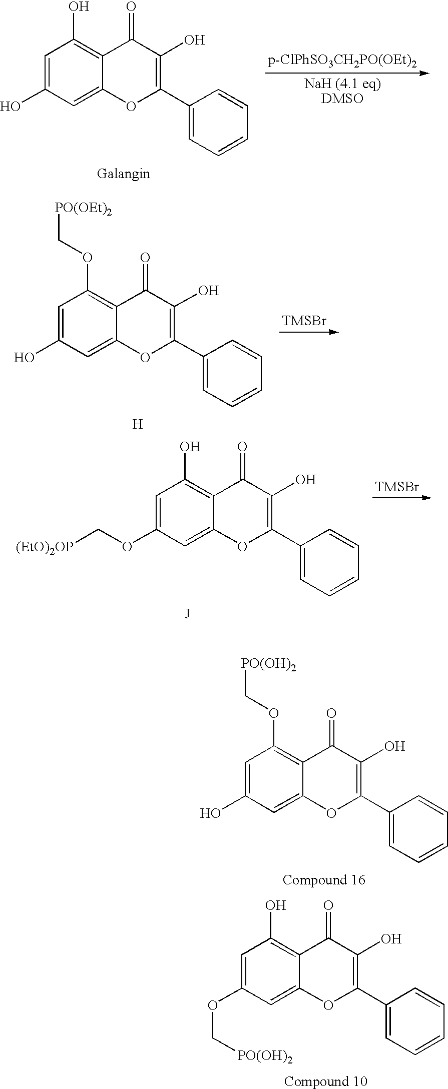

Diethyl (3,7-dihydroxy-4-oxo-2-phenyl-4H-chromen-5-yloxy)methylphosphonate (H) and diethyl (3,5-dihydroxy-4-oxo-2-phenyl-4H-chromen-7-yloxy)methylphosphonate (J): Galangin (0.5 g, 1.97 mmol was dissolved in DMSO (10 mL) and the reaction mixture was cooled to 0° C. A 60% dispersion of sodium hydride in mineral oil (0.24 g, 5.9 mmol) was added. After stirring at 0° C. for 10 mins, (diethoxyphosphoryl)methyl 4-chlorobenzenesulfonate (675 mg, 1.97 mmol) was added. The reaction was warmed to room temperature and stirred overnight. The reaction mixture was poured into water (10 mL) and 1 M hydrochloric acid was used to adjust to pH 4. The mixture was extracted with ethyl acetate (3×10 mL). The combined organic layers were washed with saturated brine, dried over sodium sulfate, and concentrated under reduced pressure to give a yellow solid. The crude was purified by an AnaLogix SF25-40 g column eluting with MeOH/DCM gradient to provide compound H (70 mg, 9%) and compound J (180 mg, 23%).

(3,7-Dihydroxy-4-oxo-2-phenyl-4H-chromen-5-yloxy)methylphosphonic acid (Compound 16): To a solution of diethyl (3,7-dihydroxy-4-oxo-2-phenyl-4H-chromen-5-yloxy)methylphosphonate (H) (70 mg, 0.17 mmol) in methylene chloride (2 mL) was added TMSBr (382.5 mg, 2.5 mmol). The mixture was stirred overnight then basified to pH ˜10 with a 1 M sodium hydroxide solution. The phases were separated and organic phase was extracted with a 1 M sodium hydroxide solution (2×2 mL). The combined aqueous phases were washed with DCM (2×2 mL) and acidified to pH ˜2 with a 3 M hydrochloric acid giving a yellow solid precipitate. The solid was filtered and dried in a vacuum oven to provide (3,7-dihydroxy-4-oxo-2-phenyl-4H-chromen-5-yloxy)methylphosphonic acid (Compound 16) as a yellow solid (50 mg, 60.7% yield): ¹H NMR (300 MHz/CD₃OD) δ 8.2 (d, 2H), 7.46 (m, 5H), 6.6 (s, 1H), 6.56 (s, 1H), 4.38 (d, 2H). MS m/z 365.1 [M+H⁺].

(3,5-Dihydroxy-4-oxo-2-phenyl-4H-chromen-7-yloxy)methylphosphonic acid (Compound 10): To a solution of diethyl (2-(3,4-dihydroxyphenyl)-3,5-dihydroxy-4-oxo-4H-chromen-7-yloxy) methylphosphonate (J) (180 mg, 0.43 mmol) in methylene chloride (5 mL) was added TMSBr (979.5 mg, 6.4 mmol). The mixture was stirred overnight then basified to pH 10 with 1 M sodium hydroxide solution. The phases were separated and the organic phase was extracted with a 1 M sodium hydroxide solution (2×5 mL). The combined aqueous phases were washed with DCM (2×5 mL) and acidified to pH 2 with a 3 M hydrochloric acid giving a yellow precipitate. (3,5-Dihydroxy-4-oxo-2-phenyl-4H-chromen-7-yloxy)methylphosphonic acid (Compound 10) was obtained as a yellow solid (120 mg, 76.9% yield): ¹H NMR (300 MHz/CD₃OD): δ 8.22 (d, 2H), 7.46 (m, 5H), 6.74 (d, 1H), 6.42 (d, 1H), 4.38 (d, 2H); MS m/z 365.1 [M+H⁺].

### Example 64

### Synthesis of (4-(3,5,7-Trihydroxy-4-oxo-4H-chromen-2-yl)phenoxy)methylphosphonic acid (Compound 11)

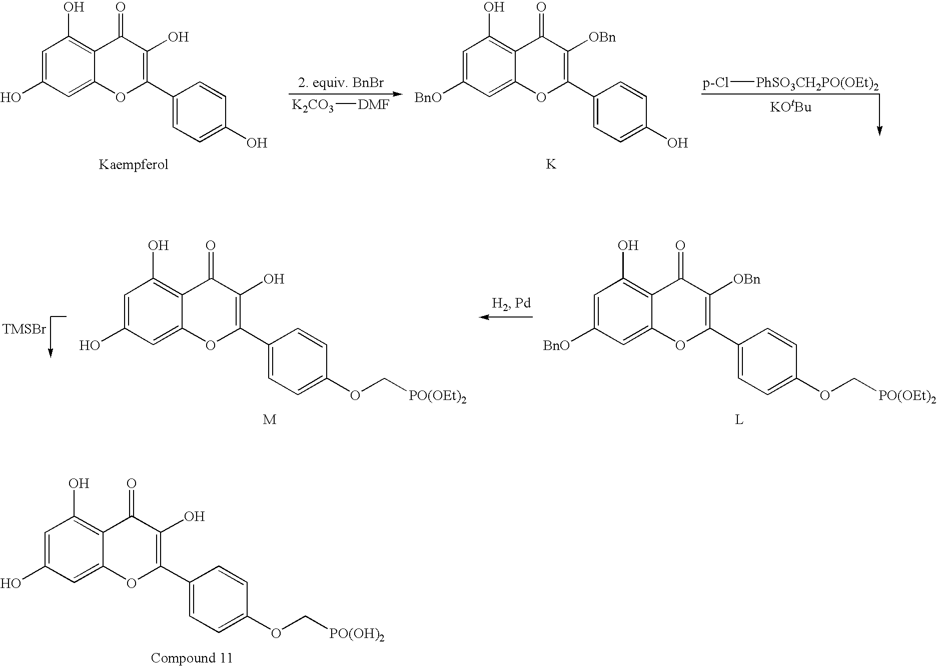

3,7-Bis(benzyloxy)-5-hydroxy-2-(4-hydroxyphenyl)-4H-chromen-4-one (K): A mixture of kaempferol (48.2 g, 168.4 mmol), potassium carbonate (47 g, 341 mmol) and DMF (850 mL) was stirred at 0° C., followed by addition of benzyl bromide (40 mL, 337 mmol) dropwise. The mixture was stirred 16 hr at room temperature. Ethyl acetate (1.2 L) was added and the mixture was washed with water (4×900 mL) and saturated brine (900 mL). The organic phase was dried over sodium sulfate, filtered and concentrated under reduced pressure to give a mixture containing 1 and two side products. The mixture was triturated with DCM (200 mL) to give 7.5 g of 1 as a yellow solid. The mother liquor from the trituration was concentrated under reduced pressure and the residue (˜65 g) was loaded on to a silica gel cartridge (SF 65-600 g) on an AnaLogix system. The cartridge was eluted with a gradient of 10-20% ethyl acetate in heptanes. One side product eluted out while some yellow material solidified within the column. The cartridge was eluted with ethyl acetate. Fractions containing product were concentrated under reduced pressure giving a brown gel-like residue which was triturated with DCM (150 mL) to give 23.5 g (28% yield) of 3,7-bis(benzyloxy)-5-hydroxy-2-(4-hydroxyphenyl)-4H-chromen-4-one (K) (−92% purity) as a yellow solid.

Diethyl (4-(3,7-bis(benzyloxy)-5-hydroxy-4-oxo-4H-chromen-2-yl)phenoxy)methylphospho-nate (L): Potassium tert-butoxide (6.6 g, 58.8 mmol) was added to a solution of 3,7-bis(benzyl-oxy)-5-hydroxy-2-(4-hydroxyphenyl)-4H-chromen-4-one (K) (13.0 g, 27.9 mmol) in dimethyl-acetamide (DMAC, 300 mL). The solution was stirred 30 min at room temperature, then a solution of (diethoxyphosphoryl)methyl 4-chlorobenzenesulfonate (9.56 g, 27.9 mmol) in DMAC (100 mL) was added. The reaction mixture was stirred 2 hr at room temperature and 3 hr at 100° C. DMAC was removed under reduced pressure and the solid residue was triturated with acetone (100 mL). The solid was collected by filtration and suspended in water (150 mL). The resulting mixture was extracted with ethyl acetate (2×200 mL). The combined organic layers were dried over sodium sulfate, filtered and concentrated under reduced pressure to give a yellow oil. The crude product was purified on an AnaLogix system eluting with a gradient of 30-60% ethyl acetate in heptanes to give 3.5 g (20% yield) of diethyl (4-(3,7-bis(benzyloxy)-5-hydroxy-4-oxo-4H-chromen-2-yl)phenoxy)methylphosphonate (L) as a pale yellow solid.

2-(4-((Diethylphosphoryl)methoxy)phenyl)-3,5,7-trihydroxy-4H-chromen-4-one (M): A mixture of diethyl (4-(3,7-bis(benzyloxy)-5-hydroxy-4-oxo-4H-chromen-2-yl)phenoxy)methyl-phosphonate (L) (3.5 g, 5.7 mmol) and palladium hydroxide (20 wt % Pd-on-carbon, 50% wet, 350 mg) in 9:1 tetrahydrofuran/water (100 mL) was hydrogenated at 30 psi for 3 hr. The solution was filtered through a Celite pad and the filtrate was concentrated to give compound 2.5 g (95% yield) of 2-(4-((diethylphosphoryl)methoxy)phenyl)-3,5,7-trihydroxy-4H-chromen-4-one (M) as a yellow solid.

(4-(3,5,7-Trihydroxy-4-oxo-4H-chromen-2-yl)phenoxy)methylphosphonic acid (Compound 11): Compound M (2.5 g, 5.7 mmol) was suspended in 1:2-methylene chloride/acetonitrile (150 mL), and TMSBr (20 mL, 144 mmol) was added and the mixture was stirred at room temperature for 16 hr. The resultant yellow solution was quenched with methanol (12 mL) and water (12 mL). After 30 min, the solvents were removed under reduced pressure and the residue was triturated with methanol (40 mL) to give 1.66 g (77% yield) of (4-(3,5,7-trihydroxy-4-oxo-4H-chromen-2-yl)phenoxy)methylphosphonic acid (Compound 11) as a yellow solid: ¹H NMR (CDCl₃, 300 MHz) δ 12.43 (s, 1H), 8.13 (d, J=9 Hz, 2H), 7.18 (d, J=9 Hz, 2H), 6.46 (s, 1H), 6.20 (s, 1H), 4.18 (d, J_{P}=10 Hz, 2H); MS m/z 381.0 [M+H⁺].

### Example 65

### Synthesis of Ethyl hydrogen (5-(3,7-dihydroxy-4-oxo-4H-chromen-2-yl)-2-hydroxyphenoxy) methylphosphonate (Compound 12)

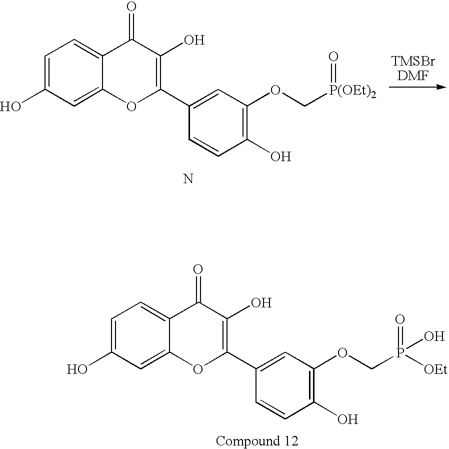

Ethyl hydrogen (5-(3,7-dihydroxy-4-oxo-4H-chromen-2-yl)-2-hydroxyphenoxy)methylphosphonate (Compound 12): A solution of ethyl (5-(3,7-dihydroxy-4-oxo-4H-chromen-2-yl)-2-hydroxyphenoxy) methylphosphonate (N) (100 mg, 0.23 mmol) and DMF (6 mL) was stirred at room temperature, while TMSBr (0.12 mL, 0.9 mmol) was added. After 16 hrs at room temperature, additional TMSBr (0.12 mL, 0.9 mmol), the reaction was quenched with methanol (5 mL) and concentrated under reduced pressure. The mixture was purified by reverse phase chromagraphy with a gradient of 0-10% methanol in water (AnaLogix SF15-30 g, RP C-18), concentrated under reduced pressure and lyophilized for 2 days to give 15 mg (16% yield) of ethyl hydrogen (5-(3,7-dihydroxy-4-oxo-4H-chromen-2-yl)-2-hydroxyphenoxy)methylphospho-nate (Compound 12) as yellow solid (>95% purity by LC/MS): ¹H NMR (300 MHz/CD₃OD) δ 7.98 (d, 1H), 7.96 (d, 1H), 7.83 (dd, 1H), 6.94-6.98 (m, 2H), 6.91 (dd, 1H), 4.37 (d, 2H), 4.18 (m, 2H), 1.33 (t, 3H); MS m/z 407.1 [M−H⁺].

### Example 66

### Synthesis of Diethyl (2-hydroxy-5-(3,5,7-trihydroxy-4-oxo-4H-chromen-2-yl)phenoxy) methylphospho-nate (Compound 17) and Ethyl hydrogen (2-hydroxy-5-(3,5,7-trihydroxy-4-oxo-4H-chromen-2-yl)phenoxy)methyl-phosphonate (Compound 14)

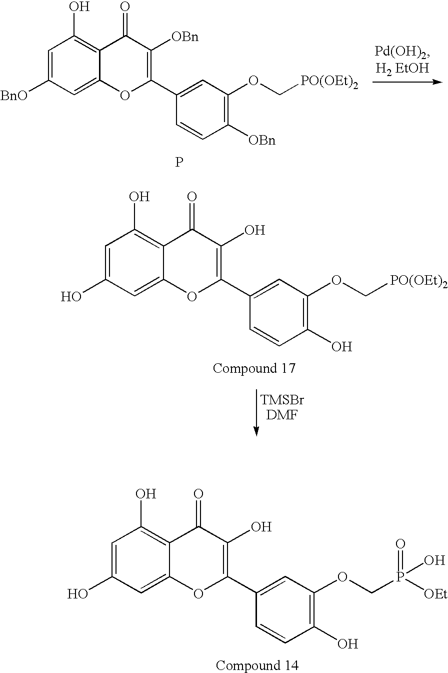

Diethyl (2-hydroxy-5-(3,5,7-trihydroxy-4-oxo-4H-chromen-2-yl)phenoxy)methylphospho-nate (Compound 17): A mixture of diethyl (2-(benzyloxy)-5-(3,7-bis(benzyloxy)-5-hydroxy-4-oxo-4H-chromen-2-yl)phenoxy)methylphosphonate (P, see synthesis of LIM-0724) (1.00 g, 1.38 mmol), palladium hydroxide (20% wt) (0.10 g) and ethanol (100 mL) was hydrogenated for 3 hr @ 20 psi. Methanol (100 mL) was added and then heated to dissolve the precipitate, filtered through Celite and concentrated under reduced pressure. The yellow solid was then stirred in deionized water (50 mL) at 35° C. for 0.5 hr and lyophilized for 2 days. Diethyl (2-hydroxy-5-(3,5,7-trihydroxy-4-oxo-4H-chromen-2-yl)phenoxy)methylphosphonate (Compound 17) was obtained as yellow solid (0.61 g, 97% yield, >92% purity by LC/MS): ¹H NMR (300 MHz/DMSO-d₆) δ 12.45 (s, 1H), 9.45 (br, 1H), 7.84 (d, 1H), 7.78 (dd, 1H), 7.00 (d, 1H), 6.48 (d, 1H), 6.19 (d, 1H), 4.47 (d, 2H), 4.14 (m, 4H), 1.27 (t, 6H); MS m/z 450.9 [M−H⁺].

Ethyl hydrogen (2-hydroxy-5-(3,5,7-trihydroxy-4-oxo-4H-chromen-2-yl)phenoxy)methyl-phosphonate (Compound 14): A solution of diethyl (2-hydroxy-5-(3,5,7-trihydroxy-4-oxo-4H-chromen-2-yl)phenoxy)methylphosphonate (Compound 14) (0.10 g, 0.22 mmol), DMF (6 mL), and TMSBr (0.15 mL, 1.11 mmol) was stirred at room temperature for 16 hr. Additional TMSBr (0.12 mL, 0.89 mmol) was added and the reaction was allowed to stir at room temperature for 16 hr. The reaction was quenched with methanol (5 mL), concentrated under reduced pressure. The mixture was purified by reverse phase chromagraphy and eluting with gradient of 0-10% methanol in water (AnaLogix SF15-30 g, RP C-18), concentrated under reduced pressure and lyophilized for 2 days to give 16 mg (17% yield) of ethyl hydrogen (2-hydroxy-5-(3,5,7-trihydroxy-4-oxo-4H-chromen-2-yl)phenoxy)methylphosphonate (Compound 14) as yellow solid (>97% purity by LC/MS): ¹H NMR (300 MHz/CD₃OD) δ 7.92 (d, 1H), 7.79 (dd, 1H), 6.95 (d, 1H), 6.42 (d, 1H), 6.18 (d, 1H), 4.35 (d, 2H), 4.17 (m, 2H), 1.33 (t, 3H); MS m/z 423.0 [M−H⁺].

### Example 67

### Synthesis of Diethyl (5-(3,7-dihydroxy-4-oxo-4H-chromen-2-yl)-2-hydroxyphenoxy) methylphosphonate (Compound 8)

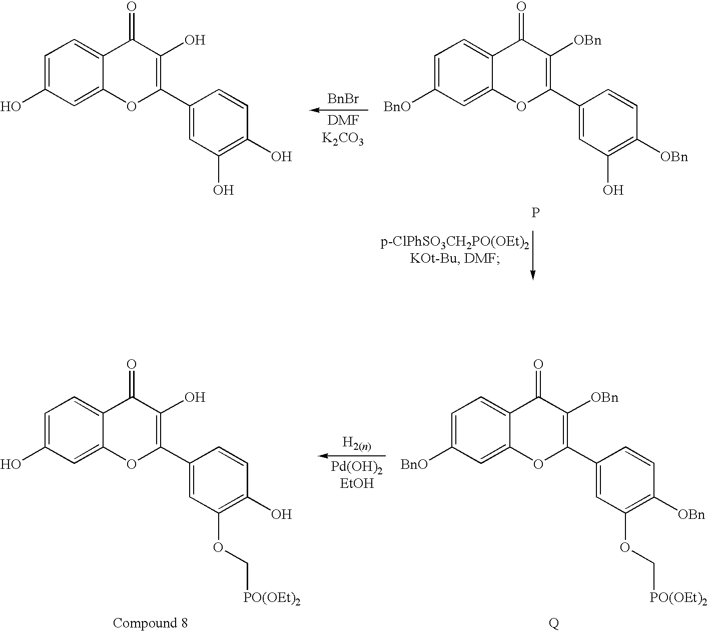

3,7-Bis(benzyloxy)-2-(4-(benzyloxy)-3-hydroxyphenyl)-4H-chromen-4-one (P) A mixture of fisetin (180.0 g, 0.63 mol), potassium carbonate (260.3 g, 1.89 mol), anhydrous DMF (4 L) was stirred under nitrogen at 0° C., and benzyl bromide (224 mL, 322.6 g, 1.89 mol) was added dropwise over 1 hour at 0° C. The mixture was allowed to warm to room temperature and stirred for 63 hours. The mixture was poured into ethyl acetate (20 L) and washed with water (3×12 L). The organic layer was dried over sodium sulfate and concentrated under vacuum to obtain a yellow solid. The resultant yellow solid was triturated with DCM (700 mL) at 38° C. for 30 minutes to break up the solid. The mixture was then stirred at 0° C. for 30 minutes, filtered, and rinsed to provide 71 g (21%) of 3,7-bis(benzyloxy)-2-(4-(benzyloxy)-3-hydroxyphenyl)-4H-chromen-4-one (P) (>95% purity by LC/MS) as a white solid. The filtrate contained ˜1:1 mixture of (a) and the tetrabenzylated product.

Diethyl (2-(benzyloxy)-5-(3,7-bis(benzyloxy)-4-oxo-4H-chromen-2-yl)phenoxy)methylphos-phonate (Q): To a cold solution of 3,7-bis(benzyloxy)-2-(4-(benzyloxy)-3-hydroxyphenyl)-4H-chromen-4-one (P) (50.0 g, 89.93 mmol) in anhydrous DMF (400 mL) was added potassium tert-butoxide (12.1 g, 107.80 mmol) in two equal portions. (Note: The solution went from yellow to dark red.) The mixture was stirred at room temperature for 30 minutes. (Diethoxyphosphoryl)-methyl 4-chlorobenzenesulfonate (40.0 g, 116.78 mmol) in anhydrous DMF (150 mL) was added dropwise over 40 minutes with cooling to maintain temperature below 25° C. during addition. After stirring at room temperature for 18 hours, the mixture was diluted with water (400 mL) and acidified with 1N hydrochloric acid to pH 3. The mixture was extracted with ethyl acetate (600 mL). After sitting over the weekend, a white, crystalline solid formed which was filtered and washed with ethyl acetate. The solid was dried in a vacuum oven at 50° C. for 18 hours to provide diethyl (2-(benzyloxy)-5-(3,7-bis(benzyloxy)-4-oxo-4H-chromen-2-yl)phenoxy)methylphospho-nate (Q) (20.20 g) as a white, crystalline solid. The filtrate was concentrated under vacuum to provide a solid which was triturated with ethanol, filtered, and dried under vacuum at 50° C. to provide a second crop of (c) (10.64 g).

The aforementioned aqueous layer contained a suspended solid which was subsequently filtered and dried under vacuum. This solid was then triturated with ethanol, filtered, and dried under vacuum at 50° C. to yield a third crop of (c) (23.86 g) The combined crops provided 54.7 g of (Q) (86% yield, >95% purity).

Diethyl (5-(3,7-dihydroxy-4-oxo-4H-chromen-2-yl)-2-hydroxyphenoxy)methylphosphonate (Compound 8): A suspension of diethyl (2-(benzyloxy)-5-(3,7-bis(benzyloxy)-4-oxo-4H-chromen-2-yl)phenoxy)methylphosphonate (Q) (49.70 g, 70.32 mmol), palladium hydroxide (7.0 g) and ethanol (1 L) was hydrogenated under a hydrogen atmosphere (50 psi) at room temperature. The mixture was filtered through Celite while rinsing with methanol until the filtrate was colorless. The filtrate was concentrated under vacuum and dried in a vacuum oven for 6 hours at 50° C. to yield 30.2 g (98% yield) of diethyl (5-(3,7-dihydroxy-4-oxo-4H-chromen-2-yl)-2-hydroxy-phenoxy)methylphosphonate (Compound 8) as a yellow solid: ¹H NMR (300 MHz/DMSO-d₆) δ 1.27 (dt, 6H), 4.15 (dq, 4H), 4.48 (d, 2H, J=8.8 Hz), 6.91 (dd, 1H, J=2.2, 8.8 Hz), 6.98 (s, 1H), 6.99 (d, 1H, J=6.1 Hz), 7.77 (dd, 1H, J=2.2, 8.5 Hz), 7.85 (d, 1H, J=2.2 Hz), 7.93 (d, 1H, J=8.8 Hz), 9.14 (s, 1H), 9.96 (s, 1H), 10.73 (s, 1H); MS m/z=435.0 (M−H⁺).

### Example 68

### Synthesis of Ethyl hydrogen (2-(3,4-dihydroxyphenyl)-7-hydroxy-4-oxo-4H-chromen-3-yloxy)methyl-phosphonate (Compound 15)

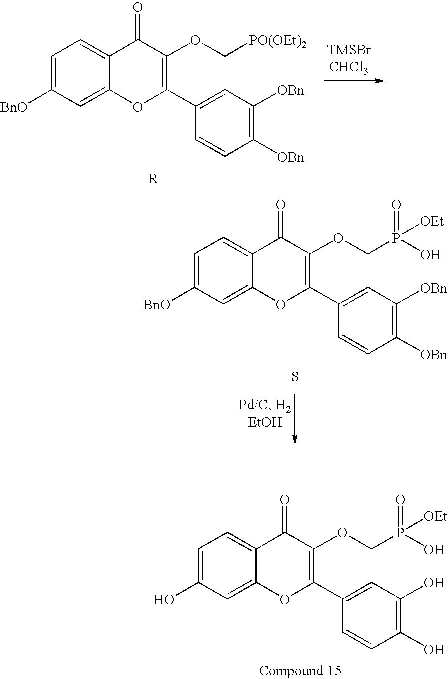

Ethyl hydrogen (7-(benzyloxy)-2-(3,4-bis(benzyloxy)phenyl)-4-oxo-4H-chromen-3-yloxy)methylphosphonate (S): Diethyl (7-(benzyloxy)-2-(3,4-bis(benzyloxy)phenyl)-4-oxo-4H-chromen-3-yloxy)methylphosphonate (R) (0.50 g, 0.71 mmol) was stirred in chloroform (20 mL) at room temperature, while TMSBr (0.19 mL, 1.41 mmol) was added. The reaction was allowed to stir at room temperature for 16 hr, quenched with MeOH (10 mL), concentrated under reduced pressure and used directly for the next step.

Ethyl hydrogen (2-(3,4-dihydroxyphenyl)-7-hydroxy-4-oxo-4H-chromen-3-yloxy)methyl-phosphonate (Compound 15): A mixture of ethyl hydrogen (7-(benzyloxy)-2-(3,4-bis(benzyloxy)-phenyl)-4-oxo-4H-chromen-3-yloxy)methylphosphonate (S) (0.50 g, crude), 10% palladium-on-carbon (50% wt) (0.2 g) and ethanol (50 mL) was hydrogenated for 6 hr @ 30 psi at room temperature. The mixture was filtered through Celite, concentrated under reduced pressure, purified by reverse phase chromagraphy and eluting with gradient of 0-8% acetonitrile in water (AnaLogix SF25-100 g, RP C-18), concentrated under reduced pressure and lyophilized for 2 days to give 26 mg (9% yield) of ethyl hydrogen (2-(3,4-dihydroxyphenyl)-7-hydroxy-4-oxo-4H-chromen-3-yloxy)methylphosphonate (Compound 15) as yellow solid (>96% purity by LC/MS): ¹H NMR (300 MHz/CD₃OD) δ 7.90 (d, 1H), 7.59 (d, 1H), 7.52 (dd, 1H), 6.81-6.86 (m, 3H), 4.25 (d, 2H), 4.00 (m, 2H), 1.18 (t, 3H); MS m/z 407.1 [M−H⁺].

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that the examples provided herein above are to be considered as illustrative and not restrictive, and are not to be limited to the details given herein, and various alternatives to the embodiments of the invention described herein may be employed in practicing the invention. It is intended that the following claims define the scope of the invention and that methods and structures within the scope of these claims and their equivalents be covered thereby.

1. A composition comprising a phosphonated pyrone analog or a pharmaceutically or veterinary acceptable salt, glycoside, ester, or prodrug thereof.

2-11. (canceled)

12. A method for treating an animal comprising administering to an animal in need of treatment, a composition comprising a phosphonated pyrone analog or a pharmaceutically or veterinary acceptable salt, glycoside, ester, or prodrug thereof, and a pharmaceutically acceptable excipient.

13-26. (canceled)

27. A kit comprising: (a) a therapeutic agent or a pharmaceutically or veterinary acceptable salt, glycoside, ester, or prodrug thereof; and (b) a phosphonated pyrone analog or a pharmaceutically or veterinary acceptable salt, glycoside, ester, or prodrug thereof.

28-48. (canceled)

49. A method for treating an animal comprising administering to an animal in need of treatment, (a) a therapeutic agent or a pharmaceutically or veterinary acceptable salt, glycoside, ester, or prodrug thereof; and (b) a phosphonated pyrone analog or a pharmaceutically or veterinary acceptable salt, glycoside, ester, or prodrug thereof.

50-65. (canceled)

66. A method of maintaining cellular physiological conditions for cell survival, comprising administering to a subject an effective amount of a phosphonated pyrone analog that modulates activity of a cellular transporter.

67-85. (canceled)

86. A method of treating a disease, comprising administering to a subject an effective amount of a phosphonated pyrone analog, wherein the phosphonated pyrone analog modulates activity of a cell surface transporter.

87-97. (canceled)

98. A method of modulating transport of a lipophilic molecule, the method comprising administering an effective amount of a phosphonated pyrone analog to a subject, wherein the phosphonated pyrone analog modulates activity of a cellular transporter.

99-128. (canceled)

129. A method of modulating a lipid, cholesterol, triglyceride, insulin or glucose level in a subject, the method comprising administering an effective amount of a phosphonated pyrone analog to the subject, wherein the phosphonated pyrone analog modulates activity of a cellular transporter.

130-144. (canceled)

145. A method of assessing a cellular protective effect in a pancreatic islet cell, comprising:
i) selecting a patient for treatment based on one or more biomolecule levels in a sample compared to a control sample;
ii) administering an effective amount of a phosphonated pyrone analog to the patient; and
iii) monitoring said one or more biomolecule levels in the patient.

146-158. (canceled)

159. A method of treating pancreatic cell stress or injury comprising administering to a subject an effective amount of at least one phosphonated pyrone analog, wherein at least one effect of stress or injury is improved in one or more cell types of the subject.

160-170. (canceled)
